(19) United States
(12) Patent Application Publication
(10) Pub. No.: US 2022/0227738 A1
(43) Pub. Date: Jul. 21, 2022

(54) KRAS G12C INHIBITORS AND USES THEREOF

(71) Applicants: California Institute of Technology, Pasadena, CA (US); 1200 Pharma LLC, Culver City, CA (US); The Regents of the University of California, Oakland, CA (US)

(72) Inventors: Justin A. Hilf, Alhambra, CA (US); Tristin E Rose, Los Angeles, CA (US); Michael D. Bartberger, Sherman Oaks, CA (US); Brendan M. O'Boyle, Monrovia, CA (US); Corey M. Reeves, Los Angeles, CA (US); Oliver C. Loson, Monrovia, CA (US); Brian M. Stoltz, San Marino, CA (US); Martina S. McDermott, Los Angeles, CA (US); Neil A. O'Brien, Los Angeles, CA (US); Dennis Slamon, Los Angeles, CA (US)

(21) Appl. No.: 17/612,972

(22) PCT Filed: May 20, 2020
(86) PCT No.: PCT/US20/33816

§ 371 Date: Nov. 19, 2021

Related U.S. Application Data
(60) Provisional application No. 62/850,289, filed on May 20, 2019.

(51) Int. Cl. C07D 403/14; C07D 471/10; C07D 487/10; C07D 491/10; C07D 495/10; C07D 498/10

(52) U.S. Cl. CPC C07D 403/14; C07D 471/10; C07D 498/10; C07D 491/10; C07D 495/10; C07D 487/10

(57) ABSTRACT

The invention relates to compounds of Formula I, and pharmaceutically acceptable salts thereof, and methods of making and using the same. The compounds of the invention are effective in inhibiting KRAS protein with a G12C mutation and are suitable for use in methods of treating cancers mediated, in whole or in part, by KRAS G12C mutation.

### CROSS-REFERENCE TO RELATED APPLICATIONS

This application claims the benefit of U.S. Provisional Application No. 62/850,289, filed May 20, 2019, which is incorporated by reference herein in its entirety.

### BACKGROUND

Mutations in KRAS are known to be oncogenic and are common in pancreatic, lung, colorectal, gall, thyroid and bile duct cancers. Mutation of Glycine 12 to Cysteine in KRAS is a relatively common genotype in non-small cell lung cancers and colorectal cancers. This mutation offers a selective, covalent inhibition strategy against mutant KRAS and spares wildtype KRAS, thus offering specificity against cancer cells. There is a need to develop new KRAS G12C inhibitors for treating KRAS G12C-mediated cancers (i.e., cancers that are mediated, entirely or partly, by KRAS G12C mutation). The compounds and compositions of the present invention provide means for selectively inhibiting KRAS G12C and for treating cancers, particularly those that are mediated by the KRAS G12C mutation.

### SUMMARY

In certain embodiments, the invention relates to a compound having

(a) the structure of Formula I:

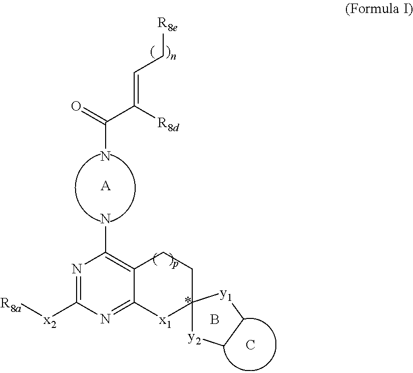

or a pharmaceutically acceptable salt thereof,
wherein:

* is the quaternary carbon atom;

A is a 4-12 membered saturated or partially saturated monocyclic, bridged or spirocyclic ring substituted with one R_{8b} and one R_{8c};

B is a 5-7 membered saturated or partially saturated cycloalkyl or heterocyclyl;

C is an aryl or heteroaryl optionally substituted with one or more R₄;

x₁ is C═O or C(R₁)(R₂);

x₂ is bond, C(R₃)₂, C═O, O, N(R₃), S, S(O), or S(O)₂;

y₁ is y₁ₐ and y₂ is y₂ₐ; or

y₁ is *-y_{1b}-y_{1c} and y₂ is y₂ₐ; or

y₁ is y₁ₐ and y₂ is *y_{2b}-y_{2c}; or

y₁ is *-y_{1d}=y₁ₑ and y₂ is y₂ₐ; or

y₁ is y₁ₐ and y₂ is *-y₂ₐ=y_{2c}; or

y₁ is *y₁ₐ-y_{1b}-y_{1c} and y₂ is bond; or

y₁ is bond and y₂ is *y₂ₐ-y_{2b}-y_{2c};

y₁ₐ and y₂ₐ are each independently bond, (C(R₁₁)₂)ₘ, C═CH₂, C═O, O, N(R₃), S, S(O), or S(O)₂;

y_{1b}, y_{1c}, y_{2b} and y_{2c} are each independently bond, (C(R₁₁)₂)ₘ, C═CH₂, C═O, O, N(R₃), S, S(O), or S(O)₂;

y_{1d}, y₁ₑ, y_{2d} and y_{2c} are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y_{1d} and y₂ₐ cannot be heteroatoms;
with the proviso that both y₁ₐ and y₂ₐ cannot be heteroatoms;
with the proviso that both y₁ₐ and y_{1b} cannot be heteroatoms, the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;

R₁ and R₂ are each independently H or F;

R₃ in each occurrence is independently H or C₁-C₄ alkyl;

R₄ in each instance is independently H, OH, F, Cl, Br, N(R₃)₂, CF₃, CH₃, OCFH₂ or OCH₃;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8b} is H, C₁-C₃ alkyl-CN or C₁-C₃ alkyl-OCH₃;

R_{8c} is H or C₁-C₄ alkyl;

R_{8d} is H, cyano, halogen, C₁-C₃ alkyl, haloalkyl, heteroalkyl, hydroxyalkyl or C(O)N(R₃)₂;

R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃;

m in each occurrence is independently 1, 2 or 3;

n is 0, 1, 2 or 3; and

p is 0 or 1; or

(b) the structure of Formula Ia:

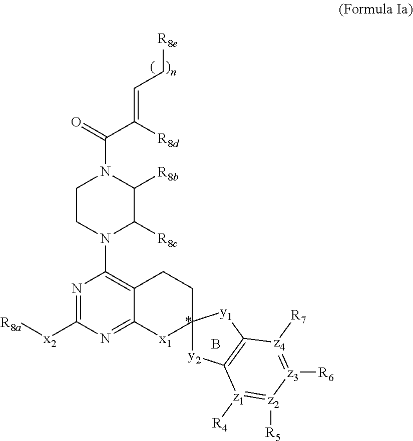

or a pharmaceutically acceptable salt thereof,
wherein:

* is the quaternary carbon atom;

B is a 5-7 membered saturated or partially saturated cycloalkyl or heterocyclyl;

x₁ is C═O or C(R₁)(R₂);

x₂ is bond, C(R₃)₂, C═O, O, N(R₃), S, S(O), or S(O)₂;

y₁ is y₁ₐ and y₂ is y₂ₐ; or

y₁ is *-y_{1b}-y_{1c} and y₂ is y₂ₐ; or

y₁ is y₁ₐ and y₂ is *y_{2b}-y_{2c}; or

y₁ is *-y_{1d}=y₁ₑ and y₂ is y₂ₐ; or

y₁ is y₁ₐ and y₂ is *-y_{2d}=y_{2c}; or

y₁ is *y₁ₐ-y_{1b}-y_{1c} and y₂ is bond; or

y₁ is bond and y₂ is *y₂ₐ-y_{2b}-y_{2c};

y₁ₐ and y₂ₐ are each independently bond, (C(R₁₁)₂)ₘ, C═CH₂, C═O, O, N(R₃), S, S(O), or S(O)₂;

y_{1b}, y_{1c}, y_{2b} and y_{2c} are each independently bond, (C(R₁₁)₂)ₘ, C═CH₂, C═O, O, N(R₃), S, S(O), or S(O)₂;

y_{1d}, y₁ₑ, y_{2d} and y_{2c} are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y₁ₐ and y₂ₐ cannot be heteroatoms;
with the proviso that both y₁ₐ and y_{2d} cannot be heteroatoms;
with the proviso that both y₁ₐ and y_{1b} cannot be heteroatoms, and the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8b} is H, C₁-C₃ alkyl-CN or C₁-C₃ alkyl-OCH₃;

R₈₆ is H or C₁-C₄ alkyl;

R_{8d} is H, cyano, halogen, C₁-C₃ alkyl, haloalkyl, heteroalkyl, hydroxyalkyl or C(O)N(R₃)₂;

R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃;

m in each occurrence is independently 1, 2 or 3; and

n is 0, 1, 2, or 3; or

(c) the structure of Formula II:

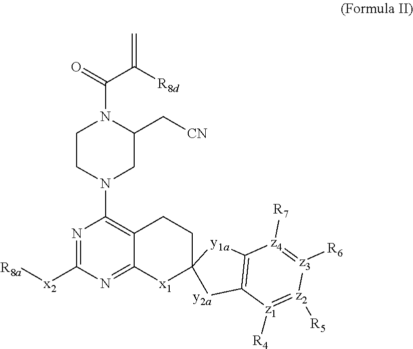

or a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y₁ₐ and y₂ₐ are each independently (C(R₁₁)₂)ₘ, C═CH₂, C═O, O, N(R₃), S, S(O), or S(O)₂, with the proviso that both y₁ₐ and y₂ₐ cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1; or

(d) the structure of Formula III:

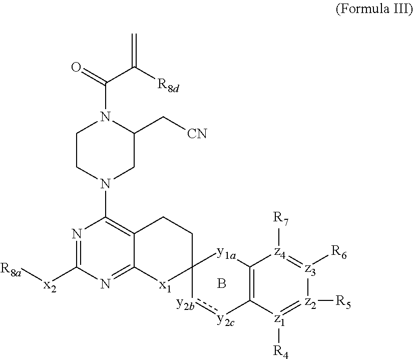

or a pharmaceutically acceptable salt thereof,
wherein:

B is a 5-7 membered saturated or partially saturated cycloalkyl or heterocyclyl;

x₁ is C═O or C(R₁)(R₂);

x₂ is bond, C(R₃)₂, C═O, O, N(R₃), S, S(O), or S(O)₂;

[symbol] is a single or double bond such that all valences are satisfied;

y₁ₐ is bond, (C(R₁₁)₂)ₘ, C═CH₂, C═O, O, N(R₃), S, S(O), or S(O)₂;

when [symbol] is a single bond, y_{2b} and y_{2c} are each independently bond, (C(R₁₁)₂)ₘ, C═CH₂, C═O, O, N(R₃), S, S(O), or S(O)₂, with the proviso that both y₁ₐ and y_{2b} cannot be heteroatoms, and the proviso that both y_{2b} and y_{2c} cannot be heteroatoms; or

when [symbol] is a double bond, y_{2b} and y_{2c} are each independently C(R₃) or N, with the proviso that both y₁ₐ and y_{2b} cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m in each occurrence is independently 1, 2 or 3; or

(e) the structure of Formula IV:

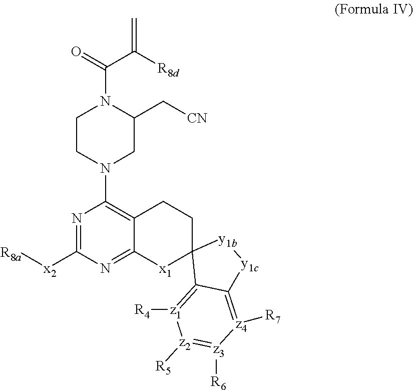

of a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y_{1b} and y_{1c} are each independently (C(R₁₁)₂)ₘ, C═CH₂, C═O, O, N(R₃), S, S(O), or S(O)₂, with the proviso that both y_{1b} and y_{1c} cannot be heteroatoms, the proviso that both y_{1b} and y_{1c} cannot be C═CH₂, and the further proviso that both y_{1b} and y_{1c} cannot be C═O;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1; or

(f) the structure of Formula V:

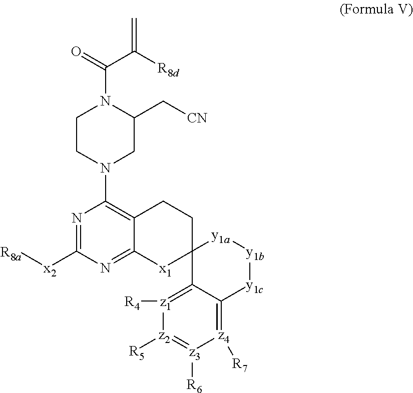

of a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y₁ₐ, y_{1b} and y_{1c} are each independently (C(R₁₁)₂)ₘ, C═CH₂, C═O, O, N(R₃), S, S(O), or S(O)₂, with the proviso that both y₁ₐ and y_{1b} cannot be heteroatoms, the proviso that both y_{1b} and y_{1c} cannot be heteroatoms, the proviso that both y₁ₐ and y_{1b} cannot be C═CH₂, the proviso that both y_{1b} and y_{1c} cannot be C═CH₂, the proviso that both y₁ₐ and y_{1b} cannot be C═O, and the further proviso that both y_{1b} and y_{1c} cannot be C═O;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1.

In other embodiments, the invention relates to a method of treating cancer in a subject in need thereof, comprising administering to the subject an effective amount of a compound disclosed herein.

### DETAILED DESCRIPTION

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkenyl,” as used herein, refers to an aliphatic group containing at least one double bond and is intended to include both “unsubstituted alkenyls” and “substituted alkenyls” the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 6 carbon atoms, preferably from 1 to about 3 unless otherwise defined. Examples of straight chained and branched alkyl groups include, but are not limited to methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, an oxo, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁-C₆ alkyl, C₃-C₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “Cₓ-C_{y},” when used in conjunction with a chemical moiety, such as, alkyl or alkoxy is meant to include groups that contain from x to y carbons in the chain. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal.

The term “alkylamino,” as used herein, refers to an amino group substituted with at least one alkyl group.

The term “alkylthio,” as used herein, refers to a thiol group substituted with an alkyl group and may be represented by the general formula alkylS—.

The term “alkynyl,” as used herein, refers to an aliphatic group containing at least one triple bond and is intended to include both “unsubstituted alkynyls” and “substituted alkynyls,” the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide,” as used herein, refers to a group

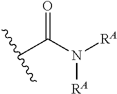

wherein each R^{A} independently represent a hydrogen, hydrocarbyl group, aryl, heteroaryl, acyl, or alkoxy, or two R^{A} are taken together with the N atom to which they are attached complete a heterocycle having from 3 to 8 atoms in the ring structure.

The terms “amine” and “amino” are art-recognized and refer to both unsubstituted and substituted amines and salts thereof, e.g., a moiety that can be represented by

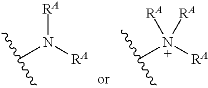

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl,” as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- to 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, aniline, and the like.

The term “carbocycle” refers to a saturated or unsaturated ring in which each atom of the ring is carbon. The term carbocycle includes both aromatic carbocycles and non-aromatic carbocycles. Non-aromatic carbocycles include both cycloalkyl and cycloalkenyl rings. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3- to about 10-carbon atoms, from 3- to 8-carbon atoms, or more typically from 3- to 6-carbon atoms unless otherwise defined. The second ring of a bicyclic cycloalkyl may be selected from saturated, unsaturated and aromatic rings. Cycloalkyl includes bicyclic molecules in which one, two, or three or more atoms are shared between the two rings (e.g., fused bicyclic compounds, bridged bicyclic compounds, and spirocyclic compounds).

A “cycloalkenyl” group is a cyclic hydrocarbon containing one or more double bonds.

The term “bridged bicyclic compound” refers to a bicyclic molecule in which the two rings share three or more atoms, separating the two bridgehead atoms by a bridge containing at least one atom. For example, norbornane, also known as bicyclo[2.2.1]heptane, can be thought of as a pair of cyclopentane rings each sharing three of their five carbon atoms.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “heteroalkyl”, as used herein, refers to a saturated or unsaturated chain of carbon atoms and at least one heteroatom, for example, wherein no two heteroatoms are adjacent.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, and combinations thereof.

The term “fused bicyclic compound” refers to a bicyclic molecule in which two rings share two adjacent atoms. In other words, the rings share one covalent bond, i.e., the so-called bridgehead atoms are directly connected (e.g., α-thujene and decalin). For example, in a fused cycloalkyl each of the rings shares two adjacent atoms with the other ring, and the second ring of a fused bicyclic cycloalkyl may be selected from saturated, unsaturated and aromatic rings.

The term “hydroxyalkyl”, as used herein, refers to an alkyl group substituted with a hydroxy group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, quinoline, quinoxaline, naphthyridine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, preferably 3- to 7-membered rings, more preferably 5- to 6-membered rings, in some instances, most preferably a 5-membered ring, in other instances, most preferably a 6-membered ring, which ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, oxazolines, imidazolines and the like.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “spirocyclic compound” refers to a bicyclic molecule in which the two rings have only one single atom, the spiro atom, in common.

The term “substituted” refers to moieties having substituents replacing a hydrogen on one or more carbons of the backbone, or substituents replacing a hydrogen on one or more nitrogens of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substitutions can be one or more and the same or different for appropriate organic compounds.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt that is suitable for or compatible with the treatment of patients.

The term “pharmaceutically acceptable acid addition salt” as used herein means any non-toxic organic or inorganic salt of any base compounds disclosed herein. Illustrative inorganic acids that form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the invention for laboratory use, or for subsequent conversion to a pharmaceutically acceptable acid addition salt.

The term “pharmaceutically acceptable basic addition salt” as used herein means any non-toxic organic or inorganic base addition salt of any acid compounds of the invention, or any of their intermediates. Illustrative inorganic bases that form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of the invention). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of the invention, or a pharmaceutically acceptable salt thereof. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

### Example Compounds

In certain embodiments, the invention relates to a compound having the structure of Formula I:

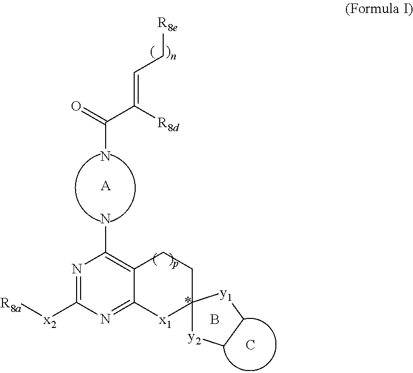

or a pharmaceutically acceptable salt thereof,
wherein:

* is the quaternary carbon atom;

A is a 4-12 membered saturated or partially saturated monocyclic, bridged or spirocyclic ring substituted with one R_{8b} and one R_{8c};

B is a 5-7 membered saturated or partially saturated cycloalkyl or heterocyclyl;

C is an aryl or heteroaryl optionally substituted with one or more R₄;

x₁ is C═O or C(R₁)(R₂);

x₂ is bond, C(R₃)₂, C═O, O, N(R₃), S, S(O), or S(O)₂;

y₁ is y₁ₐ and y₂ is y₂ₐ; or

y₁ is *-y_{1b}-y_{1c} and y₂ is y₂ₐ; or

y₁ is y₁ₐ and y₂ is *-y_{2b}-y_{2c}; or

y₁ is *-y_{1d}=y₁ₑ and y₂ is y₂ₐ; or

y₁ is y₁ₐ and y₂ is *-y_{2d}=y_{2c}; or

y₁ is *y₁ₐ-y_{1b}-y_{1c} and y₂ is bond; or

y₁ is bond and y₂ is *y₂ₐ-y_{2b}-y_{2c};

y₁ₐ and y₂ₐ are each independently bond, (C(R₁₁)₂)ₘ, C═CH₂, C═O, O, N(R₃), S, S(O), or S(O)₂;

y_{1b}, y_{1c}, y_{2b} and y_{2c} are each independently bond, (C(R₁₁)₂)ₘ, C═CH₂, C═O, O, N(R₃), S, S(O), or S(O)₂;

y_{1d}, y₁ₑ, y_{2d} and y_{2c} are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y_{1d} and y₂ₐ cannot be heteroatoms; with the proviso that both y₁ₐ and y_{2d} cannot be heteroatoms;
with the proviso that both y₁ₐ and y_{1b} cannot be heteroatoms, the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;

R₁ and R₂ are each independently H or F;

R₃ in each occurrence is independently H or C₁-C₄ alkyl;

R₄ in each instance is independently H, OH, F, Cl, Br, N(R₃)₂, CF₃, CH₃, OCFH₂ or OCH₃;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8b} is H, C₁-C₃ alkyl-CN or C₁-C₃ alkyl-OCH₃;

R_{8c} is H or C₁-C₄ alkyl;

R_{8d} is H, cyano, halogen, C₁-C₃ alkyl, haloalkyl, heteroalkyl, hydroxyalkyl or C(O)N(R₃)₂;

R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃;

m in each occurrence is independently 1, 2 or 3;

n is 0, 1, 2 or 3; and

p is 0 or 1.

In certain such embodiments, the invention relates to a compound having the structure of Formula I, or a pharmaceutically acceptable salt thereof, wherein:

y₁ is y₁ₐ and y₂ is y₂ₐ, with the proviso that both y₁ₐ and y₂ₐ cannot be heteroatoms, and the further proviso that neither y₁ₐ or y₂ₐ can be a bond when y₁ is y₁ₐ and y₂ is y₂ₐ; or

y₁ is *-y_{1b}-y_{1c} and y₂ is y₂ₐ, with the proviso that both y_{1b} and y₂ₐ cannot be heteroatoms, the proviso that both y_{1b} and y_{1c} cannot be bonds, the proviso that both y_{1b} and y_{1c} cannot be heteroatoms, the proviso that both y_{1b} and y_{1c} cannot be C═O, and the further proviso that both y_{1b} and y_{1c} cannot be C═CH₂; or

y₁ is y₁ₐ and y₂ is *-y_{2b}-y_{2c}, with the proviso that both y₁ₐ and y_{2b} cannot be heteroatoms, the proviso that both y_{2b} and y_{2c} cannot be bonds, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or

y₁ is *-y_{1d}=y₁ₑ and y₂ is y₂ₐ, with the proviso that both y_{1d} and y₂ₐ cannot be heteroatoms; or

y₁ is y₁ₐ and y₂ is *y_{2d}=y_{2c}, with the proviso that both y₁ₐ and y_{2d} cannot be heteroatoms; or

y₁ is *y₁ₐ-y_{1b}-y_{1c} and y₂ is bond, with the proviso that none of y₁ₐ, y_{1b} and y_{1c} can be a bond, the proviso that both y₁ₐ and y_{1b} cannot be heteroatoms, the proviso that both y_{1b} and y_{1c} cannot be heteroatoms, the proviso that both y₁ₐ and y_{1b} cannot be C═O, the proviso that both y_{1b} and y_{1c} cannot be C═O, the proviso that both y₁ₐ and y_{1b} cannot be C═CH₂, and the further proviso that both y_{1b} and y_{1c} cannot be C═CH₂; or

y₁ is bond and y₂ is *y₂ₐ-y_{2b}-y_{2c}, with the proviso that none of y₂ₐ, y_{2b} and y_{2c} can be a bond, the proviso that both y₂ₐ and y_{2b} cannot be heteroatoms, the proviso that both y_{2b} and y_{2c} cannot be heteroatoms, the proviso that both y₂ₐ and y_{2b} cannot be C═O, the proviso that both y_{2b} and y_{2c} cannot be C═O, the proviso that both y₂ₐ and y_{2b} cannot be C═CH₂, and the further proviso that both y_{2b} and y_{2c} cannot be C═CH₂.

In certain embodiments, n is 0.

In certain embodiments, p is 1.

In certain embodiments, B is a 5-membered saturated or partially saturated cycloalkyl or heterocyclyl. In other embodiments, B is a 6-membered saturated or partially saturated cycloalkyl or heterocyclyl.

In certain embodiments, n is 0, p is 1, and B is a 5-membered saturated or partially saturated cycloalkyl or heterocyclyl. In certain embodiments, n is 0, p is 1, and B is a 6-membered saturated or partially saturated cycloalkyl or heterocyclyl.

In preferred embodiments, A is a 6-membered saturated or partially saturated monocyclic, bridged or spirocyclic ring substituted with one R_{8b} and one R_{8c}. In more preferred embodiments, A is a 6-membered heterocyclyl. In even more preferred embodiments, A is piperazinyl.

In certain embodiments, the compounds of Formula I have the structure of Formula Ia:

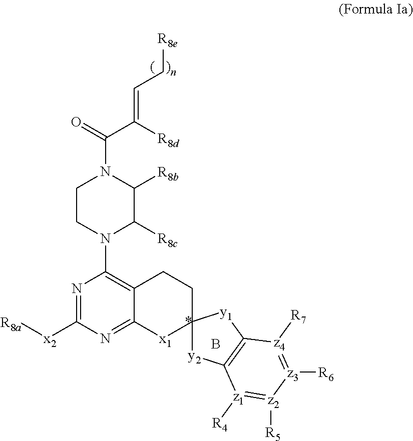

or a pharmaceutically acceptable salt thereof,
wherein:

* is the quaternary carbon atom;

B is a 5-7 membered saturated or partially saturated cycloalkyl or heterocyclyl;

x₁ is C═O or C(R₁)(R₂);

x₂ is bond, C(R₃)₂, C═O, O, N(R₃), S, S(O), or S(O)₂;

y₁ is y₁ₐ and y₂ is y₂ₐ; or

y₁ is *-y_{1b}-y_{1c} and y₂ is y₂ₐ; or

y₁ is y₁ₐ and y₂ is *-y_{2b}-y_{2c}; or

y₁ is *y₁ₐ=y₁ₑ and y₂ is y₂ₐ; or

y₁ is y₁ₐ and y₂ is *y₂ₐ=y_{2c}; or

y₁ is *y₁ₐ-y_{1b}-y_{1c} and y₂ is bond; or

y₁ is bond and y₂ is *y₂ₐ-y_{2b}-y_{2c};

y₁ₐ and y₂ₐ are each independently bond, (C(R₁₁)₂)ₘ, C═CH₂, C═O, O, N(R₃), S, S(O), or S(O)₂;

y_{1b}, y_{1c}, y_{2b} and y_{2c} are each independently bond, (C(R₁₁)₂)ₘ, C═CH₂, C═O, O, N(R₃), S, S(O), or S(O)₂;

y_{1d}, y₁ₑ, y_{2d} and y_{2c} are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y_{1d} and y₂ₐ cannot be heteroatoms;
with the proviso that both y₁ₐ and y_{2d} cannot be heteroatoms;
with the proviso that both y₁ₐ and y_{1b} cannot be heteroatoms, and the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8b} is H, C₁-C₃ alkyl-CN or C₁-C₃ alkyl-OCH₃;

R_{8c} is H or C₁-C₄ alkyl;

R_{8d} is H, cyano, halogen, C₁-C₃ alkyl, haloalkyl, heteroalkyl, hydroxyalkyl or C(O)N(R₃)₂;

R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃;

m in each occurrence is independently 1, 2 or 3; and

n is 0, 1, 2 or 3.

In certain such embodiments, the compounds of Formula I have the structure of Formula Ia, or a pharmaceutically acceptable salt thereof, wherein:

y₁ is y₁ₐ and y₂ is y₂ₐ, with the proviso that both y₁ₐ and y₂ₐ cannot be heteroatoms, and the further proviso that neither y₁ₐ or y₂ₐ can be a bond when y₁ is y₁ₐ and y₂ is y₂ₐ; or

y₁ is *-y_{1b}-y_{1c} and y₂ is y₂ₐ, with the proviso that both y_{1b} and y₂ₐ cannot be heteroatoms, the proviso that both y_{1b} and y_{1c} cannot be bonds, the proviso that both y_{1b} and y_{1c} cannot be heteroatoms, the proviso that both y_{1b} and y_{1c} cannot be C═O, and the further proviso that both y_{1b} and y_{1c} cannot be C═CH₂; or

y₁ is y₁ₐ and y₂ is *-y_{2b}-y_{2c}, with the proviso that both y₁ₐ and y_{2b} cannot be heteroatoms, the proviso that both y_{2b} and y_{2c} cannot be bonds, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or

y₁ is *-y_{1d}=y₁ₑ and y₂ is y₂ₐ, with the proviso that both y₁ₐ and y₂ₐ cannot be heteroatoms; or

y₁ is y₁ₐ and y₂ is *-y_{2d}=y₂ₑ, with the proviso that both y₁ₐ and y₂ₐ cannot be heteroatoms; or

y₁ is *y₁ₐ-y_{1b}-y_{1c} and y₂ is bond, with the proviso that none of y₁ₐ, y_{1b} and y_{1c} can be a bond, the proviso that both y₁ₐ and y_{1b} cannot be heteroatoms, the proviso that both y_{1b} and y_{1c} cannot be heteroatoms, the proviso that both y₁ₐ and y_{1b} cannot be C═O, the proviso that both y_{1b} and y_{1c} cannot be C═O, the proviso that both y₁ₐ and y_{1b} cannot be C═CH₂, and the further proviso that both y_{1b} and y_{1c} cannot be C═CH₂; or

y₁ is bond and y₂ is *y₂ₐ-y_{2b}-y_{2c}, with the proviso that none of y₂ₐ, y_{2b} and y_{2c} can be a bond, the proviso that both y₂ₐ and y_{2b} cannot be heteroatoms, the proviso that both y_{2b} and y_{2c} cannot be heteroatoms, the proviso that both y₂ₐ and y_{2b} cannot be C═O, the proviso that both y_{2b} and y_{2c} cannot be C═O, the proviso that both y₂ₐ and y_{2b} cannot be C═CH₂, and the further proviso that both y_{2b} and y_{2c} cannot be C═CH₂.

In certain embodiments, n is 0.

In certain embodiments, B is a 5-membered saturated or partially saturated cycloalkyl or heterocyclyl. In other embodiments, B is a 6-membered saturated or partially saturated cycloalkyl or heterocyclyl.

In certain embodiments, n is 0, and B is a 5-membered saturated or partially saturated cycloalkyl or heterocyclyl. In other embodiments, n is 0, and B is a 6-membered saturated or partially saturated cycloalkyl or heterocyclyl.

In other embodiments, the compounds of Formula Ia have the structure of Formula Ib:

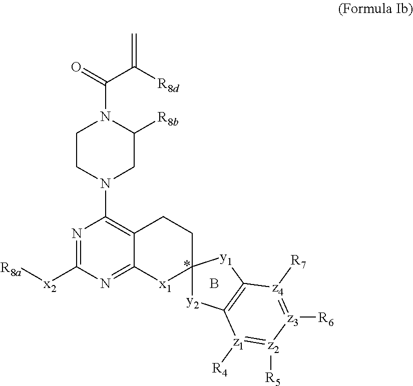

or a pharmaceutically acceptable salt thereof.

In yet other embodiments, the compounds of Formula Ia have the structure of Formula Ic:

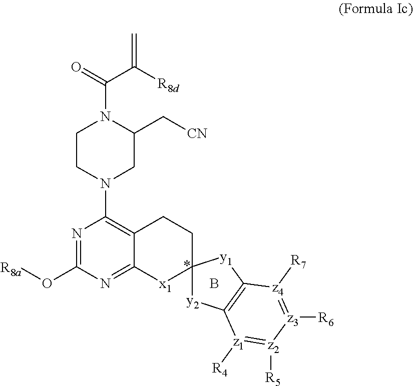

or a pharmaceutically acceptable salt thereof.

In particular embodiments, the compounds of Formula Ia have the structure of Formula Id:

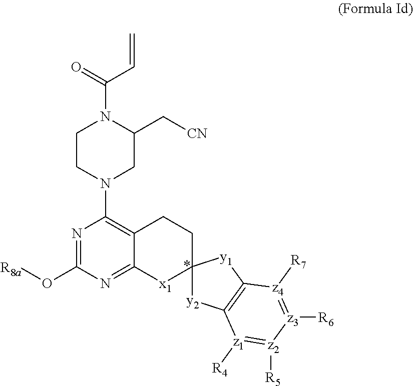

or a pharmaceutically acceptable salt thereof.

In other embodiments, the invention relates to compounds of Formula I, Ia, Ib, Ic or Id, or pharmaceutically acceptable salts thereof, wherein:

* is the quaternary carbon atom;

x₁ is C═O or C(R₁)(R₂);

y₁ is y₁ₐ and y₂ is y₂ₐ; or

y₁ is *-y_{1b}-y_{1c} and y₂ is y₂ₐ; or

y₁ is y₁ₐ and y₂ is *-y_{2b}-y_{2c}; or

y₁ is *y_{1d}=y₁ₑ and y₂ is y₂ₐ; or

y₁ is y₁ₐ and y₂ is *-y_{2d}=y₂ₑ;

y₁ₐ and y₂ₐ are each independently C(R₁₁)₂, O, N(R₃) or S;

y_{1b}, y_{1c}, y_{2b} and y_{2c} are each independently C(R₁₁)₂, O, N(R₃) or S;

y_{1d}, y₁ₑ, y_{2d} and y₂ₑ are each independently C(R₃) or N;
with the proviso that both y₁ₐ and y₂ₐ cannot be heteroatoms;
with the proviso that both y_{1b} and y₂ₐ cannot be heteroatoms, and the further proviso that both y_{1b} and y_{1c} cannot be heteroatoms;
with the proviso that both y₁ₐ and y_{2b} cannot be heteroatoms, and the further proviso that both y_{2b} and y_{2c} cannot be heteroatoms;
with the proviso that both y_{1d} and y₂ₐ cannot be heteroatoms;
with the proviso that both y₁ₐ and y_{2d} cannot be heteroatoms;

z₁, z₂, z₃ and z₄ are each independently C or N;

R₁ and R₂ are each independently H or F;

R₃ in each occurrence is independently H or CH₃;

R₄, R₅, R₆ and R₇ are each independently H, F, Cl, CH₃ or OCH₃, or each of R₄, R₅, R₆ and R₇ is absent when the respective z to which each is attached is N;

R₈ₐ is H, C₁-C₃ alkyl, cycloalkyl, heterocyclyl, aralkyl, aryl or heteroaryl, wherein each of C₁-C₃ alkyl, cycloalkyl, heterocyclyl, aralkyl, aryl and heteroaryl may be optionally substituted with one or more R₉;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl; and

R₁₁ in each occurrence is independently H, F, Cl, CH₃ or OCH₃.

In certain embodiments, the compound of Formula I has the structure of Formula II:

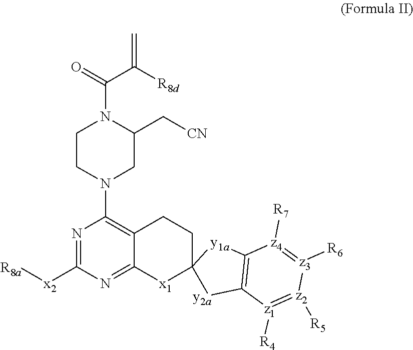

or a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y₁ₐ and y₂ₐ are each independently (C(R₁₁)₂)ₘ, C═CH₂, C═O, O, N(R₃), S, S(O), or S(O)₂, with the proviso that both y₁ₐ and y₂ₐ cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1.

In certain embodiments, R_{8d} is H or halogen (such as F). In other embodiments, R_{8d} is H or F.

In particular embodiments, the compounds of Formula II have the structure of Formula IIa:

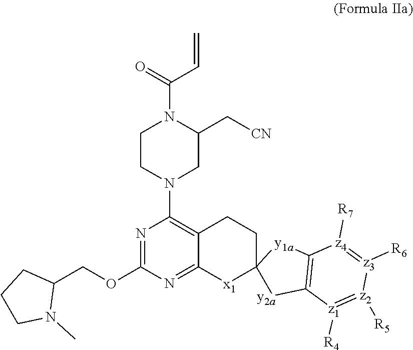

or a pharmaceutically acceptable salt thereof.

In other embodiments, the compounds of Formula II have the structure of Formula IIb:

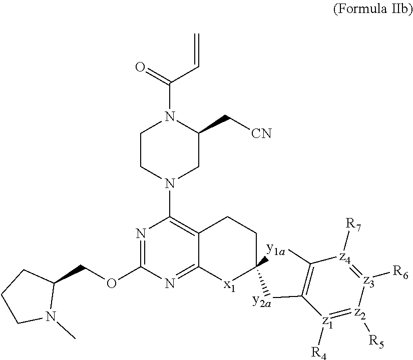

or a pharmaceutically acceptable salt thereof.

In other embodiments, the invention relates to compounds of Formula II, IIa or IIb, or pharmaceutically acceptable salts thereof, wherein:

x₁ is C═O or C(R₁)(R₂);

y₁ₐ and y₂ₐ are each independently C(R₁₁)₂, O, N(R₃) or S, with the proviso that both y₁ₐ and y₂ₐ cannot be heteroatoms;

z₁, z₂, z₃ and z₄ are each independently C or N;

R₁ and R₂ are each independently H or F;

R₃ is H or CH₃;

R₄, R₅, R₆ and R₇ are each independently H, F, Cl, CH₃ or OCH₃, or each of R₄, R₅, R₆ and R₇ is absent when the respective z to which each is attached is N; and

R₁₁ in each occurrence is independently H, F, Cl, CH₃ or OCH₃.

In certain embodiments, the compound of Formula I has the structure of Formula III:

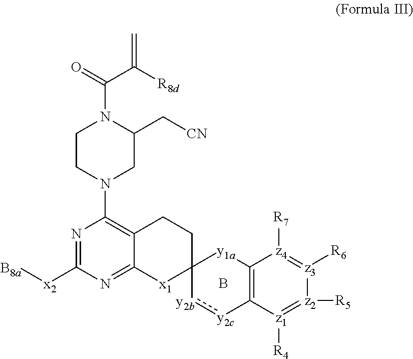

or a pharmaceutically acceptable salt thereof,
wherein:

B is a 5-7 membered saturated or partially saturated cycloalkyl or heterocyclyl;

x₁ is C═O or C(R₁)(R₂);

x₂ is bond, C(R₃)₂, C═O, O, N(R₃), S, S(O), or S(O)₂;

[symbol] is a single or double bond such that all valences are satisfied;

y₁ₐ is bond, (C(R₁₁)₂)ₘ, C═CH₂, C═O, O, N(R₃), S, S(O), or S(O)₂;

when [symbol] is a single bond, y_{2b} and y_{2c} are each independently bond, (C(R₁₁)₂)ₘ, C═CH₂, C═O, O, N(R₃), S, S(O), or S(O)₂, with the proviso that both y₁ₐ and y_{2b} cannot be heteroatoms, and the proviso that both y_{2b} and y_{2c} cannot be heteroatoms; or

when [symbol] is a double bond, y_{2b} and y_{2c} are each independently C(R₃) or N, with the proviso that both y₁ₐ and y_{2b} cannot be heteroatoms;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m in each occurrence is independently 1, 2 or 3.

In certain such embodiments, R_{8d} is H or halogen (such as F).

In other such embodiments, the compound of Formula I has the structure of Formula III, or a pharmaceutically acceptable salt thereof, wherein:

when [symbol] is a single bond, y_{2b} and y_{2c} are each independently bond, (C(R₁₁)₂)ₘ, C═CH₂, C═O, O, N(R₃), S, S(O), or S(O)₂, with the proviso that both y₁ₐ and y_{2b} cannot be bonds, the proviso that both y₁ₐ and y_{2b} cannot be heteroatoms, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or

when [symbol] is a double bond, y_{2b} and y_{2c} are each independently C(R₃) or N, with the proviso that both y₁ₐ and y_{2b} cannot be heteroatoms.

In particular embodiments, the compounds of Formula III have the structure of Formula IIIa:

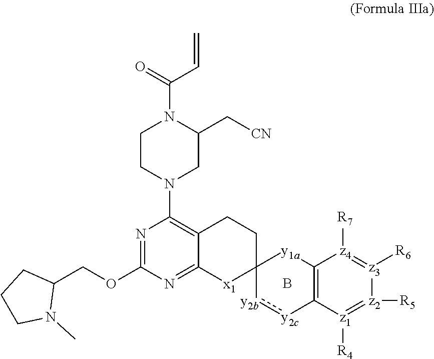

or a pharmaceutically acceptable salt thereof. In some such embodiments, B is a 6-membered saturated or partially saturated cycloalkyl or heterocyclyl.

Alternatively, the compounds of Formula III have the structure of Formula IIIb:

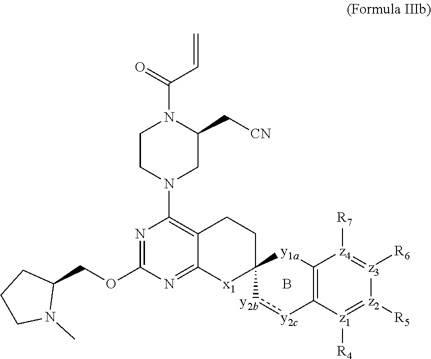

or a pharmaceutically acceptable salt thereof. In some such embodiments, B is a 6-membered saturated or partially saturated cycloalkyl or heterocyclyl.

Alternatively, the compounds of Formula III have the structure of Formula IIIc:

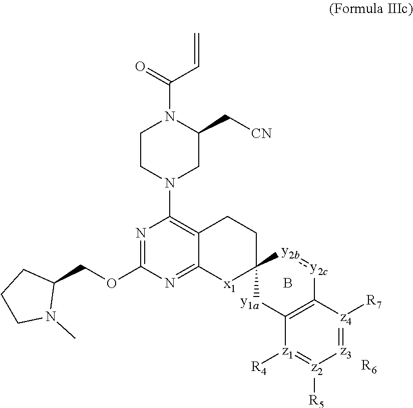

or a pharmaceutically acceptable salt thereof. In some such embodiments, B is a 6-membered saturated or partially saturated cycloalkyl or heterocyclyl.

In other embodiments, the invention relates to compounds of Formula III, IIIa, IIIb or IIIc, or pharmaceutically acceptable salts thereof, wherein:

x₁ is C═O or C(R₁)(R₂);

y₁ₐ is C(R₁₁)₂, O, N(R₃) or S;

[symbol] is a single or double bond such that all valences are satisfied;

when [symbol] is a single bond, y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms; or

when [symbol] is a double bond, y_{2b} and y_{2c} are each independently C(R₃) or N, with the proviso that both y₁ₐ and y_{2b} cannot be heteroatoms;

z₁, z₂, z₃ and z₄ are each independently C or N;

R₁ and R₂ are each independently H or F;

R₃ in each occurrence is independently H or CH₃;

R₄, R₅, R₆ and R₇ are each independently H, F, Cl, CH₃ or OCH₃, or each of R₄, R₅, R₆ and R₇ is absent when the respective z to which each is attached is N; and

R₁₁ in each occurrence is independently H, F, Cl, CH₃ or OCH₃.

In certain embodiments, the compound of Formula I has the structure of Formula IV:

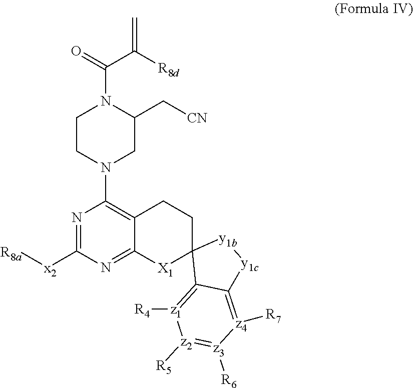

of a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y_{1b} and y_{1c} are each independently (C(R₁₁)₂)ₘ, C═CH₂, C═O, O, N(R₃), S, S(O), or S(O)₂, with the proviso that both y_{1b} and y_{1c} cannot be heteroatoms, the proviso that both y_{1b} and y_{1c} cannot be C═CH₂, and the further proviso that both y_{1b} and y_{1c} cannot be C═O;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R_{8d} is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1.

In certain embodiments, R_{8d} is H or halogen (such as F).

In particular embodiments, the compounds of Formula IV have the structure of Formula IVa:

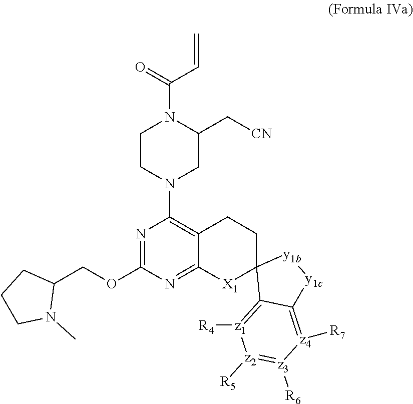

or a pharmaceutically acceptable salt thereof.

Alternatively, the compounds of Formula IV have the structure of Formula IVb:

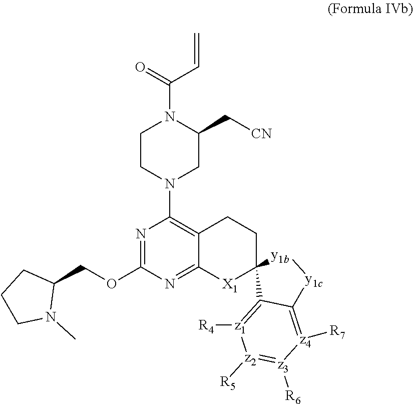

or a pharmaceutically acceptable salt thereof.

Alternatively, the compounds of Formula IV have the structure of Formula IVc:

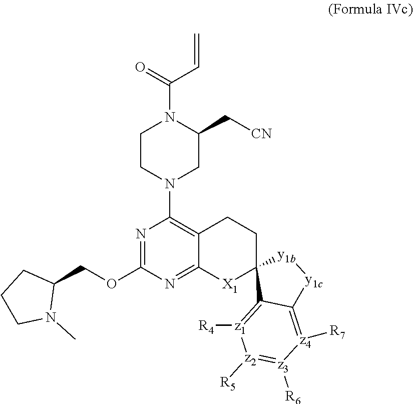

or a pharmaceutically acceptable salt thereof.

In certain embodiments, the compound of formula I has the structure of Formula V:

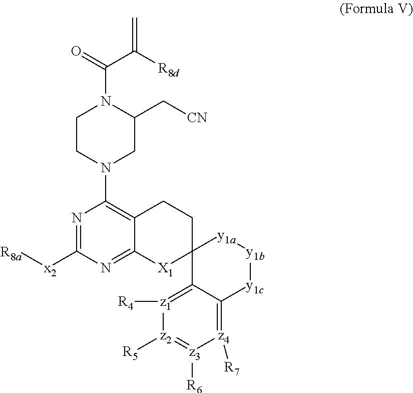

of a pharmaceutically acceptable salt thereof,
wherein:

x₁ is C═O or C(R₁)(R₂);

x₂ is bond, C(R₃)₂, C═O, O, N(R₃), S, S(O), or S(O)₂;

y₁ₐ, y_{1b} and y_{1c} are each independently (C(R₁₁)₂)ₘ, C═CH₂, C═O, O, N(R₃), S, S(O), or S(O)₂, with the proviso that both y₁ₐ and y_{1b} cannot be heteroatoms, the proviso that both y_{1b} and y_{1c} cannot be heteroatoms, the proviso that both y₁ₐ and y_{1b} cannot be C═CH₂, the proviso that both y_{1b} and y_{1c} cannot be C═CH₂, the proviso that both y₁ₐ and y_{1b} cannot be C═O, and the further proviso that both y_{1b} and y_{1c} cannot be C═O;

z₁, z₂, z₃ and z₄ are each independently C or N;

R₁ and R₂ are each independently H or F;

R₃ in each occurrence is independently H or C₁-C₄ alkyl;

R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;

R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;

R₈ₐ is H, cyano, halogen, C₁-C₃ alkyl, haloalkyl, heteroalkyl, hydroxyalkyl or C(O)N(R₃)₂;

R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;

R₁₀ in each occurrence is independently halogen, hydroxyl, C₁-C₃ alkyl, alkoxy, haloalkyl, amino, cyano, heteroalkyl or hydroxyalkyl;

R₁₁ in each occurrence is independently H, F, Cl, C₁-C₃ alkyl or OCH₃; and

m, when present, is 1.

In certain embodiments, R_{8d} is H or halogen (such as F).

In particular embodiments, the compounds of Formula V have the structure of Formula Va:

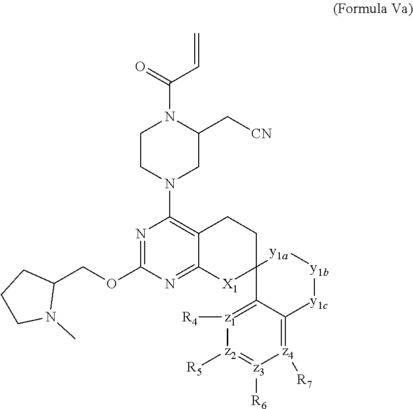

or a pharmaceutically acceptable salt thereof.

Alternatively, the compounds of Formula V have the structure of Formula Vb:

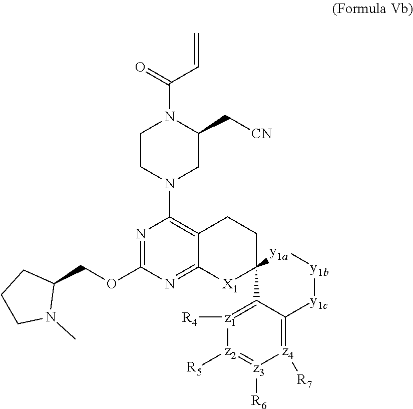

or a pharmaceutically acceptable salt thereof.

Alternatively, the compounds of Formula V have the structure of Formula Vc:

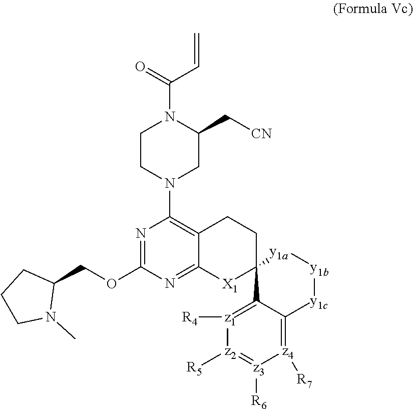

or a pharmaceutically acceptable salt thereof.

In some embodiments, the invention relates to any compound described herein, or a pharmaceutically acceptable salt thereof, wherein:

R₈ₐ is C₁-C₃ alkyl substituted with one R₉;

R₉ is cycloalkyl, heterocyclyl, aryl, or heteroaryl, and cycloalkyl, heterocyclyl, aryl, and heteroaryl are optionally substituted with one or more R₁₀; and

R₁₀ in each occurrence is independently halogen, hydroxyl, C₁-C₃ alkyl, alkoxy, haloalkyl, amino, cyano, heteroalkyl or hydroxyalkyl.

In some embodiments, the invention relates to compounds of Formula Id, IIa, IIIa, IIIb, or IIIc, or pharmaceutically acceptable salts thereof, wherein:

R₈ₐ is C₁-C₃ alkyl substituted with one R₉;

R₉ is cycloalkyl, heterocyclyl, aryl, or heteroaryl, and cycloalkyl, heterocyclyl, aryl, and heteroaryl are optionally substituted with one or more R₁₀; and

R₁₀ in each occurrence is independently halogen, hydroxyl, C₁-C₃ alkyl, alkoxy, haloalkyl, amino, cyano, heteroalkyl or hydroxyalkyl.

In some aspects, C₁-C₃ alkyl is methylene. When R_{8d}, R₈ₑ, R₉ or R₁₁ is C₁-C₃ alkyl, each independently may be methylene.

In some aspects, R₈ is C₁-C₃ alkyl, and C₁-C₃ alkyl is methylene.

In some aspects, R₉ is heterocyclyl substituted with one R₁₀, and R₁₀ is methyl.

In some aspects heterocyclyl is pyrrolidine and the N atom of pyrrolidine is methyl substituted.

In some embodiments, the invention relates to compounds of Formula IIa or IIb, or pharmaceutically acceptable salts thereof, wherein:

x₁ is C═O or C(R₁)(R₂);

y₁ₐ is CH₂;

y₂ₐ is C(R₁₁)₂, O, N(R₃) or S;

z₁, z₂, z₃ and z₄ are each C;

R₁ and R₂ are H;

R₃ is H or CH₃;

R₄, R₅, R₆ and R₇ are each independently H, F, Cl, CH₃ or OCH₃; and

R₁₁ in each occurrence is independently H, CH₃ or OCH₃.

In some aspects, y₂ₐ is C(R₁₁)₂, and R₁₁ is H in one occurrence and is H, CH₃ or OCH₃ in the other.

In other aspects, y₂ₐ is O.

In further aspects, y₂ₐ is N(R₃) and R₃ is H.

In yet further aspects, y₂ₐ is S.

In some embodiments, the invention relates to compounds of Formula IIIa, IIIb, or IIIc, or pharmaceutically acceptable salts thereof, wherein:

[symbol] is a single bond;

x₁ is C═O or C(R₁)(R₂);

y₁ₐ is C(R₁₁)₂, O, N(R₃) or S;

y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms;

z₁, z₂, z₃ and z₄ are each independently C;

R₁ and R₂ are H;

R₃ in each occurrence is independently H or CH₃;

R₄, R₅, R₆ and R₇ are each independently H, F, Cl, CH₃ or OCH₃; and

R₁₁ in each occurrence is independently H, CH₃ or OCH₃.

In some aspects, y₁ₐ is C(R₁₁)₂, and R₁₁ is H in one occurrence and is H, CH₃ or OCH₃ in the other.

In some aspects, y₁ₐ is O.

In some aspects, y₁ₐ is N(R₃).

In some aspects, y₁ₐ is S.

In other aspects, y_{2b} is C(R₁₁)₂, and y_{2c} is O, N(R₃) or S.

In some aspects, y_{2b} is C(R₁₁)₂, and R₁₁ is H in one occurrence and is H, CH₃ or OCH₃ in the other.

In further aspects, y_{2c} is O.

In further aspects, y_{2c} is N(R₃).

In further aspects, y_{2c} is S.

In other aspects, y_{2b} is O, N(R₃) or S, and y_{2c} is C(R₁₁)₂.

In some aspects, y_{2c} is C(R₁₁)₂, and R₁₁ is H in one occurrence and is H, CH₃ or OCH₃ in the other.

In further aspects, y_{2b} is O.

In further aspects, y_{2b} is N(R₃).

In further aspects, y_{2b} is S.

In other embodiments, the invention relates to a compound of Formula IIIa, IIIb or IIIc, such as IIIa, or a pharmaceutically acceptable salt thereof, wherein:

B is a 6-membered saturated cycloalkyl or heterocyclyl;

x₁ is C(R₁)(R₂);

[symbol] is a single bond;

y₁ₐ is (C(R₁₁)₂)ₘ;

y_{2b} is (C(R₁₁)₂)ₘ;

y_{2c} is (C(R₁₁)₂)ₘ or N(R₃);

z₁, z₂, z₃ and z₄ are each C;

R₁ and R₂ are each independently H;

R₃ in each occurrence is independently C₁-C₄ alkyl;

R₄, R₅, R₆ and R₇ are each independently H, F or CH₃;

R₁₁ in each occurrence is independently H; and

m in each occurrence is independently 1.

In further aspects, the compound has a has a KRASG12C k_{obs}/[i] of about 1000 M⁻¹ s⁻¹ or greater.

In yet further aspects, the compound has an average IC₅₀ of greater than 1000 nM for the drug-resistant cell lines of Table 5.

In yet further aspects, the compound has an average IC₅₀ of about 1000 nM or lower for the drug-sensitive cell lines of Table 5.

In some embodiments, the invention relates to compounds of Formula I, II, IIa, III, IIIa or IIIb, or pharmaceutically acceptable salts thereof, wherein x₁ is C═O or C(R₁)(R₂), R₁ and R₂ are H, and z₁, z₂, z₃ and z₄ are each C.

In some embodiments, the invention relates to compounds of Formula Id, IIa, IIb, IIIa, IIIb or IIIc, or pharmaceutically acceptable salts thereof, wherein x₁ is C═O or C(R₁)(R₂), R₁ and R₂ are H, and z₁, z₂, z₃ and z₄ are each C.

In other embodiments, the invention relates to compounds of Formula I, Ia, Ib, Ic, Id, II, IIa, IIb, III, IIIa, IIIb, IIIc, IV, IVa, IVb, IVc, V, Va, Vb or Vc, or pharmaceutically acceptable salts thereof, wherein x₁ is C═O or C(R₁)(R₂), R₁ and R₂ are H, and z₁, z₂, z₃ and z₄ are each C.

In some embodiments, the invention relates to compounds of Formula I, Ia, Ib, Ic, III, IIIa, IIIb or IIIc, or pharmaceutically acceptable salts thereof, wherein B is a 5- or 6-membered cycloalkyl.

In some embodiments, the invention relates to compounds of Formula I, Ia, Ib, Ic, III, IIIa, IIIb or IIIc, or pharmaceutically acceptable salts thereof, wherein B is a 5- or 6-membered heterocyclyl. In some aspects, the 5- or 6-membered heterocyclyl is selected from tetrahydrofuranyl, tetrahydrothiophenyl, sulfolanyl, pyrrolidinyl, tetrahydropyranyl, 1,4-dioxanyl, piperidinyl, piperazinyl, thiomorpholinyl, thiomorpholinyl dioxide, morpholinyl, 1,4-dithianyl, thianyl, lactamyl and lactonyl.

In some embodiments, x₂ is O.

In other embodiments, when R₃ is C₁-C₄ alkyl, C₁-C₄ alkyl is methyl or ethyl.

In some embodiments, the invention relates to a compound of Formula I, Ia, Ib, Ic, II, III, IV or V, or a pharmaceutically acceptable salt thereof, wherein R_{8d} is F. In some aspects, the invention relates to a compound of Formula I, Ia or Ib, or a pharmaceutically acceptable salt thereof, wherein R_{8b} is C₁-C₃ alkyl-CN. In further aspects, the invention relates to a compound of Formula I or Ia, or a pharmaceutically acceptable salt thereof, wherein R_{8c} is H and R₈ₑ is H.

In other embodiments, the invention relates to a compound of Formula I, Ia, Ib, Ic, Id, II, IIa, IIb, III, IIIa, IIIb, IIIc, IV, IVa, IVb, IVc, V, Va, Vb or Vc, or a pharmaceutically acceptable salt thereof, wherein R₁₁ is C₁-C₃ alkyl. In further aspects, C₁-C₃ alkyl is methyl or ethyl.

In some embodiments, the invention relates to a compound of Formula I, Ia, Ib, Ic, Id, III, IIIa, IIIb or IIIc, or a pharmaceutically acceptable salt thereof, wherein m, in each occurrence, is 1.

In some embodiments, the invention relates to a compound of formula I or Ia, or a pharmaceutically acceptable salt thereof, wherein R_{8d} is H, F, methyl, ethyl, OCH₃, CH₂OH or CH₂OCH₃, and R₈ₑ is H, methyl, ethyl, F, CF₃, CF₂H or CH₂F.

In other embodiments, the invention relates to a compound of formula Ib, Ic, II, III, IV or V, or a pharmaceutically acceptable salt thereof, wherein R_{8d} is H, F, methyl, ethyl, OCH₃, CH₂OH or CH₂OCH₃.

In some aspects, the invention relates to a compound of Formula I having a structure selected from Table 1, or a pharmaceutically acceptable salt thereof.

In particular aspects, the compound is selected from Compound 1 through Compound 50, or a pharmaceutically acceptable salt thereof.

In particular aspects, the compound is selected from Compound 1 through Compound 33, or a pharmaceutically acceptable salt thereof.

In other aspects, the compound is selected from Compound 7, 9, 11, 13, 14, 17, 21, 22, 25, 26, 27, 29, 30, 31, 33, 35, 36, 42, 44, 46, 47, 50, 51, 55, 58, 63, 70, 71, 73, 77, 87, 88, 91, 93, 95, 96, 98, 99 and 100, or a pharmaceutically acceptable salt thereof.

In further aspects, the compound is selected from Compound 7, 9, 11, 13, 17, 21, 22, 25, 26, 30, 31, 33, 35, 36, 42, 44, 46, 47, 50, 51, 55, 58, 63, 70, 71, 73, 77, 87, 88, 91, 93, 95, 96, 98, 99 and 100, or a pharmaceutically acceptable salt thereof.

In some aspects, the invention relates to a compound of Formula I having a structure selected from Table 2, or a pharmaceutically salt thereof.

In other aspects, the invention relates to a compound of Formula I having a structure selected from:

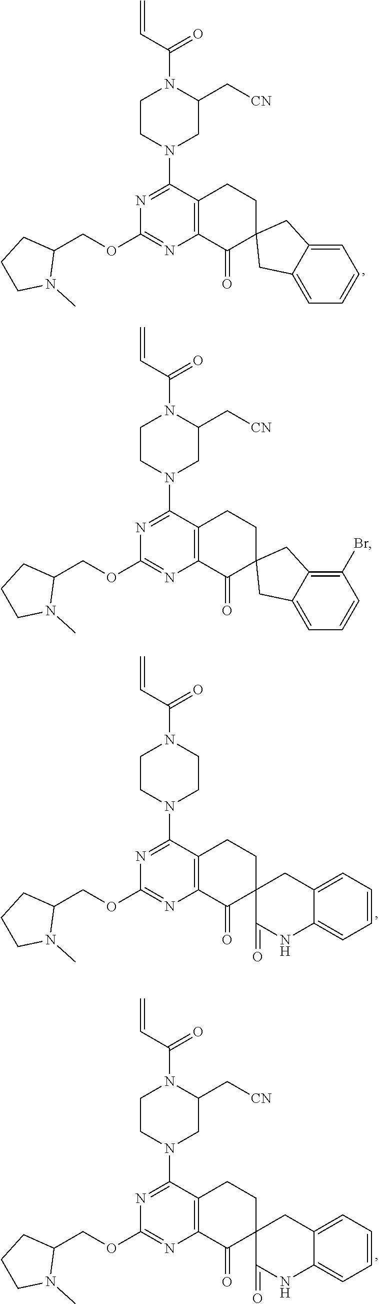 [SMILES: C=CC(=O)N1CCN(c2nc(OCC3CCCN3C)nc3c2CCC2(Cc4ccccc4C2)C3=O)CC1CC#N ; C=CC(=O)N1CCN(c2nc(OCC3CCCN3C)nc3c2CCC2(Cc4cccc(Br)c4C2)C3=O)CC1CC#N ; C=CC(=O)N1CCN(c2nc(OCC3CCCN3C)nc3c2CCC2(Cc4ccccc4NC2=O)C3=O)CC1 ; C=CC(=O)N1CCN(c2nc(OCC3CCCN3C)nc3c2CCC2(Cc4ccccc4NC2=O)C3=O)CC1CC#N]

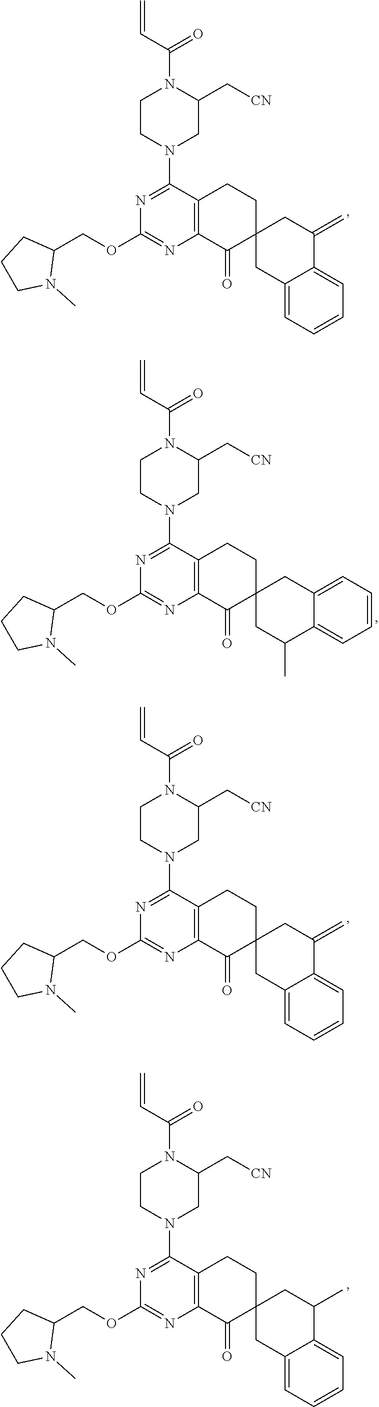 [SMILES: C=CC(=O)N1CCN(c2nc(OCC3CCCN3C)nc3c2CCC2(CC(=C)c4ccccc4C2)C3=O)CC1CC#N ; C=CC(=O)N1CCN(c2nc(OCC3CCCN3C)nc3c2CCC2(Cc4ccccc4C(C)C2)C3=O)CC1CC#N ; C=CC(=O)N1CCN(c2nc(OCC3CCCN3C)nc3c2CCC2(CC(=C)c4ccccc4C2)C3=O)CC1CC#N ; C=CC(=O)N1CCN(c2nc(OCC3CCCN3C)nc3c2CCC2(Cc4ccccc4C(C)C2)C3=O)CC1CC#N]

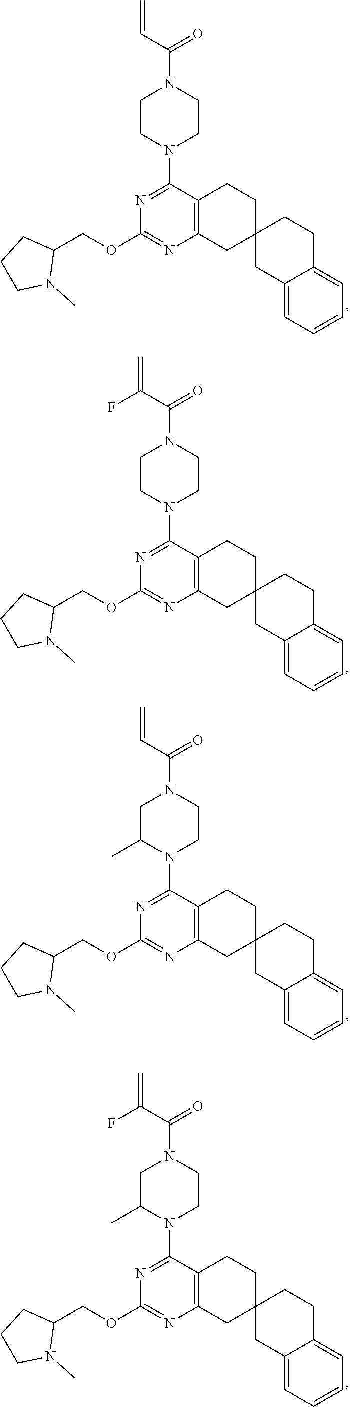 [SMILES: C=CC(=O)N1CCN(c2nc(OCC3CCCN3C)nc3c2CCC2(CCc4ccccc4C2)C3)CC1 ; C=C(F)C(=O)N1CCN(c2nc(OCC3CCCN3C)nc3c2CCC2(CCc4ccccc4C2)C3)CC1 ; C=CC(=O)N1CCN(c2nc(OCC3CCCN3C)nc3c2CCC2(CCc4ccccc4C2)C3)C(C)C1 ; C=C(F)C(=O)N1CCN(c2nc(OCC3CCCN3C)nc3c2CCC2(CCc4ccccc4C2)C3)C(C)C1]

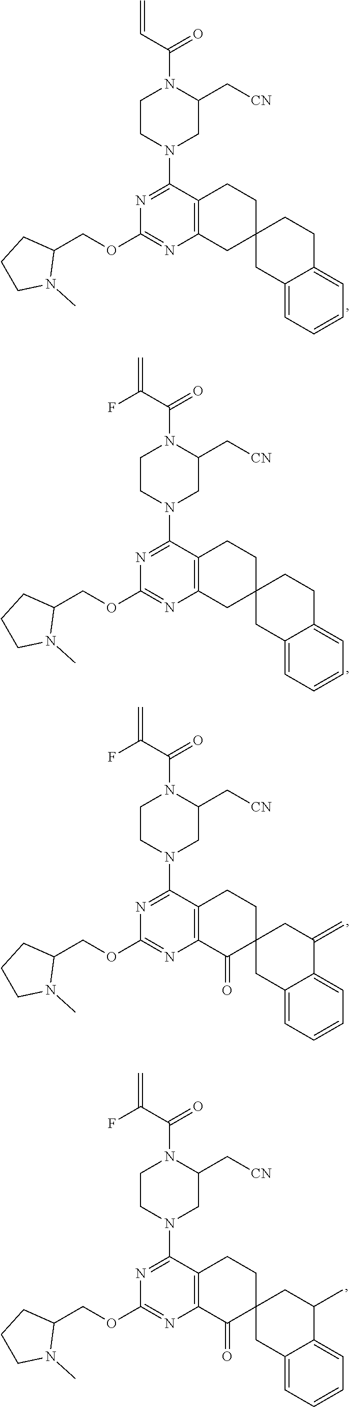 [SMILES: C=CC(=O)N1CCN(c2nc(OCC3CCCN3C)nc3c2CCC2(CCc4ccccc4C2)C3)CC1CC#N ; C=C(F)C(=O)N1CCN(c2nc(OCC3CCCN3C)nc3c2CCC2(CCc4ccccc4C2)C3)CC1CC#N ; C=C(F)C(=O)N1CCN(c2nc(OCC3CCCN3C)nc3c2CCC2(CC(=C)c4ccccc4C2)C3=O)CC1CC#N ; C=C(F)C(=O)N1CCN(c2nc(OCC3CCCN3C)nc3c2CCC2(Cc4ccccc4C(C)C2)C3=O)CC1CC#N]

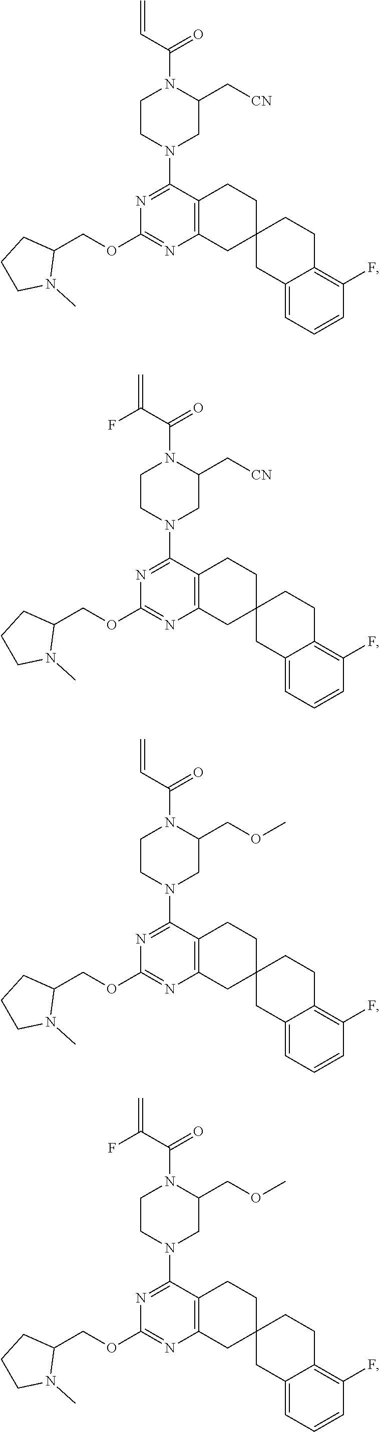 [SMILES: C=CC(=O)N1CCN(c2nc(OCC3CCCN3C)nc3c2CCC2(CCc4c(F)cccc4C2)C3)CC1CC#N ; C=C(F)C(=O)N1CCN(c2nc(OCC3CCCN3C)nc3c2CCC2(CCc4c(F)cccc4C2)C3)CC1CC#N ; C=CC(=O)N1CCN(c2nc(OCC3CCCN3C)nc3c2CCC2(CCc4c(F)cccc4C2)C3)CC1COC ; C=C(F)C(=O)N1CCN(c2nc(OCC3CCCN3C)nc3c2CCC2(CCc4c(F)cccc4C2)C3)CC1COC]

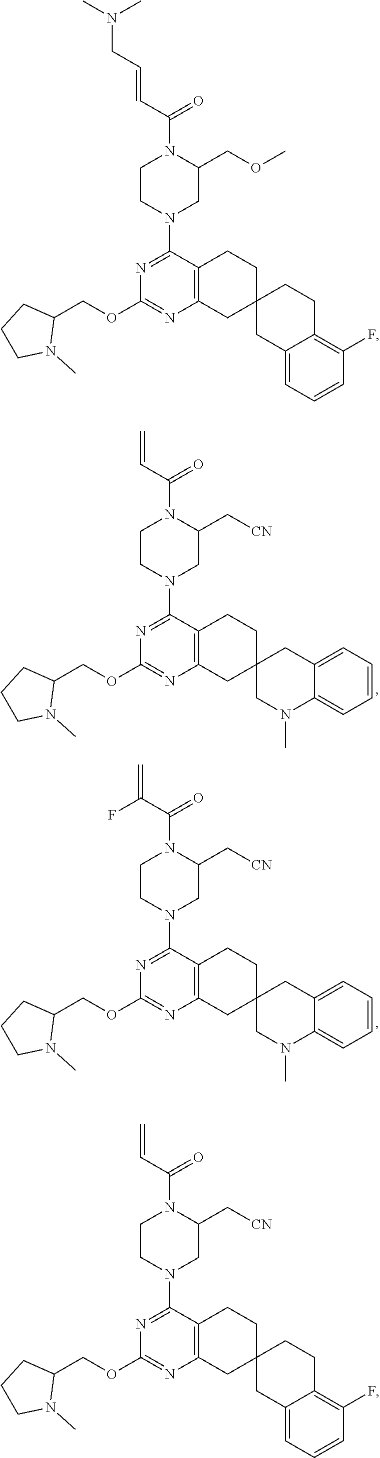 [SMILES: COCC1CN(c2nc(OCC3CCCN3C)nc3c2CCC2(CCc4c(F)cccc4C2)C3)CCN1C(=O)/C=C/CN(C)C ; C=CC(=O)N1CCN(c2nc(OCC3CCCN3C)nc3c2CCC2(Cc4ccccc4N(C)C2)C3)CC1CC#N ; C=C(F)C(=O)N1CCN(c2nc(OCC3CCCN3C)nc3c2CCC2(Cc4ccccc4N(C)C2)C3)CC1CC#N ; C=CC(=O)N1CCN(c2nc(OCC3CCCN3C)nc3c2CCC2(CCc4c(F)cccc4C2)C3)CC1CC#N]

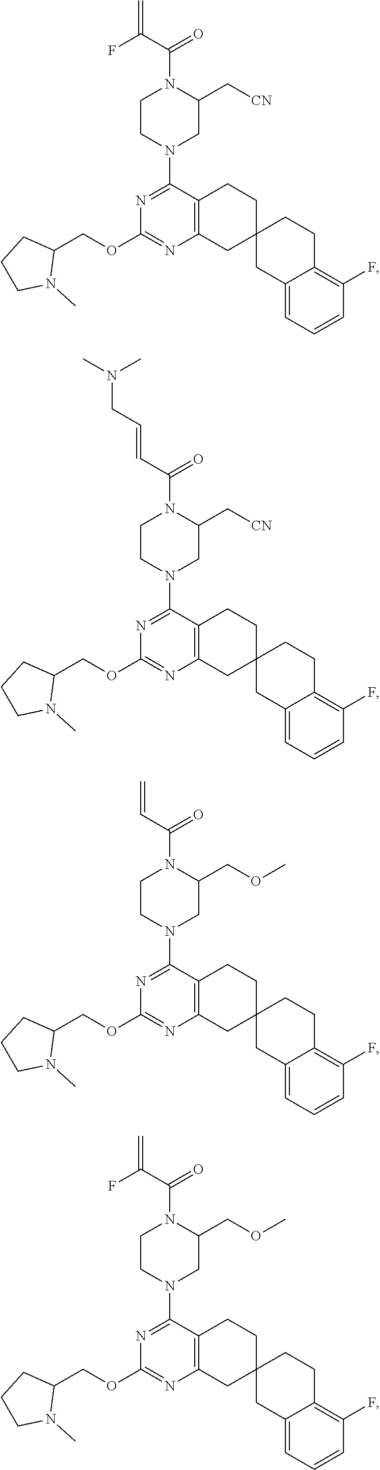 [SMILES: C=C(F)C(=O)N1CCN(c2nc(OCC3CCCN3C)nc3c2CCC2(CCc4c(F)cccc4C2)C3)CC1CC#N ; CN(C)C/C=C/C(=O)N1CCN(c2nc(OCC3CCCN3C)nc3c2CCC2(CCc4c(F)cccc4C2)C3)CC1CC#N ; C=CC(=O)N1CCN(c2nc(OCC3CCCN3C)nc3c2CCC2(CCc4c(F)cccc4C2)C3)CC1COC ; C=C(F)C(=O)N1CCN(c2nc(OCC3CCCN3C)nc3c2CCC2(CCc4c(F)cccc4C2)C3)CC1COC]

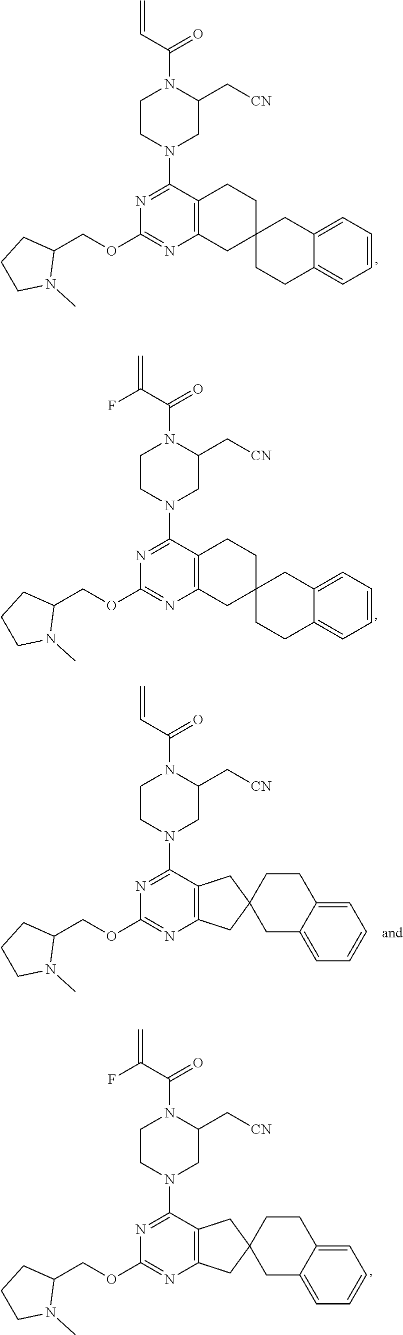 [SMILES: C=CC(=O)N1CCN(c2nc(OCC3CCCN3C)nc3c2CCC2(CCc4ccccc4C2)C3)CC1CC#N ; C=C(F)C(=O)N1CCN(c2nc(OCC3CCCN3C)nc3c2CCC2(CCc4ccccc4C2)C3)CC1CC#N ; C=CC(=O)N1CCN(c2nc(OCC3CCCN3C)nc3c2CC2(CCc4ccccc4C2)C3)CC1CC#N ; C=C(F)C(=O)N1CCN(c2nc(OCC3CCCN3C)nc3c2CC2(CCc4ccccc4C2)C3)CC1CC#N]

or a pharmaceutically salt thereof.

In other aspects, the invention relates to a compound of Formula I having a structure selected from:

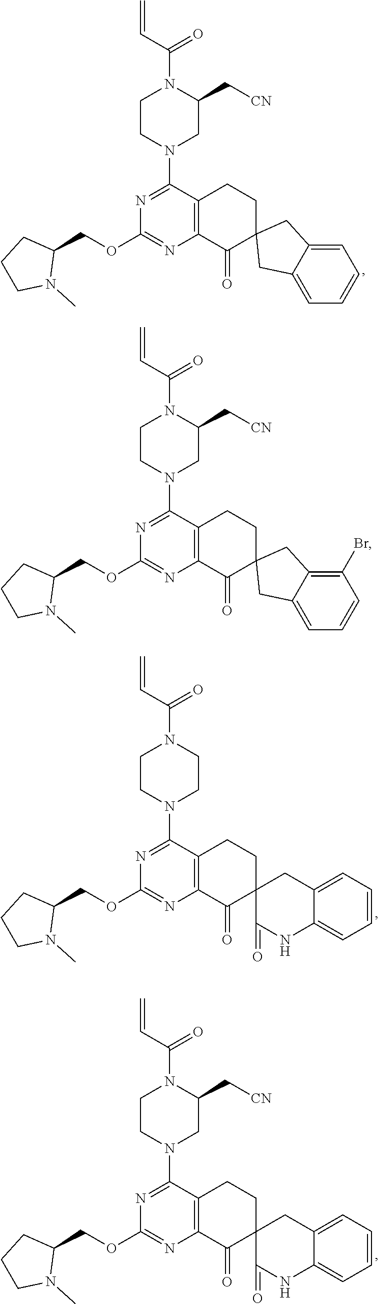 [SMILES: C=CC(=O)N1CCN(c2nc(OC[C@@H]3CCCN3C)nc3c2CCC2(Cc4ccccc4C2)C3=O)C[C@@H]1CC#N ; C=CC(=O)N1CCN(c2nc(OC[C@@H]3CCCN3C)nc3c2CCC2(Cc4cccc(Br)c4C2)C3=O)C[C@@H]1CC#N ; C=CC(=O)N1CCN(c2nc(OC[C@@H]3CCCN3C)nc3c2CCC2(Cc4ccccc4NC2=O)C3=O)CC1 ; C=CC(=O)N1CCN(c2nc(OC[C@@H]3CCCN3C)nc3c2CCC2(Cc4ccccc4NC2=O)C3=O)C[C@@H]1CC#N]

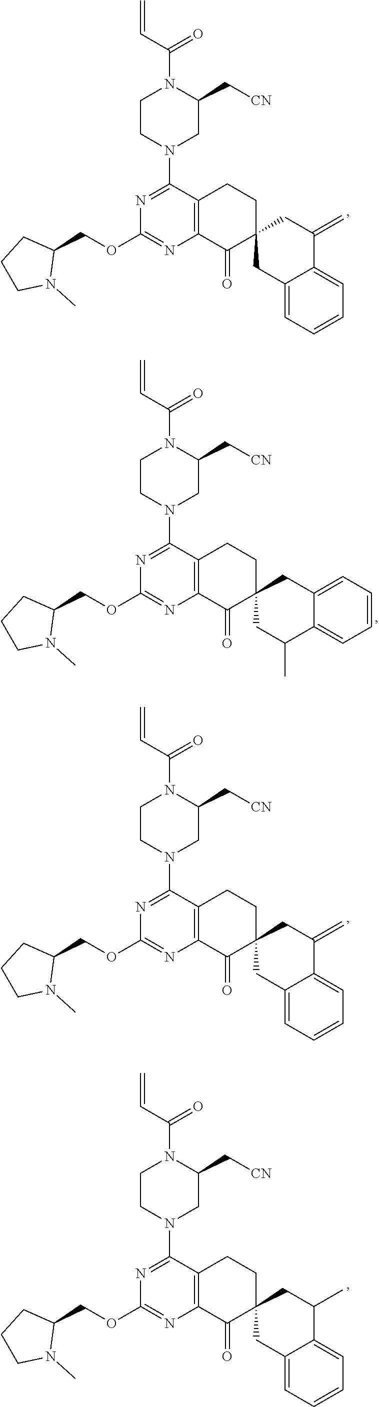

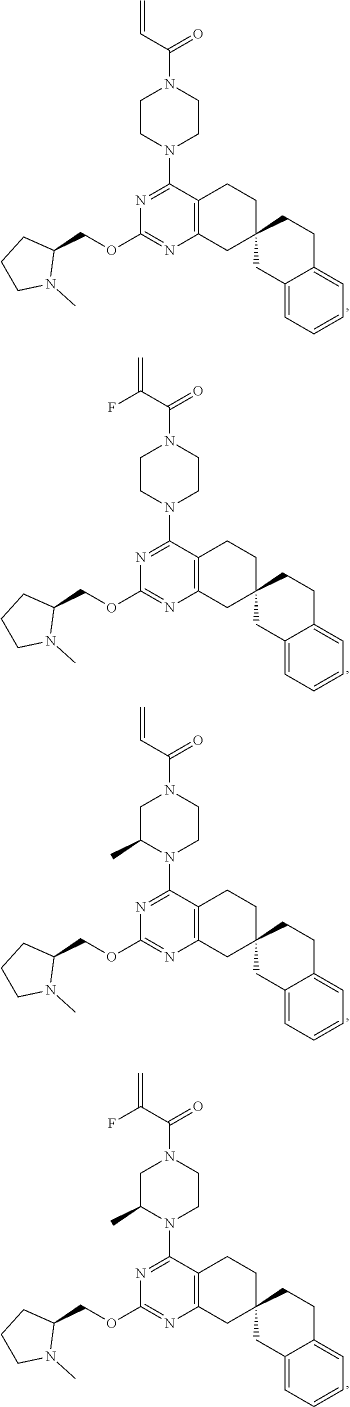 [SMILES: C=CC(=O)N1CCN(c2nc(OC[C@@H]3CCCN3C)nc3c2CC[C@@]2(CCc4ccccc4C2)C3)CC1 ; C=C(F)C(=O)N1CCN(c2nc(OC[C@@H]3CCCN3C)nc3c2CC[C@@]2(CCc4ccccc4C2)C3)CC1 ; C=CC(=O)N1CCN(c2nc(OC[C@@H]3CCCN3C)nc3c2CC[C@@]2(CCc4ccccc4C2)C3)[C@@H](C)C1 ; C=C(F)C(=O)N1CCN(c2nc(OC[C@@H]3CCCN3C)nc3c2CC[C@@]2(CCc4ccccc4C2)C3)[C@@H](C)C1]

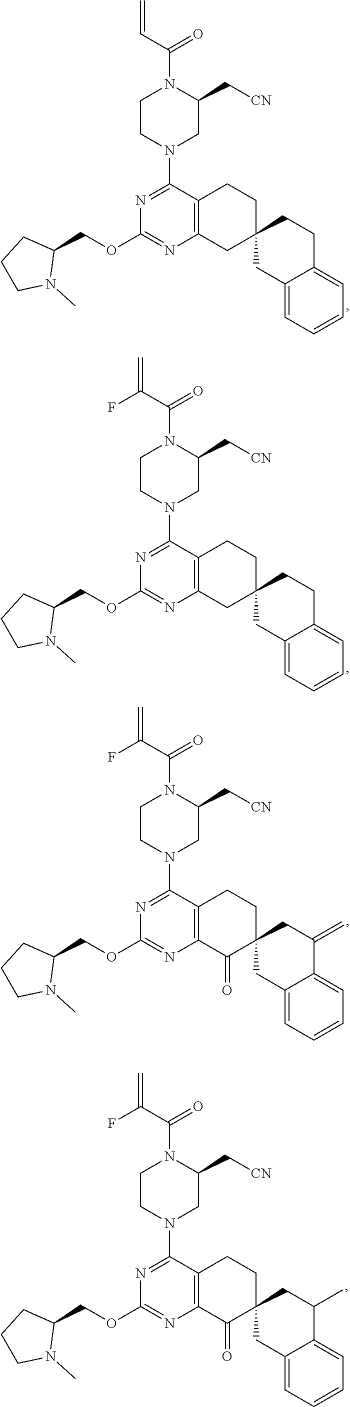

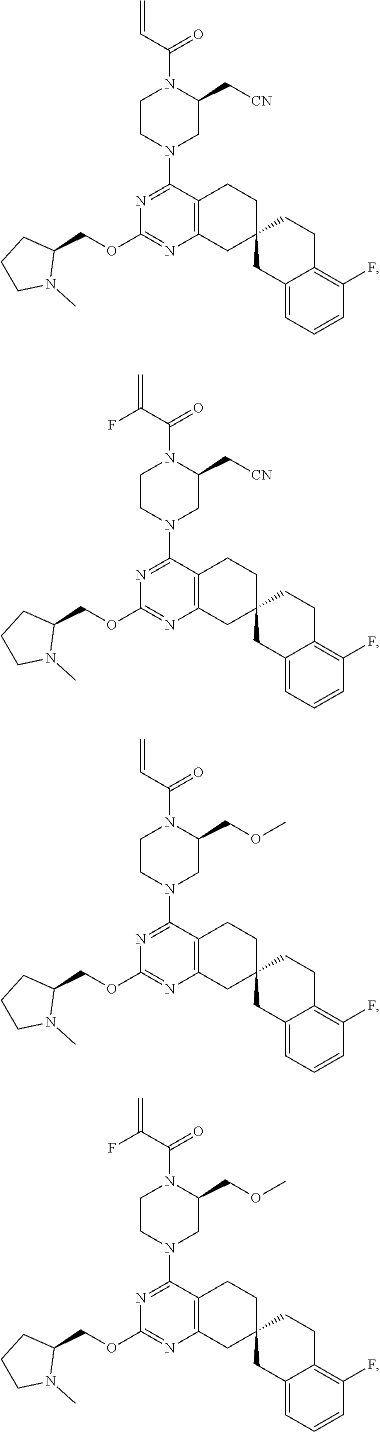 [SMILES: C=CC(=O)N1CCN(c2nc(OC[C@@H]3CCCN3C)nc3c2CC[C@]2(CCc4c(F)cccc4C2)C3)C[C@@H]1CC#N ; C=C(F)C(=O)N1CCN(c2nc(OC[C@@H]3CCCN3C)nc3c2CC[C@]2(CCc4c(F)cccc4C2)C3)C[C@@H]1CC#N ; C=CC(=O)N1CCN(c2nc(OC[C@@H]3CCCN3C)nc3c2CC[C@]2(CCc4c(F)cccc4C2)C3)C[C@@H]1COC ; C=C(F)C(=O)N1CCN(c2nc(OC[C@@H]3CCCN3C)nc3c2CC[C@]2(CCc4c(F)cccc4C2)C3)C[C@@H]1COC]

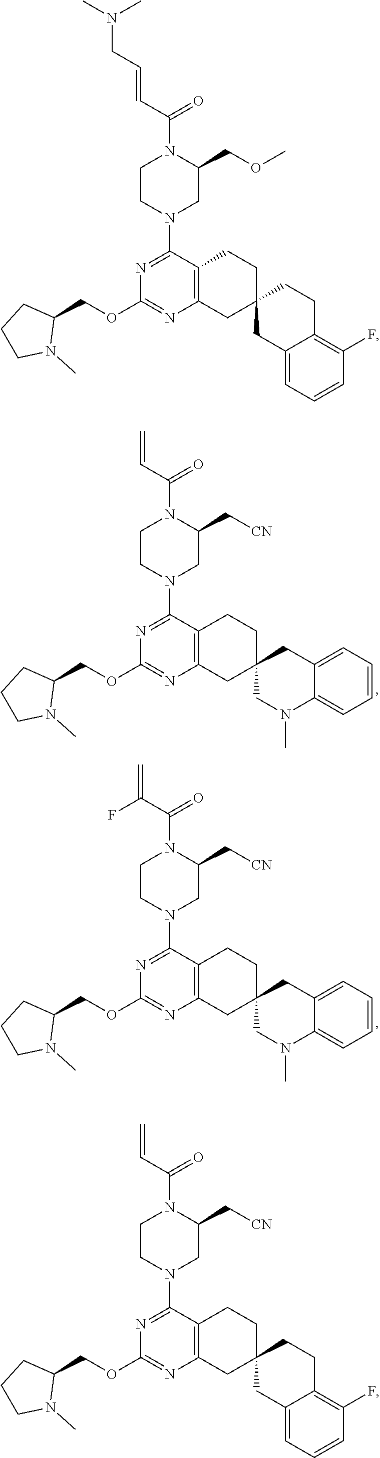

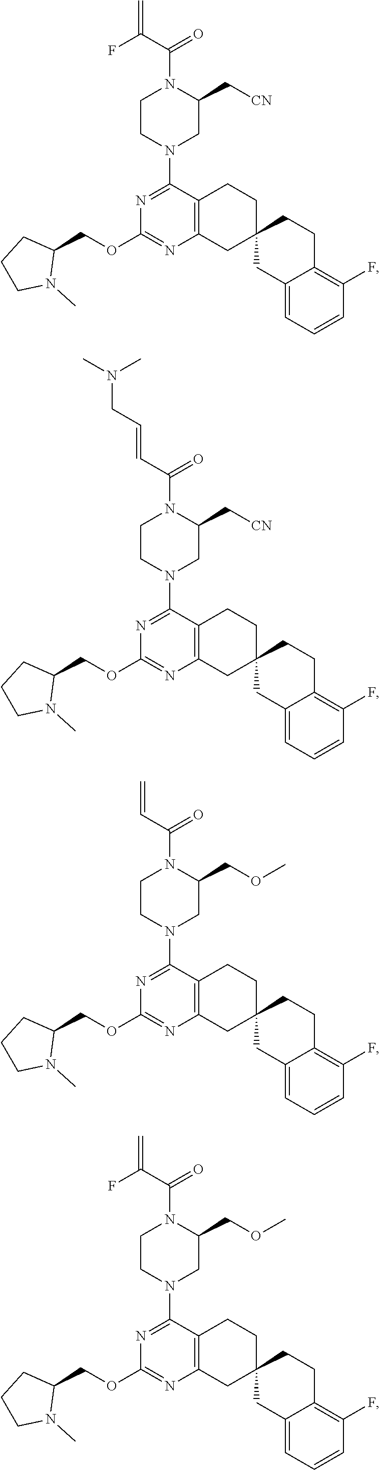 [SMILES: C=C(F)C(=O)N1CCN(c2nc(OC[C@@H]3CCCN3C)nc3c2CC[C@@]2(CCc4c(F)cccc4C2)C3)C[C@@H]1CC#N ; CN(C)C/C=C/C(=O)N1CCN(c2nc(OC[C@@H]3CCCN3C)nc3c2CC[C@@]2(CCc4c(F)cccc4C2)C3)C[C@@H]1CC#N ; C=CC(=O)N1CCN(c2nc(OC[C@@H]3CCCN3C)nc3c2CC[C@@]2(CCc4c(F)cccc4C2)C3)C[C@@H]1COC ; C=C(F)C(=O)N1CCN(c2nc(OC[C@@H]3CCCN3C)nc3c2CC[C@@]2(CCc4c(F)cccc4C2)C3)C[C@@H]1COC]

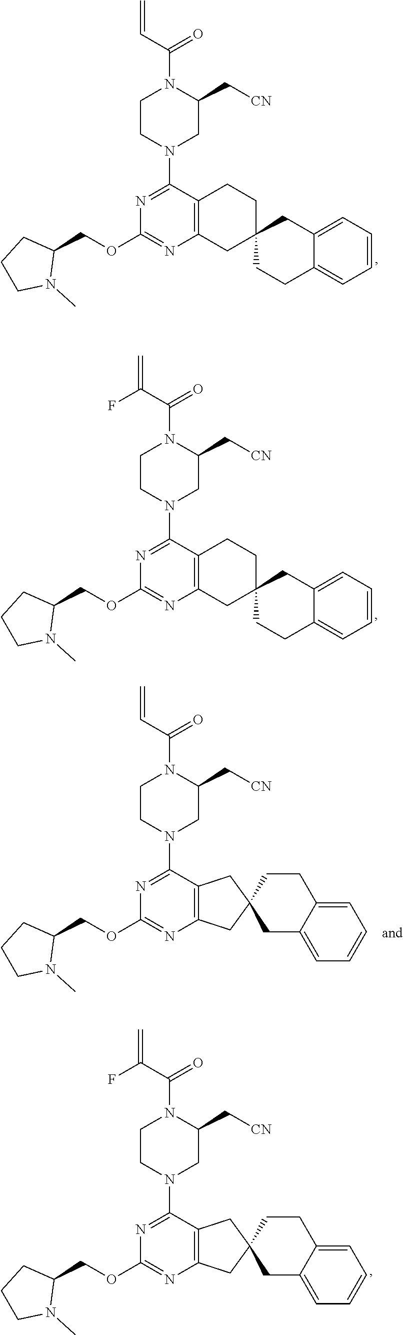 [SMILES: C=CC(=O)N1CCN(c2nc(OC[C@@H]3CCCN3C)nc3c2CC[C@]2(CCc4ccccc4C2)C3)C[C@@H]1CC#N ; C=C(F)C(=O)N1CCN(c2nc(OC[C@@H]3CCCN3C)nc3c2CC[C@]2(CCc4ccccc4C2)C3)C[C@@H]1CC#N ; C=CC(=O)N1CCN(c2nc(OC[C@@H]3CCCN3C)nc3c2C[C@]2(CCc4ccccc4C2)C3)C[C@@H]1CC#N ; C=C(F)C(=O)N1CCN(c2nc(OC[C@@H]3CCCN3C)nc3c2C[C@]2(CCc4ccccc4C2)C3)C[C@@H]1CC#N]

pharmaceutically salt thereof.

In some aspects, the invention relates to a compound of Formula IIIa having a structure selected from:

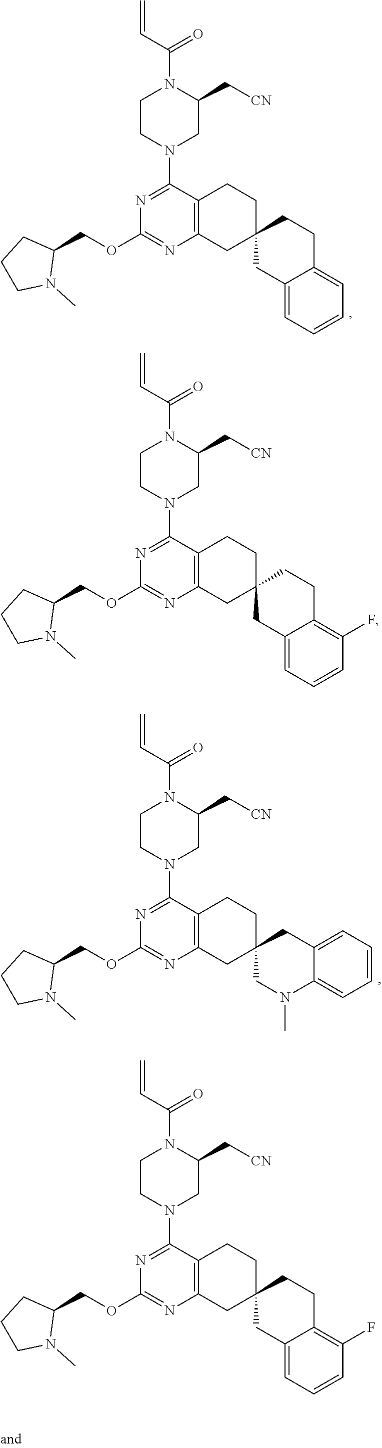 [SMILES: C=CC(=O)N1CCN(c2nc(OC[C@@H]3CCCN3C)nc3c2CC[C@@]2(CCc4ccccc4C2)C3)C[C@@H]1CC#N ; C=CC(=O)N1CCN(c2nc(OC[C@@H]3CCCN3C)nc3c2CC[C@]2(CCc4c(F)cccc4C2)C3)C[C@@H]1CC#N ; C=CC(=O)N1CCN(c2nc(OC[C@@H]3CCCN3C)nc3c2CC[C@@]2(Cc4ccccc4N(C)C2)C3)C[C@@H]1CC#N ; C=CC(=O)N1CCN(c2nc(OC[C@@H]3CCCN3C)nc3c2CC[C@@]2(CCc4c(F)cccc4C2)C3)C[C@@H]1CC#N]

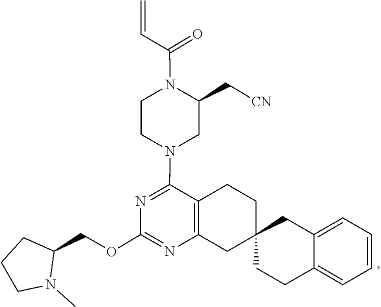 [SMILES: C=CC(=O)N1CCN(c2nc(OC[C@@H]3CCCN3C)nc3c2CC[C@]2(CCc4ccccc4C2)C3)C[C@@H]1CC#N]

or a pharmaceutically salt thereof.

In certain embodiments, the invention relates to a pharmaceutical composition comprising any of the compounds described herein and a pharmaceutically acceptable diluent or excipient.

### Example Methods of Treatment/Use

The compounds described herein are inhibitors of KRAS G12C and therefore may be useful for treating diseases wherein the underlying pathology is (at least in part) mediated by KRAS G12C. Such diseases include cancer and other diseases in which there is a disorder of transcription, cell proliferation, apoptosis, or differentiation.

In certain embodiments, the method of treating cancer in a subject in need thereof comprises administering to the subject an effective amount of any of the compounds described herein, or a pharmaceutically acceptable salt thereof. For example, the cancer may be selected from carcinoma (e.g., a carcinoma of the endometrium, bladder, breast, colon (e.g., colorectal carcinomas such as colon adenocarcinoma and colon adenoma)), sarcoma (e.g., a sarcoma such as Kaposi's, osteosarcoma, tumor of mesenchymal origin, for example fibrosarcoma or habdomyosarcoma), kidney, epidermis, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, nose, head and neck, prostate, and skin (e.g., squamous cell carcinoma), human breast cancers (e.g., primary breast tumors, node-negative breast cancer, invasive duct adenocarcinomas of the breast, non-endometrioid breast cancers), familial melanoma, and melanoma. Other examples of cancers that may be treated with a compound of the invention include hematopoietic tumors of lymphoid lineage (e.g. leukemia, acute lymphocytic leukemia, mantle cell lymphoma, chronic lymphocytic leukaemia, B-cell lymphoma (such as diffuse large B cell lymphoma), T-cell lymphoma, multiple myeloma, Hodgkin's lymphoma, non-Hodgkin's lymphoma, hairy cell lymphoma, and Burkett's lymphoma), and hematopoietic tumors of myeloid lineage, for example acute and chronic myelogenous leukemias, myelodysplastic syndrome, and promyelocytic leukemia. Other cancers include a tumor of the central or peripheral nervous system, for example astrocytoma, neuroblastoma, glioma or schwannoma; seminoma; teratocarcinoma; xeroderma pigmentosum; retinoblastoma; keratoctanthoma; and thyroid follicular cancer.

In particular embodiments, the treated cancer is selected from pancreatic cancer, gall bladder, thyroid cancer, colorectal cancer, lung cancer (including non-small cell lung cancer), gall bladder cancer, and bile duct cancer.

In other particular embodiments, the treated cancer is selected from pancreatic cancer, colorectal cancer, and lung cancer (including non-small cell lung cancer).

In some aspects, the subject is a mammal, for example, a human.

Further disclosed herein are methods of inhibiting KRAS G12C in a cell comprising contacting said cell with any of the compounds described herein, or a pharmaceutically acceptable salt thereof, such that KRAS G12C enzyme is inhibited in said cell. For example, the cell is a cancer cell. In preferred embodiments, proliferation of the cell is inhibited or cell death is induced.

Further disclosed herein is a method of treating a disease treatable by inhibition of KRAS G12C in a subject, comprising administering to the subject in recognized need of such treatment, an effective amount of any of the compounds described herein and/or a pharmaceutically acceptable salt thereof. Diseases treatable by inhibition of KRAS G12C include, for example, cancers. Further exemplary diseases include pancreatic cancer, gall bladder, thyroid cancer, colorectal cancer, lung cancer (including non-small cell lung cancer), gall bladder cancer, and bile duct cancer.

The methods of treatment comprise administering a compound of the invention, or a pharmaceutically acceptable salt thereof, to a subject in need thereof. Individual embodiments include methods of treating any one of the above-mentioned disorders or diseases by administering an effective amount of a compound of the invention, or a pharmaceutically acceptable salt thereof, to a subject in need thereof.

Certain embodiments include a method of modulating KRAS G12C activity in a subject comprising administering to the subject a compound of the invention, or a pharmaceutically acceptable salt thereof. Additional embodiments provide a method for the treatment of a disorder or a disease mediated by KRAS G12C in a subject in need thereof, comprising administering to the subject an effective amount of the compound of Formula I, Ia, Ib, Ic, Id, II, IIa, IIb, III, IIIa, IIIb, IIIc, IV, IVa, IVb, IVc, V, Va, Vb or Vc, or a pharmaceutically acceptable salt thereof. Other embodiments of the invention provide a method of treating a disorder or a disease mediated by KRAS G12C, in a subject in need of treatment thereof comprising administering an effective amount of a compound of the invention, or a pharmaceutically acceptable salt thereof, wherein the disorder or the disease is selected from carcinomas with genetic aberrations that activate KRAS activity. These include, but are not limited to, cancers.

The present method also provides the use of a compound of invention, or a pharmaceutically acceptable salt thereof, for the treatment of a disorder or disease mediated by KRAS G12C.

In some embodiments, a compound of the invention, or a pharmaceutically acceptable salt thereof, is used for the treatment of a disorder or a disease mediated by KRAS G12C.

Yet other embodiments of the present method provide a compound according to Formula I, Ia, Ib, Ic, Id, II, IIa, IIb, III, IIIa, IIIb, IIIc, IV, IVa, IVb, IVc, V, Va, Vb or Vc, or a pharmaceutically acceptable salt thereof, for use as a medicament.

Still other embodiments of the present method encompass the use of a compound of Formula I, Ia, Ib, Ic, Id, II, IIa, IIb, III, IIIa, IIIb, IIIc, IV, IVa, IVb, IVc, V, Va, Vb or Vc, or a pharmaceutically acceptable salt thereof, in the manufacture of a medicament for the treatment of a disorder or disease mediated by KRAS G12C.

### Example of Predicted Affinity for KRAS G12C of Example Compounds

The covalent KRAS G12C inhibitor MRTX1257:

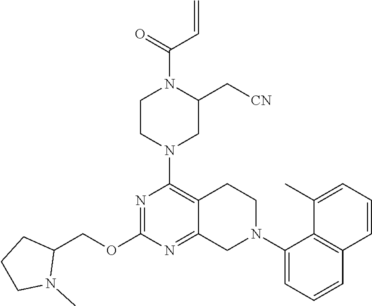 [SMILES: C=CC(=O)N1CCN(c2nc(OCC3CCCN3C)nc3c2CCN(c2cccc4cccc(C)c24)C3)CC1CC#N]

is known in the art to be potent and selective, and has been shown to have desirable pharmacokinetic properties. MRTX1257 has also been shown to have desirable efficacy in xenograft models of cancer.

Using the covalent docking protocol implemented in the computer program MOE version 2019.0101 (Molecular Operating Environment, Chemical Computing Group, Montreal, CA), compounds were covalently docked into a modified version of KRAS G12C protein (PDB accession code 6N2K). The receptor geometry was generated by minimization of the binding site residues of 6N2K in the presence of MRTX1257. Estimated binding affinities (in arbitrary units) were computed for each compound covalently docked into this modified receptor, where more negative values correspond to higher estimated predicted affinities. See Table 1. The predicted binding affinity of MRTX1257 in this receptor was −10.7148.

Using the CovDock covalent docking module in the Schrodinger computational chemistry suite (v. 2020-1, Schrodinger, LLC, New York, N.Y.) compounds of particular interest were subjected to covalent docking into the published crystal structure of KRAS G12C (PDB accession code 6N2K). Predicted docking scores and estimated binding affinities (“MMGBSA” and “CovDock”) are provided (in arbitrary units), where more negative values correspond to greater predicted affinity. See Table 2.

### Example Compounds

Specific embodiments of the invention include those compounds listed in Table 1. The identifying number (“Cmpd”), the chemical structure (“Structure”), and the predicted binding affinity for KRAS G12C (in arbitrary units, A.U.) (“Score”) are disclosed for each compound.

Additional specific embodiments of the invention include those compounds listed in Table 2. The identifying number (“Cmpd”), the chemical structure (“Structure”), and the predicted binding affinity for KRAS G12C (in arbitrary units, A.U.) (“Score”) from two distinct methods (“MMGBSA” and “CovDock”) are disclosed for each compound.

Specific embodiments of the invention include those compounds listed in Table 3. The identifying number (“Cmpd”), the chemical structure (“Structure”), and the example method used to synthesize the compound (“Method”), are disclosed for each compound.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### EXEMPLIFICATION

### Synthetic Protocols

Compounds as disclosed herein can be synthesized via a number of specific methods. The examples, which outline specific synthetic routes, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### Example 1: Synthesis of Tetrahydronaphthalene, Tetrahydroquinoline and Chromane Functionalized Compounds

### Preparation of Intermediate 1-1

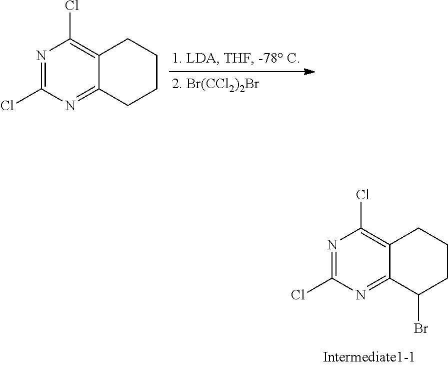 [SMILES: Clc1nc(Cl)c2c(n1)CCCC2 ; Clc1nc(Cl)c2c(n1)C(Br)CCC2]

The starting material, 2,4-dichloro-5,6,7,8-tetrahydroquinazoline (1.288 g, 6.34 mmol), was dissolved in tetrahydrofuran (25 mL) and transferred into a cold (−78° C.) solution of lithium diisopropylamide (7.3 mmoles, 0.5 M solution in tetrahydrofuran/hexane, freshly prepared from diisopropylamine/n-BuLi). After 120 minutes, a solution of tetrachlorodibromoethane (2.68 g, 76.30 mmol) in tetrahydrofuran (15 mL) was added rapidly via cannula.

After 15 minutes at constant temperature, the reaction was quenched by the addition of saturated aqueous ammonium chloride (50 mL) and diluted with methylene chloride (100 mL). The mixture was transferred to a separatory funnel. The organic phase was separated and dried over potassium carbonate, filtered and concentrated onto silica gel. The resulting solid was purified by flash chromatography (0-20% EtOAc/Hexanes) to yield Intermediate 1-1, 8-bromo-2,4-dichloro-5,6,7,8-tetrahydroquinazoline (412.3 mg, 23% yield), as a white solid.

¹H NMR (400 MHz, Chloroform-d) δ 5.24 (td, J=3.3, 2.6, 1.3 Hz, 1H), 3.05-2.94 (m, 1H), 2.71 (ddd, J=18.2, 11.3, 6.7 Hz, 1H), 2.48-2.36 (m, 1H), 2.32-2.20 (m, 1H), 2.17 (dtd, J=14.6, 2.6, 1.4 Hz, 1H), 2.09-2.00 (m, 1H) ppm

LCMS: [M+H]⁺ m/z=280.9 amu

### Preparation of Intermediate 1-2

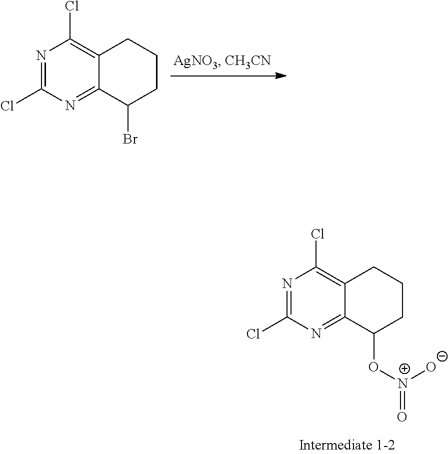 [SMILES: Clc1nc(Cl)c2c(n1)C(Br)CCC2 ; O=[N+]([O-])OC1CCCc2c(Cl)nc(Cl)nc21]

To a vial containing 8-bromo-2,4-dichloro-5,6,7,8-tetrahydroquinazoline (52.3 mg, 0.186 mmol) and silver(I)nitrate (47.6 mg, 0.28 mmol) was added acetonitrile (2 mL) under an atmosphere of nitrogen. The reaction was warmed to 50° C. and stirred for 8 hours, at which time TLC analysis indicated consumption of the starting material. Silica gel was added and the solvent was removed in vacuo to yield a white powder. Purification by flash chromatography (0→30% EtOAc/Hexanes) afforded Intermediate 1-2, 2,4-dichloro-5,6,7,8-tetrahydroquinazolin-8-yl nitrate, as a white solid.

¹H NMR (400 MHz, Chloroform-d) δ 6.00 (dd, J=5.7, 4.6 Hz, 1H), 2.93-2.81 (m, 1H), 2.72 (ddd, J=18.1, 7.7, 6.2 Hz, 1H), 2.34-1.92 (m, 4H) ppm

LCMS: [M+H]⁺ m/z=264.0 amu

### Preparation of Intermediate 1-3

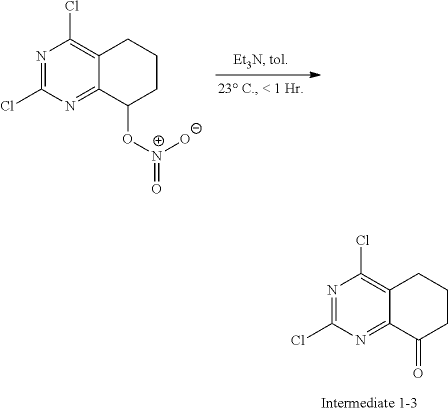 [SMILES: O=[N+]([O-])OC1CCCc2c(Cl)nc(Cl)nc21 ; O=C1CCCc2c(Cl)nc(Cl)nc21]

A solution of 2,4-dichloro-5,6,7,8-tetrahydroquinazolin-8-yl nitrate in toluene (0.025 M) was treated with triethyl amine (50% vol/vol). The reaction was stirred at ambient temperature for 90 minutes and concentrated onto silica gel. Flash chromatography was performed with refractive index detection (0→50% hexanes/EtOAc). The product fractions were pooled and concentrated to yield Intermediate 1-3, 2,4-dichloro-6,7-dihydroquinazolin-8(5H)-one, as a white solid.

¹H NMR (400 MHz, Chloroform-d) δ ¹H NMR (400 MHz, Chloroform-d) δ 3.05 (t, J=6.2 Hz, 2H), 2.91-2.76 (m, 2H), 2.38-2.16 (m, 2H) ppm

LCMS: [M+H]⁺ m/z=217.0 amu

### Preparation of Intermediate 1-4

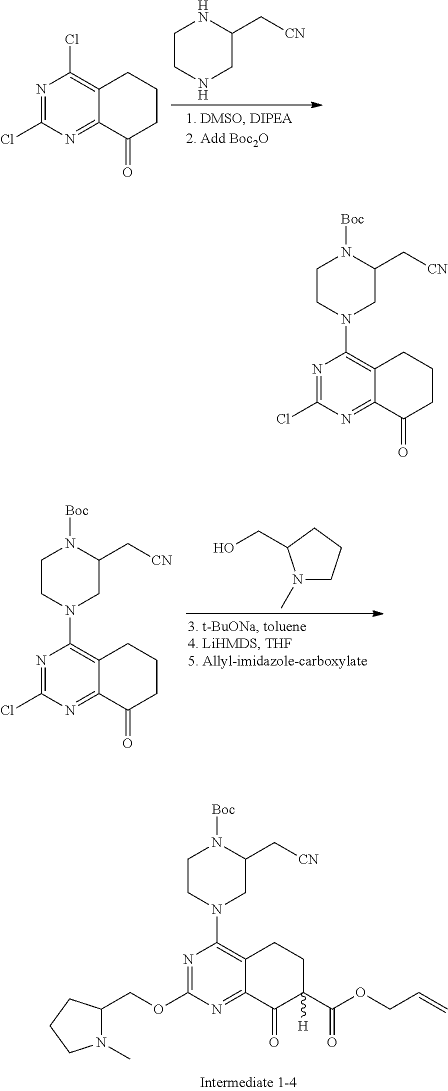

### Preparation of Intermediate 1-5

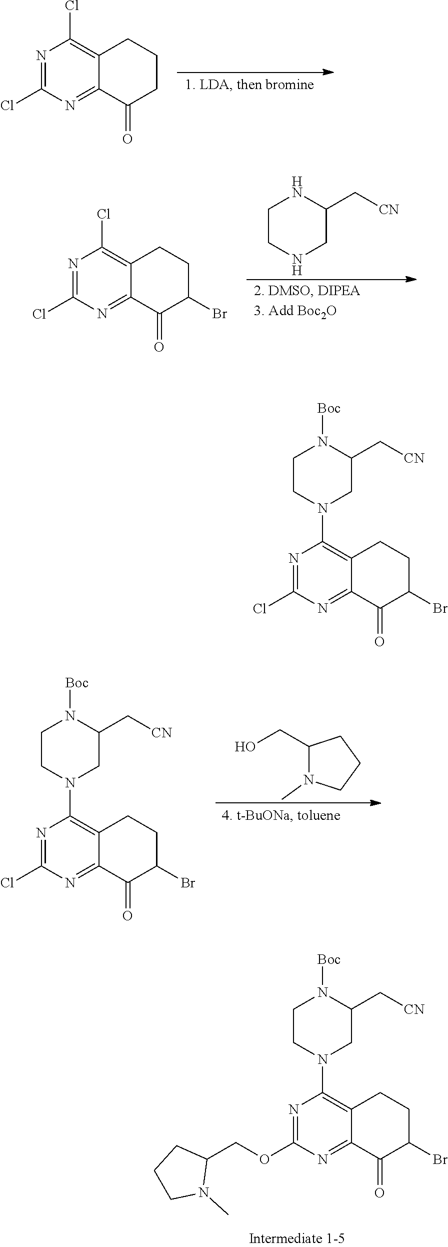

### Preparation of Tetrahydronaphthalene Functionalized Compounds

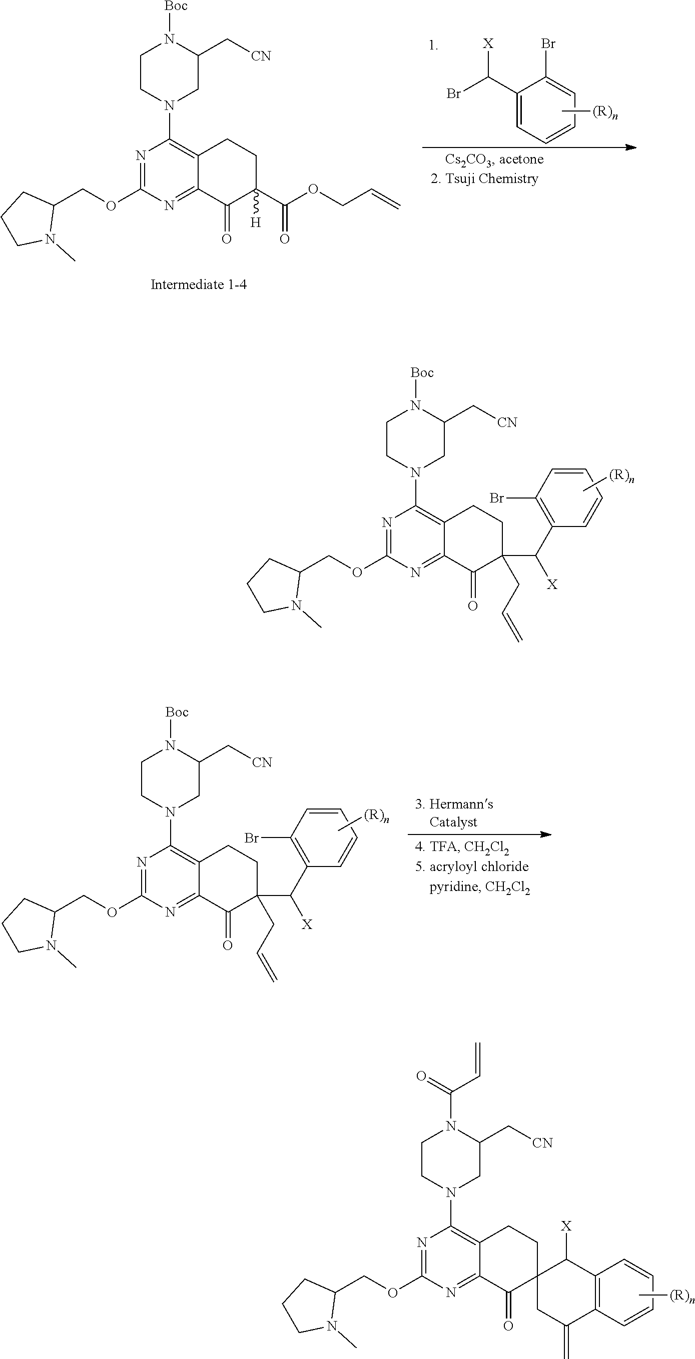

The catalyst for the Tsuji step can be chosen in an R or S configuration to yield an enantioenriched product at the quaternary stereo center. The exo-cyclic olefin can be transformed in several ways to yield analogs of this compound, as would be understood by one of ordinary skill in the art.

Compounds obtained with this synthetic route include, but are not limited to, those where X is H, Cl, F, OH, CH₃ or OCH₃, R, in each occurrence and if present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone of the cyclohexanone in compounds obtained with this synthetic route can be transformed to C(H)OH, CH₂, OCH₃, C(H)F or CF₂ using procedures that would be known to a person of ordinary skill in the art.

### Preparation of Tetrahydroquinoline Functionalized Compounds

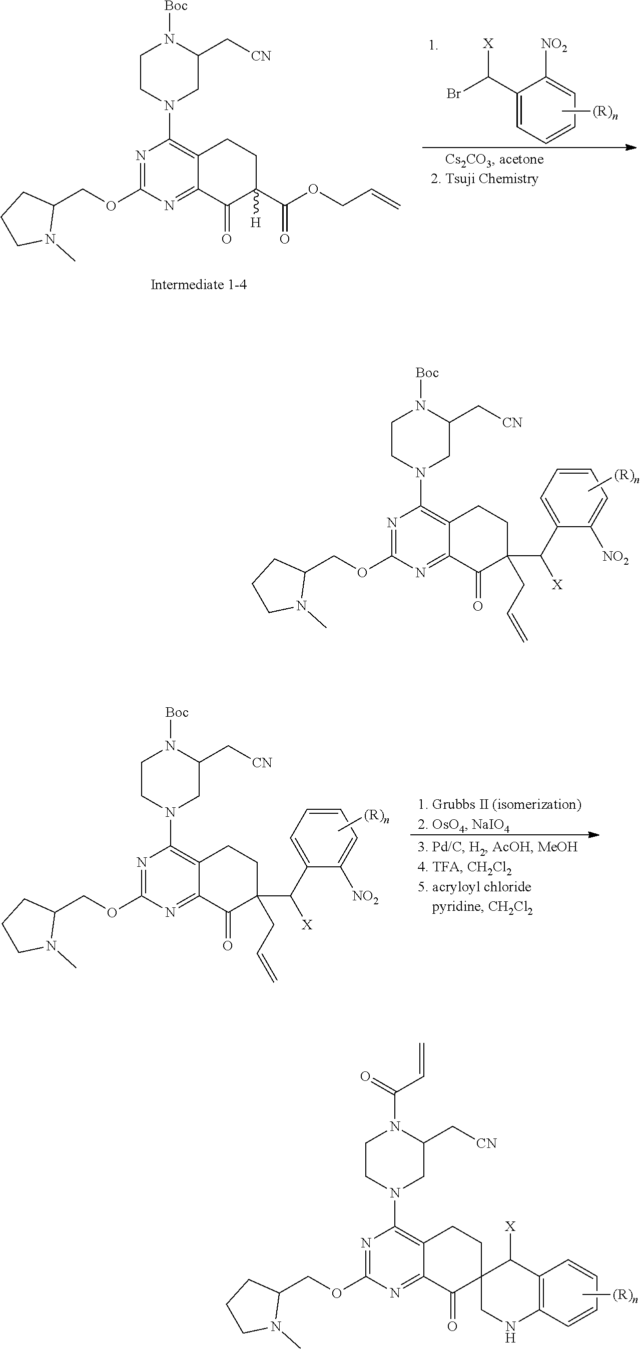

The catalyst for the Tsuji step can be chosen in an R or S configuration to yield an enantioenriched product at the quaternary stereo center. The amine in the tetrahydroquinoline can be substituted with optionally substituted alkyl using procedures that would be readily apparent to a person of ordinary skill in the art.

Compounds obtained with this synthetic route include, but are not limited to, those where X is H, Cl, F, CH₃ or OCH₃, R, in each occurrence and when present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone of the cyclohexanone in compounds obtained with this synthetic route can be transformed to C(H)OH, CH₂, OCH₃, C(H)F or CF₂ using procedures that would be known to a person of ordinary skill in the art.

### Preparation of Chromane Functionalized Compounds

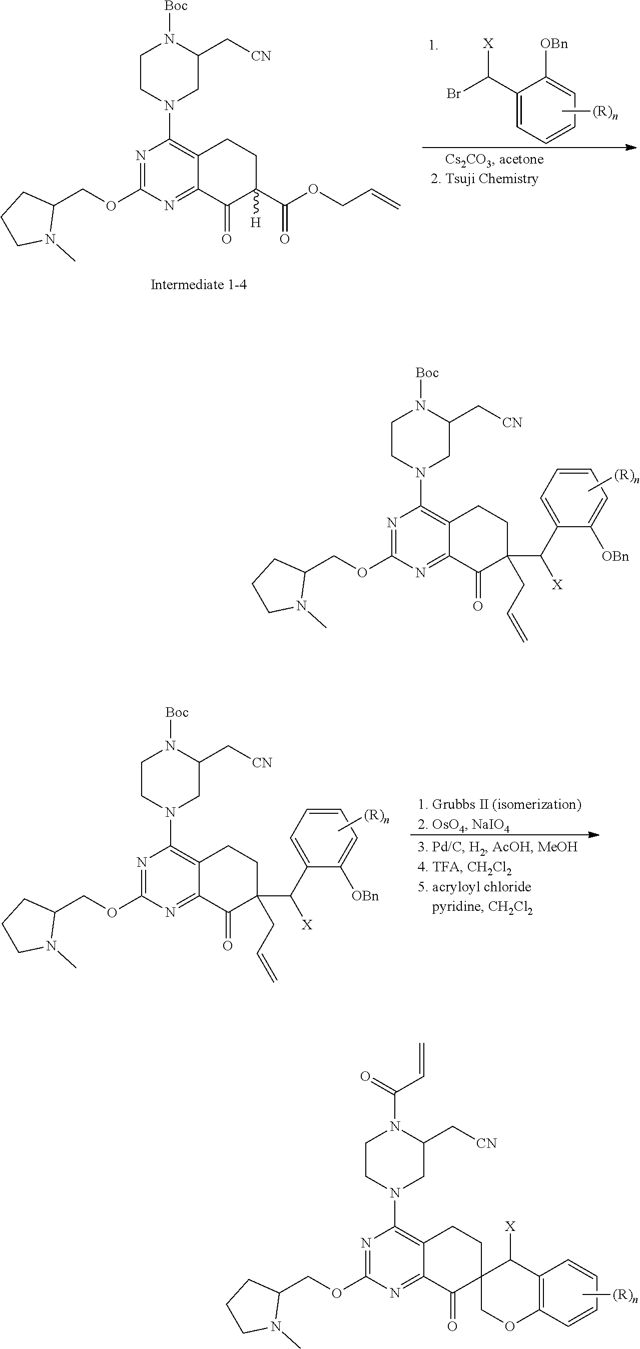

The catalyst for the Tsuji step can be chosen in an R or S configuration to yield an enantioenriched product at the quaternary stereo center.

Compounds obtained with this synthetic route include, but are not limited to, those where X is H, Cl, F, CH₃ or OCH₃, R, in each occurrence and when present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone of the cyclohexanone in compounds obtained with this synthetic route can be transformed to C(H)OH, CH₂, OCH₃, C(H)F or CF₂ using procedures that would be known to a person of ordinary skill in the art.

### Preparation of Thiochromane Functionalized Compounds

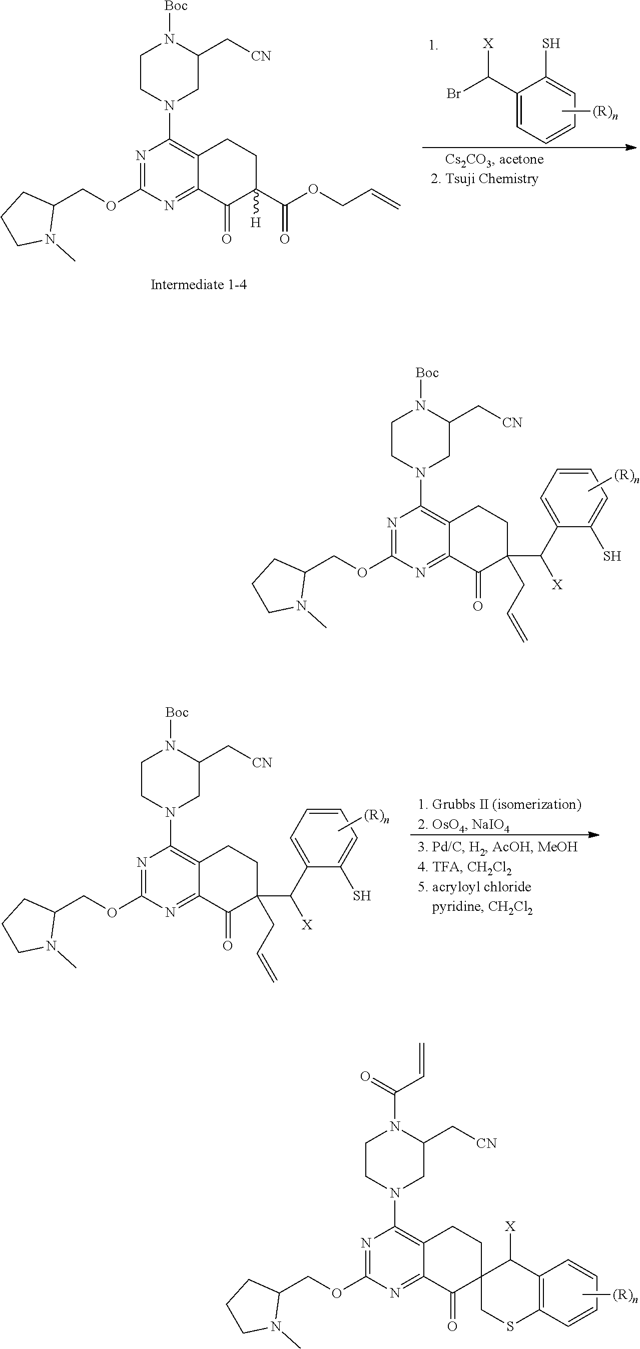

The catalyst for the Tsuji step can be chosen in an R or S configuration to yield an enantioenriched product at the quaternary stereo center.

Compounds obtained with this synthetic route include, but are not limited to, those where X is H, Cl, F, CH₃ or OCH₃, R, in each occurrence and when present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone in compounds obtained with this synthetic route can be transformed to C(H)OH, CH₂, OCH₃, C(H)F or CF₂ using procedures that would be known to a person of ordinary skill in the art.

### Preparation of Benzomorpholine Functionalized Compounds

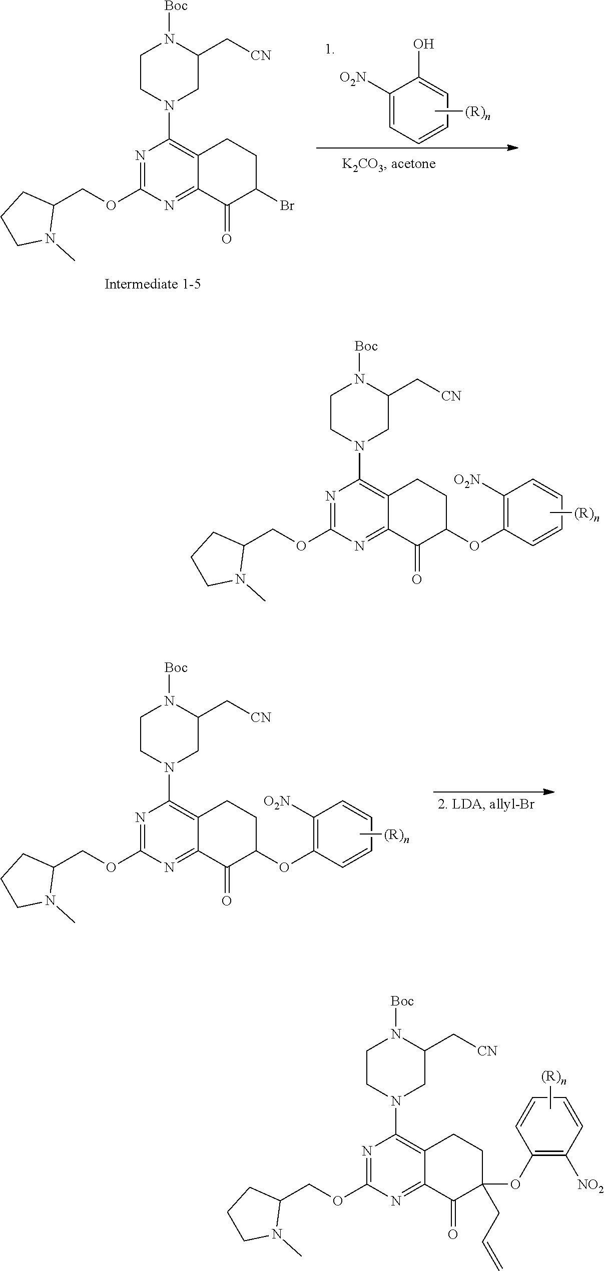

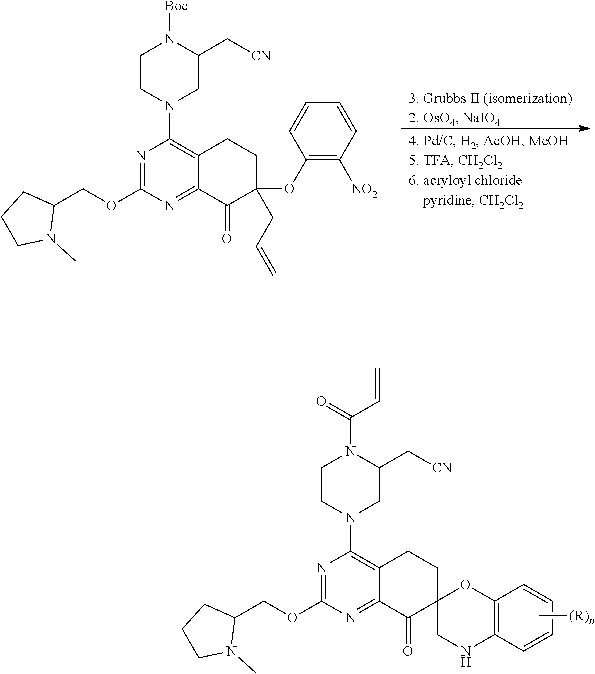

Compounds obtained with this synthetic route include, but are not limited to, those where R, in each occurrence and when present, is independently Cl, F, CH₃ or OCH₃, and n is 0, 1 or 2. Other substituents for R would be readily apparent to one of skill in the art, particularly those substituents that are found in commercially available molecules used in the first step of this synthesis.

Furthermore, the amine in the morpholine can be substituted with optionally substituted alkyl using procedures that would be readily apparent to a person of ordinary skill in the art. Additionally, the ketone in compounds obtained with this synthetic route can be transformed to C(H)OH, CH₂, OCH₃, C(H)F or CF₂ using procedures that would be known to a person of ordinary skill in the art.

### Example 2: Synthesis of Indane Functionalized Compounds

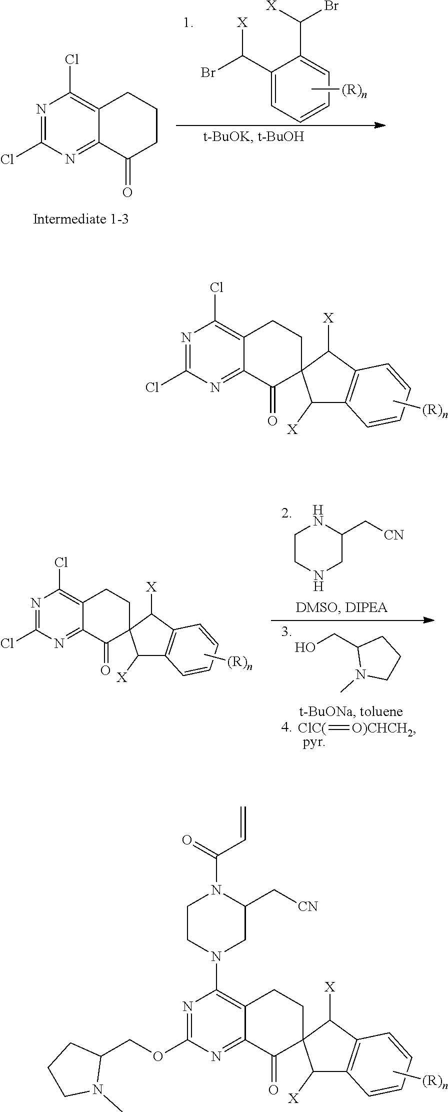

This synthesis produces racemic mixtures, and separation of the enantiomers using chiral HPLC or SFC chromatography with optimized conditions would be readily achieved by one of ordinary skill in the art.

Compounds obtained with this synthetic route include, but are not limited to, those where X is H, Cl, F, CH₃ or OCH₃, R, in each occurrence and when present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone of the cyclohexanone in compounds obtained with this synthetic route can be transformed to C(H)OH, CH₂, OCH₃, C(H)F or CF₂ using procedures that would be known to a person of ordinary skill in the art.

### Example 3: Synthesis of Coumaran Functionalized Compounds

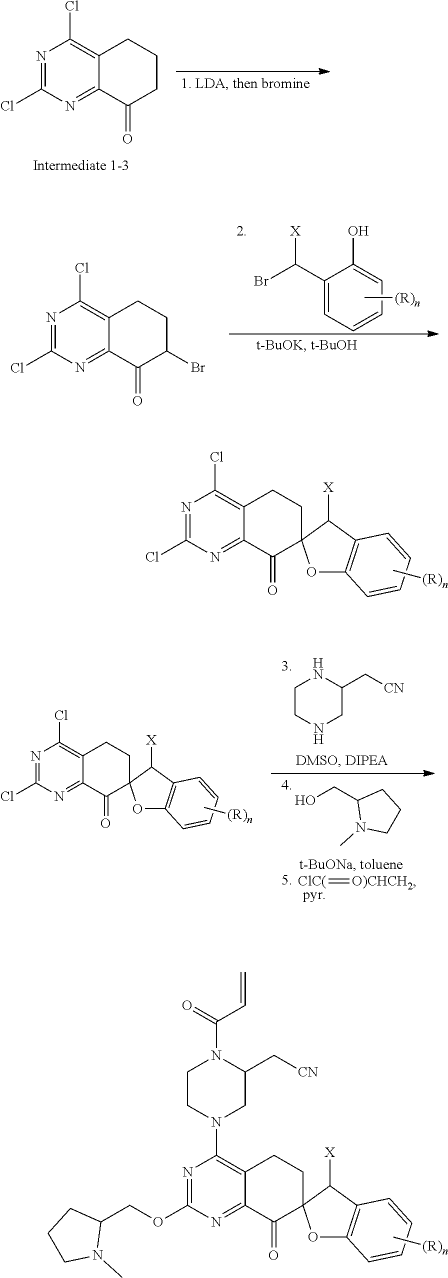

This synthesis produces racemic mixtures, and separation of the enantiomers using chiral HPLC or SFC chromatography with optimized conditions would be readily achieved by one of ordinary skill in the art.

Compounds obtained with this synthetic route include, but are not limited to, those where X is H, Cl, F, CH₃ or OCH₃, R, in each occurrence and when present, is independently Cl, F, CH₃ or OCH₃, and n is 0, 1 or 2. Other substituents for X and R would be readily apparent to one of skill in the art, particularly those substituents that are found in commercially available molecules used in the first step of this synthesis.

Additionally, the ketone of the cyclohexanone in compounds obtained with this synthetic route can be transformed to C(H)OH, CH₂, OCH₃, C(H)F or CF₂ using procedures that would be known to a person of ordinary skill in the art.

### Example 4: Synthesis of Compounds C-1 through C-8, C-15 and C-16

### Synthesis of 2,4-dichloro-5,6,7,8-tetrahydroquinazoline

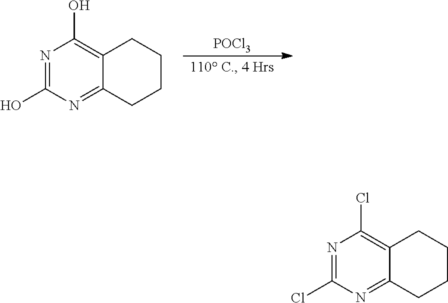 [SMILES: Oc1nc(O)c2c(n1)CCCC2 ; "POCl3" ; "110° C., 4 Hrs" ; Clc1nc(Cl)c2c(n1)CCCC2]

A solution of 5,6,7,8-tetrahydroquinazoline-2,4-diol (750 g, 4.51 mol) in POCl₃ (3.30 kg, 21.5 mol) was stirred at 110° C. for 4 hours. TLC (Dichloromethane/Methanol=10/1) indicated the 5,6,7,8-tetrahydroquinazoline-2,4-diol was consumed completely. TLC (Petroleum ether/Ethyl acetate=3/1, R_{f}=0.66) indicated one new spot was formed. The reaction mixture was cooled to 15° C., then diluted with ethyl acetate (2000 mL). The organic phase was quenched with ice water (6000 mL) and adjusted to pH=8 with NaHCO₃ solid, then extracted with ethyl acetate (2000 mL*2). The combined organic layers were dried over Na₂SO₄, filtered and concentrated under vacuum to give crude product. The residue was purified by flash silica gel chromatography (SiO₂, Petroleum ether/Ethyl acetate=1\0 to 1\1) to give 2,4-dichloro-5,6,7,8-tetrahydroquinazoline (230 g, 1.11 mol, 25% yield) as a white solid.

¹H NMR (400 MHz, Chloroform-d) δ 2.95-2.80 (m, 2H), 2.75-2.65 (m, 2H), 1.90-1.84 (m, 4H) ppm

LC/MS: [M+H]⁺ m/z=203.4 amu

### Alternative Synthesis of Intermediate 1-1

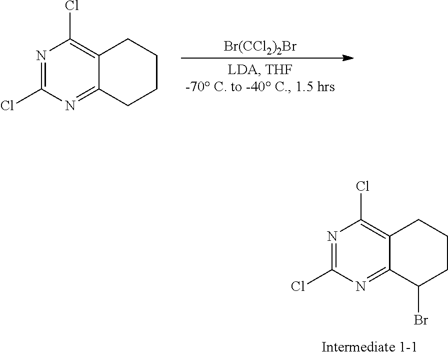

To a solution of 2,4-dichloro-5,6,7,8-tetrahydroquinazoline (150 g, 664 mmol) in THE (600 mL) was added to LDA (2 M, 499 mL) at −70° C. The mixture was stirred at −70° C. for 30 minutes. The mixture was added to a solution of tetrachlorodibromoethane (325 g, 997 mmol, 120 mL) in THE (2.80 L) under−70 to −40° C., and stirred at −40° C. for 1 hour. LCMS showed the reaction was complete. The mixture was poured into saturated NH₄Cl solution (8.00 L) at 0° C. under stirring, and then stirred at 0 C for 30 minutes. The mixture was extracted with ethyl acetate (5.00 L*3). The combined organic layers were dried over Na₂SO₄, filtered and concentrated in vacuo. The residue was purified by column chromatography (SiO₂, Petroleum ether/Ethyl acetate=500/1 to 20/1) to give intermediate 1-1, 8-bromo-2,4-dichloro-5,6,7,8-tetrahydroquinazoline (182 g, 512 mmol, 26% yield), as an off-white solid.

LC/MS: [M+H]⁺ m/z=282.9 amu

### Synthesis of Intermediate 4-1

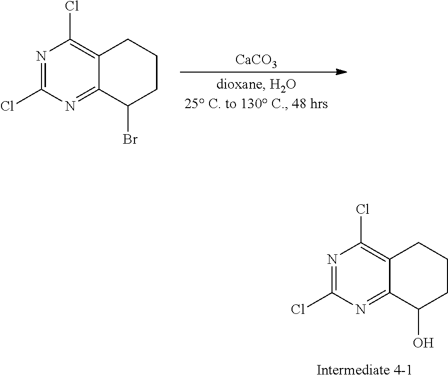 [SMILES: Clc1nc(Cl)c2c(n1)C(Br)CCC2 ; "CaCO3" ; OC1CCCc2c(Cl)nc(Cl)nc21]

To a solution of 8-bromo-2,4-dichloro-5,6,7,8-tetrahydroquinazoline (90.0 g, 253 mmol) in dioxane (1200 mL) and H₂O (1000 mL) was added CaCO₃ (76.1 g, 760 mmol) at 25° C., and the reaction was stirred at 130° C. for 48 hours. LCMS showed 35% of Intermediate 1-1 remained and 47% of desired mass was detected. To the reaction was added ethyl acetate (3000 mL), and stirred for 10 minutes. The reaction was filtered, and the filtrate was washed with brine (2000 mL*2), dried over anhydrous Na₂SO₄, filtered and concentrated in vacuo. The residue was purified by column chromatography (SiO₂, Petroleum ether/Ethyl acetate=20/1 to 5/1) to give Intermediate 4-1, 2,4-dichloro-5,6,7,8-tetrahydroquinazolin-8-ol (60.0 g, 268 mmol, 53% yield), as a yellow oil.

¹H NMR (400 MHz, Chloroform-d) δ 4.67-4.61 (m, 1H), 3.87 (d, J=2.4 Hz, 1H), 2.80-2.65 (m, 2H), 2.24-2.16 (m, 1H), 2.11-2.02 (m, 1H), 1.87-1.72 (m, 2H) ppm

LCMS: [M+H]⁺ m/z=218.8 amu

### Alternative Synthesis of Intermediate 1-3

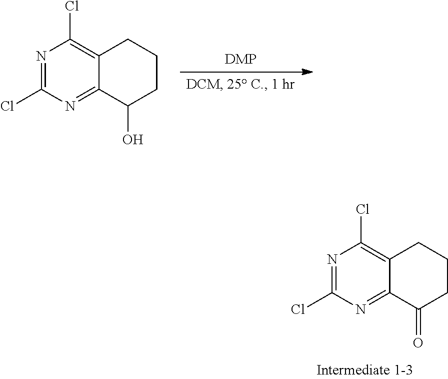

To a solution of 2,4-dichloro-5,6,7,8-tetrahydroquinazolin-8-ol (50.0 g, 223 mmol) in DCM (1000 mL) was added DMP (142 g, 335 mmol) at 25° C., and the reaction was stirred at 25° C. for 1 hour. LCMS showed the reaction was completed. To the mixture was added water (500 mL), adjusted to around pH=9 by progressively adding NaHCO₃ solution, and extracted with DCM (300 mL*2). The combined organic phases were washed with Na₂SO₃ solution (1000 mL*2), brine (1000 mL*2), dried over anhydrous Na₂SO₄, filtered and concentrated in vacuo. The residue was purified by column chromatography (SiO₂, Petroleum ether/Ethyl acetate=20/1 to 2/1) to give intermediate 1-3, 2,4-dichloro-6,7-dihydroquinazolin-8(5H)-one (26.0 g, 119 mmol, 53% yield), as a yellow solid.

¹H NMR (400 MHz, Chloroform-d) δ 3.05 (t, J=6.0 Hz, 2H), 2.84 (t, J=6.4 Hz, 2H), 2.31-2.24 (m, 2H) ppm

LCMS: [M+H]⁺ m/z=217.0 amu

### Synthesis of Intermediate 4-2

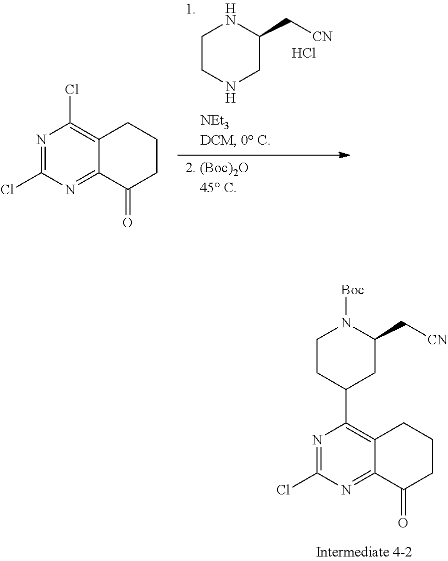 [SMILES: N#CC[C@H]1CNCCN1 ; Cl ; O=C1CCCc2c(Cl)nc(Cl)nc21 ; CC(C)(C)OC(=O)N1CCC(c2nc(Cl)nc3c2CCCC3=O)C[C@@H]1CC#N]

To a cooled (0° C.) solution of 2,4-dichloro-6,7-dihydroquinazolin-8(5H)-one (2.00 g, 9.21 mmol) in DCM (37 mL) was added triethylamine (6.4 mL, 46.01 mmol), followed by (S)-2-(piperazin-2-yl)acetonitrile.2HCl (1.49 g, 9.21 mmol). The resulting solution was stirred at 0° C. for 2 hours. After consumption of starting material was observed, di-tert-butyl dicarbonate (4.02 g, 18.43 mmol) was added and the reaction was heated to 40° C. and stirred for 1.5 hours. The reaction mixture was cooled to room temperature and diluted with H₂O (50 mL) and extracted with DCM (40 mL*3). The combined organic extracts were dried over Na₂SO₄, filtered and concentrated under vacuum. The crude product was purified using column chromatography (0→10% MeOH in DCM) to afford intermediate 4-2, tert-butyl (S)-4-(2-chloro-8-oxo-5,6,7,8-tetrahydroquinazolin-4-yl)-2-(cyanomethyl)piperazine-1-carboxylate (3.01 g, 7.41 mmol, 80% yield), as a yellow solid.

¹H NMR (400 MHz, Chloroform-d) δ 4.59 (td, J=7.2, 6.8, 3.3 Hz, 1H), 4.13 (dt, J=14.0, 2.3 Hz, 1H), 4.05 (s, 1H), 4.03-3.94 (m, 1H), 3.42 (dd, J=13.9, 4.0 Hz, 1H), 3.26 (s, 1H), 3.17 (td, J=12.1, 3.4 Hz, 1H), 2.89-2.80 (m, 2H), 2.80-2.71 (m, 3H), 2.71-2.60 (m, 1H), 2.21-2.02 (m, 2H), 1.49 (s, 9H) ppm

LCMS: [M+H]⁺ m/z=406.1/408.1 amu

### Synthesis of Compound C-1

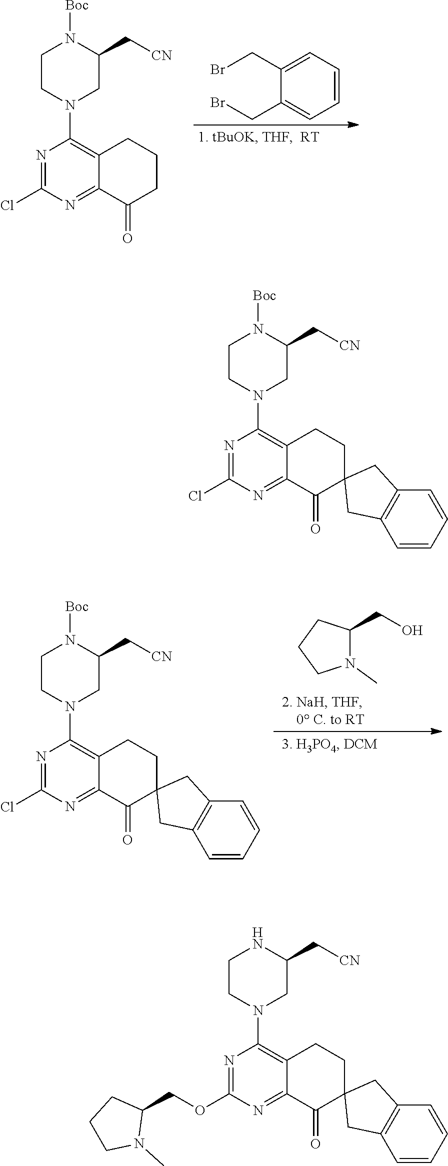

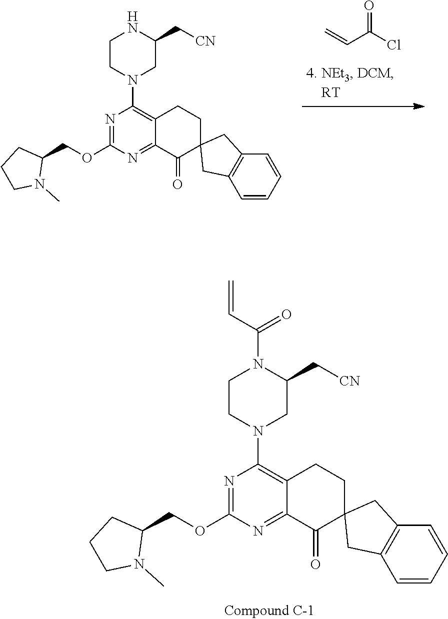

To a vial containing tert-butyl (S)-4-(2-chloro-8-oxo-5,6,7,8-tetrahydroquinazolin-4-yl)-2-(cyanomethyl)piperazine-1-carboxylate (300 mg, 0.74 mmol) and 1,2-bis(bromomethyl)benzene (195 mg, 0.74 mmol) in THF (7.2 mL) was added potassium tert-butoxide (183 mg, 1.63 mmol). The reaction was stirred at room temperature overnight. Upon completion, saturated NH₄Cl (15 mL, aq.) was added and the mixture was extracted with DCM (10 mL*3). The combined organics were dried with Na₂SO₄, filtered, and concentrated in vacuo. The crude product was purified using flash column chromatography on silica gel (20→100% EtOAc in Hexanes) to yield tert-butyl (S)-4-(2′-chloro-8′-oxo-1,3,5′,8′-tetrahydro-6′H-spiro[indene-2,7′-quinazolin]-4′-yl)-2-(cyanomethyl)piperazine-1-carboxylate (151 mg, 0.30 mmol, 40% yield) as an orange oil.

¹H NMR (400 MHz, Chloroform-d) δ 7.38 (dd, J=7.4, 1.4 Hz, 1H), 7.28 (td, J=7.5, 1.3 Hz, 1H), 7.20 (t, J=7.4 Hz, 1H), 7.12 (d, J=7.4 Hz, 1H), 4.05 (s, 1H), 3.56 (dddd, J=15.7, 9.6, 6.4, 2.6 Hz, 4H), 3.50-3.40 (m, 2H), 3.35 (ddd, J=12.5, 7.2, 2.9 Hz, 2H), 3.22 (dd, J=15.9, 7.2 Hz, 1H), 2.85 (ddd, J=16.9, 11.8, 5.5 Hz, 1H), 2.76 (dd, J=12.8, 7.5 Hz, 1H), 2.57 (dd, J=16.3, 1.8 Hz, 2H), 2.02-1.93 (m, 1H), 1.64-1.49 (m, 2H), 1.47 (s, 9H) ppm LCMS: [M+H]⁺ m/z=508.2/510.2 amu

To a cooled (0° C.) vial containing NaH (14 mg, 0.35 mmol, 60% mineral oil dispersion) was added THE (0.5 mL), followed by (S)-(1-methylpyrrolidin-2-yl)methanol (90 μL, 0.74 mmol). The mixture was stirred for 45 minutes, at which point tert-butyl (S)-4-(2′-chloro-8′-oxo-1,3,5′,8′-tetrahydro-6′H-spiro[indene-2,7′-quinazolin]-4′-yl)-2-(cyanomethyl)piperazine-1-carboxylate (75 mg, 0.15 mmol), as a solution in THE (1 mL), was added. The mixture was warmed to room temperature and stirred for 3 hours. Upon completion, the reaction was quenched with saturated NH₄Cl (5 mL, aq.) and the mixture was extracted with DCM (10 mL*3). The combined organics were dried with Na₂SO₄, filtered, and concentrated in vacuo. The crude tert-butyl (S)-2-(cyanomethyl)-4-(2′-(((S)-1-methylpyrrolidin-2-yl)methoxy)-8′-oxo-1,3,5′,8′-tetrahydro-6′H-spiro[indene-2,7′-quinazolin]-4′-yl)piperazine-1-carboxylate was taken on to the next step without further purification.

LCMS: [M+H]⁺ m/z=587.3 amu

To a vial containing crude tert-butyl (S)-2-(cyanomethyl)-4-(2′-(((S)-1-methylpyrrolidin-2-yl)methoxy)-8′-oxo-1,3,5′,8′-tetrahydro-6′H-spiro[indene-2,7′-quinazolin]-4′-yl)piperazine-1-carboxylate (30 mg, 0.05 mmol, est.) in DCM (0.5 mL) was added H₃PO₄ (20 μL, 0.33 mmol) dropwise. The reaction was stirred at room temperature for 3 hours, at which point H₂O (1 mL) was added and the solution was made basic by slow addition of 2 M NaOH solution (aq.). Once basic, the mixture was extracted with DCM (2 mL*3), and the combined organics were dried with Na₂SO₄, filtered, and concentrated in vacuo. The crude 2-((S)-4-(2′-(((S)-1-methylpyrrolidin-2-yl)methoxy)-8′-oxo-1,3,5′,8′-tetrahydro-6′H-spiro[indene-2,7′-quinazolin]-4′-yl)piperazin-2-yl) was taken on to the next step without further purification.

LCMS: [M+H]⁺ m/z=487.3 amu

To a cooled (0° C.) solution of containing crude 2-((S)-4-(2′-(((S)-1-methylpyrrolidin-2-yl)methoxy)-8′-oxo-1,3,5′,8′-tetrahydro-6′H-spiro[indene-2,7′-quinazolin]-4′-yl)piperazin-2-yl) (25 mg, 0.05 mmol, est.) in DCM (0.6 mL) was added triethylamine (70 μL, 0.51 mmol), followed by a 0.2 M solution of prop-2-enoyl chloride (1.02 mL, 0.20 mmol) in DCM. The mixture was warmed to room temperature and stirred for 1.5 hours, at which point the solution was concentrated, taken up in DMSO, filtered and purified using preparative HPLC (C18, 20→50% MeCN in H₂O+0.25% TFA). The combine fractions containing the desired product were lyophilized to yield compound C-1, 2-((S)-1-acryloyl-4-(2′-(((S)-1-methylpyrrolidin-2-yl)methoxy)-8′-oxo-1,3,5′,8′-tetrahydro-6′H-spiro[indene-2,7′-quinazolin]-4′-yl)piperazin-2-yl)acetonitrile (4.4 mg, 1.12 mmol, 20% yield, over 3 steps), as a light brown solid.

¹H NMR (400 MHz, DMSO-d₆, TFA salt) δ 10.39 (bs, 1H), 7.28-7.11 (m, 4H), 6.87 (s, 1H), 6.61 (bs, 3H), 6.20 (dd, J=16.7, 2.3 Hz, 1H), 5.79 (dd, J=10.4, 2.3 Hz, 1H), 4.66 (ddd, J=12.8, 9.1, 2.7 Hz, 1H), 4.49 (ddd, J=13.0, 6.4, 2.6 Hz, 1H), 4.17-3.97 (m, 2H), 3.76 (bs, 2H), 3.45-3.06 (m, 8H), 3.06-2.86 (m, 5H), 2.30-1.92 (m, 4H), 1.92-1.75 (m, 2H) ppm LCMS: [M+H]⁺ m/z=541.3 amu

### Synthesis of Compound C-2

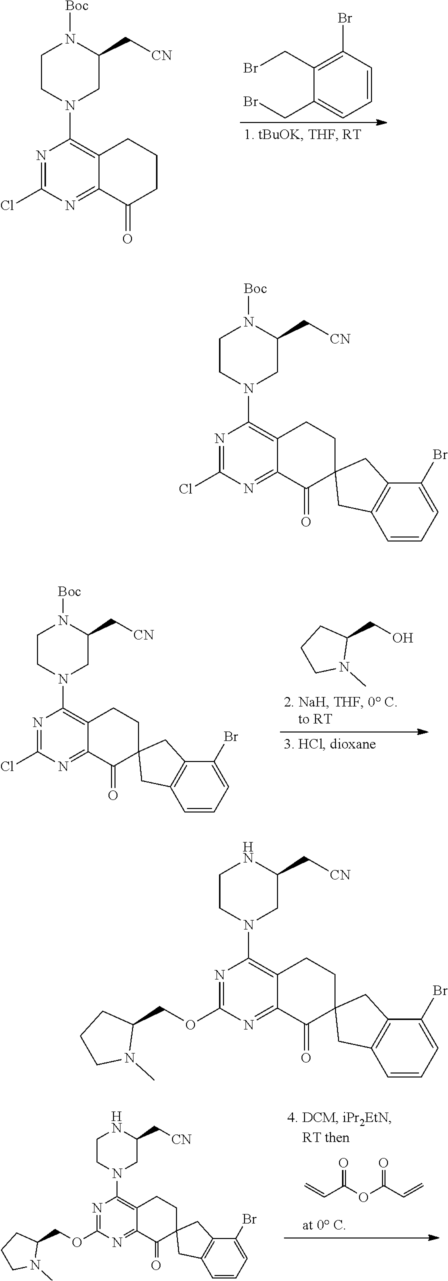

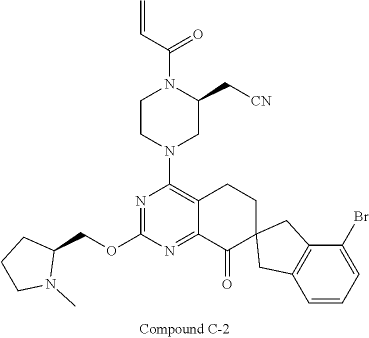 [SMILES: C=CC(=O)N1CCN(c2nc(OC[C@@H]3CCCN3C)nc3c2CCC2(Cc4cccc(Br)c4C2)C3=O)C[C@@H]1CC#N]

1-Bromo-2,3-bis(bromomethyl)benzene (127 mg, 0.37 mmol) and intermediate 4-2 (150 mg, 0.37 mmol) were dissolved in anhydrous THF (7.4 mL) and treated with KOtBu (124 mg, 1.11 mmol). The mixture was stirred for 9 hours, then partitioned between EtOAc and H₂O, and the organic phase was collected and washed with brine, dried over Na₂SO₄, concentrated, and purified by flash column chromatography on silica gel (10→30% EtOAc in hexanes) to give tert-butyl (2S)-4-(4-bromo-2′-chloro-8′-oxo-1,3,5′,8′-tetrahydro-6′H-spiro[indene-2,7′-quinazolin]-4′-yl)-2-(cyanomethyl)piperazine-1-carboxylate (39.2 mg, 18% yield) as a faintly yellow film.

LCMS: [M+H]⁺ m/z=586.1/588.1 amu (1:1)

1-Methyl-L-prolinol (21.78 mg, 0.19 mmol) was dissolved in anhydrous THF (400 μL) and treated with NaH (4.5 mg, 0.11 mmol), and the mixture was aged for 30 minutes, then added to a dry residue of tert-butyl (2S)-4-(4-bromo-2′-chloro-8′-oxo-1,3,5′,8′-tetrahydro-6′H-spiro[indene-2,7′-quinazolin]-4′-yl)-2-(cyanomethyl)piperazine-1-carboxylate (22.2 mg, 0.04 mmol). The mixture was stirred for 24 hours, then partitioned between EtOAc and 1:1 brine:1M NaOH. The organic phase was collected and washed with brine, dried over K₂CO₃, concentrated, and purified by flash column chromatography on silica gel (2→3% MeOH in DCM+1% Et₃N) to give tert-butyl (2S)-4-(4-bromo-2′-(((S)-1-methylpyrrolidin-2-yl)methoxy)-8′-oxo-1,3,5′,8′-tetrahydro-6′H-spiro[indene-2,7′-quinazolin]-4′-yl)-2-(cyanomethyl)piperazine-1-carboxylate (20.2 mg, 80% yield) as a faintly yellow film.

¹H NMR (400 MHz, Acetonitrile-d3, major diastereomer) δ 7.60-7.57 (m, 1H), 7.46 (dt, J=7.6, 1.2 Hz, 1H), 7.20-7.10 (m, 1H), 4.58 (d, J=4.4 Hz, 1H), 4.35 (ddd, J=21.3, 10.9, 5.0 Hz, 1H), 4.16 (dt, J=11.0, 6.1 Hz, 1H), 4.07-3.84 (m, 2H), 3.24 (dd, J=13.6, 3.9 Hz, 1H), 3.09-2.96 (m, 4H), 2.96-2.74 (m, 7H), 2.69-2.51 (m, 2H), 2.41 (s, 3H), 2.31-2.22 (m, 1H), 2.05-1.90 (m, 3H), 1.79-1.64 (m, 3H), 1.46 (d, J=2.8 Hz, 9H) ppm

LCMS: [M+H]⁺ m/z=619.2/621.2 amu (1:1)

tert-Butyl (2S)-4-(4-bromo-2′-(((S)-1-methylpyrrolidin-2-yl)methoxy)-8′-oxo-1,3,5′,8′-tetrahydro-6′H-spiro[indene-2,7′-quinazolin]-4′-yl)-2-(cyanomethyl)piperazine-1-carboxylate was treated with HCl and 4N in dioxane (500 μL), and the mixture was aged at room temperature (RT) for 20 minutes, then concentrated. The residue was treated with anhydrous DCM (300 μL) and iPr₂EtN (53 μL, 0.30 mmol), and stirred at room temperature for 24 hours, then cooled to 0° C. and treated with acrylic anhydride (4.2 μL, 0.04 mmol). After 15 minutes, the mixture was concentrated and purified by preparative HPLC (C18, 10→70% ACN in H₂O+0.25% TFA) to give compound C-2, 2-((2S)-1-acryloyl-4-(4-bromo-2′-(((S)-1-methylpyrrolidin-2-yl)methoxy)-8′-oxo-1,3,5′,8′-tetrahydro-6′H-spiro[indene-2,7′-quinazolin]-4′-yl)piperazin-2-yl)acetonitrile (2.8 mg, 15% yield), as a colorless film.

¹H NMR (500 MHz, CDCl₃) δ 7.57 (dt, J=8.1, 1.4 Hz, 1H), 7.35 (dt, J=7.4, 1.4 Hz, 1H), 7.09 (t, J=7.7 Hz, 1H), 6.44 (dt, J=17.3, 1.5 Hz, 1H), 6.16 (ddd, J=17.3, 10.4, 1.5 Hz, 1H), 5.86 (dt, J=10.4, 1.5 Hz, 1H), 4.34 (td, J=4.8, 1.5 Hz, 2H), 4.24 (s, 1H), 3.78 (td, J=4.9, 1.5 Hz, 4H), 3.64 (td, J=5.9, 1.5 Hz, 2H), 3.33-2.47 (m, 10H), 2.17-2.00 (m, 2H), 2.00-1.52 (m, 9H) ppm

LCMS: [M+H]⁺ m/z=619.2/621.2 amu (1:1)

### Synthesis of Compound C-3

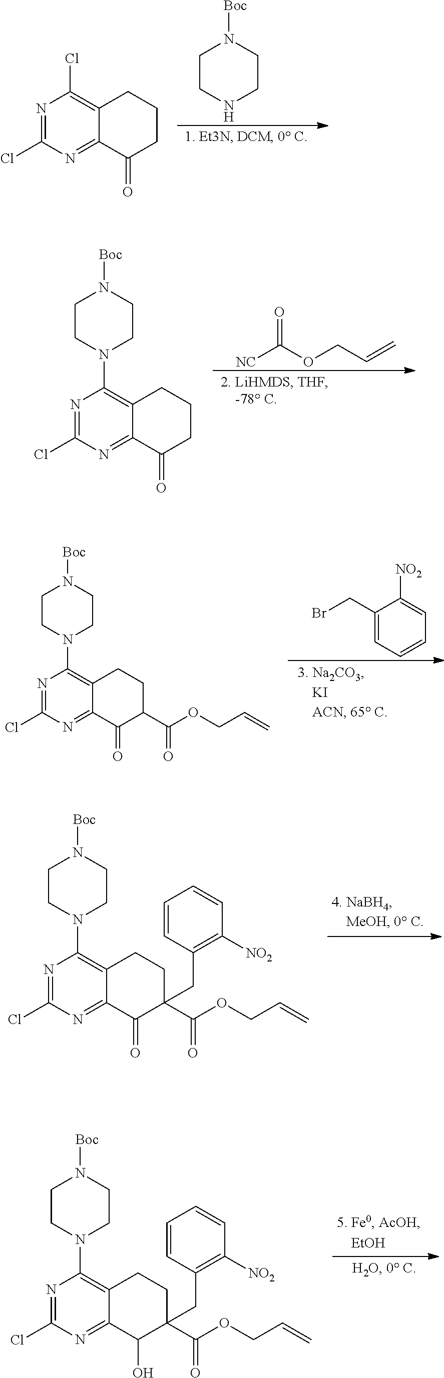

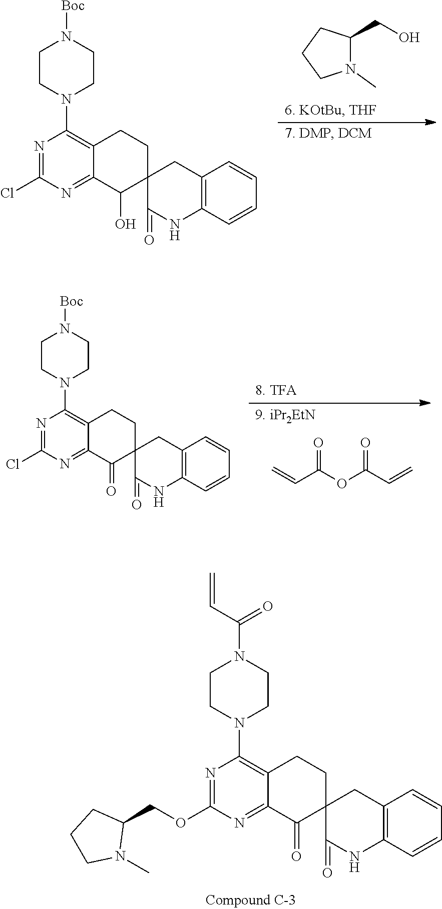

2,4-Dichloro-6,7-dihydro-5H-quinazolin-8-one (1085 mg, 5 mmol) was dissolved in anhydrous DCM (20 mL) and the mixture was cooled to 0° C. then treated with tert-butyl piperazine-1-carboxylate (931 mg, 5 mmol) and Et₃N (1.39 mL, 10 mmol). After 70 minutes, the mixture was diluted with DCM and washed with half-saturated NaHCO₃, brine, dried over Na₂SO₄, concentrated, and purified by flash column chromatography on silica gel (2→4% MeOH in DCM) to give tert-butyl 4-(2-chloro-8-oxo-5,6,7,8-tetrahydroquinazolin-4-yl)piperazine-1-carboxylate (1.811 g, 4.94 mmol, 99% yield) as a pale yellow foam (Rf=0.34 (96:4 CHCl₃:MeOH)).

¹H NMR (500 MHz, Chloroform-d) δ 3.60-3.52 (m, 8H), 2.81-2.73 (m, 4H), 2.15-2.07 (m, 2H), 1.49 (s, 9H) ppm

LCMS: [M+H]⁺ m/z=367.1/369.1 amu (3:1)

tert-Butyl 4-(2-chloro-8-oxo-5,6,7,8-tetrahydroquinazolin-4-yl)piperazine-1-carboxylate (500 mg, 1.36 mmol) was dissolved in anhydrous THF (6.8 mL) then cooled to −78° C. and treated with LiHMDS, 1.0 M in THE (1.77 mL, 1.77 mmol) followed by allyl cyanoformate (269 μL, 2.04 mmol). The mixture was stirred for 1 hour then quenched with sat NH₄Cl and partitioned between sat NH₄Cl and EtOAc. The organic phase was collected and washed with brine, dried over Na₂SO₄, filtered, concentrated, and purified by flash column chromatography on silica gel (0→50% EtOAc in hexanes) to give allyl 4-(4-(tert-butoxycarbonyl)piperazin-1-yl)-2-chloro-8-oxo-5,6,7,8-tetrahydroquinazoline-7-carboxylate (436.8 mg, 0.969 mmol, 71% yield) as a pale yellow foam (Rf=0.29 (7:3 hexanes:EtOAc+1% AcOH)).

¹H NMR (400 MHz, Chloroform-d) δ 11.91 (s, 1H), 5.96 (ddt, J=17.3, 10.4, 5.7 Hz, 1H), 5.36 (dq, J=17.2, 1.5 Hz, 1H), 5.28 (dq, J=10.5, 1.3 Hz, 1H), 4.73 (dt, J=5.7, 1.4 Hz, 2H), 3.59-3.49 (m, 4H), 3.45-3.37 (m, 4H), 2.70-2.60 (m, 2H), 2.55 (td, J=7.7, 2.1 Hz, 2H), 1.46 (s, 9H) ppm

¹³C NMR (101 MHz, CDCl₃) δ 171.02, 165.69, 161.53, 158.62, 156.49, 154.74, 131.65, 118.97, 116.99, 102.24, 80.42, 65.87, 47.97, 43.14 (br), 28.47, 23.54, 20.11 ppm

LCMS: [M+H]⁺ m/z=451.1 amu

Allyl 4-(4-(tert-butoxycarbonyl)piperazin-1-yl)-2-chloro-8-oxo-5,6,7,8-tetrahydroquinazoline-7-carboxylate (50 mg, 0.110 mmol) and 1-(bromomethyl)-2-nitro-benzene (29 mg, 0.13 mmol) were dissolved in anhydrous Toluene (550 μL) and treated with potassium tert-pentoxide, 1.7 M in toluene (78 uL, 0.13 mmol). The mixture was warmed to 65° C. and stirred for 24 hours and potassium tert-pentoxide, 1.7M in toluene (65 μL, 0.11 mmol) and 1-(bromomethyl)-2-nitro-benzene (24 mg, 0.11 mmol) were then added, and the reaction was stirred an additional 24 hours. The mixture was partitioned between EtOAc and H₂O and the organic phase was collected and washed with brine, dried over Na₂SO₄, concentrated, and purified by flash column chromatography on silica gel (0→40% Me₂CO in hexanes) to give allyl 4-(4-(tert-butoxycarbonyl)piperazin-1-yl)-2-chloro-7-(2-nitrobenzyl)-8-oxo-5,6,7,8-tetrahydroquinazoline-7-carboxylate (33.7 mg, 0.0575 mmol, 52% yield) as a yellow film.

¹H NMR (400 MHz, Chloroform-d) δ 7.85 (dd, J=8.1, 1.4 Hz, 1H), 7.48 (ddd, J=8.7, 7.2, 1.5 Hz, 1H), 7.42 (dd, J=7.8, 1.7 Hz, 1H), 7.37 (ddd, J=8.2, 7.1, 1.7 Hz, 1H), 5.77 (ddt, J=17.2, 10.4, 5.7 Hz, 1H), 5.21 (dq, J=12.5, 1.4 Hz, 1H), 5.17 (dq, J=5.8, 1.2 Hz, 1H), 4.60 (ddt, J=13.2, 5.9, 1.3 Hz, 1H), 4.53 (ddt, J=13.1, 5.7, 1.4 Hz, 1H), 4.00 (d, J=14.1 Hz, 1H), 3.68 (d, J=14.2 Hz, 1H), 3.62-3.51 (m, 4H), 3.51-3.32 (m, 4H), 2.81 (ddd, J=17.1, 11.1, 4.4 Hz, 1H), 2.61 (dt, J=17.0, 4.3 Hz, 1H), 2.48 (dt, J=13.7, 4.2 Hz, 1H), 1.84 (ddd, J=13.7, 11.1, 4.4 Hz, 1H), 1.46 (s, 9H) ppm

¹³C NMR (101 MHz, CDCl₃) δ 190.91, 169.86, 167.09, 159.38, 154.63, 150.62, 133.96, 133.00, 130.95, 130.72, 128.40, 124.99, 122.28, 119.58, 80.59, 66.72, 59.23, 48.02, 43.45, 34.82, 30.24, 28.45, 23.38 ppm

LCMS: [M+H]⁺ m/z=586.2 amu

Allyl 4-(4-(tert-butoxycarbonyl)piperazin-1-yl)-2-chloro-7-(2-nitrobenzyl)-8-oxo-5,6,7,8-tetrahydroquinazoline-7-carboxylate (8.4 mg, 0.014 mmol) was dissolved in MeOH (500 μL) and cooled to 0° C., and NaBH₄ (50 μL, 20 mg/mL, 0.029 mmol) was added as a stock solution in MeOH. The mixture was stirred for 5 minutes, quenched with AcOH (150 μL), concentrated and then co-evaporated from CHCl₃ to give the crude allyl 4-(4-(tert-butoxycarbonyl)piperazin-1-yl)-2-chloro-8-hydroxy-7-(2-nitrobenzyl)-5,6,7,8-tetrahydroquinazoline-7-carboxylate, which was carried forward without purification.

¹H NMR (400 MHz, CDCl₃, major diastereomer) δ 7.83 (dd, J=8.1, 1.5 Hz, 1H), 7.56-7.45 (m, 2H), 7.45-7.30 (m, 1H), 5.81 (ddt, J=17.4, 10.4, 5.9 Hz, 1H), 5.32-5.19 (m, 2H), 4.68-4.48 (m, 2H), 4.45-4.37 (m, 1H), 3.77 (d, J=14.4 Hz, 1H), 3.68-3.30 (m, 10H), 2.55-2.41 (m, 2H), 2.29-2.14 (m, 1H), 1.75-1.60 (m, 1H), 1.46 (d, J=2.3 Hz, 9H) ppm

¹³C NMR (101 MHz, CDCl₃) δ 176.76, 173.07, 166.23, 165.61, 157.89, 154.82, 151.05, 133.37, 132.68, 131.52, 128.25, 124.89, 119.21, 113.75, 80.48, 71.77, 65.98, 51.10, 47.89, 34.34, 28.51, 25.61, 22.86, 21.04 ppm

LCMS: [M+H]⁺ m/z=588.2/590.2 amu (3:1)

The crude allyl 4-(4-(tert-butoxycarbonyl)piperazin-1-yl)-2-chloro-8-hydroxy-7-(2-nitrobenzyl)-5,6,7,8-tetrahydroquinazoline-7-carboxylate (14.1 mg, 0.020 mmol, est.) was dissolved in EtOH (335 μL) and H₂O (84 μL), and treated with iron powder (13.4 mg, 0.240 mmol) and AcOH (6.8 μL, 0.120 mmol). The mixture was warmed to 65° C. for 30 minutes, then was cooled, diluted with EtOAc, filtered through a thin pad of silica gel, and concentrated to give tert-butyl 4-(2-chloro-8-hydroxy-2′-oxo-1′,4′,5,8-tetrahydro-2′H,6H-spiro[quinazoline-7,3′-quinolin]-4-yl)piperazine-1-carboxylate (11.1 mg, 22.2 μmol, 93% yield) as a faintly yellow film (Rf=0.37 (major), 0.53 (minor) (7:3 EtOAc:hexanes)).

LCMS: [M+H]⁺ m/z=500.2/502.2 amu

1-Methyl-L-prolinol (12 mg, 0.10 mmol) was dissolved in anhydrous THF (470 μL) and treated with KOtBu, 1.7 M in THE (47 μL, 0.08 mmol), and the mixture was stirred for 5 minutes. This solution was added to a dry residue of tert-butyl 4-(2-chloro-8-hydroxy-2′-oxo-1′,4′,5,8-tetrahydro-2′H,6H-spiro[quinazoline-7,3′-quinolin]-4-yl)piperazine-1-carboxylate (10 mg, 0.020 mmol). After 1 hour, the reaction was diluted with 1 M NaOH and extracted with EtOAc (3 times). The combined extract was washed with brine, dried over Na₂SO₄, and concentrated to give the crude tert-butyl 4-(8-hydroxy-2-(((S)-1-methylpyrrolidin-2-yl)methoxy)-2′-oxo-1′,4′,5,8-tetrahydro-2′H,6H-spiro[quinazoline-7,3′-quinolin]-4-yl)piperazine-1-carboxylate (13.2 mg, >100% yield) as a brown film, which was used in the next step without purification.

LCMS: [M+H]⁺ m/z=579.3 amu

The crude tert-butyl 4-(8-hydroxy-2-(((S)-1-methylpyrrolidin-2-yl)methoxy)-2′-oxo-1′,4′,5,8-tetrahydro-2′H,6H-spiro[quinazoline-7,3′-quinolin]-4-yl)piperazine-1-carboxylate (13.2 mg, 0.020 mmol, est.) was dissolved in DCM (460 μL) and treated with Dess-Martin periodinane (19.2 mg, 0.050 mmol). After 30 minutes, the reaction was quenched with iPrOH (2 drops), stirred for 10 minutes, and concentrated. The residue was dissolved in 94:6 CHCl₃:MeOH+1% Et₃N, and filtered through a short column of silica gel eluting with the same to give the crude tert-butyl 4-(2-(((S)-1-methylpyrrolidin-2-yl)methoxy)-2′,8-dioxo-1′,4′,5,8-tetrahydro-2′H,6H-spiro[quinazoline-7,3′-quinolin]-4-yl)piperazine-1-carboxylate (13.2 mg, 100% yield) as a pale brown oily residue, which was used in the next step without purification.

LCMS: [M+H]⁺ m/z=577.3/579.3 amu (3:1)

The crude tert-butyl 4-(2-(((S)-1-methylpyrrolidin-2-yl)methoxy)-2′,8-dioxo-1′,4′,5,8-tetrahydro-2′H,6H-spiro[quinazoline-7,3′-quinolin]-4-yl)piperazine-1-carboxylate (13.2 mg, 0.020 mmol, est.) was treated with TFA (50 μL) for 20 minutes, then concentrated and co-evaporated from DCM once and further dried under vacuum. The residue was dissolved in anhydrous MeCN (200 μL) and treated with iPr₂EtN (12 μL, 0.070 mmol) and acrylic anhydride (1.3 μL, 0.010 mmol). After 1 hour, the mixture was concentrated, re-dissolved in ACN:H₂O (1:1), and purified by preparative HPLC (C18, 5→70% ACN in H₂O+0.25% TFA) to give compound C-3, 4-(4-acryloylpiperazin-1-yl)-2-(((S)-1-methylpyrrolidin-2-yl)methoxy)-1′,4′,5,6-tetrahydro-2′H,8H-spiro[quinazoline-7,3′-quinoline]-2′,8-dione (2.04 mg, 3.8 μmol, 17% yield), as a faintly yellow film.

¹H NMR (400 MHz, CDCl₃) δ 7.91 (d, J=3.9 Hz, 1H), 7.25-7.19 (m, 1H), 7.12-7.03 (m, 1H), 6.85-6.77 (m, 1H), 6.58 (ddd, J=16.8, 10.5, 1.1 Hz, 1H), 6.34 (dt, J=16.8, 1.9 Hz, 1H), 5.77 (dt, J=10.5, 1.9 Hz, 1H), 4.82-4.68 (m, 1H), 3.99-3.54 (m, 7H), 3.10 (s, 3H), 2.93 (d, J=29.4 Hz, 1H), 2.73 (dd, J=26.6, 16.0 Hz, 2H), 2.44-2.25 (m, 2H), 2.25-2.02 (m, 2H), 1.90 (t, J=11.6 Hz, 1H), 1.71-1.51 (m, 1H), 1.50-1.37 (m, 1H), 0.96-0.78 (m, 6H) ppm

LCTOF: [M+H]⁺ m/z=531.2715 amu (calculated for C₂₉H₂₅N₆O₄⁺=531.2714).

### Synthesis of Compound C-4

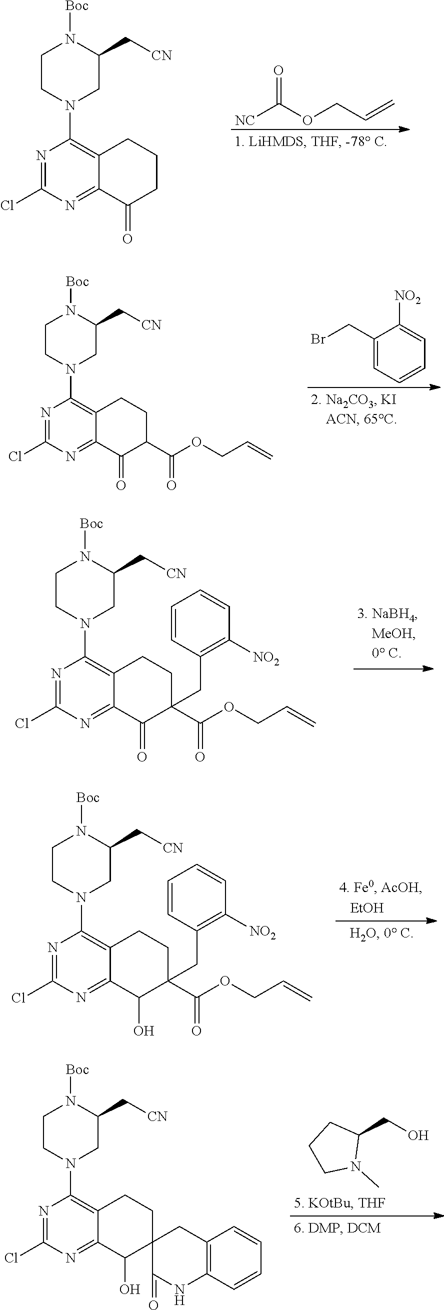

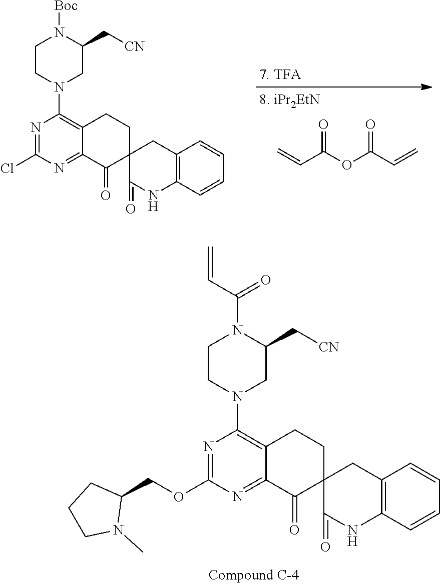

Tert-butyl (S)-4-(2-chloro-8-oxo-5,6,7,8-tetrahydroquinazolin-4-yl)-2-(cyanomethyl)piperazine-1-carboxylate (2.11 g, 5.2 mmol) and anhydrous THF (52 mL) were cooled to −78° C. and treated with LHMDS 1.0 M in THE (6.8 mL, 6.8 mmol). After 5 minutes, allyl cyanoformate (1025 μL, 7.8 mmol) was added. HPLC analysis (t=19:50) of an aliquot diluted with MeOH/AcOH showed high conversion to a major product. The reaction was quenched by the addition of saturated NaHCO₃ then partitioned between EtOAc and saturated NaHCO₃. The organic phase was collected and washed with saturated NaHCO₃, brine, dried over Na₂SO₄, concentrated, and purified by flash column chromatography on silica gel (10→70% EtOAc in hexanes) to give allyl (S)-4-(4-(tert-butoxycarbonyl)-3-(cyanomethyl)piperazin-1-yl)-2-chloro-8-hydroxy-5,6-dihydroquinazoline-7-carboxylate (1.477 g, 3.02 mmol, 58% yield) as a pale pink solid.

¹H NMR (400 MHz, Chloroform-d) δ 11.95 (s, 1H), 5.98 (ddt, J=17.2, 10.5, 5.7 Hz, 1H), 5.38 (dq, J=17.2, 1.5 Hz, 1H), 5.31 (dq, J=10.4, 1.2 Hz, 1H), 4.75 (dt, J=5.7, 1.5 Hz, 2H), 4.58 (d, J=7.8 Hz, 1H), 4.15-4.02 (m, 1H), 3.98 (dt, J=13.8, 2.1 Hz, 1H), 3.83-3.76 (m, 1H), 3.31 (dd, J=13.8, 4.0 Hz, 1H), 3.06 (td, J=12.3, 3.5 Hz, 1H), 2.81-2.49 (m, 7H), 1.50 (s, 9H) ppm

LCMS: [M+H]⁺ m/z=490.2/492.2 amu (3:1)

Allyl (S)-4-(4-(tert-butoxycarbonyl)-3-(cyanomethyl)piperazin-1-yl)-2-chloro-8-hydroxy-5,6-dihydroquinazoline-7-carboxylate (200 mg, 0.41 mmol), 1-(bromomethyl)-2-nitro-benzene (176 mg, 0.82 mmol), NaI (122 mg, 0.82 mmol), and Na₂CO₃ (173 mg, 1.6 mmol) were suspended in anhydrous MeCN (1.4 mL) and warmed to 80° C. After 5 hours, the mixture was poured into H₂O and extracted with EtOAc (2 times) and the combined extract was washed with brine, dried over Na₂SO₄, concentrated, and purified by flash column chromatography on silica gel (0→60% EtOAc in hexanes) to give allyl 4-((S)-4-(tert-butoxycarbonyl)-3-(cyanomethyl)piperazin-1-yl)-2-chloro-7-(2-nitrobenzyl)-8-oxo-5,6,7,8-tetrahydroquinazoline-7-carboxylate (199 mg, 0.319 mmol, 78% yield, Rf=0.34 (1:1 hexanes:EtOAc)).

¹H NMR (400 MHz, CDCl₃, major diastereomer) δ 7.86 (dd, J=8.1, 1.6 Hz, 1H), 7.55-7.33 (m, 3H), 5.76 (ddq, J=17.4, 10.4, 5.9 Hz, 1H), 5.26-5.13 (m, 2H), 4.55 (dt, J=5.5, 1.4 Hz, 3H), 4.23-3.76 (m, 4H), 3.67 (d, J=1.1 Hz, 1H), 3.25 (ddd, J=12.7, 7.0, 3.8 Hz, 1H), 3.15 (s, 1H), 3.05 (ddd, J=12.8, 11.2, 3.8 Hz, 1H), 2.96-2.79 (m, 2H), 2.76-2.57 (m, 2H), 2.54-2.44 (m, 1H), 1.96-1.79 (m, 1H), 1.48 (s, 9H) ppm

Allyl 4-((S)-4-(tert-butoxycarbonyl)-3-(cyanomethyl)piperazin-1-yl)-2-chloro-7-(2-nitrobenzyl)-8-oxo-5,6,7,8-tetrahydroquinazoline-7-carboxylate (40 mg, 0.064 mmol) was dissolved in MeOH (640 uL), cooled to 0° C., and treated with NaBH₄ (4.8 mg, 0.13 mmol). After 15 minutes, the reaction was quenched with AcOH (1 drop) and concentrated to give the crude allyl 4-((S)-4-(tert-butoxycarbonyl)-3-(cyanomethyl)piperazin-1-yl)-2-chloro-8-hydroxy-7-(2-nitrobenzyl)-5,6,7,8-tetrahydroquinazoline-7-carboxylate, which was carried forward without purification.

LCMS: [M+H]⁺ m/z=627.2/629.2 amu (3:1)

The crude allyl 4-((S)-4-(tert-butoxycarbonyl)-3-(cyanomethyl)piperazin-1-yl)-2-chloro-8-hydroxy-7-(2-nitrobenzyl)-5,6,7,8-tetrahydroquinazoline-7-carboxylate (40.13 mg, 0.064 mmol) was dissolved in EtOH (600 μL) and H₂O (200 μL), then treated with iron powder (35.7 mg, 0.64 mmol) and AcOH (18.3 μL, 0.32 mmol) and warmed to 65° C. After 40 minutes, the mixture was cooled, diluted with EtOAc, filtered through a short column of silica gel, and concentrated to give the crude tert-butyl (2S)-4-(2-chloro-8-hydroxy-2′-oxo-1′,4′,5,8-tetrahydro-2′H,6H-spiro[quinazoline-7,3′-quinolin]-4-yl)-2-(cyanomethyl)piperazine-1-carboxylate (37.8 mg, >100% yield) as a pale-yellow film, which was carried forward without purification.

LCMS: [M+H]⁺ m/z=539.2 amu

1-Methyl-L-prolinol (37 mg, 0.32 mmol) was dissolved in THE (1.2 mL) and treated with KOtBu, 1.7 M in THE (150 μL, 0.256 mmol). The mixture was stirred for 5 min then added to a dry residue of the crude tert-butyl (2S)-4-(2-chloro-8-hydroxy-2′-oxo-1′,4′,5,8-tetrahydro-2′H,6H-spiro[quinazoline-7,3′-quinolin]-4-yl)-2-(cyanomethyl)piperazine-1-carboxylate (34.5 mg, 0.064 mmol), and the mixture was stirred at 0° C. for 20 minutes, then at room temperature for 40 minutes. The mixture was partitioned between 1M NaOH and DCM and the aqueous phase was extracted twice more with DCM. The combined extract was washed with brine, dried over K₂CO₃, filtered, and concentrated to give the crude tert-butyl (2S)-2-(cyanomethyl)-4-(8-hydroxy-2-(((S)-1-methylpyrrolidin-2-yl)methoxy)-2′-oxo-1′,4′,5,8-tetrahydro-2′H,6H-spiro[quinazoline-7,3′-quinolin]-4-yl)piperazine-1-carboxylate (38.9 mg, 98% yield) as an oily residue, which was carried forward without purification.

LCMS: [M+H]⁺ m/z=618.3 amu

The crude tert-butyl (2S)-2-(cyanomethyl)-4-(8-hydroxy-2-(((S)-1-methylpyrrolidin-2-yl)methoxy)-2′-oxo-1′,4′,5,8-tetrahydro-2′H,6H-spiro[quinazoline-7,3′-quinolin]-4-yl)piperazine-1-carboxylate (38.9 mg, 0.0600 mmol, est.) was dissolved in DCM (700 μL) and treated with Dess-Martin periodinane (39.8 mg, 0.090 mmol) at room temperature. After 1.5 hours, the mixture was dissolved in aqueous H₃PO₄ and washed with Et₂O (2 times) then basified with K₂CO₃ and back-extracted with EtOAc (3 times). The combined extract was washed with brine, dried over Na₂SO₄, filtered, and concentrated to give the crude tert-butyl (2S)-2-(cyanomethyl)-4-(2-(((S)-1-methylpyrrolidin-2-yl)methoxy)-2′,8-dioxo-1′,4′,5,8-tetrahydro-2′H,6H-spiro[quinazoline-7,3′-quinolin]-4-yl)piperazine-1-carboxylate (33.7 mg, 87% yield) as an amber colored residue, which was carried forward without purification.

LCMS: [M+H]⁺ m/z=616.3 amu

The crude tert-butyl (2S)-2-(cyanomethyl)-4-(2-(((S)-1-methylpyrrolidin-2-yl)methoxy)-2′,8-dioxo-1′,4′,5,8-tetrahydro-2′H,6H-spiro[quinazoline-7,3′-quinolin]-4-yl)piperazine-1-carboxylate (27.4 mg, 0.040 mmol, est.) was treated with 4N HCl in dioxane (200 μL). After 35 minutes, the mixture was concentrated and co-evaporated from MeOH, then re-suspended in anhydrous MeCN (445 μL) and treated with iPr₂EtN (39 μL, 0.22 mmol) and acrylic anhydride (6.2 μL, 0.050 mmol). After 35 minutes, the reaction was concentrated, reconstituted in ACN/H₂O, and purified by prep HPLC (C18, 5→70% ACN in H₂O+0.25% TFA) to give the compound C-4, 2-((2S)-1-acryloyl-4-(2-(((S)-1-methylpyrrolidin-2-yl)methoxy)-2′,8-dioxo-1′,4′,5,8-tetrahydro-2′H,6H-spiro[quinazoline-7,3′-quinolin]-4-yl)piperazin-2-yl)acetonitrile (2.4 mg, 10% yield) as a white film.

¹H NMR (400 MHz, CDCl₃, mixture of diastereomers) δ 7.86 (d, J=5.3 Hz, 1H), 7.18-7.07 (m, 2H), 7.02-6.90 (m, 1H), 6.72 (d, J=7.7 Hz, 1H), 6.50 (dd, J=16.7, 11.2 Hz, 1H), 6.31 (dd, J=16.8, 2.0 Hz, 1H), 5.76 (dd, J=10.5, 1.9 Hz, 1H), 4.45 (d, J=11.0 Hz, 1H), 4.27 (dd, J=10.7, 5.8 Hz, 1H), 3.97 (dd, J=13.9, 2.4 Hz, 1H), 3.92-3.77 (m, 2H), 3.27-3.08 (m, 2H), 2.93-2.56 (m, 6H), 2.48 (s, 3H), 2.35-2.18 (m, 2H), 2.09-1.94 (m, 1H), 1.87-1.71 (m, 4H), 0.87-0.74 (m, 4H) ppm

LCTOF: [M+H]⁺ m/z=570.2814 amu (calculated for C₃₁H₃₆N₇O₄ 570.2823 amu)

### Synthesis of Compound C-5

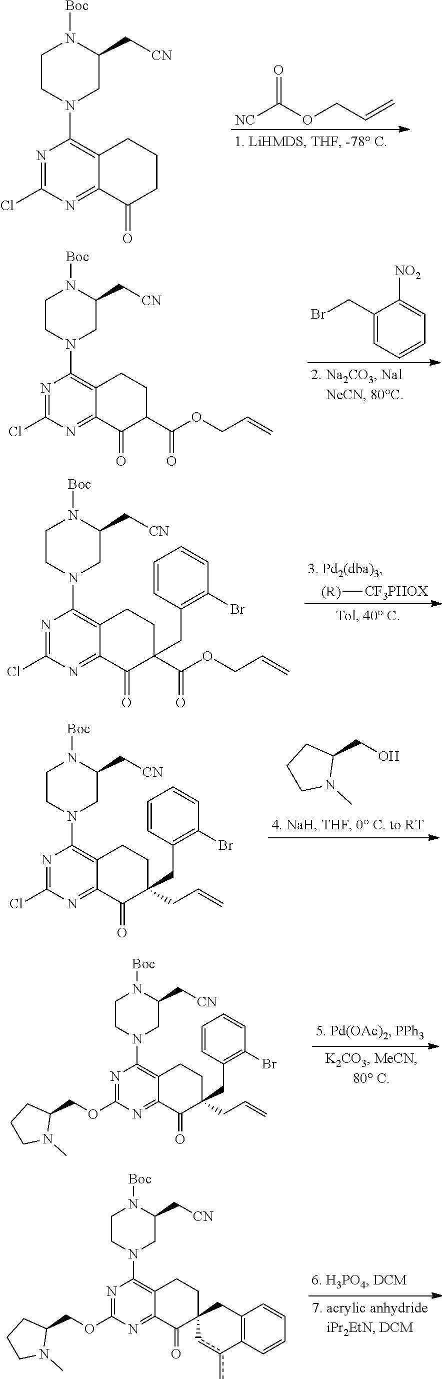

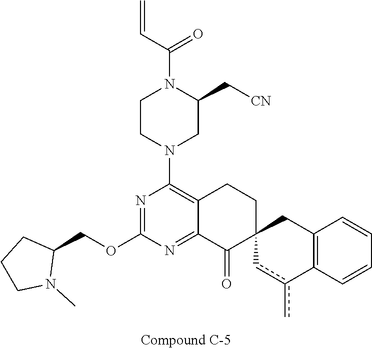

To a cooled (−78° C.) solution of tert-butyl (S)-4-(2-chloro-8-oxo-5,6,7,8-tetrahydroquinazolin-4-yl)-2-(cyanomethyl)piperazine-1-carboxylate (1.0 g, 2.5 mmol) in THF (25 mL) was added LiHMDS (3.2 mL, 3.2 mmol, 1 M in THF) dropwise. the reaction was stirred for 5 minutes before allyl cyanoformate (0.39 mL, 3.7 mmol) was added. The mixture was stirred for 2 hours, after which point the reaction was quenched using sat. NH₄Cl (50 mL, aq.) and warmed to room temperature. The mixture was extracted using DCM and the combined organics were dried with Na₂SO₄, filtered, and concentrated in vacuo. The crude product was purified using flash column chromatography on silica gel (0→15% EtOAc in hexanes) to yield allyl 4-((S)-4-(tert-butoxycarbonyl)-3-(cyanomethyl)piperazin-1-yl)-2-chloro-8-oxo-5,6,7,8-tetrahydroquinazoline-7-carboxylate (547 mg, 1.12 mmol, 45% yield) as a pale yellow solid.

LCMS: [M+H]⁺ m/z=490.2 amu

To a vial containing allyl 4-((S)-4-(tert-butoxycarbonyl)-3-(cyanomethyl)piperazin-1-yl)-2-chloro-8-oxo-5,6,7,8-tetrahydroquinazoline-7-carboxylate (233 mg, 0.48 mmol) and 1-bromo-2-(bromomethyl)benzene (0.16 mL, 1.2 mmol) in MeCN (4.8 mL) was added Na₂CO₃ (141 mg, 1.9 mmol) and NaI (143 mg, 0.96 mmol). The mixture was heated to 60° C. and stirred overnight. Upon completion, the mixture was cooled to room temperature, filtered through a cotton plug rinsing with DCM, concentrated in vacuo, and purified using flash column chromatography on silica gel (0→70% EtOAc in hexanes) to yield allyl 7-(2-bromobenzyl)-4-((S)-4-(tert-butoxycarbonyl)-3-(cyanomethyl)piperazin-1-yl)-2-chloro-8-oxo-5,6,7,8-tetrahydroquinazoline-7-carboxylate (217 mg, 0.33 mmol, 69% yield) as a white solid.

¹H NMR (400 MHz, Chloroform-d) δ 7.51 (dd, J=7.9, 1.2 Hz, 1H), 7.24 (ddd, J=7.8, 4.0, 1.8 Hz, 1H), 7.16 (td, J=7.5, 1.3 Hz, 1H), 7.05 (td, J=7.7, 1.8 Hz, 1H), 5.82 (dddt, J=17.2, 10.4, 6.8, 5.7 Hz, 1H), 5.31-5.15 (m, 2H), 4.69-4.47 (m, 3H), 4.22-3.88 (m, 2H), 3.81-3.45 (m, 3H), 3.30-3.14 (m, 1H), 3.10-2.54 (m, 6H), 1.97-1.80 (m, 1H), 1.76-1.54 (m, 1H), 1.48 (d, J=4.2 Hz, 10H) ppm

LCMS: [M+H]⁺ m/z=658.1/660.1 amu

To an oven-dried vial containing allyl 7-(2-bromobenzyl)-4-((S)-4-(tert-butoxycarbonyl)-3-(cyanomethyl)piperazin-1-yl)-2-chloro-8-oxo-5, 6,7,8-tetrahydroquinazoline-7-carboxylate (219 mg, 0.33 mmol) was added Pd₂(dba)₃ (15 mg, 0.02 mmol) and (R)-α-(CF₃)₃-t-BuPHOX (39 mg, 0.07 mmol), followed by toluene (11 mL). The headspace was purged with argon and the vial was capped. The mixture was stirred at room temperature for 30 minutes before being warmed to 40° C. and stirring overnight. Upon completion, the mixture was cooled, diluted with DCM (5 mL), and filtered through a plug of celite, which was washed with more DCM (20 mL). The solvent was removed in vacuo and the crude product was purified using flash column chromatography on silica gel (0→50% EtOAc in hexanes) to yield tert-butyl (S)-4-((R)-7-allyl-7-(2-bromobenzyl)-2-chloro-8-oxo-5,6,7,8-tetrahydroquinazolin-4-yl)-2-(cyanomethyl)piperazine-1-carboxylate (183 mg, 0.30 mmol, 92% yield) as an off white solid.

LCMS: [M+H]⁺ m/z=614.2/616.2 amu

To a cooled (0° C.) vial containing NaH (24 mg, 0.60 mmol, 60% mineral oil dispersion) was added THE (1 mL) followed by (S)-(1-methylpyrrolidin-2-yl)methanol (142 μL, 1.20 mmol). The mixture was stirred for 45 minutes, at which point tert-butyl (S)-4-((R)-7-allyl-7-(2-bromobenzyl)-2-chloro-8-oxo-5,6,7,8-tetrahydroquinazolin-4-yl)-2-(cyanomethyl)piperazine-1-carboxylate (147 mg, 0.24 mmol), as a solution in THE (1.4 mL), was added. The mixture was warmed to room temperature and stirred for 3 hours. Upon completion, the reaction was quenched with saturated NH₄Cl (10 mL, aq.) and the mixture was extracted with DCM (10 mL*3). The combined organics were dried with Na₂SO₄, filtered, and concentrated in vacuo. The crude tert-butyl (S)-4-((R)-7-allyl-7-(2-bromobenzyl)-2-(((S)-1-methylpyrrolidin-2-yl)methoxy)-8-oxo-5,6,7,8-tetrahydroquinazolin-4-yl)-2-(cyanomethyl)piperazine-1-carboxylate was taken on to the next step without further purification.

LCMS: [M+H]⁺ m/z=693.2 amu

To an oven-dried vial containing the crude tert-butyl (S)-4-((R)-7-allyl-7-(2-bromobenzyl)-2-(((S)-1-methylpyrrolidin-2-yl)methoxy)-8-oxo-5,6,7,8-tetrahydroquinazolin-4-yl)-2-(cyanomethyl)piperazine-1-carboxylate (99 mg, 0.14 mmol, est.) was added K₂CO₃ (40 mg, 0.29 mmol), followed by PPh₃ (8 mg, 0.03 mmol) and finally Pd(OAc)₂ (3 mg, 0.01 mmol). The headspace was purged with argon, MeCN (4 mL) was added, and the vial was capped. The mixture was warmed to 80° C. and stirred overnight. Upon completion, the mixture was cooled, diluted with DCM (5 mL), and filtered through a plug of celite, which was washed with more DCM (20 mL). The solvent was removed in vacuo and the crude tert-butyl (S)-2-(cyanomethyl)-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate was used in the next step without further purification.

LCMS: [M+H]⁺ m/z=613.3 amu

To a vial containing the crude tert-butyl (S)-2-(cyanomethyl)-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate (4.2 mg, 0.007 mmol, est.) in DCM (0.2 mL) was added H₃PO₄ (5 μL, 0.07 mmol) dropwise. The reaction was stirred at room temperature for 3 hours, at which point H₂O (1 mL) was added and the solution was made basic by slow addition of 2 M NaOH solution (aq.). Once basic, the mixture was extracted with DCM (2 mL*3), and the combined organics were dried with Na₂SO₄, filtered, and concentrated in vacuo. The crude 2-((S)-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile was taken on to the next step without further purification.

LCMS: [M+H]⁺ m/z=513.3 amu

To a cooled (0° C.) solution of the 2-((S)-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (3 mg, 0.006 mmol, est.) in DCM (0.3 mL) was added N,N-diisopropylethylamine (10 μL, 0.09 mmol), followed by acrylic anhydride (6 μL, 0.05 mmol). The mixture was warmed to room temperature and stirred for 2 hours, at which point the solution was concentrated in vacuo, taken up in DMSO, filtered and purified using preparative HPLC (C18, 20→60% MeCN in H₂O+0.25% TFA). The combine fractions containing the desired product were lyophilized to yield compound C-5, 2-((S)-1-acryloyl-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (0.8 mg, 0.001 mmol, 4% yield, over 4 steps), as a light brown solid and as a mixture of exo and endo olefin isomers.

¹H NMR of the product was consistent with reported diagnostic peaks for the epimer LCMS: [M+H]⁺ m/z=567.3 amu

### Synthesis of Compound C-6

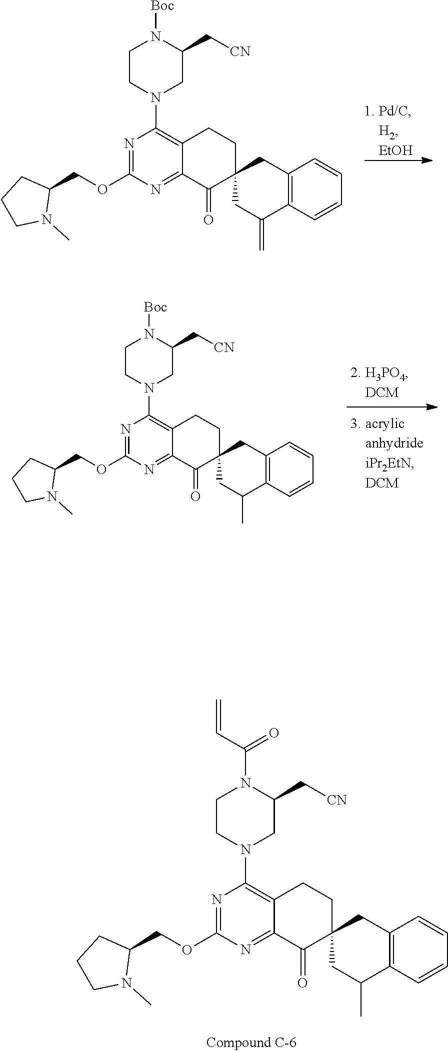

To a vial containing tert-butyl (S)-2-(cyanomethyl)-4-((R)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate (20 mg, 0.03 mmol, crude est.) in ethanol (0.5 mL) was added 10% palladium on carbon (7 mg, 0.007 mmol). The vial was sealed and placed under a hydrogen atmosphere using a balloon. The reaction was vigorously stirred overnight. Upon completion, the reaction mixture was diluted with DCM (2 mL) and filtered through a plug of celite, washing with more DCM (10 mL). The solvent was removed in vacuo and the crude tert-butyl (2S)-2-(cyanomethyl)-4-((2R)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate was used in the next step without further purification.

LCMS: [M+H]⁺ m/z=615.3 amu

To a vial containing the crude tert-butyl (2S)-2-(cyanomethyl)-4-((2R)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate (20 mg, 0.03 mmol, est.) in DCM (0.7 mL) was added H₃PO₄ (20 μL, 0.33 mmol) dropwise. The reaction was stirred at room temperature for 2 hours, at which point H₂O (2 mL) was added and the solution was made basic by slow addition of 2 M NaOH solution (aq.). Once basic, the mixture was extracted with DCM (2 mL*3), and the combined organics were dried with Na₂SO₄, filtered, and concentrated in vacuo. The crude 2-((2S)-4-((2R)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile was taken on to the next step without further purification.

LCMS: [M+H]⁺ m/z=515.3 amu

To a cooled (0° C.) solution of the crude 2-((2S)-4-((2R)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (17 mg, 0.03 mmol, est.) in DCM (0.4 mL) was added N,N-diisopropylethylamine (57 μL, 0.33 mmol), followed by acrylic anhydride (20 μL, 0.17 mmol). The mixture was warmed to room temperature and stirred for 2 hours, at which point the solution was concentrated in vacuo, taken up in DMSO, filtered, and purified using preparative HPLC (C18, 20→60% MeCN in H₂O+0.25% TFA). The combined fractions containing the desired product were lyophilized to yield compound C-6, 2-((2S)-1-acryloyl-4-((2R)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (2.6 mg, 0.005 mmol, 14% yield, over 5 steps), as a fluffy white solid and mixture of epimers at the benzylic methyl center.

¹H NMR of the product mixture was consistent with reported diagnostic peaks for the epimer

LCMS: [M+H]⁺ m/z=569.3 amu

### Synthesis of Compound C-7

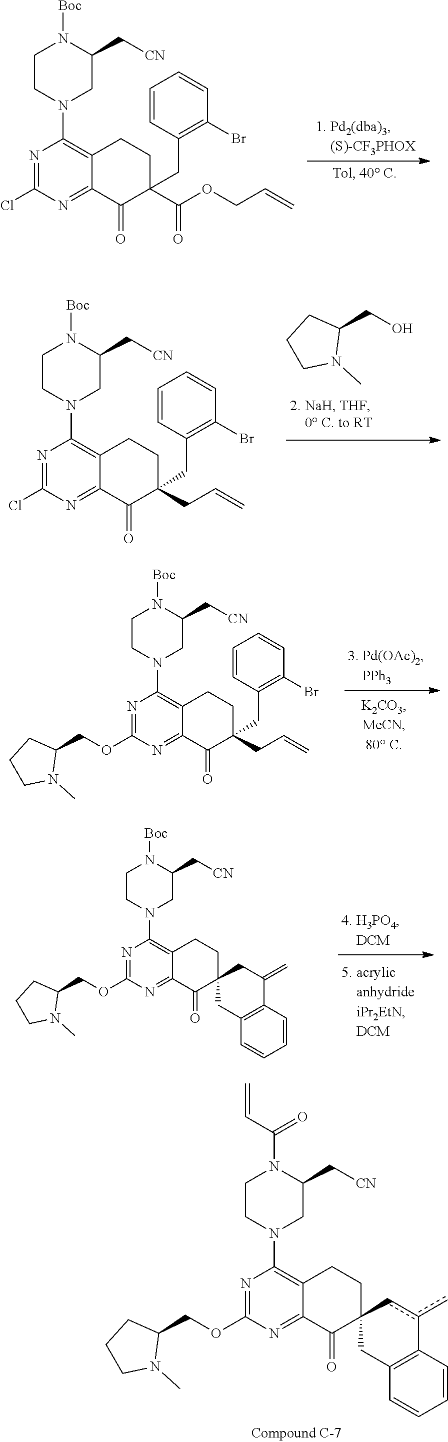

To an oven-dried vial containing allyl 7-(2-bromobenzyl)-4-((S)-4-(tert-butoxycarbonyl)-3-(cyanomethyl)piperazin-1-yl)-2-chloro-8-oxo-5,6,7,8-tetrahydroquinazoline-7-carboxylate (297 mg, 0.44 mmol) was added Pd₂(dba)₃ (20 mg, 0.02 mmol) and (S)-p-(CF₃)₃-t-BuPHOX (52 mg, 0.09 mmol), followed by toluene (15 mL). The headspace was purged with argon and the vial was capped. The mixture was stirred at room temperature for 30 minutes before being warmed to 40° C. and stirred overnight. Upon completion, the mixture was cooled, diluted with DCM (15 mL), and filtered through a plug of celite, which was washed with more DCM (30 mL). The solvent was removed in vacuo and the crude product was purified using flash column chromatography on silica gel (0→50% EtOAc in hexanes) to yield tert-butyl (S)-4-((S)-7-allyl-7-(2-bromobenzyl)-2-chloro-8-oxo-5,6,7,8-tetrahydroquinazolin-4-yl)-2-(cyanomethyl)piperazine-1-carboxylate (233 mg, 0.38 mmol, 86% yield) as an off white solid.

LCMS: [M+H]⁺ m/z=614.1/616.1 amu

Tert-butyl (S)-4-((S)-7-allyl-7-(2-bromobenzyl)-2-chloro-8-oxo-5,6,7,8-tetrahydroquinazolin-4-yl)-2-(cyanomethyl)piperazine-1-carboxylate and the crude products produced by subsequent steps 2 through 4 were carried forward using the procedures and reagents detailed for the synthesis of compound C-5. For the last step, the combined fractions containing the desired product were lyophilized to yield compound C-7, 2-((S)-1-acryloyl-4-((S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (7 mg, 0.013 mmol, 19% yield, over 4 steps), as a fluffy pale yellow solid and mixture of exo and endo olefin isomers.

¹H NMR, reporting diagnostic peaks (21H of 41H) of the major isomer of the complex mixture: (400 MHz, DMSO-d₆, TFA salt) δ 10.31 (s, 1H), 7.37-7.17 (m, 4H), 6.96-6.76 (m, 1H), 6.19 (dd, J=16.7, 2.3 Hz, 1H), 5.88 (d, J=1.7 Hz, 1H), 5.79 (dd, J=10.3, 2.3 Hz, 1H), 4.95 (s, 1H), 4.78 (s, 1H), 4.66 (dd, J=13.0, 2.8 Hz, 1H), 4.51 (dd, J=12.9, 6.4 Hz, 1H), 3.84-3.73 (m, 1H), 3.57 (dd, J=11.7, 5.9 Hz, 1H), 2.96 (d, J=4.5 Hz, 3H), 2.06 (d, J=1.5 Hz, 3H) ppm

LCMS: [M+H]⁺ m/z=567.3 amu

### Synthesis of Compound C-15

To a cooled (0° C.) solution of the crude 2-((S)-4-((S)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (35 mg, 0.07 mmol, est.) in DCM (1.4 mL) was added N,N-diisopropylethylamine (120 μL, 0.68 mmol), followed by 2-fluoroacrylic anhydride (55 mg, 0.34 mmol). The mixture was warmed to room temperature and stirred for 2 hours, at which point the solution was concentrated in vacuo, taken up in DMSO, filtered, and purified using preparative HPLC (C18, 25→65% MeCN in H₂O+0.25% TFA). The combine fractions containing the desired product were lyophilized to yield compound C-15, 2-((S)-1-(2-fluoroacryloyl)-4-((S)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (13.6 mg, 0.023 mmol, 34% yield, over 4 steps), as a fluffy white solid and as a mixture of exo and endo olefin isomers.

¹H NMR; internal olefin reported: (400 MHz, Acetonitrile-d₃, TFA salt) δ 12.18 (bs, 1H), 7.41-7.19 (m, 4H), 5.85 (t, J=1.5 Hz, 1H), 5.37-5.14 (m, 2H), 4.84 (bs, 1H), 4.68 (dd, J=14.3, 1.2 Hz, 1H), 4.53 (dd, J=14.3, 5.9 Hz, 1H), 4.24 (dt, J=14.1, 2.3 Hz, 1H), 4.19-4.03 (m, 2H), 3.72-3.50 (m, 2H), 3.43 (dd, J=14.0, 3.7 Hz, 1H), 3.35 (d, J=15.8 Hz, 1H), 3.32-3.18 (m, 1H), 3.18-3.06 (m, 1H), 3.03-2.70 (m, 8H), 2.34-2.21 (m, 1H), 2.15-1.98 (m, 5H), 1.91-1.75 (m, 2H) ppm

LCMS: [M+H]⁺ m/z=585.3 amu

### Synthesis of Compound C-8

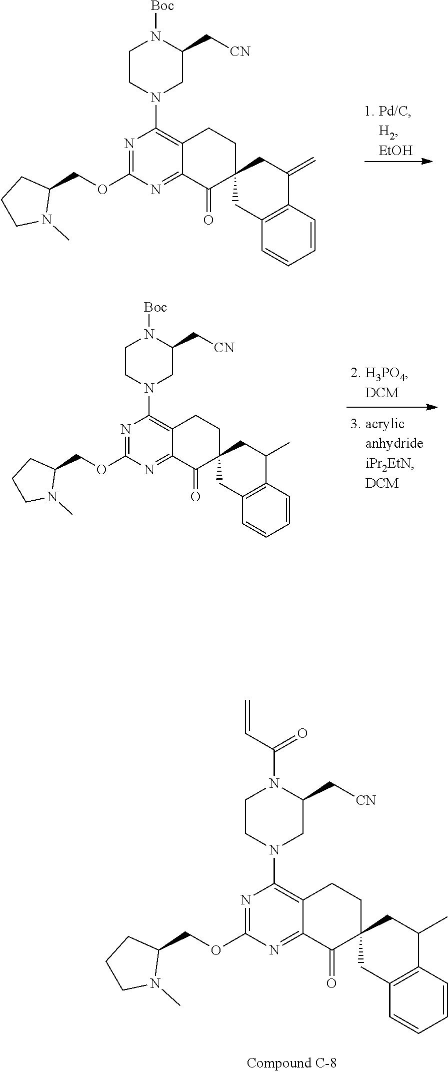

To a vial containing the crude tert-butyl (S)-2-(cyanomethyl)-4-((S)-4-methylene-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate (84 mg, 0.14 mmol, est.) in ethanol (3.5 mL) was added 10% palladium on carbon (29 mg, 0.03 mmol). The vial was sealed and placed under a hydrogen atmosphere using a balloon. The reaction was vigorously stirred overnight. Upon completion, the reaction mixture was diluted with DCM (5 mL) and filtered through a plug of celite, washing with more DCM (20 mL). The solvent was removed in vacuo and the crude tert-butyl (S)-2-(cyanomethyl)-4-((S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate was used in the next step without further purification.

LCMS: [M+H]⁺ m/z=615.3 amu

The crude tert-butyl (S)-2-(cyanomethyl)-4-((S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazine-1-carboxylate from the previous step and the crude product produced by subsequent step 2 were carried forward using the procedures and reagents detailed for the synthesis of compound C-6. For the last step, the combined fractions containing the desired product were lyophilized to yield compound C-8, 2-((S)-1-acryloyl-4-((S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (4.7 mg, 0.008 mmol, 12% yield, over 5 steps), as a fluffy off-white solid and as a mixture of epimers at the benzylic methyl center.

¹H NMR, reporting diagnostic peaks (18H of 41H) of the complex mixture: (400 MHz, DMSO-d₆, TFA salt) δ 10.37 (broad d, J=68.4 Hz, 1H), 7.47-6.99 (m, 4H), 6.97-6.76 (m, 1H), 6.20 (d, J=16.6 Hz, 1H), 5.79 (d, J=10.5 Hz, 1H), 4.94 (bs, 1H), 4.78 (bs, 1H), 4.65 (ddd, J=21.7, 13.0, 2.7 Hz, 1H), 4.49 (td, J=13.1, 6.3 Hz, 1H), 2.95 (dd, J=21.2, 4.6 Hz, 3H), 1.31 (dd, J=10.8, 6.7 Hz, 3H) ppm

LCMS: [M+H]⁺ m/z=569.3 amu

### Synthesis of Compound C-16

To a cooled (0° C.) solution of the crude 2-((S)-4-((S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (35 mg, 0.07 mmol, est.) in DCM (1.4 mL) was added N,N-diisopropylethylamine (120 μL, 0.68 mmol), followed by 2-fluoroacrylic anhydride (55 mg, 0.34 mmol). The mixture was warmed to room temperature and stirred for 2 hours, at which point the solution was concentrated in vacuo, taken up in DMSO, filtered, and purified using preparative HPLC (C18, 25→65% MeCN in H₂O+0.25% TFA). The combine fractions containing the desired product were lyophilized to yield compound C-16, 2-((2S)-1-(2-fluoroacryloyl)-4-((2S)-4-methyl-2′-(((S)-1-methylpyrrolidin-2-yl)methoxy)-8′-oxo-3,4,5′,8′-tetrahydro-1H,6′H-spiro[naphthalene-2,7′-quinazolin]-4′-yl)piperazin-2-yl)acetonitrile (6.3 mg, 0.010 mmol, 16% yield, over 5 steps), as a fluffy white solid and as a mixture of epimers at the benzylic methyl center.

¹H NMR reported as a mixture of epimers at the methyl center (400 MHz, Acetonitrile-d₃, TFA salt) δ 12.26 (bs, 1H), 7.45-6.97 (m, 4H), 5.42-5.06 (m, 2H), 4.83 (bs, 1H), 4.66 (ddd, J=16.1, 14.3, 1.2 Hz, 1H), 4.51 (dt, J=14.2, 6.2 Hz, 1H), 4.41-3.79 (m, 6H), 3.79-3.30 (m, 5H), 3.30-2.56 (m, 11H), 2.44-2.15 (m, 2H), 2.15-1.98 (m, 2H), 1.88-1.76 (m, 1H), 1.35 (dd, J=11.1, 6.8 Hz, 3H) ppm

LCMS: [M+H]⁺ m/z=587.3 amu

### Example 5: Synthesis of Compounds C-9 through C-14

### Synthesis of Intermediate 5-1

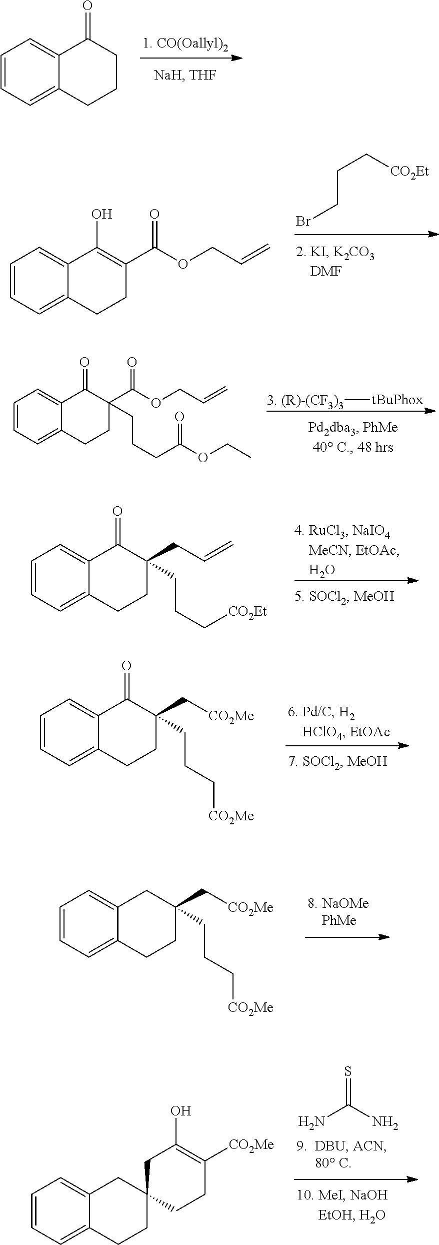

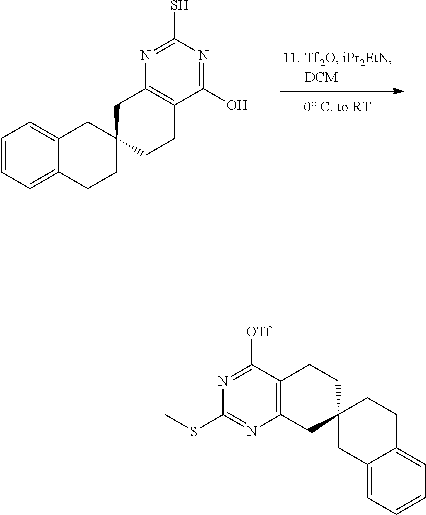 [SMILES: Oc1nc(S)nc2c1CC[C@@]1(CCc3ccccc3C1)C2 ; CSc1nc2c(c(OS(=O)(=O)C(F)(F)F)n1)CC[C@@]1(CCc3ccccc3C1)C2]

### Intermediate 5-1

NaH (2.74 g, 68 mmol) was suspended in anhydrous THF (45 mL) and cooled to 0° C. Tetralin-1-one (3.64 mL, 27 mmol) was added and the mixture was warmed to room temperature and treated with diallyl carbonate (5.89 mL, 41 mmol). The mixture was stirred for 12 hours then carefully quenched by the addition of sat NH₄Cl then extracted with EtOAc (3 times). The combined extract was washed with brine, dried over Na₂SO₄, filtered through a thin pad of silica gel, and concentrated. The residue was purified by flash column chromatography on silica gel (0→15% EtOAc in hexanes) to give allyl 1-hydroxy-3,4-dihydronaphthalene-2-carboxylate (6.211 g, 26.97 mmol, 99% yield) as a colorless oil.

LCTOF: [M+H]⁺ m/z=231.1019 amu

Allyl 1-hydroxy-3,4-dihydronaphthalene-2-carboxylate (2.98 g, 13 mmol) and ethyl 4-bromobutanoate (2.78 mL, 19 mmol) were dissolved in anhydrous DMF (39.8 mL) and treated with and K₂CO₃ (3.58 g, 26 mmol), and the mixture was stirred at 50° C. for 4 hours. The mixture was poured into H₂O and extracted with EtOAc (3 times) and the combined extract was washed sequentially with dilute Na₂S₂O₃, brine, dried over Na₂SO₄, and concentrated. The residue was purified by flash column chromatography on silica gel (0→25% EtOAc in hexanes) to give allyl 2-(4-ethoxy-4-oxobutyl)-1-oxo-1,2,3,4-tetrahydronaphthalene-2-carboxylate (3.27 g, 9.49 mmol, 73% yield).

¹H NMR (400 MHz, CDCl₃) δ 8.00 (dd, J=7.9, 1.5 Hz, 1H), 7.43 (td, J=7.5, 1.5 Hz, 1H), 7.30-7.24 (m, 1H), 7.18 (d, J=7.8 Hz, 1H), 5.77 (ddt, J=17.2, 10.8, 5.5 Hz, 1H), 5.16-5.07 (m, 2H), 4.55 (ddt, J=5.6, 3.2, 1.5 Hz, 2H), 4.07 (qd, J=7.1, 1.8 Hz, 2H), 3.04 (ddd, J=17.5, 9.5, 4.8 Hz, 1H), 2.92 (dt, J=17.5, 5.3 Hz, 1H), 2.56 (ddd, J=13.7, 5.7, 4.6 Hz, 1H), 2.34-2.27 (m, 2H), 2.16 (ddd, J=13.9, 9.6, 4.9 Hz, 1H), 2.03-1.84 (m, 2H), 1.79-1.59 (m, 2H), 1.25-1.16 (m, 3H) ppm

LCMS: [M+H]⁺ m/z=345.1 amu

Anhydrous toluene was sparged with N2 for 20 minutes before use. A flame dried 250 mL round bottom flask was charged with (R)-p-(CF₃)₃-t-BuPHOX (449 mg, 0.76 mmol) and Pd₂(dba)₃ (261 mg, 0.28 mmol), then evacuated and backfilled with N2 (3 times). Toluene (80 mL) was added and the mixture was stirred for 30 minutes at room temperature. Separately, allyl 2-(4-ethoxy-4-oxobutyl)-1-oxo-1,2,3,4-tetrahydronaphthalene-2-carboxylate (3.27 g, 9.5 mmol) was dissolved in toluene (40 mL) and sparged for 20 minutes, then added to the catalyst mixture and stirring continued for 15 hours. The reaction was opened to air and amended with a small amount of silica gel and stirred for 5 minutes, then filtered through a thin pad of silica gel rinsing with 8:2 hexanes:EtOAc. The filtrate was concentrated and purified by flash column chromatography on silica gel (0→15% EtOAc in hexanes) to give ethyl (R)-4-(2-allyl-1-oxo-1,2,3,4-tetrahydronaphthalen-2-yl)butanoate (2.91 g, 9.69 mmol, >100% yield) as a yellow oil.

¹H NMR (400 MHz, CDCl₃) δ 7.89 (dd, J=7.8, 1.6 Hz, 1H), 7.31 (td, J=7.5, 1.5 Hz, 1H), 7.18-7.11 (m, 1H), 7.07 (dd, J=7.7, 0.9 Hz, 1H), 5.69-5.57 (m, 1H), 4.96-4.89 (m, 2H), 3.95 (q, J=7.1 Hz, 2H), 2.84 (t, J=6.4 Hz, 2H), 2.35 (ddt, J=13.9, 7.1, 1.3 Hz, 1H), 2.22-2.16 (m, 1H), 2.13 (t, J=7.1 Hz, 2H), 1.91 (t, J=6.4 Hz, 2H), 1.64-1.36 (m, 4H), 1.07 (t, J=7.2 Hz, 3H) ppm

¹³C NMR (101 MHz, CDCl₃) δ 201.12, 173.41, 143.17, 134.00, 133.17, 131.84, 128.75, 128.06, 126.70, 118.25, 60.31, 47.66, 39.10, 34.70, 33.76, 30.79, 25.10, 19.44, 14.28 ppm

LCMS: [M+H]⁺ m/z=301.2 amu

Ethyl (R)-4-(2-allyl-1-oxo-1,2,3,4-tetrahydronaphthalen-2-yl)butanoate (2.85 g, 9.5 mmol) was dissolved in MeCN (14 mL) and EtOAc (14 mL), then treated with H₂O (21 mL), NaIO₄ (10.15 g, 48 mmol), and RuCl₃.xH₂O (43 mg, 0.21 mmol) and stirred vigorously at room temperature. After 90 minutes, a second charge of NaIO₄ (2 g) was added. After an additional 30 minutes, the mixture was poured into 0.5 M NaHSO₄ and extracted with EtOAc (3 times). The combined extract was washed with brine, dried over Na₂SO₄, filtered through Celite, and concentrated. The residue was reconstituted in MeOH (48 mL) and treated with SOCl₂ (8.3 mL, 114 mmol) dropwise at 0° C. The mixture was warmed to room temperature and stirred for 7 hours, then quenched with H₂O, stirred for 15 minutes, then poured into H₂O and extracted with EtOAc (3 times). The combined extract was washed with sat. NaHCO₃, brine, dried over Na₂SO₄, and concentrated. The residue was purified by flash column chromatography on silica gel (0→30% EtOAc in hexanes) to give methyl (R)-4-(2-(2-methoxy-2-oxoethyl)-1-oxo-1,2,3,4-tetrahydronaphthalen-2-yl)butanoate (2.34 g, 7.36 mmol, 78% yield).

¹H NMR (400 MHz, CDCl₃) δ 8.03 (dd, J=7.9, 1.7 Hz, 1H), 7.44 (td, J=7.5, 1.5 Hz, 1H), 7.31-7.26 (m, 1H), 7.23-7.16 (m, 1H), 3.62 (s, 3H), 3.60 (s, 3H), 3.13-3.02 (m, 1H), 3.01-2.83 (m, 2H), 2.51 (d, J=15.9 Hz, 1H), 2.42 (ddd, J=13.7, 11.6, 5.1 Hz, 1H), 2.31-2.22 (m, 2H), 2.09-2.02 (m, 1H), 1.78-1.65 (m, 2H), 1.61-1.51 (m, 2H) ppm

¹³C NMR (101 MHz, CDCl₃) δ 200.15, 173.59, 172.07, 142.92, 133.37, 131.36, 128.79, 128.23, 126.81, 51.60, 46.83, 39.46, 34.13, 33.34, 30.60, 25.04, 19.46 ppm

LCMS: [M+H]⁺ m/z=319.1 amu

Methyl (R)-4-(2-(2-methoxy-2-oxoethyl)-1-oxo-1,2,3,4-tetrahydronaphthalen-2-yl)butanoate (2.34 g, 7.4 mmol) was dissolved in EtOAc (35 mL) and treated with HClO₄, 60% (120 uL, 1.1 mmol). Pd/C, 10 wt % (wetted) (460 mg) was added under N₂ atmosphere and the vessel was then charged with H₂ (4 times) and stirred vigorously at room temperature for 12 hours. The mixture was filtered through Celite, concentrated, further dried in vacuo, then taken up in MeOH (30 mL) and treated with SOCl₂ (5 mL, 68.92 mmol) at 0° C. and warmed to room temperature and stirred for 1 hour. The mixture was concentrated and the residue was purified by flash column chromatography on silica gel (5→35% EtOAc in hexanes) to give methyl (S)-4-(2-(2-methoxy-2-oxoethyl)-1,2,3,4-tetrahydronaphthalen-2-yl)butanoate (1.96 g, 6.44 mmol, 88% yield) as a colorless oil.

LC/MS, ESI [M+H]⁺=305.1 m/z.

A mixture of NaOMe (7.73 mL, 7.7 mmol) in anhydrous toluene (40 mL) was warmed to 100° C. and a solution of methyl (S)-4-(2-(2-methoxy-2-oxoethyl)-1,2,3,4-tetrahydronaphthalen-2-yl)butanoate (1.96 g, 6.4 mmol) in toluene (25 mL) was added dropwise over a period of approximately 60 minutes. Heating was continued for 4.5 hours after the mixture was cooled to room temperature and poured into sat. NH₄Cl and extracted with EtOAc (3 times). The combined extract was washed with brine, dried over Na₂SO₄, filtered through a thin pad of silica gel, and concentrated to give the crude methyl (S)-3-hydroxy-3′,4′-dihydro-1′H-spiro[cyclohexane-1,2′-naphthalen]-3-ene-4-carboxylate (1.78 g, >100% yield) as a faintly yellow oil, which was used in the next step without further purification.

¹H NMR (400 MHz, CDCl₃) δ 12.12 (s, 1H), 7.22-6.99 (m, 4H), 3.80-3.75 (m, 3H), 2.83 (t, J=6.8 Hz, 2H), 2.72-2.62 (m, 1H), 2.56 (d, J=16.3 Hz, 1H), 2.44-2.23 (m, 3H), 2.22-2.08 (m, 1H), 1.79-1.36 (m, 4H) ppm

¹³C NMR (101 MHz, CDCl₃) δ 172.98, 171.00, 135.75, 135.08, 129.79, 128.87, 125.88, 125.86, 96.77, 51.56, 40.09, 39.84, 33.12, 32.09, 31.73, 25.73, 19.41 ppm

LCMS: [M+Na]⁺ m/z=295.1 amu

The crude methyl (S)-3-hydroxy-3′,4′-dihydro-1′H-spiro[cyclohexane-1,2′-naphthalen]-3-ene-4-carboxylate (485.7 mg, 1.8 mmol, est.) was dissolved in anhydrous MeCN (8.9 mL) and treated with thiourea (163 mg, 2.1 mmol) and DBU (399 μL, 2.7 mmol) and the mixture was warmed to 80° C. for 18 hours, then cooled and concentrated to approximately 1 mL, then diluted into H₂O. The resulting solids were collected by filtration then re-dissolved in EtOH (3.6 mL) and treated with 1M NaOH (1.96 mL, 2.0 mmol) followed by MeI (122.1 uL, 2.0 mmol). The mixture was stirred vigorously at room temperature for 45 minutes then additional 1M NaOH (500 μL) and MeI (40 μL) were added, and after 12 hours the mixture was poured into aqueous NaH₂PO₄ and extracted with CHCl₃ (3 times). The combined extract was washed with brine, dried over Na₂SO₄, amended with 0.05 vol MeOH, filtered through a thin pad of silica gel rinsing with 95:5 CHCl₃:MeOH, and concentrated to give the crude (R)-2′-(methylthio)-3,4,5′,8′-tetrahydro-1H,6′H-spiro[naphthalene-2,7′-quinazolin]-4′-ol (495 mg, 1.58 mmol, 89% yield) as a white solid, which was used in the next step without further purification.

LCMS: [M+H]⁺ m/z=313.1 amu

The crude (R)-2′-(methylthio)-3,4,5′,8′-tetrahydro-1H,6′H-spiro[naphthalene-2,7′-quinazolin]-4′-ol (495 mg, 1.6 mmol, est.) was suspended in anhydrous DCM (3.2 mL) and treated with iPr₂EtN (552 μL, 3.2 mmol) and the mixture was cooled to 0° C., then triflic anhydride, 1M in DCM (2.38 mL, 2.4 mmol) was added dropwise. The cooling bath was removed and the mixture was stirred at room temperature for 2 hours. The mixture was diluted with 2 vol. hexanes, and filtered through a thin pad of silica gel rinsing with 9:1 hexanes:EtOAc. The residue was dissolved in DCM:hexanes and purified by flash column chromatography on silica gel (0→15% EtOAc in hexanes) to give intermediate 5-1, (R)-2′-(methylthio)-3,4,5′,8′-tetrahydro-1H,6′H-spiro[naphthalene-2,7′-quinazolin]-4′-yl trifluoromethanesulfonate (480 mg, 1.08 mmol, 68.1% yield) as a faintly yellow vitreous glass.

LCMS: [M+H]⁺ m/z=445.1 amu

### Synthesis of Intermediate 5-2

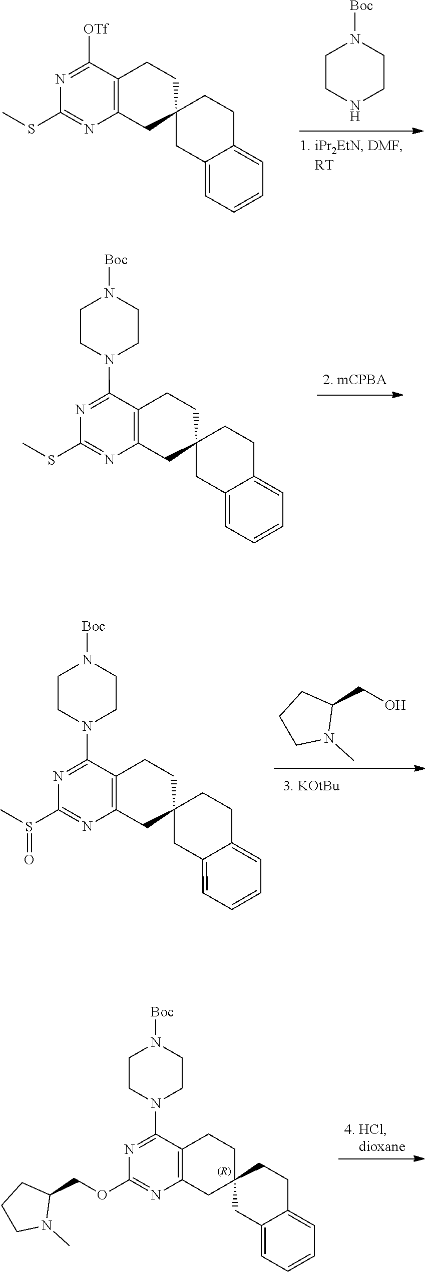

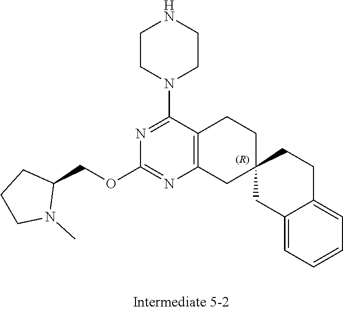

Intermediate 5-1, (R)-2′-(methylthio)-3,4,5′,8′-tetrahydro-1H,6′H-spiro[naphthalene-2,7′-quinazolin]-4′-yl trifluoromethanesulfonate (160 mg, 0.36 mmol) was dissolved in anhydrous DMF (1 mL) and treated with iPr₂EtN (0.19 mL, 1.1 mmol) and tert-butyl piperazine-1-carboxylate (74 mg, 0.40 mmol), and the mixture was stirred at room temperature overnight. The mixture was poured into sat. NaHCO₃ and extracted with EtOAc (3 times). The combined extract was washed with brine, dried over Na₂SO₄, filtered through a thin pad of silica gel, concentrated, and purified by flash column chromatography on silica gel (5→40% EtOAc in hexanes) to give tert-butyl (R)-4-(2′-(methylthio)-3,4,5′,8′-tetrahydro-1H,6′H-spiro[naphthalene-2,7′-quinazolin]-4′-yl)piperazine-1-carboxylate (162.8 mg, 0.339 mmol, 94% yield) as a white foam.

LCMS: [M+H]⁺ m/z=481.3 amu

tert-Butyl (R)-4-(2′-(methylthio)-3,4,5′,8′-tetrahydro-1H,6′H-spiro[naphthalene-2,7′-quinazolin]-4′-yl)piperazine-1-carboxylate (162.8 mg, 0.34 mmol) was dissolved in DCM, cooled to 0° C., and treated with mCPBA (101 mg, 0.44 mmol). The mixture was stirred for 30 minutes, then diluted with Et₂O (R_{f}=0.47 (Et₂O)), and washed with half-saturated NaHCO₃ (3 times), brine, then dried over Na₂SO₄, and concentrated to give the crude tert-butyl 4-((2R)-2′-(methylsulfinyl)-3,4,5′,8′-tetrahydro-1H,6′H-spiro[naphthalene-2,7′-quinazolin]-4′-yl)piperazine-1-carboxylate (169.6 mg, 0.342 mmol, 100% yield) as a white foam, which was used in the next step without further purification.

LCMS: [M+H]⁺ m/z=497.3 amu

1-Methyl-L-prolinol (79 mg, 0.68 mmol) was dissolved in anhydrous THF (2 mL) and treated with KOtBu, 1.7M in THE (400 μL, 0.68 mmol). The mixture was aged for 5 minutes, then added to a solution of the crude tert-butyl 4-((2R)-2′-(methylsulfinyl)-3,4,5′,8′-tetrahydro-1H,6′H-spiro[naphthalene-2,7′-quinazolin]-4′-yl)piperazine-1-carboxylate (169.6 mg, 0.34 mmol, est.) in anhydrous THF (1 mL) at 0° C. The mixture was stirred for 30 minutes then poured into aqueous K₂CO₃ and extracted with Et₂O (3 times). The combined extract was washed with brine, dried over Na₂SO₄, and concentrated to give the crude tert-butyl 4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (187.3 mg, 0.342 mmol, 100% yield) as a white foam, which was used in the next step without further purification.

LCMS: [M+H]⁺ m/z=548.4 amu

The crude tert-butyl 4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (187 mg, 0.34 mmol, est.) was treated with 4N HCl in dioxane (2.5 mL, 10 mmol) at room temperature for 1 hour. The mixture was concentrated then dissolved in 1N HCl and washed with Et₂O (2 times). The ethereal wash was extracted with 1N HCl once, and the combined aqueous was basified with K₂CO₃ and back-extracted with EtOAc (3 times). The combined extract was washed with brine, dried over K₂CO₃, filtered, and concentrated to give Intermediate 5-2, (R)-2′-(((S)-1-methylpyrrolidin-2-yl)methoxy)-4′-(piperazin-1-yl)-3,4,5′,8′-tetrahydro-1H,6′H-spiro[naphthalene-2,7′-quinazoline] (155.4 mg, 0.347 mmol, >100% yield), as a vitreous glass, which was used in the next step without further purification.

LCMS: [M+H]⁺ m/z=448.3 amu

### Synthesis of Compound C-9

Intermediate 5-2, (R)-2′-(((S)-1-methylpyrrolidin-2-yl)methoxy)-4′-(piperazin-1-yl)-3,4,5′,8′-tetrahydro-1H,6′H-spiro[naphthalene-2,7′-quinazoline] (77.7 mg, 0.17 mmol), was dissolved in anhydrous MeCN (1.5 mL) and treated with acrylic anhydride (30 μL, 0.26 mmol) and stirred at room temperature for 30 minutes. The mixture was diluted with aqueous 0.25% TFA and purified by preparative HPLC (C18, 10→70% ACN in H₂O+0.25% TFA) to give compound C-9, 1-(4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-1-yl)prop-2-en-1-one (62.4 mg, 0.124 mmol, 72% yield), as a white foam.

¹H NMR (400 MHz, Acetonitrile-d₃) δ 12.48 (s, 1H), 7.17-7.01 (m, 4H), 6.75-6.65 (m, 1H), 6.21 (dt, J=16.9, 1.9 Hz, 1H), 5.73 (dt, J=10.5, 1.8 Hz, 1H), 4.77 (dd, J=12.5, 4.6 Hz, 1H), 4.69 (dd, J=12.5, 3.2 Hz, 1H), 4.06-3.90 (m, 4H), 3.82-3.61 (m, 6H), 3.18-3.05 (m, 1H), 2.91 (s, 3H), 2.85 (q, J=6.6 Hz, 2H), 2.81-2.59 (m, 6H), 2.36-2.23 (m, 1H), 2.21-1.91 (m, 3H), 1.86-1.75 (m, 1H), 1.75-1.53 (m, 3H) ppm

LCMS: [M+H]⁺ m/z=502.3 amu

### Synthesis of Compound C-10

2-Fluoroacrylic acid (164.6 mg, 1.83 mmol) was suspended in anhydrous DCM (2.7 mL) and cooled to 0° C., then treated with DCC (189 mg, 0.910 mmol). The mixture was stirred for 3 hours, then filtered through Celite and concentrated to give 2-fluoroacrylic anhydride (139 mg, 0.860 mmol, 47% yield) as a brown solid, which was used without purification.

Intermediate 5-2, (R)-2′-(((S)-1-methylpyrrolidin-2-yl)methoxy)-4′-(piperazin-1-yl)-3,4,5′,8′-tetrahydro-1H,6′H-spiro[naphthalene-2,7′-quinazoline] (77.7 mg, 0.17 mmol) was dissolved in anhydrous MeCN (1.5 mL) and treated with 2-fluoroacrylic anhydride (48 mg, 0.30 mmol) and stirred at room temperature for 1 hour, then diluted with aqueous 0.25% TFA and purified by preparative HPLC (C18, 10→50% ACN in H₂O+0.25% TFA) to give compound C-10, 2-Fluoro-1-(4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-1-yl)prop-2-en-1-one (63 mg, 0.12 mmol, 70% yield) as a white foam.

¹H NMR (400 MHz, Acetonitrile-d₃) δ 12.44 (s, 1H), 7.21-6.87 (m, 4H), 5.27 (q, J=3.8 Hz, 1H), 5.19 (dd, J=25.4, 3.8 Hz, 1H), 4.77 (dd, J=12.5, 4.4 Hz, 1H), 4.68 (dd, J=12.5, 3.2 Hz, 1H), 4.13-3.85 (m, 4H), 3.81-3.49 (m, 6H), 3.11 (d, J=5.1 Hz, 1H), 2.94-2.59 (m, 10H), 2.37-2.26 (m, 1H), 1.96 (s, 4H), 1.87-1.54 (m, 4H) ppm

LCMS: [M+H]⁺ m/z=520.2 amu

### Synthesis of Intermediate 5-3

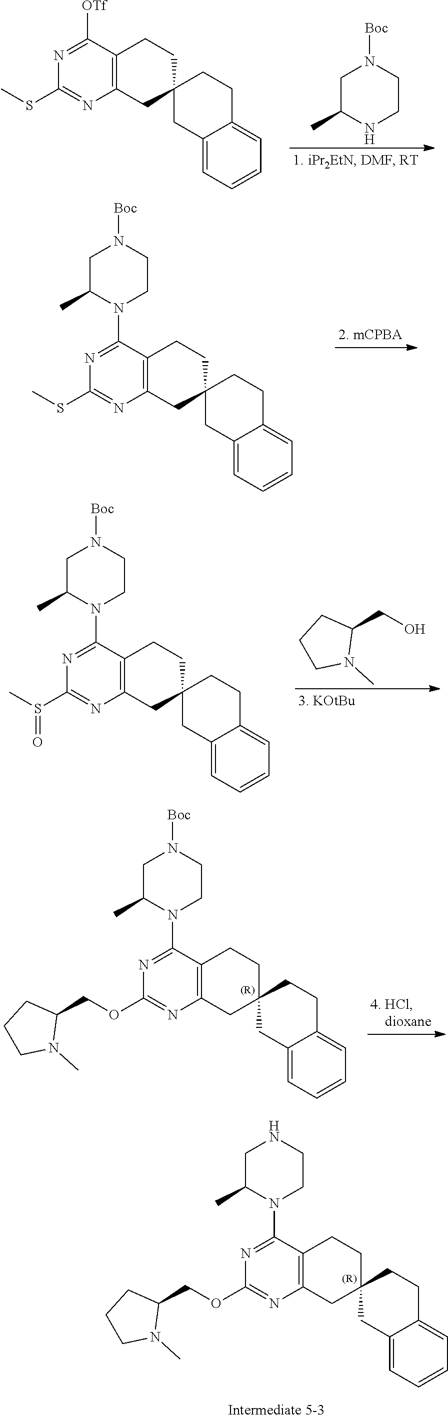

Intermediate 5-1, (R)-2′-(methylthio)-3,4,5′,8′-tetrahydro-1H,6′H-spiro[naphthalene-2,7′-quinazolin]-4′-yl trifluoromethanesulfonate (160 mg, 0.36 mmol), was dissolved in anhydrous DMF (1 mL) and treated with iPr₂EtN (0.19 mL, 1.1 mmol) followed by tert-butyl (3S)-3-methylpiperazine-1-carboxylate (79.3 mg, 0.40 mmol), and the mixture was warmed to 60° C. After 13 hours, the mixture was cooled and poured into sat NaHCO₃ and extracted with EtOAc (3 times). The combined extract was washed with brine, dried over Na₂SO₄, filtered through a thin pad of silica gel, and concentrated. The residue was purified by flash column chromatography on silica gel (5→40% EtOAc in hexanes) to give tert-butyl (S)-3-methyl-4-((R)-2′-(methylthio)-3,4,5′,8′-tetrahydro-1H,6′H-spiro[naphthalene-2,7′-quinazolin]-4′-yl)piperazine-1-carboxylate (158.9 mg, 0.321 mmol, 89% yield) as a white foam.

LCMS: [M+H]⁺ m/z=495.3 amu

tert-Butyl (S)-3-methyl-4-((R)-2′-(methylthio)-3,4,5′,8′-tetrahydro-1H,6′H-spiro[naphthalene-2,7′-quinazolin]-4′-yl)piperazine-1-carboxylate (158.9 mg, 0.32 mmol) was dissolved in DCM, cooled to 0° C., and treated with mCPBA (96.1 mg, 0.42 mmol). After 20 minutes, the mixture was diluted with Et₂O and washed with half-saturated NaHCO₃ (3 times), brine, then dried over Na₂SO₄, and concentrated to give the crude tert-butyl (3S)-3-methyl-4-((2R)-2′-(methylsulfinyl)-3,4,5′,8′-tetrahydro-1H,6′H-spiro[naphthalene-2,7′-quinazolin]-4′-yl)piperazine-1-carboxylate (167 mg, >100% yield) as a white foam, which was carried forward without purification.

LCMS: [M+H]⁺ m/z=511.3 amu

1-Methyl-L-prolinol (75.3 mg, 0.65 mmol) was dissolved in anhydrous THF (2 mL) and treated with KOtBu, 1.7M in THF (385 uL, 0.66 mmol). The mixture was aged for 5 minutes, then added to a solution of the crude tert-butyl (3S)-3-methyl-4-((2R)-2′-(methylsulfinyl)-3,4,5′,8′-tetrahydro-1H,6′H-spiro[naphthalene-2,7′-quinazolin]-4′-yl)piperazine-1-carboxylate (167 mg, 0.33 mmol, est.) in anhydrous THF (1 mL) at 0° C. After 30 minutes, the mixture was poured into aqueous K₂CO₃ and extracted with Et₂O (3 times). The combined extract was washed with brine, dried over Na₂SO₄, and concentrated to give the crude tert-butyl (S)-3-methyl-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (178.4 mg, 0.3176 mmol, 97% yield), which was carried forward without purification.

LCMS: [M+H]⁺ m/z=562.4 amu

The crude tert-butyl (S)-3-methyl-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (178.4 mg, 0.318 mmol, est.) was treated with 4N HCl in dioxane (2.5 mL) and aged at room temperature. After 50 minutes, the mixture was dissolved in 1N HCl and washed with Et₂O (2 times). The ethereal wash was back-extracted with 1N HCl once, and the combined aqueous was basified with K₂CO₃ and back-extracted with EtOAc (3 times). The combined extract was washed with brine, dried over K₂CO₃, filtered, and concentrated to give the intermediate 5-3, (R)-4′-((S)-2-methylpiperazin-1-yl)-2′-(((S)-1-methylpyrrolidin-2-yl)methoxy)-3,4,5′,8′-tetrahydro-1H,6′H-spiro[naphthalene-2,7′-quinazoline] (136.2 mg, 0.295 mmol, 93% yield) as a vitreous glass, which was carried forward without purification. LCMS: [M+H]⁺ m/z=462.3 amu

### Synthesis of Compound C-11

Intermediate 5-3, (R)-4′-((S)-2-methylpiperazin-1-yl)-2′-(((S)-1-methylpyrrolidin-2-yl)methoxy)-3,4,5′,8′-tetrahydro-1H,6′H-spiro[naphthalene-2,7′-quinazoline] (68.1 mg, 0.15 mmol est.), was dissolved in anhydrous MeCN (750 μL) and treated with acrylic anhydride (25.5 μL, 0.22 mmol). After 10 minutes, the mixture was diluted with aqueous 0.25% TFA and purified by preparative HPLC (C18, 10→55% ACN in H₂O+0.25% TFA) to give compound C-11, 1-((S)-3-methyl-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-1-yl)prop-2-en-1-one (61.1 mg, 0.119 mmol, 80% yield), as a white foam.

¹H NMR (400 MHz, Acetonitrile-d3) δ 11.56-10.71 (m, 1H), 6.34-6.00 (m, 4H), 5.85-5.68 (m, 1H), 5.27 (d, J=16.8 Hz, 1H), 4.78 (d, J=10.1 Hz, 1H), 4.04-3.64 (m, 3H), 3.54-3.25 (m, 2H), 3.21-2.38 (m, 5H), 2.30-1.60 (m, 13H), 1.47-0.52 (m, 8H), 0.37 (d, J=4.3 Hz, 3H) ppm

LCMS: [M+H]⁺ m/z=516.3 amu

### Synthesis of Compound C-12

Intermediate 5-3, (R)-4′-((S)-2-methylpiperazin-1-yl)-2′-(((S)-1-methylpyrrolidin-2-yl)methoxy)-3,4,5′,8′-tetrahydro-1H,6′H-spiro[naphthalene-2,7′-quinazoline] (68.1 mg, 0.148 mmol, est.), was dissolved in anhydrous MeCN (750 μL) and treated with 2-fluoroacrylic anhydride (35.9 mg, 0.22 mmol). After 10 minutes, the mixture was diluted with aqueous 0.25% TFA and purified by preparative HPLC to give compound C-12, 2-fluoro-1-((S)-3-methyl-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-1-yl)prop-2-en-1-one (52.4 mg, 0.0982 mmol, 67% yield), as a white foam.

¹H NMR (400 MHz, Acetonitrile-d₃) δ 12.63 (s, 1H), 7.16-7.03 (m, 4H), 5.32-5.25 (m, 1H), 5.19 (dd, J=22.7, 3.9 Hz, 1H), 4.78 (dd, J=12.4, 4.8 Hz, 1H), 4.68 (dd, J=12.3, 3.2 Hz, 1H), 4.37 (dt, J=13.8, 3.2 Hz, 1H), 4.29-4.00 (m, 2H), 3.78-3.63 (m, 2H), 3.55 (ddd, J=14.2, 11.7, 3.4 Hz, 1H), 3.10 (d, J=9.4 Hz, 1H), 2.91 (s, 3H), 2.90-2.67 (m, 5H), 2.67-2.56 (m, 4H), 2.35-2.23 (m, 1H), 2.18-1.92 (m, 5H), 1.89-1.78 (m, 1H), 1.78-1.60 (m, 2H), 1.52 (ddd, J=13.7, 8.4, 5.4 Hz, 1H), 1.34 (d, J=6.7 Hz, 3H) ppm

LCMS: [M+H]⁺ m/z=534.3 amu

### Synthesis of Intermediate 5-4

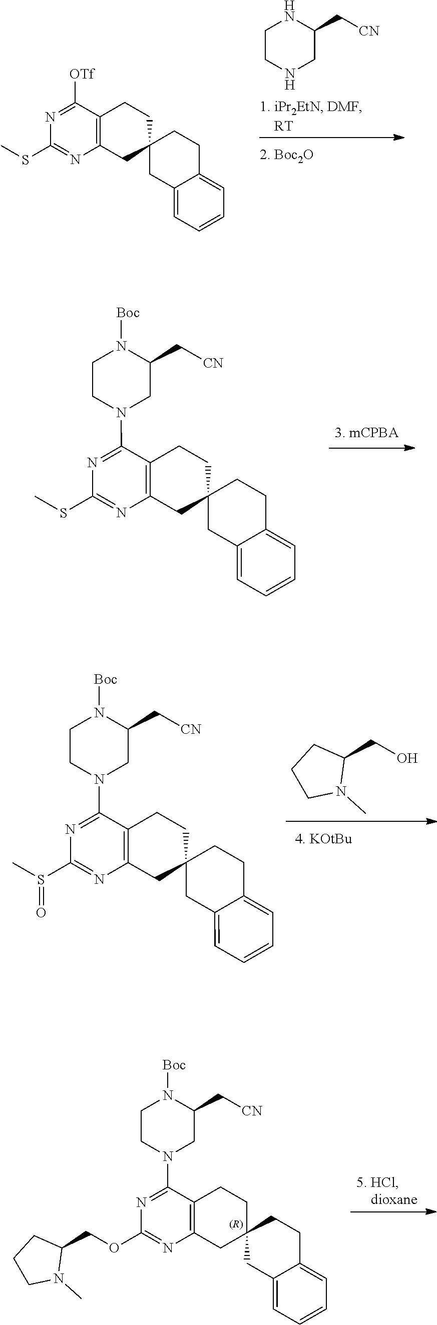

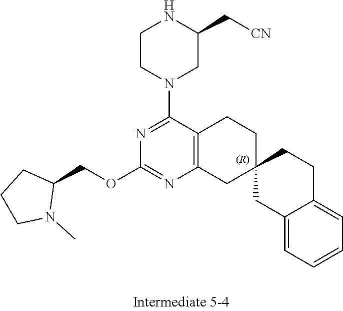

Intermediate 5-1, (R)-2′-(methylthio)-3,4,5′,8′-tetrahydro-1H,6′H-spiro[naphthalene-2,7′-quinazolin]-4′-yl trifluoromethanesulfonate (160 mg, 0.36 mmol), was dissolved in anhydrous DMF (1 mL) and treated with iPr₂EtN (188 μL, 1.1 mmol) and 2-[(2S)-piperazin-2-yl]acetonitrile dihydrochloride (78 mg, 0.40 mmol), and stirred at room temperature for 20 minutes, then treated with Boc₂O (118 mg, 0.54 mmol) and stirred for 16 hours. The mixture was poured into sat NaHCO₃ and extracted with EtOAc (3 times), and the combined extract was washed with brine, dried over Na₂SO₄, filtered through a thin pad of silica gel, and concentrated. The residue was purified by flash column chromatography on silica gel (5→40% EtOAc in hexanes) to give tert-butyl (S)-2-(cyanomethyl)-4-((R)-2′-(methylthio)-3,4,5′,8′-tetrahydro-1H,6′H-spiro[naphthalene-2,7′-quinazolin]-4′-yl)piperazine-1-carboxylate (223 mg, 0.429 mmol, >100% yield) as a white foam.

LCMS: [M+H]⁺ m/z=520.3 amu

tert-Butyl (S)-2-(cyanomethyl)-4-((R)-2′-(methylthio)-3,4,5′,8′-tetrahydro-1H,6′H-spiro[naphthalene-2,7′-quinazolin]-4′-yl)piperazine-1-carboxylate (223 mg, 0.43 mmol) was dissolved in DCM, cooled to 0° C., and treated with mCPBA (128 mg, 0.56 mmol). The mixture was stirred for 20 minutes then diluted with Et₂O and washed with half-saturated NaHCO₃ (3 times), brine, then dried over Na₂SO₄, and concentrated to give the crude tert-butyl (2S)-2-(cyanomethyl)-4-((2R)-2′-(methylsulfinyl)-3,4,5′,8′-tetrahydro-1H,6′H-spiro[naphthalene-2,7′-quinazolin]-4′-yl)piperazine-1-carboxylate (225.9 mg, 0.4217 mmol, 98% yield) as a white foam, which was carried forward without purification.

LCMS: [M+H]⁺ m/z=536.3 amu

1-Methyl-L-prolinol (97 mg, 0.84 mmol) was dissolved in anhydrous THF (2.5 mL) and treated with KOtBu, 1.7M in THE (496 μL, 0.84 mmol). The mixture was aged for 5 minutes, then added to a solution of the crude tert-butyl (2S)-2-(cyanomethyl)-4-((2R)-2′-(methylsulfinyl)-3,4,5′,8′-tetrahydro-1H,6′H-spiro[naphthalene-2,7′-quinazolin]-4′-yl)piperazine-1-carboxylate (226 mg, 0.42 mmol, est.) in anhydrous THF (1.5 mL) at 0° C., and the mixture was stirred for 30 minutes, then poured into aqueous K₂CO₃ and extracted with Et₂O (3 times). The combined extract was washed with brine, dried over Na₂SO₄, and concentrated to give the crude tert-butyl (S)-2-(cyanomethyl)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (230 mg, 0.392 mmol, 93.0% yield) as an oily residue, which was carried forward without further purification.

LCMS: [M+H]⁺ m/z=587.4 amu

The crude tert-butyl (S)-2-(cyanomethyl)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (230 mg, 0.39 mmol) was treated with 4N HCl in dioxane (3 mL, 12 mmol) and aged at room temperature for 1 hour. The mixture was concentrated then partitioned between 1N HCl and Et₂O, and the aqueous phase was collected and washed once more with Et₂O. The ethereal wash was back-extracted with 1N HCl once, and the combined aqueous was basified with K₂CO₃ and back-extracted with EtOAc (3 times). The combined extract was washed with brine, dried over K₂CO₃, filtered, and concentrated to give the Intermediate 5-4, 2-((S)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (171 mg, 0.351 mmol, 89% yield), as an oily residue, which was carried forward without purification.

LCMS: [M+H]⁺ m/z=487.3 amu

### Synthesis of Compound C-13

Intermediate 5-4, 2-((S)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (85.4 mg, 0.18 mmol), was dissolved in anhydrous MeCN (1.5 mL) and treated with acrylic anhydride (30 μL, 0.26 mmol). The mixture was stirred for 20 minutes then diluted with 0.25% TFA in H₂O, filtered, and purified by preparative HPLC (C18, 5→55% ACN in H₂O+0.25% TFA) to give compound C-13, 2-((S)-1-acryloyl-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (26.9 mg, 0.0498 mmol, 28% yield).

¹H NMR (400 MHz, Acetonitrile-d₃) δ 10.44 (d, J=126.7 Hz, 1H), 7.15-7.02 (m, 4H), 6.71 (s, 1H), 6.31-6.20 (m, 1H), 5.78 (dd, J=10.5, 2.1 Hz, 1H), 4.84-4.64 (m, 2H), 4.64-4.50 (m, 1H), 4.39 (s, 1H), 4.11-3.90 (m, 1H), 3.78-3.65 (m, 2H), 3.63-3.46 (m, 2H), 3.15-3.04 (m, 1H), 2.91 (s, 3H), 2.89-2.62 (m, 11H), 2.37-2.23 (m, 1H), 2.14-1.94 (m, 4H), 1.87-1.76 (m, 1H), 1.76-1.54 (m, 3H) ppm

LCMS: [M+H]⁺ m/z=541.3 amu

### Synthesis of Compound C-14

Intermediate 5-4, 2-((S)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (85.4 mg, 0.18 mmol), was dissolved in anhydrous MeCN (1.5 mL) and treated with 2-fluoroacrylic anhydride (42.7 mg, 0.26 mmol). After 9 hours, the mixture was diluted with aqueous 0.25% TFA and purified by preparative HPLC in three injections. (C18, 10→55% ACN in H₂O+0.25% TFA) to give compound C-14, 2-((S)-1-(3-fluorobuta-1,3-dien-2-yl)-4-((R)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (55.6 mg, 0.0995 mmol, 57% yield), as a faintly yellow glassy solid.

¹H NMR (400 MHz, Acetonitrile-d₃) δ 12.43 (s, 1H), 7.21-7.03 (m, 4H), 5.38-5.19 (m, 2H), 4.94-4.67 (m, 3H), 4.65-4.53 (m, 1H), 4.40 (d, J=8.7 Hz, 1H), 3.72 (ddd, J=11.7, 7.5, 4.7 Hz, 2H), 3.62-3.38 (m, 3H), 3.14-3.02 (m, 1H), 2.92 (s, 3H), 2.90-2.60 (m, 11H), 2.36-2.23 (m, 1H), 2.17-1.93 (m, 5H), 1.85-1.76 (m, 1H), 1.74-1.55 (m, 3H) ppm

LCMS: [M+H]⁺ m/z=559.3 amu

### Example 6: Synthesis of Compounds C-17 through C-21

### Synthesis of Intermediate 6-1

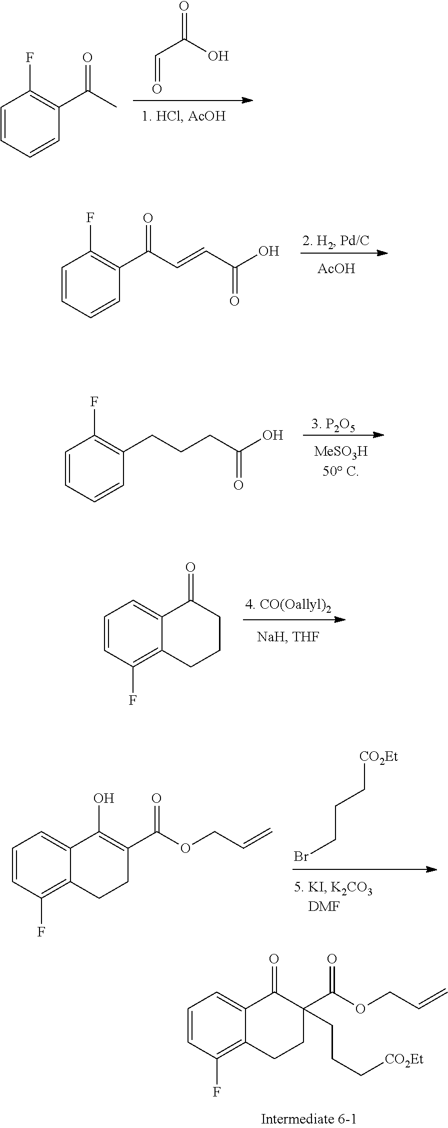

2-Fluoroacetophenone (6.91 g, 50 mmol) was dissolved in glacial AcOH (150 mL) and treated with glyoxylic acid, 50% in H₂O (8.3 mL, 75 mmol) followed by concentrated HCl (7.9 mL, 100 mmol), and the mixture was heated to reflux under N₂ atmosphere for 24 hours, then cooled to room temperature and concentrated. The crude isolate was purified by flash column chromatography on silica gel (8:2 hexanes:EtOAc) to give (E)-4-(2-fluorophenyl)-4-oxobut-2-enoic acid (6.97 g, 35.9 mmol, 72% yield) as a yellow solid.

¹H NMR (400 MHz, CDCl₃) δ 7.89-7.81 (m, 2H), 7.60 (dddd, J=8.4, 7.1, 5.1, 1.9 Hz, 1H), 7.29 (td, J=7.5, 1.1 Hz, 1H), 7.19 (ddd, J=10.9, 8.3, 1.1 Hz, 1H), 6.84 (dd, J=15.6, 1.3 Hz, 1H) ppm

(E)-4-(2-fluorophenyl)-4-oxobut-2-enoic acid (6.97 g, 36 mmol) was dissolved in acetic acid (105 mL) and treated with Pd/C, 10 wt % (wetted) (1.2 g, 3.6 mmol). The vessel was evacuated and backfilled with H₂ then heated to 90° C. for 2 hours. The mixture was cooled, filtered through Celite, concentrated, and co-evaporated from toluene once, then further dried in vacuo to give the crude 4-(2-fluorophenyl)butanoic acid (6.40 g, 35.1 mmol, 98% yield). Rf=0.39 (7:3 hexanes:EtOAc+2% AcOH), which was carried on to the next step without further purification.

¹H NMR (500 MHz, Chloroform-d) δ 11.59 (s, 1H), 7.18 (q, J=6.3, 5.2 Hz, 2H), 7.10-6.98 (m, 2H), 2.73 (t, J=7.6 Hz, 2H), 2.41 (t, J=7.5 Hz, 2H), 1.99 (q, J=7.5 Hz, 2H) ppm

The crude 4-(2-fluorophenyl)butanoic acid (6.2 g, 34 mmol) was treated with Eaton's reagent (34 mL) and the mixture was warmed to 50° C. for 1 hour. The mixture was cooled to room temperature and poured into ice water and extracted with DCM (3 times). The combined extract was washed with sat NaHCO₃, brine, then dried over Na₂SO₄, concentrated, and purified by flash column chromatography on silica gel (0→15% EtOAc in hexanes) to give 5-fluoro-3,4-dihydronaphthalen-1(2H)-one (4.403 g, 26.8 mmol, 79% yield).

¹H NMR (500 MHz, CDCl₃) δ 7.84 (dd, J=7.7, 1.2 Hz, 1H), 7.32-7.23 (m, 2H), 7.21 (dd, J=8.1, 1.3 Hz, 1H), 2.96 (t, J=6.2 Hz, 2H), 2.67 (dd, J=7.4, 5.7 Hz, 2H), 2.16 (p, J=6.4 Hz, 2H) ppm

5-fluoro-3,4-dihydronaphthalen-1(2H)-one (4.40 g, 27 mmol) was dissolved in anhydrous THF (45 mL) and cooled to 0° C. then treated with NaH (2.68 g, 67 mmol). The mixture was allowed to warm to room temperature and diallyl carbonate (5.77 mL, 40 mmol) was added and stirring continued for 21 hours. The reaction was cooled in an ice bath and quenched by dropwise addition of sat NH₄Cl then diluted with H₂O and extracted with EtOAc (3 times). The combined extract was washed with brine, dried over Na₂SO₄, filtered through a thin pad of silica gel, concentrated, and purified by flash column chromatography on silica gel (0→15% EtOAc in hexanes) to give allyl 5-fluoro-1-hydroxy-3,4-dihydronaphthalene-2-carboxylate (6.04 g, 24.3 mmol, 91% yield) as a pale yellow oil.

¹H NMR (400 MHz, CDCl₃, major tautomer) δ 12.38 (s, 1H), 7.61 (dd, J=7.8, 1.4 Hz, 1H), 7.30-7.20 (m, 1H), 7.09 (ddd, J=9.3, 8.3, 1.2 Hz, 1H), 5.99 (ddq, J=17.1, 10.5, 5.7 Hz, 1H), 5.43-5.33 (m, 1H), 5.29 (dt, J=10.4, 1.3 Hz, 1H), 4.74 (dt, J=5.5, 1.4 Hz, 2H), 2.85 (t, J=8.0 Hz, 2H), 2.61 (t, J=7.6 Hz, 2H) ppm

LCMS: [M+H]⁺ m/z=249.1 amu

Allyl 5-fluoro-1-hydroxy-3,4-dihydronaphthalene-2-carboxylate (3.97 g, 16 mmol) was dissolved in anhydrous DMF (48 mL) and treated with ethyl 4-bromobutanoate (3.4 mL, 24 mmol), KI (2.65 g, 16 mmol), and K₂CO₃ (4.42 g, 32 mmol), and the mixture was heated to 50° C. for 3 hours. The mixture was poured into H₂O and extracted with EtOAc (3 times). The combined extract was washed with dilute Na₂S₂O₃, brine, then dried over Na₂SO₄, filtered through a thin pad of silica gel, concentrated, and purified by flash column chromatography on silica gel (5→30% EtOAc in hexanes) to give intermediate 6-1, allyl 2-(4-ethoxy-4-oxobutyl)-5-fluoro-1-oxo-1,2,3,4-tetrahydronaphthalene-2-carboxylate (4.601 g, 12.7 mmol, 79.4% yield), as a colorless oil.

LCMS: [M+H]⁺ m/z=363.1 amu

### Synthesis of Intermediate 6-2

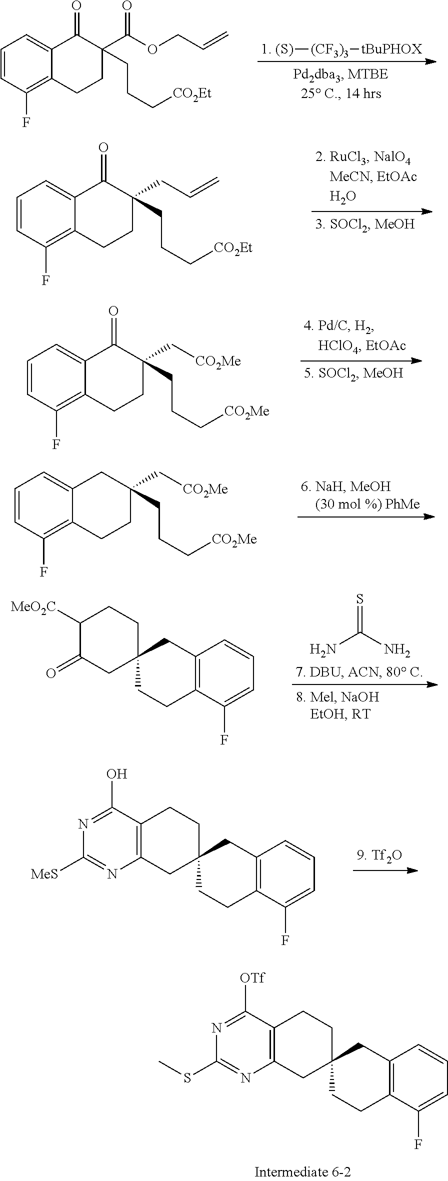

Pd₂(dba)₃ (174 mg, 0.19 mmol) and (S)-p-(CF₃)₃-t-BuPHOX (300 mg, 0.51 mmol) were suspended in anhydrous, degassed MTBE (40 mL) under N₂ atmosphere. The mixture was warmed to 25° C. and stirred for 45 minutes. Separately, intermediate 6-1, allyl 2-(4-ethoxy-4-oxobutyl)-5-fluoro-1-oxo-1,2,3,4-tetrahydronaphthalene-2-carboxylate (2.3 g, 6.4 mmol), was dissolved in MTBE (40 mL) and sparged for 20 minutes then added to the catalyst mixture. After 16 hours, the reaction was opened to air and amended with 0.3 vol hexanes and a small amount of silica gel. The mixture was stirred for 10 minutes then filtered through a thin pad of silica gel, concentrated, and purified by flash column chromatography on silica gel (0→15% EtOAc in hexanes) to give ethyl (S)-4-(2-allyl-5-fluoro-1-oxo-1,2,3,4-tetrahydronaphthalen-2-yl)butanoate (1.954 g, 6.14 mmol, 97% yield) as a pale yellow viscous oil.

LCMS: [M+H]⁺ m/z=319.1 amu

ethyl (S)-4-(2-allyl-5-fluoro-1-oxo-1,2,3,4-tetrahydronaphthalen-2-yl)butanoate (1.95 g, 6.1 mmol) was dissolved in EtOAc (12 mL) and MeCN (12 mL) and treated with H₂O (19 mL), NaIO₄ (6.56 g, 31 mmol) and RuCl₃.xH₂O (28.0 mg, 0.14 mmol), and the mixture was stirred vigorously at room temperature for 2 hours. The mixture was then diluted with 0.5M NaHSO₄ and EtOAc, stirred for 5 minutes, then filtered through Celite. The organic phase was collected and the aqueous was extracted twice more with EtOAc. The combined extract was washed with brine, dried over Na₂SO₄, and filtered through Celite, concentrated, and further dried in vacuo. The oily residue was taken up in MeOH (35 mL), cooled to 0° C., and treated with SOCl₂ (4.3 mL, 59 mmol) dropwise. The cooling bath was removed and the mixture was stirred at room temperature for 2 hours then concentrate. The residue was taken up in Et₂O and washed with NaHCO₃ (2 times), brine, then dried over Na₂SO₄, and concentrated to give the crude methyl (S)-4-(5-fluoro-2-(2-methoxy-2-oxoethyl)-1-oxo-1,2,3,4-tetrahydronaphthalen-2-yl)butanoate (2.05 g, 99% yield) as a viscous oil, which was used in the next step without further purification.

LCMS: [M+H]⁺ m/z=337.1 amu

The crude methyl (S)-4-(5-fluoro-2-(2-methoxy-2-oxoethyl)-1-oxo-1,2,3,4-tetrahydronaphthalen-2-yl)butanoate (2.05 g, 6.1 mmol, est.) was dissolved in EtOAc (31 mL) and treated with Pd/C, 10 wt % (410 mg, 6.1 mmol) and HClO₄, 60% (100 μL, 0.91 mmol) and the vessel was charged with H₂. The mixture was stirred vigorously for 12 hours then filtered through Celite, concentrated, and purified by flash column chromatography on silica gel (5→40% EtOAc in hexanes) to give methyl (R)-4-(5-fluoro-2-(2-methoxy-2-oxoethyl)-1,2,3,4-tetrahydronaphthalen-2-yl)butanoate (1.349 g, 69% yield) as a colorless oil.

¹H NMR (400 MHz, Chloroform-d) δ 7.11-7.01 (m, 1H), 6.87-6.78 (m, 2H), 3.66 (s, 3H), 3.65 (s, 3H), 2.85-2.71 (m, 3H), 2.70-2.63 (m, 1H), 2.37 (d, J=14.2 Hz, 1H), 2.32-2.26 (m, 3H), 1.82-1.64 (m, 4H), 1.54-1.32 (m, 2H) ppm

LCMS: [M+H]⁺ m/z=323.2 amu

NaH (39.5 mg, 1.0 mmol) was suspended in anhydrous toluene (1.5 mL) and treated with MeOH (8.3 uL, 0.21 mmol) and the mixture was stirred until gas evolution ceased. A solution of methyl (R)-4-(5-fluoro-2-(2-methoxy-2-oxoethyl)-1,2,3,4-tetrahydronaphthalen-2-yl)butanoate (265 mg, 0.82 mmol) in anhydrous toluene (2 mL) was added dropwise, and the mixture was warmed to 70° C. After 50 minutes, a second charge of NaH (20 mg) and MeOH (8.3 μL, 0.206 mmol) was added, and stirring maintained for an additional 6 hours. The mixture was cooled to room temperature and poured into sat NH₄Cl and extracted with EtOAc (3 times). The combined extract was washed with brine, dried over Na₂SO₄, filtered through a thin pad of silia gel, concentrated, and purified by flash column chromatography on silica gel (0→15% EtOAc in hexanes) to give methyl (1R)-5′-fluoro-3-oxo-3′,4′-dihydro-1′H-spiro[cyclohexane-1,2′-naphthalene]-4-carboxylate (188 mg, 0.648 mmol, 79% yield) as a colorless, vitreous oil.

LCMS: [M+H]⁺ m/z=291.1 amu

methyl (1R)-5′-fluoro-3-oxo-3′,4′-dihydro-1′H-spiro[cyclohexane-1,2′-naphthalene]-4-carboxylate (188 mg, 0.65 mmol) was dissolved in anhydrous MeCN (3.2 mL) and treated with thiourea (59.2 mg, 0.78 mmol) and DBU (145 μL, 0.97 mmol) and the mixture was heated to 80° C. for 11.5 hours. The mixture was cooled and concentrated to approximately 500 μL total volume then diluted with aq. NaH₂PO₄ and the resulting solids were collected by centrifugation.

LCMS: [M+H]⁺ m/z=317.1 amu

The still wet material was suspended in EtOH (2 mL) and treated with 1M NaOH (712 μL, 0.71 mmol) and treated with MeI (48 μL, 0.78 mmol) and stirred vigorously at room temperature for 7 hours. The mixture was poured into aqueous NaH₂PO₄ and extracted with CHCl₃ (3 times). The combined extract was washed with brine, dried over Na₂SO₄, filtered, concentrated, and purified by flash column chromatography on silica gel (0→10% MeOH in CH₂Cl₂) to give (R)-5-fluoro-2′-(methylthio)-3,4,5′,8′-tetrahydro-1H,6′H-spiro[naphthalene-2,7′-quinazolin]-4′-ol (131.9 mg, 0.399 mmol, 62% yield) as a white solid.

LCMS: [M+H]⁺ m/z=331.1 amu

(R)-5-fluoro-2′-(methylthio)-3,4,5′,8′-tetrahydro-1H,6′H-spiro[naphthalene-2,7′-quinazolin]-4′-ol (132 mg, 0.40 mmol) was suspended in anhydrous DCM (1 mL) and treated with freshly distilled iPr₂EtN (139 μL, 0.80 mmol) then the mixture was cooled to 0° C. and triflic anhydride, 1M in DCM (599 μL, 0.60 mmol) was added dropwise. The cooling bath was removed and the mixture was stirred at room temperature for 2.5 hours. The mixture was then diluted with 2 vol hexanes and filtered through a short column of silica gel rinsing with 9:1 hexanes:EtOAc and concentrated. The residue was purified by flash column chromatography on silica gel (0→15% EtOAc in hexanes) to give intermediate 6-2, (R)-5-fluoro-2′-(methylthio)-3,4,5′,8′-tetrahydro-1H,6′H-spiro[naphthalene-2,7′-quinazolin]-4′-yl trifluoromethanesulfonate (132.7 mg, 0.287 mmol, 71.9% yield), as a colorless residue.

LCMS: [M+H]⁺ m/z=463.1 amu

### Synthesis of Intermediate 6-3

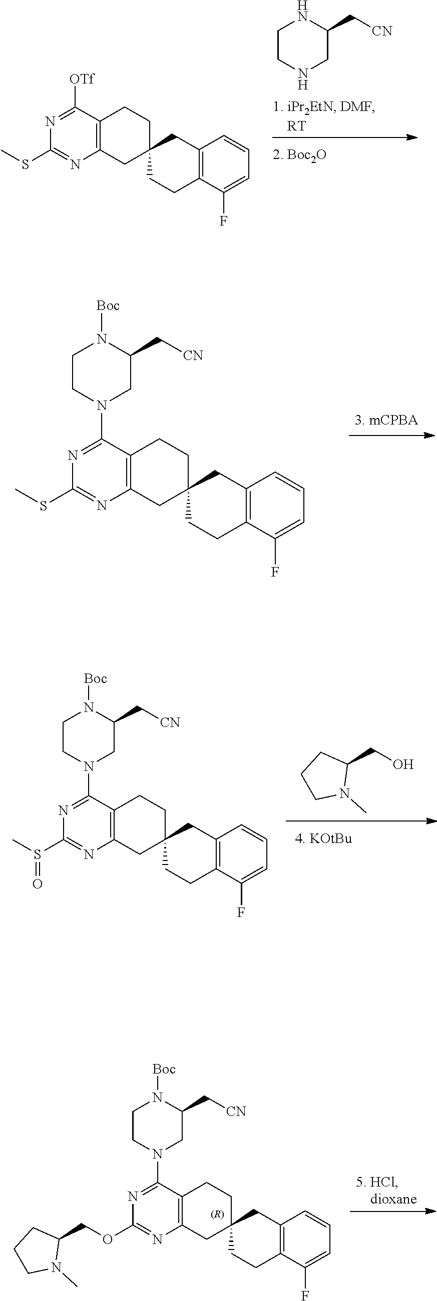

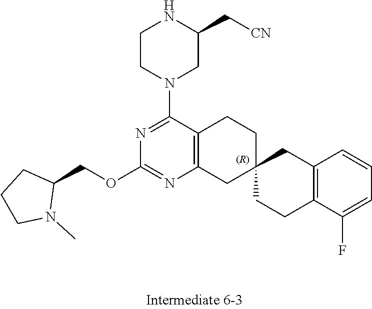

Intermediate 6-2, (R)-5-fluoro-2′-(methylthio)-3,4,5′,8′-tetrahydro-1H,6′H-spiro[naphthalene-2,7′-quinazolin]-4′-yl trifluoromethanesulfonate (66.4 mg, 0.14 mmol), was dissolved in anhydrous DMF (410 μL) and treated with iPr₂EtN (75 μL, 0.43 mmol) and 2-[(2S)-piperazin-2-yl]acetonitrile dihydrochloride (31.3 mg, 0.16 mmol) and the mixture was stirred at room temperature. After 15 minutes, Boc₂O (50 μL, 0.22 mmol) was added and stirring was continued for 16 hours. The mixture was diluted with EtOAc and washed with sat NH₄Cl, brine, then dried over Na₂SO₄, concentrated, and purified by flash column chromatography on silica gel (0→30% EtOAc in hexanes) to give tert-butyl (S)-2-(cyanomethyl)-4-((R)-5-fluoro-2′-(methylthio)-3,4,5′,8′-tetrahydro-1H,6′H-spiro[naphthalene-2,7′-quinazolin]-4′-yl)piperazine-1-carboxylate (154.8 mg, >100% yield) as a white foam, which was carried forward without further purification.

LCMS: [M+H]+=538.3 m/z.

The crude tert-butyl (S)-2-(cyanomethyl)-4-((R)-5-fluoro-2′-(methylthio)-3,4,5′,8′-tetrahydro-1H,6′H-spiro[naphthalene-2,7′-quinazolin]-4′-yl)piperazine-1-carboxylate (impure, 0.14 mmol) was dissolved in DCM (480 μL), cooled to 0° C., and treated with mCPBA (43 mg, 0.19 mmol). After 30 minutes, the mixture was diluted with Et₂O and washed with half-saturated NaHCO₃ (3 times), brine, then dried over Na₂SO₄, and concentrated to give the crude tert-butyl (2S)-2-(cyanomethyl)-4-((2R)-5-fluoro-2′-(methylsulfinyl)-3,4,5′,8′-tetrahydro-1H,6′H-spiro[naphthalene-2,7′-quinazolin]-4′-yl)piperazine-1-carboxylate (140 mg, >100% yield) as a white foam, which was carried forward without purification.

LCMS: [M+H]⁺ m/z=554.3 amu

1-Methyl-L-prolinol (33 mg, 0.287 mmol) was dissolved in anhydrous THF (1 mL) and treated with KOtBu, 1.7M in THE (169 μL, 0.287 mmol) and the mixture was stirred for 5 minutes then added to a solution of the crude tert-butyl (2S)-2-(cyanomethyl)-4-((2R)-5-fluoro-2′-(methylsulfinyl)-3,4,5′,8′-tetrahydro-1H,6′H-spiro[naphthalene-2,7′-quinazolin]-4′-yl)piperazine-1-carboxylate (impure, 0.14 mmol) in anhydrous THF (500 μL) at 0° C. After 1 hour, the mixture was poured into aqueous K₂CO₃ and extracted with Et₂O (3 times). The combined extract was washed with brine, dried over Na₂SO₄, filtered, concentrated, and purified by flash column chromatography on basic alumina (0→100% CH₂Cl₂ in hexanes followed by 100% EtOAc) to give tert-butyl (S)-2-(cyanomethyl)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (86.1 mg, 0.142 mmol, 99% yield).

LCMS: [M+H]⁺ m/z=605.4 amu

tert-Butyl (S)-2-(cyanomethyl)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (86.1 mg, 0.14 mmol) was treated with 4N HCl in dioxane (1 mL) at room temperature for 30 minutes. The mixture was then concentrated, dissolved in 1N HCl, and washed with Et₂O (2 times), then basified with K₂CO₃ and back-extracted with EtOAc (3 times). The combined extract was dried over K₂CO₃, filtered, and concentrated to give intermediate 6-3, 2-((S)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (54.8 mg, 0.109 mmol, 76% yield), as a colorless film.

LCMS: [M+H]⁺ m/z=505.3 amu

### Synthesis of Compound C-17

Intermediate 6-3, 2-((S)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (27.4 mg, 0.054 mmol), was dissolved in MeCN (360 μL) and treated with acrylic anhydride (9.4 μL, 0.081 mmol). After 30 minutes, the mixture was diluted with aqueous 0.25% TFA and purified by preparative HPLC (C18, 5→65% ACN in H₂O+0.25% TFA) to give compound C-17, 2-((S)-1-acryloyl-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (11.7 mg, 39% yield), as a colorless film.

¹H NMR (400 MHz, Acetonitrile-d₃) δ 10.45 (s, 1H), 7.14 (td, J=8.1, 5.9 Hz, 1H), 6.95-6.85 (m, 2H), 6.72 (s, 1H), 6.30-6.20 (m, 1H), 5.78 (dd, J=10.5, 2.1 Hz, 1H), 4.81-4.66 (m, 2H), 4.58 (dt, J=14.2, 2.5 Hz, 1H), 3.76-3.66 (m, 2H), 3.55 (d, J=7.5 Hz, 3H), 3.16-3.06 (m, 1H), 2.91 (s, 3H), 2.85-2.61 (m, 11H), 2.35-2.23 (m, 1H), 2.14-1.92 (m, 4H), 1.88-1.78 (m, 1H), 1.78-1.54 (m, 4H) ppm

¹⁹F NMR (376 MHz, Acetonitrile-d₃) δ −119.81 (dd, J=10.1, 5.9 Hz) ppm

LCMS: [M+H]⁺ m/z=559.3 amu

### Synthesis of Compound C-18

Intermediate 6-3, 2-((S)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (27.4 mg, 0.054 mmol), was dissolved in MeCN (400 μL) and treated with 2-fluoroacrylic anhydride (13 mg, 0.081 mmol). After 30 minutes, the mixture was diluted with aqueous 0.25% TFA and purified by preparative HPLC (C18 10→60% ACN in H₂O+0.25% TFA) to give compound C-18, 2-((S)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-1-(2-fluoroacryloyl)piperazin-2-yl)acetonitrile (25.3 mg, 0.0439 mmol, 81% yield), as a colorless film.

¹H NMR (400 MHz, Acetonitrile-d₃) δ 10.69 (s, 1H), 7.21-7.11 (m, 1H), 6.92 (td, J=8.6, 1.5 Hz, 2H), 5.38-5.22 (m, 2H), 4.80 (dd, J=12.3, 5.1 Hz, 1H), 4.73 (dd, J=12.3, 3.2 Hz, 1H), 4.62 (dt, J=14.3, 2.3 Hz, 1H), 4.44 (d, J=8.8 Hz, 1H), 3.79-3.67 (m, 2H), 3.59 (d, J=13.1 Hz, 1H), 3.44 (d, J=24.8 Hz, 2H), 3.18-3.05 (m, 1H), 2.95 (s, 3H), 2.92-2.64 (m, 11H), 2.38-2.26 (m, 1H), 2.19-1.92 (m, 4H), 1.92-1.81 (m, 1H), 1.80-1.65 (m, 2H), 1.65-1.54 (m, 1H) ppm

¹⁹F NMR (376 MHz, Acetonitrile-d₃) δ −107.54, −119.80 (dd, J=10.1, 5.9 Hz) ppm

LCMS: [M+H]⁺ m/z=577.3 amu

### Synthesis of Intermediate 6-4

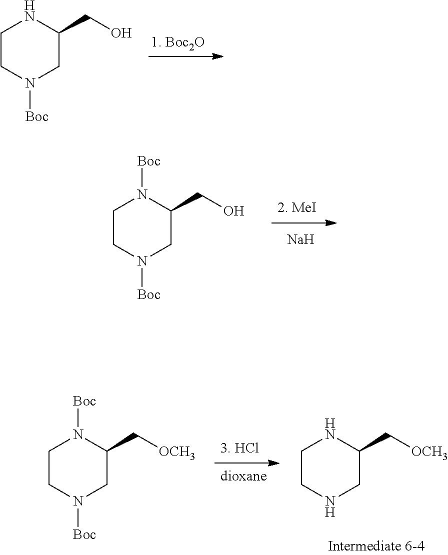

tert-butyl (3R)-3-(hydroxymethyl)piperazine-1-carboxylate (2.16 g, 10 mmol) was dissolved in DCM (32 mL), cooled to 0° C., and treated with Et₃N (1.67 mL, 12 mmol) and Boc₂O (2.52 mL, 11 mmol). The cooling bath was removed and the mixture was stirred at room temperature for 2.5 hours. The mixture was then washed with 0.5M NaHSO₄, brine, dried over Na₂SO₄, concentrated, and purified by flash column chromatography on silica gel (15→60% EtOAc in hexanes) to give di-tert-butyl (R)-2-(hydroxymethyl)piperazine-1,4-dicarboxylate (2.828 g, 8.94 mmol, 90% yield) as a white solid.

LCMS: [M+Na]⁺ m/z=339.2 amu

di-tert-Butyl (R)-2-(hydroxymethyl)piperazine-1,4-dicarboxylate (297 mg, 0.94 mmol) was dissolved in anhydrous THF (1.9 mL) and treated with MeI (234 μL, 3.8 mmol). The mixture was cooled to 0° C., NaH (45.06 mg, 1.1 mmol) was added, and the mixture was allowed to warm to room temperature. After 90 minutes, the mixture was poured into sat NH₄Cl and extracted with EtOAc (2 times). The combined extract was washed with dilute Na₂S₂O₃, brine, dried over Na₂SO₄, concentrated, and purified by flash column chromatography on silica gel (5→60% EtOAc in hexanes) to give di-tert-butyl (R)-2-(methoxymethyl)piperazine-1,4-dicarboxylate (203.1 mg, 0.615 mmol, 66% yield) as a colorless oil which crystallized upon standing.

¹H NMR (400 MHz, CDCl₃) δ 4.12 (d, J=19.8 Hz, 1H), 3.99 (dt, J=13.6, 2.0 Hz, 1H), 3.95-3.81 (m, 1H), 3.73 (d, J=12.3 Hz, 1H), 3.26 (d, J=7.5 Hz, 2H), 3.23 (s, 3H), 2.82 (dt, J=13.2, 4.6 Hz, 2H), 2.77-2.60 (m, 1H), 1.35 (s, 18H) ppm

¹³C NMR (101 MHz, CDCl₃) δ 154.77 (2), 79.95, 79.73, 69.02, 58.87, 50.04, 43.44, 42.46, 39.27, 28.25, 28.23 ppm

LCMS: [M+Na]⁺ m/z=353.2 amu

di-tert-Butyl (R)-2-(methoxymethyl)piperazine-1,4-dicarboxylate (203.1 mg, 0.62 mmol) was treated with 4N HCl in dioxane (2 mL) at room temperature for 90 minutes. A gelatinous solid resulted, which was suspended in Et₂O, filtered, and dried in vacuo to give intermediate 6-4, (R)-2-(methoxymethyl)piperazine dihydrochloride (105.3 mg, 0.519 mmol, 84% yield) as a white, hygroscopic solid.

¹H NMR (600 MHz, D₂O) δ 3.91-3.86 (m, 1H), 3.81-3.72 (m, 5H), 3.72-3.68 (m, 1H), 3.56-3.47 (m, 1H), 3.45-3.43 (m, 3H), 3.43-3.37 (m, 1H) pp,

### Synthesis of Intermediate 6-5

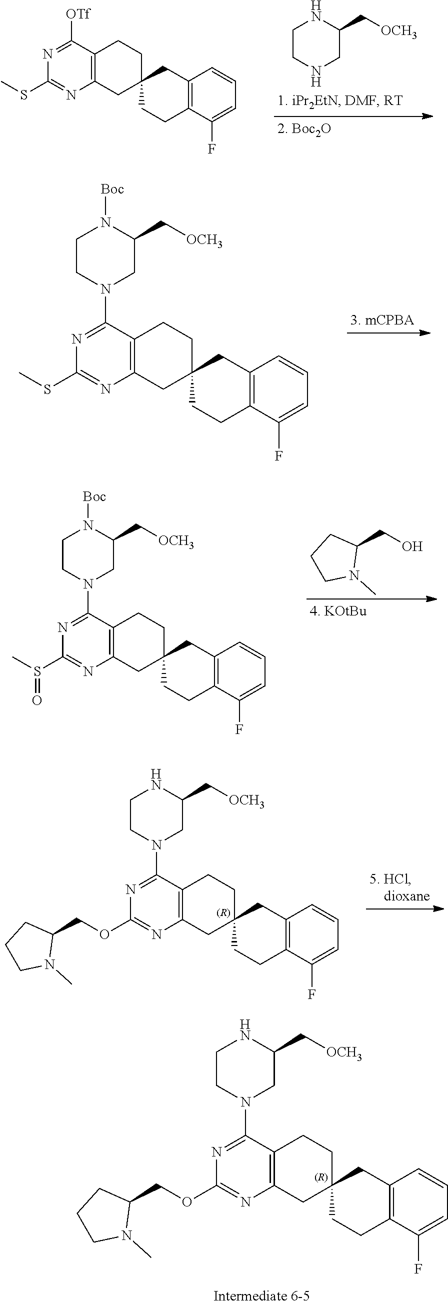

Intermediate 6-2, (R)-5-Fluoro-2′-(methylthio)-3,4,5′,8′-tetrahydro-1H,6′H-spiro[naphthalene-2,7′-quinazolin]-4′-yl trifluoromethanesulfonate (66.4 mg, 0.14 mmol), was dissolved in anhydrous DMF (410 μL) and treated with iPr₂EtN (75 μL, 0.43 mmol) and intermediate 6-4, (R)-2-(methoxymethyl)piperazine dihydrochloride (35 mg, 0.17 mmol), and the mixture was stirred at room temperature. After 90 minutes, Boc₂O (49 μL, 0.21 mmol) was added and stirring continued for 2 hours. The mixture was then diluted with EtOAc and washed with sat NH₄Cl, brine, dried over Na₂SO₄, concentrated, and purified by flash column chromatography on silica gel (0→30% EtOAc in hexanes) to give tert-butyl (R)-4-((R)-5-fluoro-2′-(methylthio)-3,4,5′,8′-tetrahydro-1H,6′H-spiro[naphthalene-2,7′-quinazolin]-4′-yl)-2-(methoxymethyl)piperazine-1-carboxylate (84.3 mg, >100% yield) as a white foam.

LCMS: [M+H]⁺ m/z=543.3 amu

tert-Butyl (R)-4-((R)-5-fluoro-2′-(methylthio)-3,4,5′,8′-tetrahydro-1H,6′H-spiro[naphthalene-2,7′-quinazolin]-4′-yl)-2-(methoxymethyl)piperazine-1-carboxylate (84.3 mg, 0.16 mmol) was dissolved in DCM (520 μL), cooled to 0° C., and treated with mCPBA (46.5 mg, 0.20 mmol). After 40 minutes, the mixture was diluted with Et₂O and washed with half-saturated NaHCO₃ (2 times), brine, dried over Na₂SO₄, and concentrated to give the crude tert-butyl (2R)-4-((2R)-5-fluoro-2′-(methylsulfinyl)-3,4,5′,8′-tetrahydro-1H,6′H-spiro[naphthalene-2,7′-quinazolin]-4′-yl)-2-(methoxymethyl)piperazine-1-carboxylate (87.8 mg, >100% yield) as a white foam. The crude product was carried forward without further purification.

LCMS: [M+H]⁺ m/z=559.3 amu

1-Methyl-L-prolinol (36 mg, 0.31 mmol) was dissolved in THF (1 mL) and treated with KOtBu, 1.7M in THE (183 μL, 0.31 mmol) and the mixture was stirred for 5 minutes then added to a solution of the crude tert-butyl (2R)-4-((2R)-5-fluoro-2′-(methylsulfinyl)-3,4,5′,8′-tetrahydro-1H,6′H-spiro[naphthalene-2,7′-quinazolin]-4′-yl)-2-(methoxymethyl)piperazine-1-carboxylate (86.8 mg, 0.16 mmol, est.) in anhydrous THF (500 μL) at 0° C. After 50 minutes, the mixture was poured into aqueous K₂CO₃ and extracted with EtOAc (3 times). The combined extract was washed with brine, dried over Na₂SO₄, filtered, and concentrated to give the crude tert-butyl (R)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazine-1-carboxylate (110.6 mg, >100% yield) as a pale yellow vitreous oil, which was carried forward without further purification.

LCMS: [M+H]⁺ m/z=610.4 amu

The crude tert-butyl (R)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazine-1-carboxylate (94.7 mg, 0.16 mmol) was treated with 4N HCl in dioxane (2 mL) at room temperature. After 60 minutes, the mixture was concentrated and the residue was dissolved in 1N HCl and washed with Et₂O (2 times) then basified by K₂CO₃ and back-extracted with EtOAc (3 times). The combined extract was dried over anhydrous K₂CO₃, filtered, and concentrated to give intermediate 6-5, (R)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (78.2 mg, 99% yield), as a faintly yellow oily residue.

LCMS: [M+H]⁺ m/z=510.3 amu

### Synthesis of Compound C-19

Intermediate 6-5, (R)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (26.1 mg, 0.051 mmol), was dissolved in anhydrous MeCN (340 μL) and treated with acrylic anhydride (8.9 μL, 0.077 mmol) at 0° C. then allowed to warm to RT. After 10 minutes, the mixture was diluted with aqueous 0.25% TFA and purified by preparative HPLC (C18 10→60% ACN in H₂O+0.25% TFA) to give compound C-19, 1-((R)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazin-1-yl)prop-2-en-1-one (14.4 mg, 0.0255 mmol, 50% yield), as a colorless film.

¹H NMR (400 MHz, Acetonitrile-d3) δ 10.48 (s, 1H), 7.05 (td, J=7.9, 5.8 Hz, 1H), 6.86-6.76 (m, 2H), 6.62 (t, J=13.0 Hz, 1H), 6.12 (dd, J=16.8, 2.2 Hz, 1H), 5.62 (dd, J=10.5, 2.2 Hz, 1H), 4.74-4.64 (m, 1H), 4.61-4.48 (m, 2H), 4.35 (d, J=35.1 Hz, 2H), 3.69-3.53 (m, 3H), 3.51-3.42 (m, 1H), 3.21 (s, 3H), 3.05-2.96 (m, 1H), 2.82 (s, 3H), 2.80-2.51 (m, 11H), 2.28-2.14 (m, 1H), 2.07-1.83 (m, 4H), 1.77-1.60 (m, 2H), 1.55 (t, J=6.5 Hz, 2H) ppm

¹⁹F NMR (376 MHz, Acetonitrile-d3) δ −119.75 (t, J=9.8, 5.8 Hz) ppm

LCMS: [M+H]⁺ m/z=564.3 amu

### Synthesis of Compound C-20

Intermediate 6-5, (R)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (31.0 mg, 0.061 mmol), was dissolved in MeCN (610 μL) and treated with 2-fluoroacrylic anhydride (14.8 mg, 0.091 mmol). After 1 hour, HPLC analysis showed complete conversion to a major product. The mixture was diluted with aqueous 0.25% TFA and purified by preparative HPLC (C18, 10→55% ACN in H₂O+0.25% TFA) to give compound C-20, 2-fluoro-1-((R)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazin-1-yl)prop-2-en-1-one (29.2 mg, 0.0502 mmol, 83% yield), as a colorless film.

¹H NMR (400 MHz, Acetonitrile-d₃) δ 10.49 (s, 1H), 7.17 (td, J=8.0, 5.9 Hz, 1H), 6.93 (td, J=8.6, 1.7 Hz, 2H), 5.29 (q, J=3.9 Hz, 1H), 5.20 (dd, J=24.2, 3.9 Hz, 1H), 4.81 (dd, J=12.3, 4.5 Hz, 1H), 4.73-4.64 (m, 2H), 4.57 (d, J=9.8 Hz, 2H), 3.82-3.66 (m, 2H), 3.63-3.35 (m, 5H), 3.33 (s, 3H), 3.20-3.08 (m, 1H), 2.94 (s, 3H), 2.90-2.62 (m, 8H), 2.40-2.26 (m, 1H), 2.20-1.94 (m, 4H), 1.91-1.72 (m, 2H), 1.72-1.63 (m, 2H) ppm

LCMS: [M+H]⁺ m/z=582.3 amu

### Synthesis of Compound C-21

Intermediate 6-5, (R)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (6.53 mg, 0.013 mmol) was dissolved in anhydrous MeCN (85 μL) and treated with trans-4-dimethylaminocrotonic acid hydrochloride (4.2 mg, 0.026 mmol), EDC.HCl (4.9 mg, 0.026 mmol), and iPr₂EtN (4.5 μL, 0.026 mmol). After 15 minutes, the mixture was diluted with aqueous 0.25% TFA and purified by preparative HPLC (C18, 10→55% ACN in H₂O+0.25% TFA) to give compound C-21, (E)-4-(dimethylamino)-1-((R)-4-((R)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazin-1-yl)but-2-en-1-one (7.3 mg, 0.0118 mmol, 92% yield), as a faintly yellow film.

¹H NMR (400 MHz, Acetonitrile-d₃) δ 10.38 (s, 1H), 7.05 (td, J=8.0, 5.9 Hz, 1H), 6.86-6.77 (m, 2H), 6.71 (d, J=14.7 Hz, 1H), 6.60 (dt, J=15.3, 6.8 Hz, 1H), 4.69 (dd, J=12.5, 4.5 Hz, 1H), 4.62-4.22 (m, 4H), 3.70 (d, J=6.5 Hz, 2H), 3.67-3.57 (m, 2H), 3.47 (d, J=13.3 Hz, 1H), 3.33 (d, J=30.6 Hz, 3H), 3.23-3.18 (m, 3H), 3.07-2.98 (m, 1H), 2.82 (s, 3H), 2.79-2.47 (m, 15H), 2.25-2.14 (m, 1H), 2.07-1.81 (m, 4H), 1.76-1.61 (m, 2H), 1.59-1.51 (m, 2H) ppm

LCMS: [M+H]⁺ m/z=582.3 amu

### Example 7: Synthesis of Compounds C-22 and C-23

### Synthesis of Intermediate 7-1

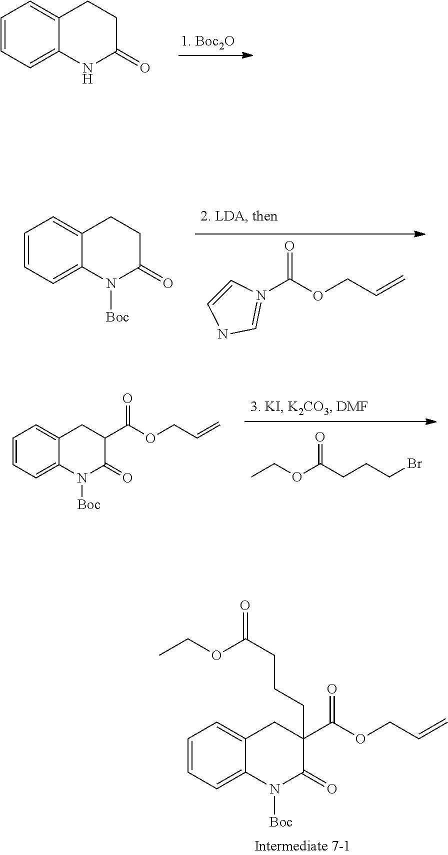

3,4-Dihydroquinolin-2(1H)-one (5.0 g, 34 mmol) was dissolved in anhydrous MeCN (68 mL) and treated with di-tert-butyl dicarbonate (8.15 g, 37 mmol) and DMAP (830 mg, 6.8 mmol), and the mixture was stirred at room temperature. After 13 hours, TLC analysis showed complete conversion to a single major product. The mixture was concentrated and purified by flash column chromatography on silica gel (15→20% EtOAc in hexanes) to give tert-butyl 2-oxo-3,4-dihydroquinoline-1(2H)-carboxylate (8.26 g, 33.4 mmol, 98% yield) as a colorless oil which crystallized upon standing.

¹H NMR (400 MHz, CDCl₃) δ 7.25-7.14 (m, 2H), 7.05 (td, J=7.4, 1.3 Hz, 1H), 6.94 (dd, J=8.1, 1.3 Hz, 1H), 2.98-2.90 (m, 2H), 2.69-2.61 (m, 2H), 1.60 (s, 9H) ppm

¹³C NMR (101 MHz, CDCl₃) δ 169.37, 151.85, 137.16, 128.06, 127.40, 125.94, 124.19, 117.02, 85.05, 32.37, 27.76, 25.55 ppm

Freshly prepared LDA, 1M in THE (4.85 mmol) was cooled to −78° C., and tert-butyl 2-oxo-3,4-dihydroquinoline-1(2H)-carboxylate (1.00 g, 4.04 mmol) was added dropwise as a solution in THE (10 mL), and the mixture was stirred for 40 minutes before adding allyl imidazole-1-carboxylate (738 mg, 4.85 mmol) as a solution in THF (10 mL). After 30 minutes, the cooling bath was removed and the mixture was allowed to warm to room temperature and stirred for 30 minutes, then quenched with saturated NH₄Cl. The mixture was partitioned between saturated NH₄Cl and EtOAc and the organic phase was collected and washed with saturated NH₄Cl, brine, dried over Na₂SO₄, filtered through a thin pad of silica gel, concentrated, and purified by flash column chromatography on silica gel (0→50% EtOAc in hexanes) to give 3-allyl 1-(tert-butyl) 2-oxo-3,4-dihydroquinoline-1,3(2H)-dicarboxylate (649.6 mg, 1.96 mmol, 49% yield) as a colorless oil.

¹H NMR (500 MHz, CDCl₃) δ 7.23 (t, J=8.1 Hz, 1H), 7.20 (d, J=8.1 Hz, 1H), 7.08 (td, J=7.5, 1.2 Hz, 1H), 6.93 (d, J=8.1 Hz, 1H), 5.84 (ddt, J=17.3, 10.7, 5.6 Hz, 1H), 5.28 (dq, J=17.1, 1.6 Hz, 1H), 5.20 (dq, J=10.5, 1.3 Hz, 1H), 4.71-4.58 (m, 2H), 3.67 (dd, J=10.0, 5.5 Hz, 1H), 3.40 (dd, J=15.7, 10.1 Hz, 1H), 3.11 (dd, J=15.7, 5.6 Hz, 1H), 1.61 (s, 9H) ppm

¹³C NMR (126 MHz, CDCl₃) δ 168.20, 165.33, 151.31, 136.47, 131.49, 128.43, 127.90, 124.67, 123.86, 118.61, 117.15, 85.64, 66.27, 48.62, 28.89, 27.74 ppm

3-allyl 1-(tert-butyl) 2-oxo-3,4-dihydroquinoline-1,3(2H)-dicarboxylate (3.45 g, 10 mmol) was dissolved in anhydrous DMF (20 mL) and treated with ethyl 4-bromobutanoate (2.23 mL, 16 mmol), KI (1.73 g, 10.4 mmol), and K₂CO₃ (4.3 g, 31 mmol), and the mixture was stirred at room temperature. After 23 hours, the mixture was diluted with H₂O and extracted with EtOAc (3 times). The combined extract was washed with brine, dried over Na₂SO₄, filtered through a thin pad of silica gel, and concentrated. The residue was purified by flash column chromatography on silica gel (0→40% EtOAc in hexanes) to give intermediate 7-1, 3-allyl 1-(tert-butyl) 3-(4-ethoxy-4-oxobutyl)-2-oxo-3,4-dihydroquinoline-1,3(2H)-dicarboxylate (4.36 g, 9.79 mmol, 94% yield), as a colorless oil.

LCMS: [M+2H−Boc]⁺ m/z=346.1 amu

### Synthesis of Intermediate 7-2

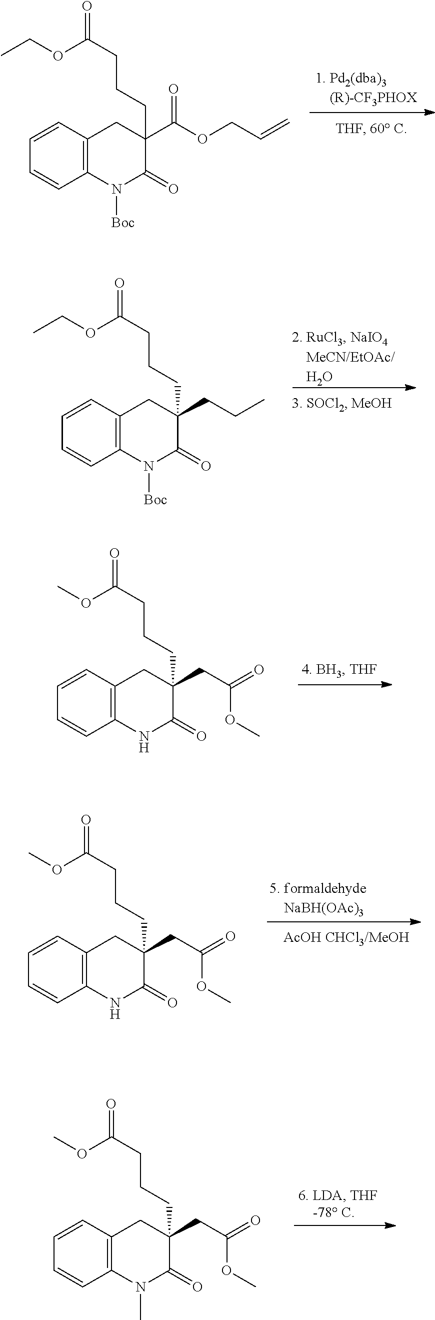

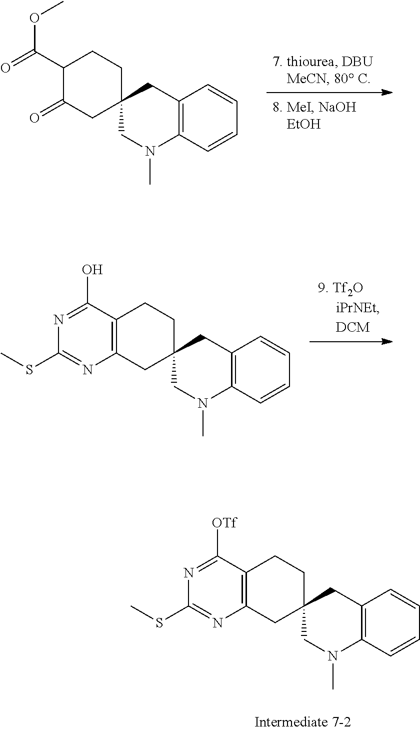 [SMILES: COC(=O)C1CC[C@]2(CC1=O)Cc1ccccc1N(C)C2 ; CSc1nc(O)c2c(n1)C[C@@]1(CC2)Cc2ccccc2N(C)C1 ; CSc1nc2c(c(OS(=O)(=O)C(F)(F)F)n1)CC[C@@]1(Cc3ccccc3N(C)C1)C2]

To an oven-dried flask containing intermediate 7-1, 3-allyl 1-(tert-butyl) 3-(4-ethoxy-4-oxobutyl)-2-oxo-3,4-dihydroquinoline-1,3(2H)-dicarboxylate (2.22 mg, 5.0 mmol), was added Pd₂(dba)₃ (228 mg, 0.25 mmol) and (R)-p-(CF₃)₃-t-BuPHOX (590 mg, 1.0 mmol), followed by THF (50 mL). The headspace was purged with argon and the flask was fitted with a condenser. The mixture was stirred at room temperature for 30 minutes before being warmed to 50° C. and stirring overnight. Upon completion, the mixture was cooled, diluted with DCM (50 mL), and filtered through a plug of celite, which was washed with more DCM (100 mL). The solvent was removed in vacuo and the mixture was purified using flash column chromatography on silica gel (0-60% EtOAc in hexanes) to yield tert-butyl (S)-3-allyl-3-(4-ethoxy-4-oxobutyl)-2-oxo-3,4-dihydroquinoline-1(2H)-carboxylate (1.78 mg, 4.43 mmol, 89% yield) as an off white solid.

LCMS: [M+H]⁺ m/z=402.2 amu

To a solution of tert-butyl (S)-3-allyl-3-(4-ethoxy-4-oxobutyl)-2-oxo-3,4-dihydroquinoline-1(2H)-carboxylate (1.78 g, 4.4 mmol) in MeCN (7.2 mL) and EtOAc (7.2 mL) was added H₂O (9.5 mL) followed by NaIO₄ (3.8 g, 17 mmol) and finally RuCl₃ xH₂O (28 mg, 0.13 mmol). The mixture was vigorously stirred at room temperature for 20 minutes, at which point an additional 2 equivalents of NaIO₄ was added. After 20 more minutes, an additional 1 equivalent of NaIO₄ was added and the reaction was stirred for a final 1 hour. Upon completion, the reaction mixture was cooled to room temperature and poured into a half-saturated solution of Na₂S₂O₃ (30 mL). The mixture was extracted using EtOAc (30 mL*3) and the combined organics were dried using Na₂SO₄, filtered, and concentrated to afford the crude acid, which was taken on without further purification.

LCMS: [M+H]⁺ m/z=420.2 amu

The crude acid was taken up in MeOH (45 mL) and cooled to 0° C. To the cooled solution was added SOCl₂ (3.9 mL, 53 mmol) dropwise, and the reaction was warmed to room temperature and stirred overnight. Upon completion, H₂O (100 mL) was slowly added before being extracted with EtOAc (60 mL*3). The combined organics were dried using Na₂SO₄, filtered, and concentrated to afford the crude methyl (R)-4-(3-(2-methoxy-2-oxoethyl)-2-oxo-1,2,3,4-tetrahydroquinolin-3-yl)butanoate, which was taken on to the next step without further purification.

LCMS: [M+H]⁺ m/z=320.1 amu

To a solution of the crude methyl (R)-4-(3-(2-methoxy-2-oxoethyl)-2-oxo-1,2,3,4-tetrahydroquinolin-3-yl)butanoate (1.42 g, 4.43 mmol, est.) in THE (45 mL) was added BH₃.THF (13.3 mL, 13 mmol, 1 M in THF). The reaction was heated to 50° C. and stirred overnight. Upon completion, 1 M HCl was slowly added dropwise to quench the reaction until no more gas bubbles were observed. After an additional 20 minutes of stirring, the aqueous was made basic using 2 M NaOH. the mixture was extracted with DCM (100 mL*3) and the combined organics were dried using Na₂SO₄, filtered, and concentrated in vacuo to afford the crude methyl (R)-4-(3-(2-methoxy-2-oxoethyl)-1,2,3,4-tetrahydroquinolin-3-yl)butanoate, which was taken on without further purification.

LCMS: [M+H]⁺ m/z=306.1 amu

To a cooled (0° C.) solution of the crude methyl (R)-4-(3-(2-methoxy-2-oxoethyl)-1,2,3,4-tetrahydroquinolin-3-yl)butanoate (1.35 g, 4.4 mmol, est.) in CHCl₃/MeOH (2:1, 45 mL) was added AcOH (2.5 mL, 44 mmol) followed by formaldehyde solution (1.8 mL, 22 mmol, 37% in H₂O). The mixture was stirred for 1 hour before NaBH(OAc)₃ (1.88 g, 8.9 mmol) was added and the mixture was warmed to room temperature. After 4 hours of additional stirring, the reaction was quenched with half-saturated NaHCO₃ (100 mL) and extracted using DCM (60 mL*3). The combined organics were dried over Na₂SO₄, filtered, and concentrated in vacuo. The mixture was purified using flash column chromatography on silica gel (10→80% EtOAc in hexanes) to yield methyl (R)-4-(3-(2-methoxy-2-oxoethyl)-1-methyl-1,2,3,4-tetrahydroquinolin-3-yl)butanoate (270 mg, 0.94 mmol, 75% yield) as a pale-yellow foam.

¹H NMR (400 MHz, Chloroform-d) δ 7.09 (ddd, J=8.2, 7.3, 1.7 Hz, 1H), 6.95 (dd, J=7.3, 1.1 Hz, 1H), 6.68-6.52 (m, 2H), 3.66 (s, 3H), 3.65 (s, 3H), 3.14 (dd, J=11.5, 1.7 Hz, 1H), 3.00 (d, J=11.5, 1H), 2.90 (s, 3H), 2.78-2.58 (m, 2H), 2.41 (d, J=14.7 Hz, 1H), 2.37-2.23 (m, 3H), 1.78-1.64 (m, 2H), 1.55-1.33 (m, 2H) ppm

LCMS: [M+H]⁺ m/z=320.1 amu

To a cooled (−78° C.) solution of methyl (R)-4-(3-(2-methoxy-2-oxoethyl)-1-methyl-1,2,3,4-tetrahydroquinolin-3-yl)butanoate (398 mg, 1.3 mmol) in THE (12.5 mL) was added LDA (1.38 mL, 2.5 mmol, 1.8 M in hexanes). The mixture was warmed to room temperature and stirred for 2 hours. The reaction was then quenched with saturated NH₄Cl (30 mL) and extracted with DCM (20 mL*3). The combined organics were dried over Na₂SO₄, filtered, and concentrated in vacuo. The mixture was purified using flash column chromatography on silica gel (0→40% EtOAc in hexanes) to yield methyl (1R)-1′-methyl-3-oxo-1′,4′-dihydro-2′H-spiro[cyclohexane-1,3′-quinoline]-4-carboxylate (270 mg, 0.94 mmol, 75% yield) as a pale yellow-foam.

LCMS: [M+H]⁺ m/z=288.1 amu

To a vial containing a solution of methyl (1R)-1′-methyl-3-oxo-1′,4′-dihydro-2′H-spiro[cyclohexane-1,3′-quinoline]-4-carboxylate (135 mg, 0.47 mmol) in MeCN (2.4 mL) was added thiourea (43 mg, 0.56 mmol) followed by DBU (105 μL, 0.70 mmol). The vial was sealed and the reaction was stirred overnight. Upon completion, the mixture was cooled to room temperature, poured into saturated NaHCO₃ (10 mL), and extracted with DCM (3×10 mL). The combined organics were dried over Na₂SO₄, filtered, and concentrated in vacuo. The crude (R)-2-mercapto-1′-methyl-1′,4′,5,8-tetrahydro-2′H,6H-spiro[quinazoline-7,3′-quinolin]-4-ol was taken on to the next step without further purification.

LCMS: [M+H]⁺ m/z=314.1 amu

To a vial containing the crude (R)-2-mercapto-1′-methyl-1′,4′,5,8-tetrahydro-2′H,6H-spiro[quinazoline-7,3′-quinolin]-4-ol (147 mg, 0.47 mmol, est.) was added EtOH (1.7 mL) followed by 1M NaOH (0.52 mL, 0.52 mmol, aq.). Once the substrate was fully dissolved, MeI (33 μL, 0.52 mmol) was added. The reaction was stirred for 1 hour, after which saturated NaHCO₃ (10 mL) was added and the mixture was extracted with DCM (10 mL*3). The combined organics were dried over Na₂SO₄, filtered, and concentrated in vacuo. The crude (R)-1′-methyl-2-(methylthio)-1′,4′,5,8-tetrahydro-2′H,6H-spiro[quinazoline-7,3′-quinolin]-4-ol was taken on to the next step without further purification.

LCMS: [M+H]⁺ m/z=328.1 amu

To a solution of the crude (R)-1′-methyl-2-(methylthio)-1′,4′,5,8-tetrahydro-2′H,6H-spiro[quinazoline-7,3′-quinolin]-4-ol (83 mg, 0.25 mmol) in DCM (1 mL) was added N,N-diisopropylethylamine (88 μL, 0.51 mmol). After stirring for 5 minutes, the mixture was cooled to 0° C. and triflic anhydride (380 μL, 0.38 mmol, 1M in DCM) was added. The reaction was stirred for 2 hours, after which hexanes (2 mL) was added and the mixture was passed through a plug of silica gel, rinsing with 30% EtOAc in hexanes (20 mL). The combined organics were concentrated in vacuo and intermediate 7-2, (R)-1′-methyl-2-(methylthio)-1′,4′,5,8-tetrahydro-2′H,6H-spiro[quinazoline-7,3′-quinolin]-4-yl trifluoromethanesulfonate, was used in subsequent reaction without further purification.

LCMS: [M+H]⁺ m/z=460.1 amu

### Synthesis of Intermediate 7-3

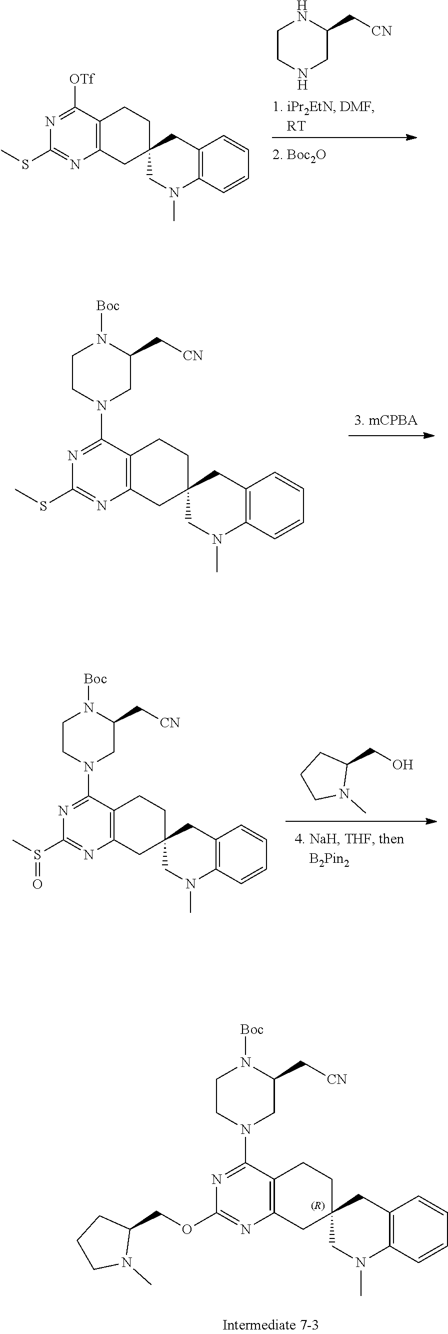

To a cooled (0° C.) solution of intermediate 7-2, (R)-1′-methyl-2-(methylthio)-1′,4′,5,8-tetrahydro-2′H,6H-spiro[quinazoline-7,3′-quinolin]-4-yl trifluoromethanesulfonate (126 g, 0.27 mmol), in DCM (3 mL) was added triethylamine (191 μL, 1.4 mmol), followed by (S)-2-(piperazin-2-yl)acetonitrile 2HCl (79 mg, 0.49 mmol). The resulting solution was warmed to room temperature and stirred for 6 hours. After consumption of starting material was observed, di-tert-butyl dicarbonate (240 mg, 1.1 mmol) was added and the reaction was heated to 40° C. and stirred for 2 hours. The reaction mixture was cooled to room temperature and poured into saturated NaHCO₃ (15 mL, aq.) and extracted with DCM (10 mL*3). The combined organic extracts were dried over Na₂SO₄, filtered and concentrated in vacuo. The mixture was purified using column chromatography (10→80% EtOAc in hexanes) to afford tert-butyl (S)-2-(cyanomethyl)-4-((R)-1′-methyl-2-(methylthio)-1′,4′,5,8-tetrahydro-2′H,6H-spiro[quinazoline-7,3′-quinolin]-4-yl)piperazine-1-carboxylate (119 mg, 0.22 mmol, 81% yield) as a white foam.

LCMS: [M+H]⁺ m/z=535.2 amu

To a cooled (0° C.) solution of tert-butyl (S)-2-(cyanomethyl)-4-((R)-1′-methyl-2-(methylthio)-1′,4′,5,8-tetrahydro-2′H,6H-spiro[quinazoline-7,3′-quinolin]-4-yl)piperazine-1-carboxylate (119 mg, 0.22 mmol) in DCM (2.2 mL) was added mCPBA (154 mg, 0.66 mmol). The mixture was stirred for 30 minutes, after which half-saturated NaHCO₃ (5 mL, aq.) was added and the mixture was extracted with DCM (5 mL*3). The combined organics were dried with Na₂SO₄, filtered, and concentrated in vacuo. The crude (7R)-4-((S)-4-(tert-butoxycarbonyl)-3-(cyanomethyl)piperazin-1-yl)-1′-methyl-2-(methylsulfonyl)-1′,4′,5,8-tetrahydro-2′H,6H-spiro[quinazoline-7,3′-quinoline] 1′-oxide was taken on to the next step without further purification.

LCMS: [M+H]⁺ m/z=583.2 amu

To a cooled (0° C.) vial containing NaH (26 mg, 0.68 mmol, 60% mineral oil dispersion) was added THE (1 mL) followed by (S)-(1-methylpyrrolidin-2-yl)methanol (132 μL, 1.11 mmol). The mixture was stirred for 45 minutes, at which point the crude (7R)-4-((S)-4-(tert-butoxycarbonyl)-3-(cyanomethyl)piperazin-1-yl)-1′-methyl-2-(methylsulfonyl)-1′,4′,5,8-tetrahydro-2′H,6H-spiro[quinazoline-7,3′-quinoline] 1-oxide (126 mg, 0.22 mmol, est.), as a solution in THE (1.2 mL), was added. The mixture was warmed to room temperature and stirred for 3 hours. Upon completion, the reaction was quenched with saturated NH₄Cl (5 mL, aq.) and the mixture was extracted with DCM (5 mL*3). The combined organics were dried with Na₂SO₄, filtered, and concentrated in vacuo. The crude (7R)-4-((S)-4-(tert-butoxycarbonyl)-3-(cyanomethyl)piperazin-1-yl)-1′-methyl-2-(((S)-1-methylpyrrolidin-2-yl)methoxy)-1′,4′,5,8-tetrahydro-2′H,6H-spiro[quinazoline-7,3′-quinoline] 1-oxide was taken on to the next step without further purification.

LCMS: [M+H]⁺ m/z=618.3 amu

To a vial containing the crude (7R)-4-((S)-4-(tert-butoxycarbonyl)-3-(cyanomethyl)piperazin-1-yl)-1′-methyl-2-(((S)-1-methylpyrrolidin-2-yl)methoxy)-1′,4′,5,8-tetrahydro-2′H,6H-spiro[quinazoline-7,3′-quinoline] 1-oxide (138 mg, 0.22 mmol, est.) in DCM (2.2 mL) was added B₂Pin₂ (28 mg, 0.11 mmol). The reaction was stirred at room temperature for 1 hour, at which point sat. NaHCO₃ (5 mL, aq.) was added and the mixture was extracted with DCM (5 mL*3). The combined organics were dried with Na₂SO₄, filtered, and concentrated in vacuo. Intermediate 7-3, tert-butyl (S)-2-(cyanomethyl)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazine-1-carboxylate, was used in subsequent reactions without further purification.

LCMS: [M+H]⁺ m/z=602.3 amu

### Synthesis of Compound C-22

To a vial containing intermediate 7-3, tert-butyl (S)-2-(cyanomethyl)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazine-1-carboxylate (134 mg, 0.22 mmol, est.) in DCM (4.5 mL) was added H₃PO₄ (137 μL, 2.2 mmol) dropwise. The reaction was stirred at room temperature for 2 hours, at which point H₂O (5 mL) was added and the solution was made basic by slow addition of 2 M NaOH solution (aq.). Once basic, the mixture was extracted with DCM (5 mL*3), and the combined organics were dried with Na₂SO₄, filtered, and concentrated in vacuo. The crude 2-((S)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazin-2-yl)acetonitrile was taken on to the next step without further purification.

LCMS: [M+H]⁺ m/z=502.3 amu

To a cooled (0° C.) solution of the crude 2-((S)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazin-2-yl)acetonitrile (57 mg, 0.11 mmol, est.) in DCM (2.3 mL) was added N,N-diisopropylethylamine (200 μL, 1.1 mmol), followed by acrylic anhydride (40 μL, 0.34 mmol). The mixture was warmed to room temperature and stirred for 2 hours, at which point the solution was concentrated in vacuo, taken up in DMSO, filtered, and purified using preparative HPLC (C18, 20→60% MeCN in H₂O+0.25% TFA). The combined fractions containing the desired product were lyophilized to yield compound C-22, 2-((S)-1-acryloyl-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazin-2-yl)acetonitrile (7.9 mg, 0.014 mmol, 13% yield, over 5 steps), as a fluffy off-white solid.

¹H NMR (400 MHz, Acetonitrile-d₃, TFA salt) δ 10.48 (s, 1H), 7.14-7.00 (m, 1H), 6.94 (dd, J=7.4, 1.6 Hz, 1H), 6.82-6.64 (m, 2H), 6.61 (td, J=7.3, 1.1 Hz, 1H), 6.25 (dd, J=16.7, 2.1 Hz, 1H), 5.77 (dd, J=10.6, 2.1 Hz, 1H), 5.00 (bs, 1H), 4.81-4.61 (m, 2H), 4.49 (d, J=14.1 Hz, 1H), 4.33 (bs, 1H), 4.12-3.88 (m, 1H), 3.78-3.63 (m, 2H), 3.62-3.38 (m 2H), 3.15-3.00 (m, 3H), 2.97-2.85 (m, 5H), 2.80 (bs, 2H), 2.75-2.51 (m, 6H), 2.50 (s, 14H), 2.34-2.23 (m, 1H), 2.15-1.96 (m, 3H), 1.69-1.54 (m, 2H) ppm

LCMS: [M+H]⁺ m/z=556.3 amu

### Synthesis of Compound C-23

To a cooled (0° C.) solution of the crude 2-((S)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazin-2-yl)acetonitrile (57 mg, 0.11 mmol, crude est.) in DCM (2.3 mL) was added N,N-diisopropylethylamine (200 μL, 1.1 mmol), followed by 2-fluoroacrylic anhydride (55 mg, 0.34 mmol). The mixture was warmed to room temperature and stirred for 2 hours, at which point the solution was concentrated in vacuo, taken up in DMSO, filtered, and purified using preparative HPLC (C18, 20→60% MeCN in H₂O+0.25% TFA). The combine fractions containing the desired product were lyophilized to yield compound C-23, 2-((S)-1-(2-fluoroacryloyl)-4-((R)-1′-methyl-2-(((S)-1-methylpyrrolidin-2-yl)methoxy)-1′,4′,5,8-tetrahydro-2′H,6H-spiro[quinazoline-7,3′-quinolin]-4-yl)piperazin-2-yl)acetonitrile (10.5 mg, 0.018 mmol, 16% yield, over 5 steps), as a fluffy off-white solid.

¹H NMR (400 MHz, Acetonitrile-d₃, TFA salt) δ 10.65 (s, 1H), 7.07 (ddd, J=8.2, 7.3, 1.6 Hz, 1H), 6.93 (dd, J=7.4, 1.6 Hz, 1H), 6.67 (dd, J=8.3, 1.1 Hz, 1H), 6.60 (td, J=7.3, 1.1 Hz, 1H), 5.42-5.09 (m, 2H), 4.84 (bs, 1H), 4.70 (qd, J=12.4, 4.2 Hz, 2H), 4.47 (d, J=14.1 Hz, 1H), 4.32 (d, J=12.1 Hz, 1H), 3.87 (bs, 4H), 3.76-3.61 (m, 2H), 3.52 (d, J=14.1 Hz, 1H), 3.43-3.30 (m, 1H), 3.16-2.98 (m, 3H), 2.98-2.80 (m, 7H), 2.80-2.50 (m, 6H), 2.37-2.22 (m, 1H), 2.18-1.96 (m, 2H), 1.70-1.55 (m, 2H) ppm

LCMS: [M+H]⁺ m/z=574.3 amu

### Example 8: Synthesis of Compounds C-24 Through C-30

### Synthesis of Intermediate 8-1

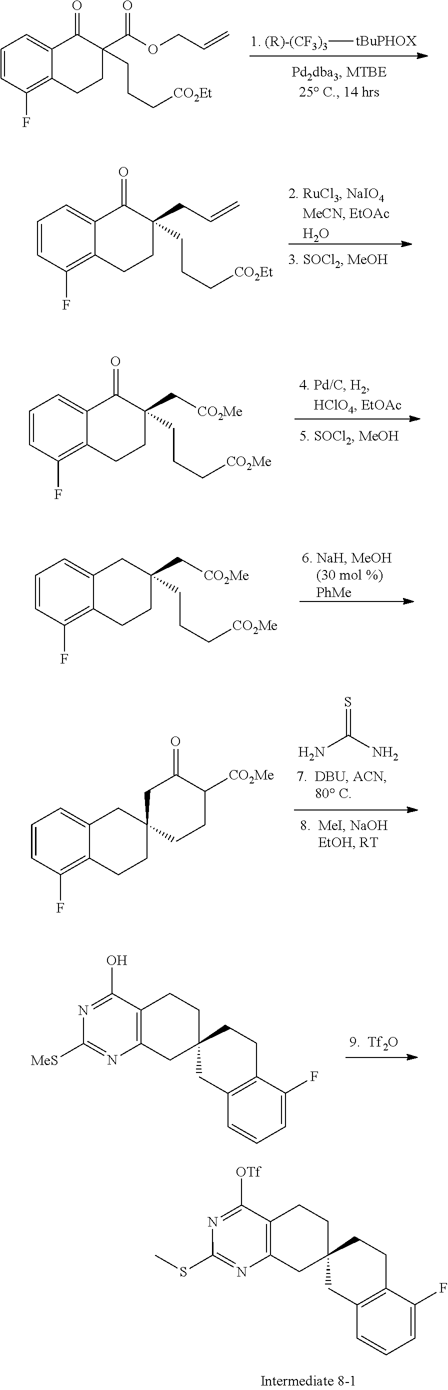

Pd₂(dba)₃ (174 mg, 0.19 mmol) and (R)-p-(CF₃)₃-t-BuPHOX (300 mg, 0.51 mmol) were suspended in degassed anhydrous MTBE (40 mL) under N₂ atmosphere and the mixture was warmed to 25° C. and stirred for 45 minutes. Separately, intermediate 6-1, allyl 2-(4-ethoxy-4-oxo-butyl)-5-fluoro-1-oxo-tetralin-2-carboxylate (2.3 g, 6.4 mmol) was dissolved in MTBE (40 mL) and sparged with N₂ for 20 minutes, then added to the catalyst mixture. After 13 hours, the reaction was opened to air and amended with 0.3 vol hexanes and a small amount of silica gel, and the mixture was stirred for 10 minutes, then filtered through a thin pad of silica gel and concentrated. The residue was purified by flash column chromatography on silica gel (0→15% EtOAc in hexanes) to give ethyl (R)-4-(2-allyl-5-fluoro-1-oxo-1,2,3,4-tetrahydronaphthalen-2-yl)butanoate (1.89 g, 5.94 mmol, 94% yield) as a pale yellow viscous oil.

LCMS: [M+H]⁺ m/z=319.2 amu

Ethyl (R)-4-(2-allyl-5-fluoro-1-oxo-1,2,3,4-tetrahydronaphthalen-2-yl)butanoate (1.89 g, 5.9 mmol) was dissolved in EtOAc (11.6 mL) and MeCN (11.6 mL) and treated with H₂O (18.2 mL), NaIO₄ (6.35 g, 30 mmol) and RuCl₃.xH₂O (27.1 mg, 0.13 mmol), and the mixture was stirred vigorously at room temperature. After 2 hours, the mixture was diluted with 0.5M NaHSO₄ and EtOAc and stirred for 5 minutes, then filtered through Celite. The organic phase was collected and the aqueous was extracted twice more with EtOAc. The combined extract was washed with brine, dried over Na₂SO₄, filtered through Celite, concentrated and further dried in vacuo. The residue was taken up in MeOH (35 mL), cooled to 0° C., and treated with SOCl₂ (4.3 mL, 59 mmol) dropwise. The cooling bath was removed and the mixture was stirred at room temperature. After 2 hours, the mixture was concentrated and reconstituted in 7:3 Et₂O:hexanes, filtered through a thin pad of silica gel, and concentrated to give methyl (R)-4-(5-fluoro-2-(2-methoxy-2-oxoethyl)-1-oxo-1,2,3,4-tetrahydronaphthalen-2-yl)butanoate (1.89 g, 95% yield) as a faint yellow oil. R_{f}=0.39 (1:1 hexanes:Et₂O).

LCMS: [M+H]⁺ m/z=337.1 amu

Methyl (R)-4-(5-fluoro-2-(2-methoxy-2-oxoethyl)-1-oxo-1,2,3,4-tetrahydronaphthalen-2-yl)butanoate (1.64 g, 4.9 mmol) was dissolved in EtOAc (25 mL) and treated with Pd/C, 10 wt % (320 mg) and HClO₄, 60% (80 μL, 0.52 mmol) and the vessel was charged with H₂. After 11 hours, the mixture was filtered through Celite and concentrated. The residue was taken up in MeOH (28 mL) and treated with SOCl₂ (2.0 mL, 28 mmol) dropwise at 0° C. The cooling bath was removed and the mixture was stirred for 2 hours then concentrated, diluted with H₂O, and extracted with Et₂O (3 times). The combined extract was washed with sat NaHCO₃, brine, dried over Na₂SO₄, filtered through a thin pad of silica gel, and concentrated to give methyl (S)-4-(5-fluoro-2-(2-methoxy-2-oxoethyl)-1,2,3,4-tetrahydronaphthalen-2-yl)butanoate (1.69 g, 5.24 mmol, 93% yield). R_{f}=0.43 (8:2 hexanes:EtOAc).

LCMS: [M+H]⁺ m/z=322.2 amu

NaH (251.64 mg, 6.3 mmol) was suspended in anhydrous toluene (20 mL) and treated with MeOH (53 μL, 1.3 mmol). The mixture was stirred until gas evolution ceased, then a solution of methyl (S)-4-(5-fluoro-2-(2-methoxy-2-oxoethyl)-1,2,3,4-tetrahydronaphthalen-2-yl)butanoate (1.69 g, 5.2 mmol) in toluene (10 mL) was added and the mixture was heated to 70° C. After 4 hours, the mixture was poured into sat NH₄Cl and extracted with EtOAc (3 times). The combined extract was washed with brine, dried over Na₂SO₄, filtered, and concentrated to give the crude methyl (1S)-5′-fluoro-3-oxo-3′,4′-dihydro-1′H-spiro[cyclohexane-1,2′-naphthalene]-4-carboxylate (1.16 g, 76% yield) as a pale yellow oil, which was taken on to the next step without further purification.

¹H NMR (600 MHz, Chloroform-d) δ 12.12 (d, J=1.2 Hz, 1H), 7.09-7.04 (m, 1H), 6.86-6.81 (m, 2H), 3.79-3.75 (m, 3H), 2.77 (t, J=6.9 Hz, 2H), 2.67 (dd, J=16.4, 0.9 Hz, 1H), 2.56 (d, J=16.2 Hz, 1H), 2.36-2.26 (m, 2H), 2.19 (dq, J=18.2, 1.5 Hz, 1H), 2.12 (dq, J=18.2, 1.4 Hz, 1H), 1.72 (dtt, J=13.5, 6.7, 1.2 Hz, 1H), 1.63 (dtd, J=13.5, 6.7, 1.2 Hz, 1H), 1.59-1.52 (m, 1H), 1.51-1.43 (m, 1H) ppm

LCMS: [M+H]⁺ m/z=291.1 amu

The crude methyl (1S)-5′-fluoro-3-oxo-3′,4′-dihydro-1′H-spiro[cyclohexane-1,2′-naphthalene]-4-carboxylate (488 mg, 1.7 mmol) was dissolved in anhydrous MeCN (8.4 mL) and treated with thiourea (154 mg, 2.0 mmol) and DBU (376 μL, 2.5 mmol), and the mixture was heated to 80° C. After 3 hours, the mixture was cooled to room temperature, concentrated to approximately 1 mL, and diluted with aqueous NaH₂PO₄. The resulting precipitate was collected by filtration and the still wet material was suspended in EtOH (8.4 mL) and treated with 1M NaOH (1.85 mL, 1.9 mmol) and MeI (126 μL, 2.0 mmol), and the mixture was stirred vigorously at room temperature for 19 hours. The mixture was poured into aqueous NaH₂PO₄ and extracted with CHCl₃ (3 times). The combined extract was washed with brine, dried over Na₂SO₄, and purified by flash column chromatography on silica gel (0→10% MeOH in CH₂Cl₂) (Rf=0.37 (95:5 CHCl₃:MeOH)) to give (S)-5-fluoro-2′-(methylthio)-3,4,5′,8′-tetrahydro-1H,6′H-spiro[naphthalene-2,7′-quinazolin]-4′-ol (403.8 mg, 1.22 mmol, 72.7% yield) as a white solid.

LCMS: [M+H]⁺ m/z=331.1 amu

(S)-5-fluoro-2′-(methylthio)-3,4,5′,8′-tetrahydro-1H,6′H-spiro[naphthalene-2,7′-quinazolin]-4′-ol (229 mg, 0.69 mmol) was suspended in anhydrous DCM (1.7 mL) and treated with freshly distilled iPr₂EtN (241 μL, 1.4 mmol) then the mixture was cooled to 0° C., and triflic anhydride, 1M in DCM (1040 μL, 1.0 mmol) was added dropwise. The cooling bath was removed and the mixture was stirred at room temperature for 1 hour, then diluted with 2 vol hexanes and filtered through a pipet column of silica gel rinsing with 9:1 hexanes:EtOAc. The filtrate was concentrated and purified by flash column chromatography on silica gel (0→15% EtOAc in hexanes) (Rf=0.39 (9:1 hexanes:EtOAc)) to give intermediate 8-1, (S)-5-fluoro-2′-(methylthio)-3,4,5′,8′-tetrahydro-1H,6′H-spiro[naphthalene-2,7′-quinazolin]-4′-yl trifluoromethanesulfonate (256.8 mg, 0.555 mmol, 80% yield) as a colorless vitreous oil.

LCMS: [M+H]+=463.1 amu

### Synthesis of Intermediate 8-2

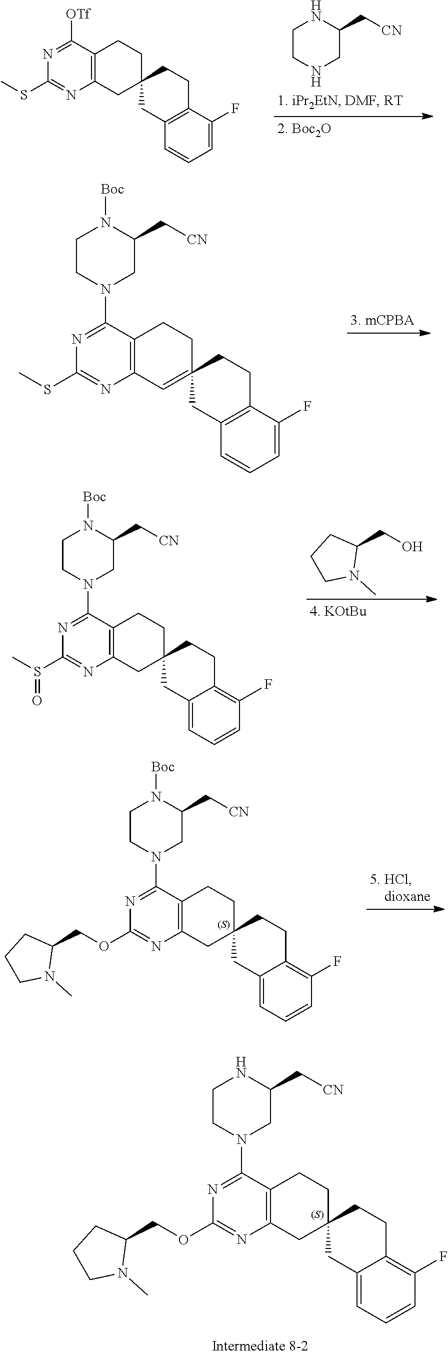

Intermediate 8-1, (S)-5-fluoro-2′-(methylthio)-3,4,5′,8′-tetrahydro-1H,6′H-spiro[naphthalene-2,7′-quinazolin]-4′-yl trifluoromethanesulfonate (114.5 mg, 0.25 mmol) was dissolved in anhydrous DMF (707 μL) and treated with iPr₂EtN (129 μL, 0.74 mmol) and 2-[(2S)-piperazin-2-yl]acetonitrile dihydrochloride (58.9 mg, 0.30 mmol), and the mixture was stirred at room temperature for 30 minutes. Boc₂O (85.3 μL, 0.37 mmol) was added and the mixture was stirred for 15 hours then diluted with EtOAc and washed with sat NH₄Cl, brine, dried over Na₂SO₄, filtered through a thin pad of silica gel, and concentrated. The crude isolate was purified by flash column chromatography on silica gel (0→30% EtOAc in hexanes) to give tert-butyl (S)-2-(cyanomethyl)-4-((S)-5-fluoro-2′-(methylthio)-3,4,5′,8′-tetrahydro-1H,6′H-spiro[naphthalene-2,7′-quinazolin]-4′-yl)piperazine-1-carboxylate (167.8 mg, >100% yield) as a white foam.

LCMS: [M+H]⁺ m/z=538.3 amu

tert-Butyl (S)-2-(cyanomethyl)-4-((S)-5-fluoro-2′-(methylthio)-3,4,5′,8′-tetrahydro-1H,6′H-spiro[naphthalene-2,7′-quinazolin]-4′-yl)piperazine-1-carboxylate (133.1 mg, 0.25 mmol) was dissolved in DCM (825 μL), cooled to 0° C., and treated with mCPBA (62.7 mg, 0.27 mmol). After 20 minutes, the mixture was diluted with Et₂O and washed sequentially with half-saturated NaHCO₃ (2 times), brine, dried over Na₂SO₄, filtered, and concentrated to yield the crude tert-butyl (2S)-2-(cyanomethyl)-4-((2S)-5-fluoro-2′-(methylsulfinyl)-3,4,5′,8′-tetrahydro-1H,6′H-spiro[naphthalene-2,7′-quinazolin]-4′-yl)piperazine-1-carboxylate, which was taken forward without further purification.

LCMS: [M+H]⁺ m/z=554.3 amu

1-Methyl-L-prolinol (57 mg, 0.50 mmol) was dissolved in anhydrous THF (1.5 mL) and treated with KOtBu, 1.7M in THE (291 μL, 0.50 mmol) and the mixture was stirred for 5 minutes, then added to a solution of the crude tert-butyl (2S)-2-(cyanomethyl)-4-((2S)-5-fluoro-2′-(methylsulfinyl)-3,4,5′,8′-tetrahydro-1H,6′H-spiro[naphthalene-2,7′-quinazolin]-4′-yl)piperazine-1-carboxylate (137.1 mg, 0.25 mmol) in anhydrous THF (1 mL) at 0° C. After 30 minutes, the mixture was poured into aqueous K₂CO₃ and extracted with EtOAc (3 times). The combined extract was washed with brine, dried over Na₂SO₄, filtered, concentrated, and purified by flash column chromatography on basic alumina (0→100% Et₂O in hexanes followed by 100% EtOAc) to give tert-butyl (S)-2-(cyanomethyl)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (113.5 mg, 0.188 mmol, 76% yield) as a pale yellow foam.

LCMS: [M+H]⁺ m/z=605.4 amu

tert-Butyl (S)-2-(cyanomethyl)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (113.5 mg, 0.19 mmol) was treated with 4N HCl in dioxane (2 mL) at room temperature for 30 minutes. The mixture was then concentrated, dissolved in 1N HCl, and washed with Et₂O (2 times), and the combined ethereal wash was extracted with 1N HCl once. The combined aqueous was basified with K₂CO₃ and back-extracted with EtOAc (3 times) and the combined extract was dried over K₂CO₃, filtered, and concentrated to give intermediate 8-2, 2-((S)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (86.5 mg, 0.171 mmol, 91% yield).

LCMS: [M+H]⁺ m/z=504.4 amu

### Synthesis of Compound C-24

Intermediate 8-2, 2-((S)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (21.6 mg, 0.043 mmol), was dissolved in anhydrous MeCN (400 μL) and treated with acrylic anhydride (7.4 μL, 0.064 mmol) at 0° C. then allowed to warm to room temperature. After 10 minutes, the mixture was diluted with aqueous 0.25% TFA and purified by preparative HPLC (C18 10→60% ACN in H₂O+0.25% TFA) to give compound C-24, 2-((S)-1-acryloyl-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (7.6 mg, 32% yield) as a colorless film.

¹H NMR (400 MHz, Acetonitrile-d₃) δ 10.74 (s, 1H), 7.14 (td, J=8.1, 5.9 Hz, 1H), 6.90 (dd, J=9.7, 7.4 Hz, 2H), 6.72 (s, 1H), 6.25 (dd, J=16.7, 2.1 Hz, 1H), 5.77 (dd, J=10.6, 2.1 Hz, 1H), 4.80-4.64 (m, 2H), 4.51 (dt, J=14.1, 2.4 Hz, 1H), 4.42-4.25 (m, 1H), 3.96 (d, J=24.6 Hz, 1H), 3.77-3.62 (m, 2H), 3.63-3.40 (m, 2H), 3.14-3.02 (m, 1H), 2.91 (s, 3H), 2.88-2.60 (m, 11H), 2.35-2.23 (m, 1H), 2.16-1.91 (m, 4H), 1.88-1.78 (m, 1H), 1.76-1.63 (m, 2H), 1.57 (dt, J=12.7, 6.2 Hz, 1H) ppm

LCMS: [M+H]⁺ m/z=559.3 amu

### Synthesis of Compound C-25

Intermediate 8-2, 2-((S)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (21.6 mg, 0.043 mmol), was dissolved in anhydrous MeCN (400 μL) and treated with 2-fluoroacrylic anhydride (10.4 mg, 0.0643 mmol) at room temperature. After 25 minutes, the mixture was diluted with aqueous 0.25% TFA and purified by preparative HPLC (C18 10-60% ACN in H₂O+0.25% TFA) to give compound C-25, 2-((S)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-1-(2-fluoroacryloyl)piperazin-2-yl)acetonitrile (20.1 mg, 0.0349 mmol, 81% yield).

¹H NMR (400 MHz, Acetonitrile-d₃) δ 9.90 (s, 1H), 6.15 (td, J=8.1, 5.9 Hz, 1H), 5.97-5.87 (m, 2H), 4.38-4.18 (m, 2H), 3.86 (s, 1H), 3.77 (dd, J=12.3, 5.7 Hz, 1H), 3.70 (dd, J=12.3, 3.4 Hz, 1H), 3.56 (dt, J=14.2, 2.3 Hz, 1H), 3.38 (d, J=9.8 Hz, 1H), 3.11 (s, 1H), 2.79-2.65 (m, 2H), 2.60-2.50 (m, 1H), 2.48-2.31 (m, 1H), 2.17-2.04 (m, 1H), 1.94 (s, 3H), 1.92-1.85 (m, 2H), 1.85-1.75 (m, 3H), 1.74-1.61 (m, 5H), 1.38-1.25 (m, 1H), 1.18-0.90 (m, 4H), 0.90-0.79 (m, 1H), 0.71 (dq, J=20.4, 6.6 Hz, 2H), 0.60 (dt, J=13.1, 6.1 Hz, 1H) ppm

LCMS: [M+H]⁺ m/z=577.3 amu

### Synthesis of Compound C-26

Intermediate 8-2, 2-((S)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (21.6 mg, 0.043 mmol), was dissolved in anhydrous MeCN (400 μL) and treated with iPr₂EtN (14.9 μL, 0.086 mmol), trans-4-dimethylaminocrotonic acid hydrochloride (14.2 mg, 0.086 mmol), and EDC.HCl (16.4 mg, 0.086 mmol), and the mixture was stirred at room temperature. After 16 hours, the mixture was diluted with aqueous 0.25% TFA and purified by preparative HPLC (C18 10→60% ACN in H₂O+0.25% TFA) to give compound C-26, 2-((S)-1-((E)-4-(dimethylamino)but-2-enoyl)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (20.5 mg, 0.0333 mmol, 78% yield), as a light brown colored film.

¹H NMR (400 MHz, Acetonitrile-d₃) δ 12.13 (s, 1H), 10.63 (s, 1H), 7.14 (td, J=8.2, 5.9 Hz, 1H), 6.94-6.85 (m, 2H), 6.79-6.69 (m, 1H), 4.82-4.64 (m, 2H), 4.60-4.28 (m, 2H), 4.12-3.94 (m, 1H), 3.81 (d, J=6.2 Hz, 2H), 3.77-3.31 (m, 5H), 3.20-3.01 (m, 2H), 2.92 (s, 3H), 2.87-2.60 (m, 16H), 2.35-2.23 (m, 1H), 2.16-1.90 (m, 4H), 1.88-1.77 (m, 1H), 1.71 (dt, J=13.4, 7.0 Hz, 2H), 1.59 (s, 1H) ppm

LCMS: [M+H]⁺ m/z=616.4 amu

### Synthesis of Intermediate 8-3

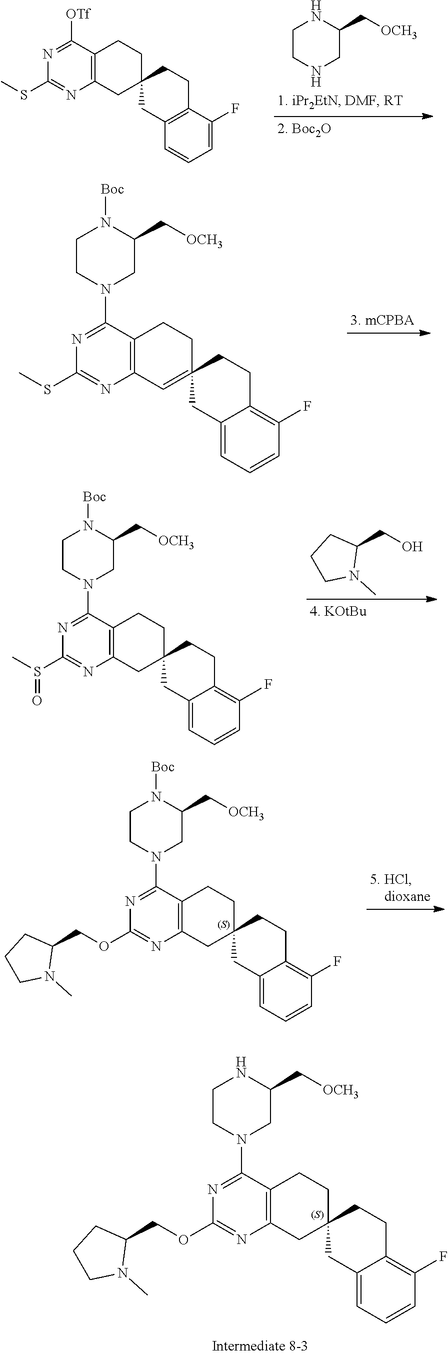

Intermediate 8-1, (S)-5-Fluoro-2′-(methylthio)-3,4,5′,8′-tetrahydro-1H,6′H-spiro[naphthalene-2,7′-quinazolin]-4′-yl trifluoromethanesulfonate (114.5 mg, 0.25 mmol), was dissolved in anhydrous DMF (710 μL) and treated with iPr₂EtN (129 μL, 0.74 mmol) and the dihydrochloride salt of intermediate 6-4, (2R)-2-(methoxymethyl)piperazine dihydrochloride (60.3 mg, 0.30 mmol), and the mixture was stirred at room temperature for 30 minutes. Boc₂O (85 μL, 0.37 mmol) was then added and stirring continued for 16 hours. The mixture was diluted with EtOAc and washed with half-saturated NaHCO₃ (2 times), brine, dried over Na₂SO₄, filtered through a thin pad of silica gel, and concentrated, and purified by flash column chromatography on silica gel (0→30% EtOAc in hexanes) to give tert-butyl (R)-4-((S)-5-fluoro-2′-(methylthio)-3,4,5′,8′-tetrahydro-1H,6′H-spiro[naphthalene-2,7′-quinazolin]-4′-yl)-2-(methoxymethyl)piperazine-1-carboxylate (148.1 mg, >100% yield) as a white foam.

LCMS: [M+H]⁺ m/z=543.3 amu

tert-Butyl (R)-4-((S)-5-fluoro-2′-(methylthio)-3,4,5′,8′-tetrahydro-1H,6′H-spiro[naphthalene-2,7′-quinazolin]-4′-yl)-2-(methoxymethyl)piperazine-1-carboxylate (134.4 mg, 0.25 mmol) was dissolved in DCM (825 μL), cooled to 0° C., and treated with mCPBA (62.7 mg, 0.27 mmol). After 20 minutes, the mixture was diluted with Et₂O and washed with half-saturated NaHCO₃ (2 times), brine, dried over Na₂SO₄, and concentrated to give the crude tert-butyl (2R)-4-((2S)-5-fluoro-2′-(methylsulfinyl)-3,4,5′,8′-tetrahydro-1H,6′H-spiro[naphthalene-2,7′-quinazolin]-4′-yl)-2-(methoxymethyl)piperazine-1-carboxylate (150.1 mg, >100% yield) as a white foam, which was used in the next step without purification.

LCMS: [M+H]⁺ m/z=559.3 amu

1-Methyl-L-prolinol (57.0 mg, 0.50 mmol) was dissolved in anhydrous THF (1.5 mL) and treated with KOtBu, 1.7M in THE (291.29 μL, 0.50 mmol) and the mixture was stirred for 5 minutes, then added to a solution of the crude tert-butyl (2R)-4-((2S)-5-fluoro-2′-(methylsulfinyl)-3,4,5′,8′-tetrahydro-1H,6′H-spiro[naphthalene-2,7′-quinazolin]-4′-yl)-2-(methoxymethyl)piperazine-1-carboxylate (138.3 mg, 0.25 mmol, est.) in anhydrous THF (1 mL) at 0° C. After 1 hour, the mixture was poured into aqueous K₂CO₃ and extracted with EtOAc (3 times). The combined extract was washed with brine, dried over Na₂SO₄, filtered, and concentrated to give the crude tert-butyl (R)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazine-1-carboxylate (165.9 mg, >100% yield) as a faintly yellow foam, which was used in the subsequent step without purification. Rf=0.45 (95:5 CHCl₃:MeOH+2% Et₃N).

LCMS: [M+H]⁺ m/z=605.4 amu

The crude tert-butyl (R)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazine-1-carboxylate (165.9 mg, 0.27 mmol) was treated with 4N HCl in dioxane (2 mL) at room temperature for 30 minutes. The mixture was dissolved in 1N HCl and washed with Et₂O (2 times) and the combined ethereal layer was extracted with 1N HCl once. The combined aqueous was basified with K₂CO₃ and back-extracted with EtOAc (3. times), and the combined extract was dried over K₂CO₃, filtered, and concentrated to give intermediate 8-3, (S)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (123.9 mg, 0.243 mmol, 89% yield).

LCMS: [M+H]⁺ m/z=510.3 amu

### Synthesis of Compound C-27

Intermediate 8-3, (S)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (31.0 mg, 0.061 mmol), was dissolved in MeCN (610 μL) and treated with acrylic anhydride (10.5 μL, 0.091 mmol). After 1 hour, the mixture was diluted with aqueous 0.25% TFA and purified by preparative HPLC (C18, 10→55% ACN in H₂O+0.25% TFA) to give compound C-27, 1-((R)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazin-1-yl)prop-2-en-1-one (29.2 mg, 0.0502 mmol, 83% yield) as a colorless film.

¹H NMR (400 MHz, Acetonitrile-d₃) δ 10.59 (s, 1H), 7.05 (td, J=8.0, 5.9 Hz, 1H), 6.86-6.76 (m, 2H), 6.62 (t, J=13.2 Hz, 1H), 6.12 (dd, J=16.8, 2.2 Hz, 1H), 5.62 (dd, J=10.6, 2.2 Hz, 1H), 4.74-4.61 (m, 1H), 4.62-4.46 (m, 2H), 4.37 (s, 2H), 3.70-3.53 (m, 2H), 3.46 (dd, J=13.9, 4.0 Hz, 2H), 3.32 (d, J=28.1 Hz, 3H), 3.18 (s, 3H), 3.01 (dt, J=12.1, 8.3 Hz, 1H), 2.82 (s, 3H), 2.76-2.49 (m, 8H), 2.20 (ddd, J=12.6, 8.3, 5.4 Hz, 1H), 2.11-1.81 (m, 4H), 1.76 (dt, J=12.5, 6.5 Hz, 1H), 1.70-1.55 (m, 2H), 1.49-1.40 (m, 1H) ppm

LCMS: [M+H]⁺ m/z=582.3 amu

### Synthesis of Compound C-28

Intermediate 8-3, (S)-5-fluoro-4′-((R)-3-(methoxymethyl)piperazin-1-yl)-2′-(((S)-1-methylpyrrolidin-2-yl)methoxy)-3,4,5′,8′-tetrahydro-1H,6′H-spiro[naphthalene-2,7′-quinazoline] (31.0 mg, 0.061 mmol), was dissolved in MeCN (610 μL) and treated with 2-fluoroacrylic anhydride (14.8 mg, 0.091 mmol). After 90 minutes, the mixture was diluted with aqueous 0.25% TFA and purified by preparative HPLC (C18, 10→55% ACN in H₂O+0.25% TFA) to give compound C-28, 2-fluoro-1-((R)-4-((S)-5-fluoro-2′-(((S)-1-methylpyrrolidin-2-yl)methoxy)-3,4,5′,8′-tetrahydro-1H,6′H-spiro[naphthalene-2,7′-quinazolin]-4′-yl)-2-(methoxymethyl)piperazin-1-yl)prop-2-en-1-one (29.2 mg, 0.0502 mmol, 83% yield) as a colorless film.

¹H NMR (400 MHz, Acetonitrile-d₃) δ 10.72 (s, 1H), 7.05 (td, J=8.0, 5.9 Hz, 1H), 6.85-6.76 (m, 2H), 5.20-5.03 (m, 2H), 4.69 (dd, J=12.3, 5.3 Hz, 1H), 4.61-4.35 (m, 4H), 3.70-3.54 (m, 2H), 3.47-3.31 (m, 3H), 3.30-3.20 (m, 2H), 3.18 (s, 3H), 3.00 (dt, J=11.7, 8.4 Hz, 1H), 2.83 (s, 3H), 2.75-2.48 (m, 8H), 2.27-2.17 (m, 1H), 2.07-1.81 (m, 4H), 1.80-1.68 (m, 1H), 1.68-1.51 (m, 2H), 1.45 (ddd, J=13.2, 7.9, 5.1 Hz, 1H) ppm

LCMS: [M+H]⁺ m/z=582.3 amu

### Example 9: Synthesis of Compounds C-29 and C-30

### Synthesis of Intermediate 9-1

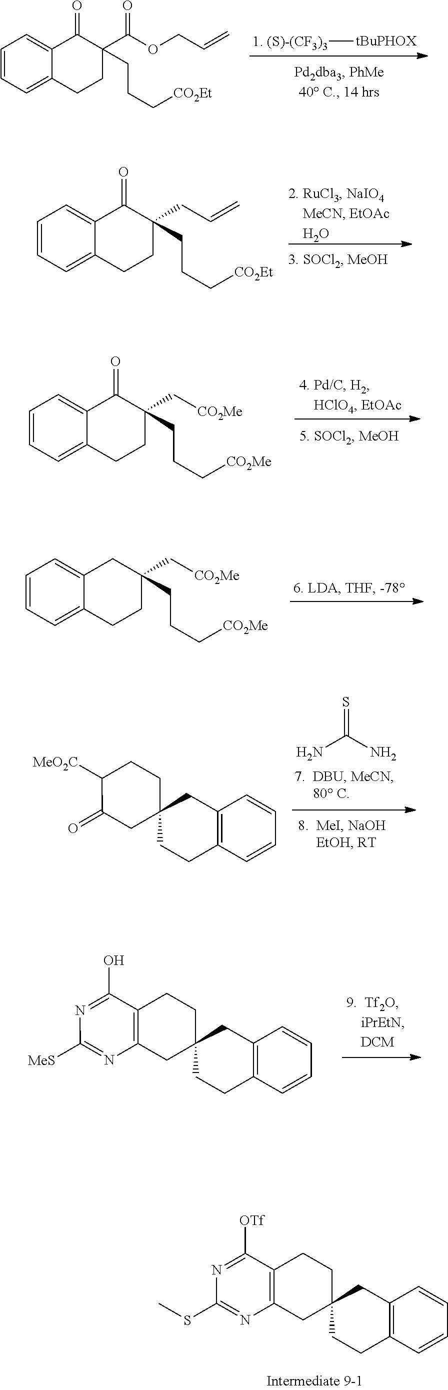

(S)-p-(CF₃)₃-t-BuPHOX (368 mg, 0.62 mmol) and Pd₂(dba)₃ (214 mg, 0.23 mmol) were dissolved in degassed anhydrous toluene (68 mL) under N₂ atmosphere and the mixture was stirred for 30 minutes at room temperature. Separately, allyl 2-(4-ethoxy-4-oxobutyl)-1-oxo-1,2,3,4-tetrahydronaphthalene-2-carboxylate (2.68 g, 7.8 mmol) was dissolved in toluene (30 mL) and sparged with N₂ for 20 minutes then added to the catalyst mixture. After 13 hours, the reaction was warmed to 40° C. After an additional 24 hours, the mixture was cooled, opened to air, and amended with a small amount of silica gel and stirred for 10 minutes, then filtered through a thin pad of silica gel. The filtrate was concentrated and purified by flash column chromatography on silica gel (0→10% EtOAc in hexanes) to give ethyl (S)-4-(2-allyl-1-oxo-1,2,3,4-tetrahydronaphthalen-2-yl)butanoate (2.39 g, >100% yield) as a yellow oil.

¹H NMR matched that of the R enantiomer.

LCMS: [M+H]⁺ m/z=301.2 amu

Ethyl (S)-4-(2-allyl-1-oxo-1,2,3,4-tetrahydronaphthalen-2-yl)butanoate (1.76 g, 5.9 mmol) was dissolved in EtOAc (12 mL) and MeCN (12 mL) then treated with H₂O (18 mL), RuCl₃.xH₂O (27 mg, 0.13 mmol), and NaIO₄ (5 g, 23 mmol) and the mixture was stirred vigorously at room temperature. After 1 hour, NaIO₄ (1.25 g, 5.9 mmol) was added.

After 90 minutes, the mixture was poured into 0.5M NaHSO₄ and extracted with EtOAc (3 times). The combined extract was washed with brine, dried over Na₂SO₄, filtered through Celite, and concentrated. The residue was dissolved in Methanol (35 mL), cooled to 0° C., and SOCl₂ (5.3 mL, 73 mmol) was added dropwise. The mixture stirred at room temperature for 90 minutes, amended with H₂O (10 mL) and stirred for 15 minutes, then poured into H₂O and extracted with Et₂O (3 times). The combined extract was washed with NaHCO₃ (3 times), brine, dried over Na₂SO₄, filtered through a thin pad of silica gel, concentrated, and purified by flash column chromatography on silica gel (0→30% EtOAc in hexanes) to give methyl (S)-4-(2-(2-methoxy-2-oxoethyl)-1-oxo-1,2,3,4-tetrahydronaphthalen-2-yl)butanoate (1.22 g, 3.84 mmol, 66% yield) as a pale yellow oil.

¹H NMR matched that of the R enantiomer.

LCMS: [M+H]⁺ m/z=319.1 amu

Methyl (S)-4-(2-(2-methoxy-2-oxoethyl)-1-oxo-1,2,3,4-tetrahydronaphthalen-2-yl)butanoate (1.22 g, 3.8 mmol) was dissolved in EtOAc (10 mL) and treated with Pd/C, 10 wt % (wetted) (240 mg) and HClO₄ (62 μL, 0.57 mmol) and the vessel was charged with H₂. After 17 hours, the reaction mixture was filtered through Celite and concentrated. The residue was taken up in MeOH (10 mL), cooled to 0° C., and treated with SOCl₂ (1.5 mL, 19 mmol), then warmed to room temperature. After 1.5 hours, the mixture was concentrated, diluted with H₂O, and extracted with Et₂O (3 times). The combined extract was washed with sat NaHCO₃, brine, dried over Na₂SO₄, filtered through a pad of silica gel, and concentrated. The residue was purified by flash column chromatography on silica gel (0→30% EtOAc in hexanes) to give methyl (R)-4-(2-(2-methoxy-2-oxoethyl)-1,2,3,4-tetrahydronaphthalen-2-yl)butanoate (1.02 g, 3.36 mmol, 88% yield).

¹H NMR matched that of the S enantiomer.

LCMS: [M+H]⁺ m/z=305.2 amu

To a cooled (−78° C.) solution of methyl (R)-4-(2-(2-methoxy-2-oxoethyl)-1,2,3,4-tetrahydronaphthalen-2-yl)butanoate (287 mg, 0.94 mmol) in THE (9.5 mL) was added LDA (0.79 mL, 1.42 mmol, 1.8 M in hexanes). The mixture was warmed to room temperature and stirred for 2 hours. The reaction was then quenched with saturated NH₄Cl (20 mL) and extracted with DCM (15 mL*3). The combined organics were dried over Na₂SO₄, filtered, and concentrated in vacuo. The crude methyl (1R)-3-oxo-3′,4′-dihydro-1′H-spiro[cyclohexane-1,2′-naphthalene]-4-carboxylate was used in the next step without further purification.

LCMS: [M+H]⁺ m/z=273.1 amu

To a vial containing a solution of the crude methyl (1R)-3-oxo-3′,4′-dihydro-1′H-spiro[cyclohexane-1,2′-naphthalene]-4-carboxylate (257 mg, 0.94 mmol, est.) in MeCN (4.7 mL) was added thiourea (86 mg, 1.13 mmol) followed by DBU (211 μL, 1.41 mmol). The vial was sealed and the reaction was stirred overnight. Upon completion, the mixture was cooled to room temperature, poured into saturated NaHCO₃ (15 mL), and extracted with DCM (15 mL*3). The combined organics were dried over Na₂SO₄, filtered, and concentrated in vacuo. The crude (S)-2′-mercapto-3,4,5′,8′-tetrahydro-1H,6′H-spiro[naphthalene-2,7′-quinazolin]-4′-ol was taken on to the next step without further purification.

LCMS: [M+H]⁺ m/z=299.1 amu

To a vial containing the crude (S)-2′-mercapto-3,4,5′,8′-tetrahydro-1H,6′H-spiro[naphthalene-2,7′-quinazolin]-4′-ol (281 mg, 0.94 mmol, est.) was added EtOH (4 mL) followed by 1M NaOH (1.05 mL, 1.05 mmol, aq.). Once the substrate was fully dissolved, MeI (65 μL, 1.04 mmol) was added. The reaction was stirred for 1 h, after which saturated NaHCO₃ (15 mL) was added and the mixture was extracted with DCM (15 mL*3). The combined organics were dried over Na₂SO₄, filtered, and concentrated in vacuo. The crude (S)-2′-(methylthio)-3,4,5′,8′-tetrahydro-1H,6′H-spiro[naphthalene-2,7′-quinazolin]-4′-ol was taken on to the next step without further purification.

LCMS: [M+H]⁺ m/z=313.1 amu

To solution of the crude (S)-2′-(methylthio)-3,4,5′,8′-tetrahydro-1H,6′H-spiro[naphthalene-2,7′-quinazolin]-4′-ol (90 mg, 0.29 mmol, est.) in DCM (1.2 mL) was added N,N-diisopropylethylamine (100 μL, 0.58 mmol). After stirring for 5 minutes, the mixture was cooled to 0° C. and triflic anhydride (432 μL, 0.43 mmol, 1M in DCM) was added. The reaction was stirred for 2 hours, after which hexanes (2.4 mL) was added and the mixture was passed through a plug of silica gel, rinsing with 30% EtOAc in hexanes (20 mL). The combined organics were concentrated in vacuo to give intermediate 9-1, (S)-2′-(methylthio)-3,4,5′,8′-tetrahydro-1H,6′H-spiro[naphthalene-2,7′-quinazolin]-4′-yl trifluoromethanesulfonate, which was used in subsequent reactions without further purification.

LCMS: [M+H]⁺ m/z=445.1 amu

### Synthesis of Intermediate 9-2

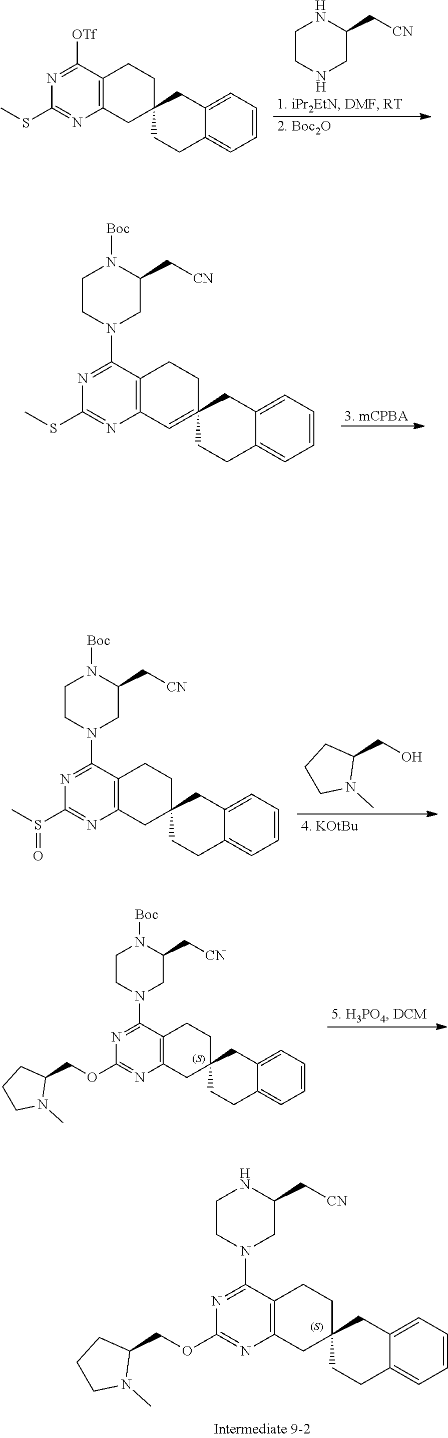

To a cooled (0° C.) solution of intermediate 9-1, (S)-2′-(methylthio)-3,4,5′,8′-tetrahydro-1H,6′H-spiro[naphthalene-2,7′-quinazolin]-4′-yl trifluoromethanesulfonate (128 g, 0.29 mmol), in DCM (3.2 mL) was added triethylamine (201 μL, 1.44 mmol), followed by (S)-2-(piperazin-2-yl)acetonitrile 2HCl (84 mg, 0.52 mmol). The resulting solution warmed to room temperature and stirred for 5 hours. After consumption of starting material was observed, di-tert-butyl dicarbonate (252 mg, 1.16 mmol) was added and the reaction was heated to 40° C. and stirred for 2 hours. The reaction mixture was cooled to room temperature and poured into saturated NaHCO₃ (15 mL, aq.) and extracted with DCM (10 mL*3). The combined organic extracts were dried over Na₂SO₄, filtered and concentrated in vacuo. The mixture was purified using column chromatography (0→50% EtOAc in hexanes) to afford tert-butyl (S)-2-(cyanomethyl)-4-((S)-2′-(methylthio)-3,4,5′,8′-tetrahydro-1H,6′H-spiro[naphthalene-2,7′-quinazolin]-4′-yl)piperazine-1-carboxylate (150 mg, 0.29 mmol, quant.) as a white foam.

LCMS: [M+H]⁺ m/z=520.2 amu

To a cooled (0° C.) solution of tert-butyl (S)-2-(cyanomethyl)-4-((S)-2′-(methylthio)-3,4,5′,8′-tetrahydro-1H,6′H-spiro[naphthalene-2,7′-quinazolin]-4′-yl)piperazine-1-carboxylate (150 mg, 0.29 mmol) in DCM (2.9 mL) was added mCPBA (73 mg, 0.32 mmol). The mixture was stirred for 30 minutes, after which half-saturated NaHCO₃ (10 mL, aq.) was added and the mixture was extracted with DCM (10 mL*3). The combined organics were dried with Na₂SO₄, filtered, and concentrated in vacuo. The crude tert-butyl (2S)-2-(cyanomethyl)-4-((2S)-2′-(methylsulfinyl)-3,4,5′,8′-tetrahydro-1H,6′H-spiro[naphthalene-2,7′-quinazolin]-4′-yl)piperazine-1-carboxylate was taken on to the next step without further purification.

LCMS: [M+H]⁺ m/z=536.2 amu

To a cooled (0° C.) vial containing NaH (35 mg, 0.86 mmol, 60% mineral oil dispersion) was added THE (1 mL) followed by (S)-(1-methylpyrrolidin-2-yl)methanol (171 μL, 1.44 mmol). The mixture was stirred for 45 minutes, at which point the crude tert-butyl (2S)-2-(cyanomethyl)-4-((2S)-2′-(methylsulfinyl)-3,4,5′,8′-tetrahydro-1H,6′H-spiro[naphthalene-2,7′-quinazolin]-4′-yl)piperazine-1-carboxylate (159 mg, 0.29 mmol, est.), as a solution in THE (3 mL), was added. The mixture was warmed to room temperature and stirred for 3 hours. Upon completion, the reaction was quenched with saturated NH₄Cl (10 mL, aq.) and the mixture was extracted with DCM (10 mL*3). The combined organics were dried with Na₂SO₄, filtered, and concentrated in vacuo to afforded the crude tert-butyl (S)-2-(cyanomethyl)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate, which was used in subsequent step without further purification.

LCMS: [M+H]⁺ m/z=587.3 amu

To a vial containing the crude tert-butyl (S)-2-(cyanomethyl)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazine-1-carboxylate (141 mg, 0.24 mmol, est.) in DCM (4.8 mL) was added H₃PO₄ (147 μL, 2.4 mmol) dropwise. The reaction was stirred at room temperature for 2 hours, at which point H₂O (10 mL) was added and the solution was made basic by slow addition of 2 M NaOH solution (aq.). Once basic, the mixture was extracted with DCM (10 mL*3), and the combined organics were dried with Na₂SO₄, filtered, and concentrated in vacuo to afford intermediate 9-2, 2-((S)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile, which was used in subsequent reactions without further purification.

LCMS: [M+H]⁺ m/z=487.3 amu

### Synthesis of Compound C-29

To a cooled (0° C.) solution of intermediate 9-2, 2-((S)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (61 mg, 0.13 mmol, est.), in DCM (2.5 mL) was added N,N-diisopropylethylamine (220 μL, 1.25 mmol), followed by acrylic anhydride (47 μL, 0.38 mmol). The mixture was warmed to room temperature and stirred for 2 hours, at which point the solution was concentrated in vacuo, taken up in DMSO, filtered, and purified using preparative HPLC (C18, 20→60% MeCN in H₂O+0.25% TFA). The combine fractions containing the desired product were lyophilized to yield compound C-29, 2-((S)-1-acryloyl-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (11.7 mg, 0.022 mmol, 17% yield, over 5 steps), as a fluffy off-white solid.

¹H NMR (400 MHz, Acetonitrile-d₃, TFA salt) δ 10.64 (s, 1H), 7.24-6.91 (m, 4H), 6.87-6.59 (m, 1H), 6.25 (d, J=16.9 Hz, 1H), 5.77 (d, J=10.6 Hz, 1H), 5.39-4.17 (m, 10H), 4.17-3.84 (m, 1H), 3.78-3.63 (m, 2H), 3.63-3.39 (m, 2H), 3.18-3.03 (m, 1H), 3.03-2.43 (m, 10H), 2.43-2.22 (m, 1H), 2.22-1.98 (m, 2H), 1.85-1.50 (m, 4H) ppm

LCMS: [M+H]⁺ m/z=541.3 amu

### Synthesis of C-30

To a cooled (0° C.) solution of intermediate 9-2, 2-((S)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (61 mg, 0.13 mmol, est.), in DCM (2.5 mL) was added N,N-diisopropylethylamine (220 μL, 1.26 mmol), followed by 2-fluoroacrylic anhydride (31 mg, 0.19 mmol). The mixture was warmed to room temperature and stirred for 2 hours, at which point the solution was concentrated in vacuo, taken up in DMSO, filtered, and purified using preparative HPLC (C18, 10→55% MeCN in H₂O+0.25% TFA). The combine fractions containing the desired product were lyophilized to yield compound C-30, 2-((S)-1-(2-fluoroacryloyl)-4-((S)-2′-(((S)-1-methylpyrrolidin-2-yl)methoxy)-3,4,5′,8′-tetrahydro-1H,6′H-spiro[naphthalene-2,7′-quinazolin]-4′-yl)piperazin-2-yl)acetonitrile (16.2 mg, 0.029 mmol, 23% yield, over 5 steps), as a fluffy off-white solid.

¹H NMR (400 MHz, Acetonitrile-d₃, TFA salt) δ 10.88 (s, 1H), 7.21-6.94 (m, 4H), 6.13-5.11 (m, 5H), 4.97-4.61 (m, 3H), 4.50 (d, J=14.2 Hz, 1H), 4.35 (d, J=12.0 Hz, 1H), 4.08 (s, 1H), 3.78-3.61 (m, 2H), 3.59-3.25 (m, 3H), 3.14-2.98 (m, 1H), 2.92 (s, 3H), 2.90-2.81 (m, 3H), 2.77 (d, J=16.4 Hz, 1H), 2.72-2.57 (m, 4H), 2.37-2.18 (m, 1H), 2.16-1.96 (m, 2H), 1.87-1.49 (m, 4H) ppm

LCMS: [M+H]⁺ m/z=559.3 amu

### Example 10: Synthesis of Compounds C-31 and C-32

### Synthesis of Intermediate 10-1

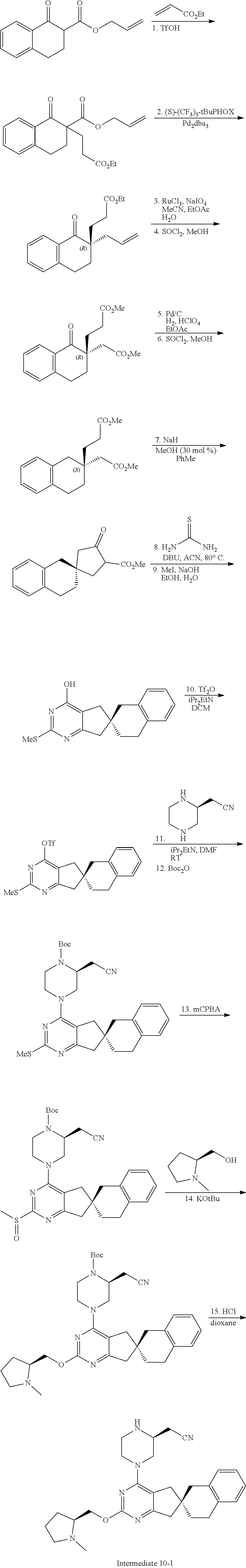

A mixture of allyl 1-hydroxy-3,4-dihydronaphthalene-2-carboxylate (207 mg, 0.90 mmol) and ethyl acrylate (115 μL, 1.1 mmol) was treated with TfOH (24 μL, 0.27 mmol) and stirred at room temperature. After 2 hours, the mixture was poured into saturated NaHCO₃ and extracted with EtOAc (3 times). The combined extract was washed with brine, dried over Na₂SO₄, filtered through a thin pad of silica gel, concentrated, and purified by flash column chromatography on silica gel (0→15% EtOAc in hexanes) to give allyl 2-(3-ethoxy-3-oxopropyl)-1-oxo-1,2,3,4-tetrahydronaphthalene-2-carboxylate (255.1 mg, 86% yield) as a colorless oil.

¹H NMR (400 MHz, CDCl₃) δ 8.03 (dd, J=7.9, 1.8 Hz, 1H), 7.47 (td, J=7.4, 1.5 Hz, 1H), 7.30 (t, J=7.6 Hz, 1H), 7.21 (d, J=7.7 Hz, 1H), 5.78 (ddt, J=17.1, 10.5, 5.5 Hz, 1H), 5.16 (dq, J=8.6, 1.4 Hz, 1H), 5.14-5.12 (m, 1H), 4.63-4.52 (m, 2H), 4.11 (q, J=7.1 Hz, 2H), 3.05 (ddd, J=17.5, 9.7, 5.0 Hz, 1H), 2.95 (dt, J=17.6, 5.3 Hz, 1H), 2.63-2.50 (m, 2H), 2.46-2.28 (m, 2H), 2.23 (ddd, J=13.9, 10.9, 5.1 Hz, 1H), 2.12 (ddd, J=13.7, 9.7, 5.0 Hz, 1H), 1.23 (t, J=7.2 Hz, 3H) ppm

¹³C NMR (101 MHz, CDCl₃) δ 195.11, 173.15, 171.40, 142.89, 133.71, 132.04, 131.50, 128.84, 128.16, 126.98, 118.52, 65.88, 60.62, 56.86, 31.26, 30.01, 28.97, 25.93, 14.31 ppm

(S)-p-(CF₃)₃-t-BuPHOX (36.5 mg, 0.062 mmol) and Pd₂(dba)₃ (21.2 mg, 0.023 mmol) were suspended in degassed anhydrous MTBE (5 mL) and stirred for 30 minutes. Separately, allyl 2-(3-ethoxy-3-oxopropyl)-1-oxo-1,2,3,4-tetrahydronaphthalene-2-carboxylate (255.1 mg, 0.77 mmol) was dissolved in MTBE (5 mL) and sparged for 20 minutes, then added to the catalyst mixture and the reaction was stirred at 25° C. After 14 hours, the reaction was opened to air and amended with a small amount of silica gel and stirred for 10 minutes then filtered through a thin pad of silica gel rinsing with 1:1 hexanes:EtOAc. The filtrate was concentrated and purified by flash column chromatography on silica gel (0→20% EtOAc in hexanes) to give ethyl (R)-3-(2-allyl-1-oxo-1,2,3,4-tetrahydronaphthalen-2-yl)propanoate (216.8 mg, 98% yield) as a pale yellow oil.

¹H NMR (400 MHz, CDCl₃) δ 8.03 (dd, J=7.8, 1.7 Hz, 1H), 7.46 (td, J=7.5, 1.4 Hz, 1H), 7.30 (t, J=7.5 Hz, 1H), 7.21 (d, J=7.6 Hz, 1H), 5.83-5.69 (m, 1H), 5.14-5.10 (m, 1H), 5.09-5.05 (m, 1H), 4.08 (q, J=7.2 Hz, 2H), 3.00 (t, J=6.5 Hz, 2H), 2.47 (ddt, J=14.1, 7.2, 1.2 Hz, 1H), 2.42-2.22 (m, 3H), 2.10-1.89 (m, 4H), 1.21 (t, J=7.2 Hz, 3H) ppm

¹³C NMR (101 MHz, CDCl₃) δ 200.73, 173.69, 143.12, 133.53, 133.38, 131.77, 129.09, 128.84, 128.52, 128.16, 126.84, 118.75, 60.53, 47.14, 38.97, 31.09, 29.20 (2), 25.07, 14.29 ppm

LCMS: [M+H]⁺ m/z=287.2 amu

Ethyl (R)-3-(2-allyl-1-oxo-1,2,3,4-tetrahydronaphthalen-2-yl)propanoate (216.8 mg, 0.76 mmol) was dissolved in EtOAc (1.5 mL) and MeCN (1.5 mL) and treated with H₂O (2.3 mL), NaIO₄ (831 mg, 3.9 mmol), and RuCl₃.xH₂O (3.45 mg, 0.020 mmol), and the mixture was stirred vigorously at room temperature. After 4 hours, the mixture was poured into 0.5M NaHSO₄ and extracted with EtOAc (3 times). The combined extract was washed with brine, dried over Na₂SO₄, filtered through Celite, and concentrated. The residue was taken up in MeOH (4.5 mL), cooled to 0° C., and SOCl₂ (550 μL, 7.6 mmol) was added dropwise. The mixture was then stirred at room temperature. After 90 minutes, the reaction was amended with H₂O (1 mL) and stirred for 15 minutes then poured into H₂O and extracted with Et₂O (3 times). The combined extract was washed with NaHCO₃ (3 times), brine, dried over Na₂SO₄, filtered through a thin pad of silica gel, concentrated, and purified by flash column chromatography on silica gel (0→25% EtOAc in hexanes) to give methyl (R)-3-(2-(2-methoxy-2-oxoethyl)-1-oxo-1,2,3,4-tetrahydronaphthalen-2-yl)propanoate (176 mg, 76% yield) as a colorless oil.

¹H NMR (400 MHz, CDCl₃) δ 8.03 (dd, J=7.9, 1.7 Hz, 1H), 7.46 (td, J=7.5, 1.5 Hz, 1H), 7.30 (t, J=7.8, 7.3 Hz, 2H), 7.22 (d, J=7.5 Hz, 1H), 3.64 (s, 3H), 3.62 (s, 3H), 3.12 (ddd, J=17.4, 11.5, 4.9 Hz, 1H), 2.92 (dt, J=17.5, 4.5 Hz, 1H), 2.86 (d, J=15.7 Hz, 1H), 2.51 (d, J=15.7 Hz, 1H), 2.48-2.38 (m, 2H), 2.28 (ddd, J=16.1, 10.6, 5.7 Hz, 1H), 2.12-1.95 (m, 3H) ppm

¹³C NMR (101 MHz, CDCl₃) δ 199.52, 173.61, 171.83, 142.82, 133.60, 131.26, 128.88, 128.31, 126.94, 51.83, 51.75, 46.18, 39.23, 31.46, 28.92, 28.74, 24.98 ppm

LCMS: [M+H]⁺ m/z=305.1 amu

Methyl (R)-3-(2-(2-methoxy-2-oxoethyl)-1-oxo-1,2,3,4-tetrahydronaphthalen-2-yl)propanoate (176 mg, 0.58 mmol) was dissolved in EtOAc (2.9 mL) and treated with Pd/C 10% (wetted) (40 mg). The vessel was charged with H₂ and stirred for 15 hours then filtered through Celite and concentrated. The residue was dissolved in Methanol (5 mL), cooled to 0° C., and treated with SOCl₂ (340 μL, 4.6 mmol) then warmed to room temperature. After 70 minutes, the mixture was poured into H₂O and extracted with EtOAc (2 times). The combined extract was washed with saturated NaHCO₃, brine, dried over Na₂SO₄, filtered through a thin pad of silica gel, concentrated, and purified by flash column chromatography on silica gel (0→30% EtOAc in hexanes) to give methyl (S)-3-(2-(2-methoxy-2-oxoethyl)-1,2,3,4-tetrahydronaphthalen-2-yl)propanoate (134.7 mg, 80% yield) as a colorless oil.

¹H NMR (400 MHz, CDCl₃) δ 7.14-6.99 (m, 4H), 3.66 (s, 3H), 3.65 (s, 3H), 2.83 (q, J=7.3, 6.7 Hz, 2H), 2.74 (d, J=16.4 Hz, 1H), 2.65 (d, J=16.4 Hz, 1H), 2.45-2.38 (m, 2H), 2.35 (d, J=14.1 Hz, 1H), 2.26 (d, J=14.2 Hz, 1H), 1.89-1.74 (m, 3H), 1.74-1.63 (m, 1H) ppm

¹³C NMR (101 MHz, CDCl₃) δ 174.00, 171.99, 135.09, 134.52, 129.54, 128.74, 125.83, 125.79, 51.56, 51.29, 40.59, 40.09, 34.60, 32.36, 31.79, 28.59, 25.49 ppm

LCMS: [M+H]⁺ m/z=291.1 amu

A mixture of NaOMe, 1M in MeOH (560 μL, 0.56 mmol) in anhydrous toluene (3 mL) was heated to 100° C. and methyl (S)-3-(2-(2-methoxy-2-oxoethyl)-1,2,3,4-tetrahydronaphthalen-2-yl)propanoate (134.7 mg, 0.46 mmol) was added as a solution in toluene (2 mL) dropwise over a period of approximately 15 minutes. After 2.5 hours, the mixture was cooled to room temperature and poured into saturated NH₄Cl and extracted with EtOAc (3 times). The combined extract was washed with brine, dried over Na₂SO₄, filtered through a thin pad of silica gel, concentrated, and purified by flash column chromatography on silica gel (0→20% EtOAc in hexanes) to give methyl (1R)-4-oxo-3′,4′-dihydro-1′H-spiro[cyclopentane-1,2′-naphthalene]-3-carboxylate (93.1 mg, 78% yield).

¹H NMR (400 MHz, CDl₃, mixture of diastereomers) δ 7.18-6.99 (m, 4H), 3.75 (d, J=1.0 Hz, 3H), 3.54-3.40 (m, 1H), 3.02-2.76 (m, 3H), 2.76-2.62 (m, 1H), 2.42-2.17 (m, 4H), 1.96-1.80 (m, 1H), 1.74 (t, J=6.8 Hz, 1H) ppm

¹³C NMR (101 MHz, CDCl₃, mixture of diastereomers) δ 210.82, 210.77, 169.75, 169.74, 135.26, 134.79, 134.78, 134.04, 129.61, 129.37, 128.94, 128.82, 126.21, 126.09, 125.92, 125.90, 53.48, 53.33, 52.55, 52.53, 50.14, 49.85, 41.89, 40.41, 37.67, 37.59, 37.16, 36.97, 34.52, 32.25, 26.59, 26.13 ppm

LCMS: [M+H]⁺ m/z=259.1 amu

methyl (1R)-4-oxo-3′,4′-dihydro-1′H-spiro[cyclopentane-1,2′-naphthalene]-3-carboxylate (88.3 mg, 0.34 mmol) was dissolved in anhydrous MeCN (1.7 mL) and treated with thiourea (31.2 mg, 0.41 mmol) and DBU (76.5 μL, 0.51 mmol) and the mixture was warmed to 70° C. After 48 hours, the mixture was cooled to room temperature and concentrated. The residue was treated with 0.2M NaH₂PO₄ and the resulting solids were collected by centrifugation. The still wet crude isolate was suspended in EtOH (690 μL) and 1M NaOH (375 μL, 0.38 mmol) and treated with MeI (24 μL, 0.39 mmol), and the mixture was sonicated to dissolve, then aged at room temperature. After 30 minutes, the mixture was diluted with 0.1M NaH₂PO₄ and extracted with CHCl₃ (3 times). The combined extract was dried over Na₂SO₄, amended with 0.05 vol MeOH and filtered through a thin pad of silica gel rinsing with 95:5 CHCl₃:MeOH and concentrated to give (R)-2-(methylthio)-3′,4′,5,7-tetrahydro-1′H-spiro[cyclopenta[d]pyrimidine-6,2′-naphthalen]-4-ol (95.2 mg, 93.3% yield) as a white solid.

LCMS: [M+H]⁺ m/z=299.1 amu

(R)-2-(methylthio)-3′,4′,5,7-tetrahydro-1′H-spiro[cyclopenta[d]pyrimidine-6,2′-naphthalen]-4-ol (95.2 mg, 0.32 mmol) was suspended in anhydrous DCM (640 μL) and treated with iPr₂EtN (111 μL, 0.64 mmol). The mixture was cooled to 0° C. and triflic anhydride, 1M in DCM (479 μL, 0.48 mmol) was added dropwise then the cooling bath was removed. After 45 minutes, the mixture was diluted with hexanes and filtered through a pipet column of silica gel rinsing with 9:1 hexanes:EtOAc and concentrated to give (R)-2-(methylthio)-3′,4′,5,7-tetrahydro-1′H-spiro[cyclopenta[d]pyrimidine-6,2′-naphthalen]-4-yl trifluoromethanesulfonate (90.7 mg, 66% yield).

LCMS: [M+H]⁺ m/z=431.1 amu

(R)-2-(methylthio)-3′,4′,5,7-tetrahydro-1′H-spiro[cyclopenta[d]pyrimidine-6,2′-naphthalen]-4-yl trifluoromethanesulfonate (90.7 mg, 0.21 mmol) was dissolved in anhydrous DMF (420 μL) and treated with 2-[(2S)-piperazin-2-yl]acetonitrile dihydrochloride (45.9 mg, 0.23 mmol) and iPr₂EtN (110 μL, 0.63 mmol). After 30 minutes, Boc₂O (77.2 mg, 0.35 mmol) was added and the mixture was stirred overnight. The mixture was poured into saturated NaHCO₃ and extracted with EtOAc (3 times). The combined extract was washed with brine, dried over Na₂SO₄, filtered through a thin pad of silica gel, concentrated, and purified by flash column chromatography on silica gel (5→40% EtOAc in hexanes). The fractions with the desired product were combined to give tert-butyl (S)-2-(cyanomethyl)-4-((R)-2-(methylthio)-3′,4′,5,7-tetrahydro-1′H-spiro[cyclopenta[d]pyrimidine-6,2′-naphthalen]-4-yl)piperazine-1-carboxylate (92.3 mg, 87% yield) as a white foam.

LCMS: [M+H]⁺ m/z=506.2 amu

tert-Butyl (S)-2-(cyanomethyl)-4-((R)-2-(methylthio)-3′,4′,5,7-tetrahydro-1′H-spiro[cyclopenta[d]pyrimidine-6,2′-naphthalen]-4-yl)piperazine-1-carboxylate (92.3 mg, 0.18 mmol) was dissolved in DCM (910 μL), cooled to 0° C., and treated with mCPBA (54.6 mg, 0.24 mmol). The mixture was stirred for 30 minutes then diluted with Et₂O and washed with half-saturated NaHCO₃ (3 times), brine, dried over Na₂SO₄, and concentrated to give the crude tert-butyl (2S)-2-(cyanomethyl)-4-((6R)-2-(methylsulfinyl)-3′,4′,5,7-tetrahydro-1′H-spiro[cyclopenta[d]pyrimidine-6,2′-naphthalen]-4-yl)piperazine-1-carboxylate (169.6 mg, 0.342 mmol, 100% yield) as a white foam. The crude product was taken forward without further purification.

LCMS: [M+H]⁺ m/z=522.2 amu

1-Methyl-L-prolinol (39.25 mg, 0.34 mmol) was dissolve in anhydrous THF (500 μL) and treated with KOtBu, 1.7M in THE (200 μL, 0.34 mmol). The mixture was aged for 5 minutes, then added to a solution of the crude tert-butyl (2S)-2-(cyanomethyl)-4-((6R)-2-(methylsulfinyl)-3′,4′,5,7-tetrahydro-1′H-spiro[cyclopenta[d]pyrimidine-6,2′-naphthalen]-4-yl)piperazine-1-carboxylate (88.9 mg, 0.17 mmol) in anhydrous THF (500 μL) at 0° C. After 30 minutes, the mixture was poured into aqueous K₂CO₃ and extracted with Et₂O (3 times). The combined extract was washed with brine, dried over Na₂SO₄, and concentrated to give the crude tert-butyl (S)-2-(cyanomethyl)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazine-1-carboxylate (85.8 mg, 0.150 mmol, 88% yield) as a pale brown foam.

LCMS: [M+H]⁺ m/z=573.4 amu

The crude tert-butyl (S)-2-(cyanomethyl)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazine-1-carboxylate (85.8 mg, 0.15 mmol) was treated with 4N HCl in dioxane (1.5 mL) at room temperature. After 1 hour, the mixture was concentrated and the residue was dissolved in 1N HCl and washed with Et₂O (2 times). The wash was back-extracted with 1N HCl once, and the combined aqueous was basified with K₂CO₃ and back-extracted with EtOAc (3 times). The combined extract was dried over anhydrous K₂CO₃, filtered, and concentrated to give intermediate 10-1, 2-((S)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazin-2-yl)acetonitrile (79.2 mg, >100% yield), as a brown oil.

LCMS: [M+H]⁺ m/z=473.3 amu

### Synthesis of Compound C-31

Intermediate 10-1, 2-((S)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazin-2-yl)acetonitrile (39.6 mg, 0.084 mmol), was dissolved in anhydrous MeCN (500 μL) and treated with iPr₂EtN (14.5 μL, 0.0832 mmol) and acrylic anhydride (14.5 μL, 0.13 mmol). After 15 minutes, the mixture was diluted with 0.25% TFA in H₂O, filtered, and purified by preparative HPLC (C18, 5→60% ACN in H₂O+0.25% TFA) to give compound C-31, 2-((S)-1-acryloyl-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazin-2-yl)acetonitrile (7.8 mg, 0.0148 mmol, 18% yield), as a colorless film.

¹H NMR (400 MHz, Methanol-d4) δ 7.15-7.02 (m, 4H), 7.01-6.95 (m, 1H), 6.27 (d, J=16.9 Hz, 1H), 5.89-5.75 (m, 1H), 4.55-4.31 (m, 4H), 3.31 (p, J=1.7 Hz, 2H), 3.06-2.83 (m, 7H), 2.82-2.55 (m, 10H), 2.24-2.11 (m, 1H), 1.98-1.85 (m, 6H), 1.85-1.74 (m, 1H) ppm

LCMS: [M+H]⁺ m/z=527.3 amu

### Synthesis of Compound C-32

Intermediate 10-1, 2-((S)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazin-2-yl)acetonitrile (39.6 mg, 0.084 mmol) was dissolved in anhydrous MeCN (500 μL) and treated with iPr₂EtN (14.5 μL, 0.0832 mmol) and 2-fluoroacrylic anydride (19.8 mg, 0.13 mmol). After 2 hours, the mixture was diluted with aqueous 0.25% TFA and purified by preparative HPLC (C18, 5→55% ACN in H₂O+0.25% TFA) to give compound C-32, 2-((S)-1-(2-fluoroacryloyl)-4-((R)-2-(((S)-1-methylpyrrolidin-2-yl)methoxy)-3′,4′,5,7-tetrahydro-1′H-spiro[cyclopenta[d]pyrimidine-6,2′-naphthalen]-4-yl)piperazin-2-yl)acetonitrile (28.4 mg, 62% yield).

¹H NMR (400 MHz, CD₃CN) δ 10.74 (s, 1H), 7.18-7.06 (m, 3H), 7.06-6.98 (m, 1H), 5.36-5.15 (m, 2H), 4.86-4.65 (m, 4H), 4.36 (d, J=10.5 Hz, 1H), 4.08 (d, J=16.0 Hz, 1H), 3.72 (tdd, J=10.3, 7.8, 4.3 Hz, 2H), 3.54-3.31 (m, 3H), 3.16-2.69 (m, 15H), 2.37-2.24 (m, 1H), 2.15-1.86 (m, 4H) ppm

¹⁹F NMR (376 MHz, CD₃CN) δ −107.56 ppm

LCMS: [M+H]⁺ m/z=545.3 amu

### Example 11: Synthesis of Spiro-tetrahydronaphthalene and Spiro-indane Compounds

### Preparation of Functionalized Spiro-Tetrahydronaphthalene Compounds

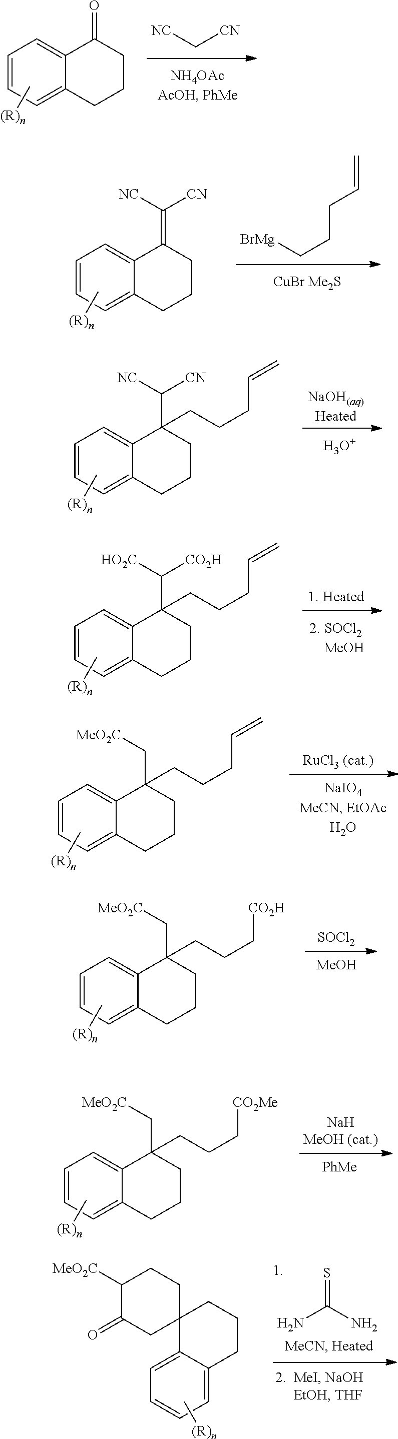

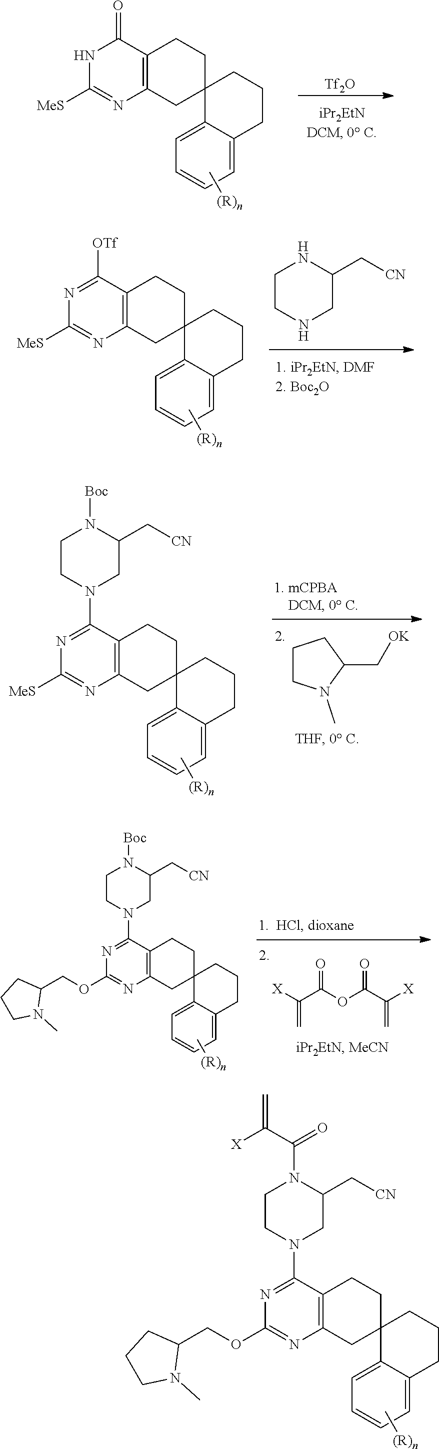

Individual stereoisomers of the spirocyclic center may be prepared by catalytic and/or stereoselective variants of the above reaction sequence, or may be resolved from the racemic form by chiral chromatography or other conventional techniques.

Compounds obtained by this synthetic route include, but are not limited to, those where X is H, F, CH₃, or OCH₃; R, in each occurrence and when present, is independently OH, F, Cl, Br, N(R)₂, CF₃, CH₃, OCF₃, OCF₂H, OCFH₂ or OCH₃; and n is 0, 1 or 2. Other substituents for X and R would be readily apparent to one of skill in the art, particularly those substituents that are found in commercially available molecules used in the first step of this synthesis.

Additionally, heterocyclic and/or heteroaryl analogs may be prepared by adaptation of the generalized synthetic sequences detailed above, for example, those detailing the synthesis of intermediates 5-1, 6-2 and 7-2, and particularly the synthesis of intermediate 6-1.

### Preparation of Functionalized Spiro-Indane Compounds

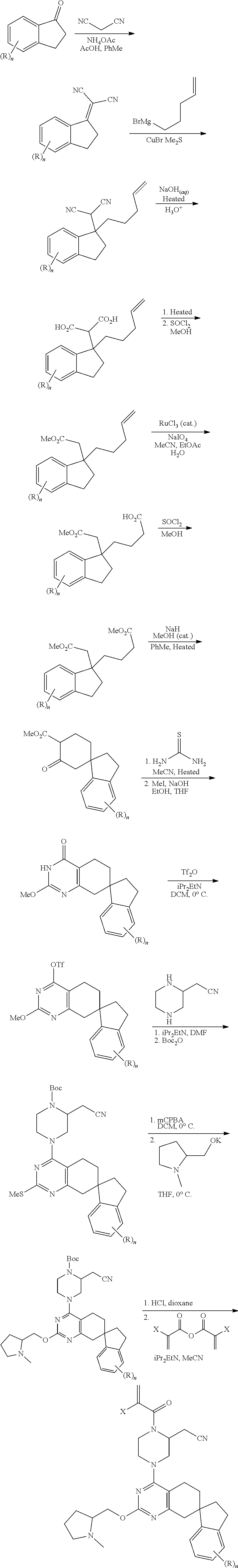

Individual stereoisomers of the spirocyclic center may be prepared by catalytic and/or stereoselective variants of the above reaction sequence, or may be resolved from the racemic form by chiral chromatography or other conventional techniques.

Compounds obtained by this synthetic route include, but are not limited to, those where X is H, F, CH₃, or OCH₃; R, in each occurrence and when present, is independently OH, F, Cl, Br, N(R)₂, CF₃, CH₃, OCF₃, OCF₂H, OCFH₂ or OCH₃; and n is 0, 1 or 2. Other substituents for X and R would be readily apparent to one of skill in the art, particularly those substituents that are found in commercially available molecules used in the first step of this synthesis.

Additionally, heterocyclic and/or heteroaryl analogs may be prepared by adaptation of the generalized synthetic sequences detailed above, for example, those detailing the synthesis of intermediates 5-1, 6-2 and 7-2, and particularly the synthesis of intermediate 6-1.

### BIOLOGICAL EXPERIMENTS

### KRAS G12C Kinetic Modification Assay

Test compounds were assayed for reactivity towards His 6-tagged KRASG12C (2-185) protein (hereinafter in this section, “KRASG12C”) using an HPLC-MS assay as described by Patricelli et al (Cancer Discov. 2016, 6 (3), 316). KRASG12C (1 μM) was incubated at 22° C. with test compounds at a final concentration of 10 μM in a buffer containing 20 mM HEPES, 150 mM NaCl, 1 mM MgCl₂, 1 mM DTT, pH 7.5 and a final DMSO concentration of 2% vol. Aliquots were removed at 0, 1, 3, 5, and 30 minutes, quenched by dilution into 0.1 volume of 6.2% formic acid, and analyzed by HPLC-MS using a Water Acquity equipped with a Waters LCT Premier XE. Mass spectra were deconvoluted using MaxEnt and the extent of inhibitor incorporation was measured ratiometrically. The pseudo-first k_{obs}/[I] (M⁻¹·s⁻¹) order rate constant was calculated from the rate determined by non-linear least squares fitting to the first order rate equation:

[E]ₜ=[E]₀^{-k}^{_{obs}}^{t}

### Cell Line Growth Retardation Assay

Cells were seeded at densities of 1,000-5,000 cells per well in 48-well tissue culture plates. After a 24 h rest period, cells were treated with compound at 10 μM, 1 μM, 0.4 μM, 0.08 μM, 0.016 μM, and 0.0032 μM. A group of cells were treated with the vehicle in which the compound was prepared and served as a control. Prior to treatment, cells were counted and this count was used as a baseline for the calculation of growth inhibition. The cells were grown in the presence of compounds for 6 days and were counted on day 6. All cell counting was performed using a Synentec Cellavista plate imager. Growth inhibition was calculated as a ratio of cell population doublings in the presence of compound versus the absence of compound. If treatment resulted in a net loss of cells from baseline, percent lethality was defined as the decrease in cell numbers in treated wells compared with counts on day 1 of non-treated wells post-seeding. IC₅₀ values for each compound were calculated by fitting curves to data points from each dose-response assay using the Proc NLIN function in SAS for Windows version 9.2 (SAS Institute, Inc.).

### Designation of Sensitive and Resistant Cohorts and Calculation of Average IC₅₀ Values

Human cancer cell lines were grouped as “sensitive” or “resistant” to KRAS G12C inhibition based on whether their growth was retarded by AMG-510 (i.e., 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one) or MRTX-849 (i.e., 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile) (data not shown; see Table 5). These sensitive and resistant cohorts were interrogated for response to each compound, and IC₅₀ s were calculated for each cell line using the same technique described above. Average IC₅₀ s for the sensitive and resistant cohorts were calculated as arithmetic means of the group. See Table 4. “A” represents an IC₅₀ of 1 μM or less, “B” represents an IC₅₀ of greater than 1 μM, and “C” represents an IC₅₀ of greater than 5 μM.

### Caco-2 Assay (Pₐₚₚ A to B)

The degree of bi-directional human intestinal permeability for compounds was estimated using a Caco-2 cell permeability assay. Caco-2 cells were seeded onto polyethylene membranes in 96-well plates. The growth medium was refreshed every 4 to 5 days until cells formed a confluent cell monolayer. HBSS with 10 mM HEPES at pH 7.4 was used as the transport buffer. Compounds were tested at 2 μM bi-directionally in duplicate. Digoxin, nadolol and metoprolol were included as standards. Digoxin was tested at 10 μM bi-directionally in duplicate, while nadolol and metoprolol were tested at 2 μM in the A to B direction in duplicate. The final DMSO concentration was adjusted to less than 1% for all experiments. The plate was incubated for 2 hours in a CO₂ incubator at 37° C., with 5% CO₂ at saturated humidity. After incubation, all wells were mixed with acetonitrile containing an internal standard, and the plate was centrifuged at 4,000 rpm for 10 minutes. 100 μL supernatant was collected from each well and diluted with 100 μL distilled water for LC/MS/MS analysis. Concentrations of test and control compounds in starting solution, donor solution, and receiver solution were quantified by LC/MS/MS, using peak area ratio of analyte to internal standard.

The apparent permeability coefficient Pₐₚₚ (cm/s) was calculated using the equation:

Pₐₚₚ=(dCᵣ/dt)×Vᵣ/(A×C₀),

where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, which is 0.0804 cm² for the area of the monolayer; and C₀ is the initial concentration in the donor chamber (μM).

The efflux ratio was calculated using the equation:

Efflux Ratio=Pₐₚₚ(BA)/Pₐₚₚ(AB)

Percent recovery was calculated using the equation:

% Recovery=100×[(Vᵣ×Cᵣ)+(V_{d}×C_{d})]/(V_{d}×C₀),

where Vd is the volume in the donor chambers, which are 0.075 mL on the apical side and 0.25 mL on the basolateral side; and C_{d} and Cᵣ are the final concentrations of transport compound in donor and receiver chambers, respectively.

### Measurement of Compound Metabolic Stability

The metabolic stability of compounds was determined in hepatocytes from human, mice and rats. Compounds were diluted to 5 μM in Williams' Medium E from 10 mM stock solutions. 10 μL of each compound was aliquoted into a well of a 96-well plate and reactions were started by aliquoting 40 μL of a 625,000 cells/mL suspension into each well. The plate was incubated at 37° C. with 5% C₀₂. At each corresponding time point, the reaction was stopped by quenching with ACN containing internal standards (IS) at a 1:3. Plates were shaken at 500 rpm for 10 min, and then centrifuged at 3,220×g for 20 minutes. Supernatants were transferred to another 96-well plate containing a dilution solution. Supernatants were analyzed by LC/MS/MS.

The remaining percent of compound after incubation was calculated using the following equation:

% Remaining Compound=Peak Area Ratios of Tested Compound vs. Internal Standard at End Point/Peak Area Ratios of Tested Compound vs. Internal Standard at Start Point

Compound half-life and CLᵢₙₜ were calculated using the following equations:

Cₜ=C₀*e^{-k*t}(first order kinetics);when Cₜ=½C₀,t_{1/2}=ln 2/k=0.693/k; and

CLᵢₙₜ=k/(1,000,000 cells/mL)

### Activity-Guided Selection of Inhibitors

Subgenera of KRAS G12C inhibitors having desirable properties were identified using a combination of in vitro data.

In particular, the results from the assays described above (e.g., Cell Line Growth Retardation Assay, KRAS Kinetic Modification Assay, Caco-2 Assay (Pₐₚₚ A to B), Measurement of Compound Metabolic Stability, and Designation of Sensitivity and Resistant Cohorts and Calculation of Average IC₅₀ Values) were used to select compounds having structural and functional features defined in the subgenera of Formula (IIIa).

In particular, a desirable property of compounds examined in sensitive and resistant cell lines, as described above, is having an average IC₅₀ for the drug-sensitive cell lines of Table 5 of about 1 μM or lower and having an average IC₅₀ for the drug-resistant cell lines of Table 5 of greater than 1 μM.

The skilled artisan would readily recognize that the results of additional in vitro assays (e.g., CYP enzymatic inhibition, hERG inhibition, compound solubility, target-specificity analysis), as well as the results of in vivo assays (e.g., rodent xenograft studies, rodent pharmacokinetic and single-dose saturation studies, rodent maximum tolerated dose studies, and oral bioavailability) could be used to identify other subgenera of KRAS G12C inhibitors, or to narrow subgenera determined using other results, for example, the subgenera of Formula (IIIa).

Lengthy table referenced here
US20220227738A1-20220721-T00001
Please refer to the end of the specification for access instructions.

Lengthy table referenced here
US20220227738A1-20220721-T00002
Please refer to the end of the specification for access instructions.

Lengthy table referenced here
US20220227738A1-20220721-T00003
Please refer to the end of the specification for access instructions.

Lengthy table referenced here
US20220227738A1-20220721-T00004
Please refer to the end of the specification for access instructions.

Lengthy table referenced here
US20220227738A1-20220721-T00005
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20220227738A1). An electronic copy of the table will also be available from the USPTO upon request and payment of the fee set forth in 37 CFR 1.19(b)(3).

We claim:

1. A compound having the structure of Formula Id: 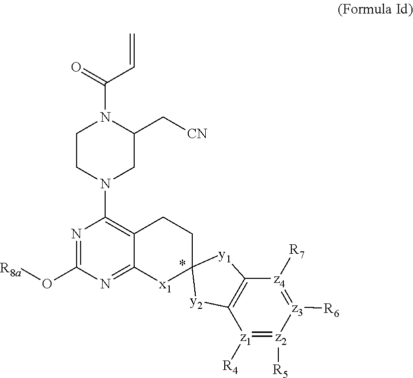
or a pharmaceutically acceptable salt thereof,
wherein:
* is the quaternary carbon atom;
x₁ is C═O or C(R₁)(R₂);
y₁ is y₁ₐ and y₂ is y₂ₐ, with the proviso that both y₁ₐ and y₂ₐ cannot be heteroatoms;
or
y₁ is *-y_{1b}-y_{1c} and y₂ is y₂ₐ, with the proviso that both y_{1b} and y₂ₐ cannot be heteroatoms, and the further proviso that both y_{1b} and y_{1c} cannot be heteroatoms; or
y₁ is y₁ₐ and y₂ is *-y_{2b}-y_{2c}, with the proviso that both y₁ₐ and y_{2b} cannot be heteroatoms, and the further proviso that both y_{2b} and y_{2c} cannot be heteroatoms; or
y₁ is *y_{1d}=y₁ₑ and y₂ is y₂ₐ, with the proviso that both y_{1d} and y₂ₐ cannot be heteroatoms; or
y₁ is y₁ₐ and y₂ is *-y_{2d}=y₂ₑ, with the proviso that both y₁ₐ and y_{2d} cannot be heteroatoms;
y₁ₐ and y₂ₐ are each independently C(R₁₁)₂, O, N(R₃) or S;
y_{1b}, y_{1c}, y_{2b} and y_{2c} are each independently C(R₁₁)₂, O, N(R₃) or S;
y_{1d}, y₁ₑ, y_{2d} and y₂ₑ are each independently C(R₃) or N;
z₁, z₂, z₃ and z₄ are each independently C or N;
R₁ and R₂ are each independently H or F;
R₃ in each occurrence is independently H or CH₃;
R₄, R₅, R₆ and R₇ are each independently H, F, Cl, CH₃ or OCH₃, or each of R₄, R₅, R₆ and R₇ is absent when the respective z to which each is attached is N;
R₈ₐ is H, C₁-C₃ alkyl, cycloalkyl, heterocyclyl, aralkyl, aryl or heteroaryl, wherein each of C₁-C₃ alkyl, cycloalkyl, heterocyclyl, aralkyl, aryl and heteroaryl may be optionally substituted with one or more R₉;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl; and
R₁₁ in each occurrence is independently H, F, Cl, CH₃ or OCH₃.

2. A compound having the structure of Formula IIa: 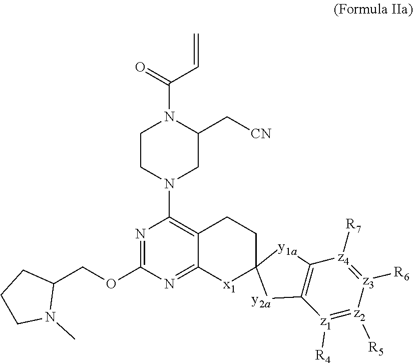
or a pharmaceutically acceptable salt thereof,
wherein:
x₁ is C═O or C(R₁)(R₂);
y₁ₐ and y₂ₐ are each independently C(R₁₁)₂, O, N(R₃) or S, with the proviso that both y₁ₐ and y₂ₐ cannot be heteroatoms;
z₁, z₂, z₃ and z₄ are each independently C or N;
R₁ and R₂ are each independently H or F;
R₃ is H or CH₃;
R₄, R₅, R₆ and R₇ are each independently H, F, Cl, CH₃ or OCH₃, or each of R₄, R₅, R₆ and R₇ is absent when the respective z to which each is attached is N; and
R₁₁ in each occurrence is independently H, F, Cl, CH₃ or OCH₃.

3. The compound of claim 2 having the structure of Formula IIb: 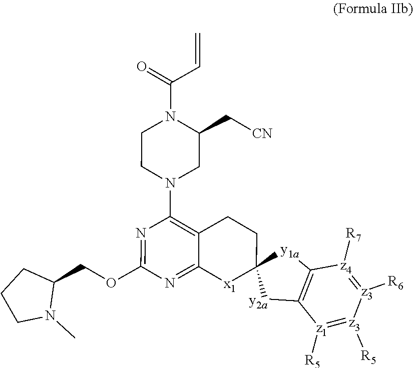
or a pharmaceutically acceptable salt thereof.

4. A compound having the structure of Formula IIIa: 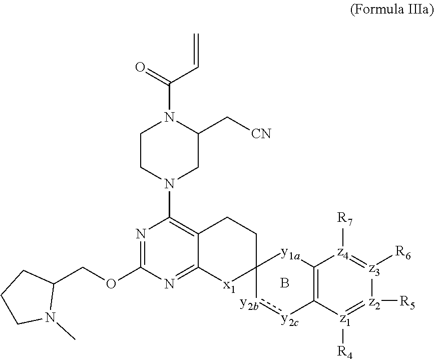
or a pharmaceutically acceptable salt thereof,
wherein:
x₁ is C═O or C(R₁)(R₂);
y₁ₐ is C(R₁₁)₂, O, N(R₃) or S; [symbol]
is a single or double bond such that all valences are satisfied;
when [symbol] is a single bond, y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms; or
when [symbol] is a double bond, y_{2b} and y_{2c} are each independently C(R₃) or N, with the proviso that both y₁ₐ and y_{2b} cannot be heteroatoms;
z₁, z₂, z₃ and z₄ are each independently C or N;
R₁ and R₂ are each independently H or F;
R₃ in each occurrence is independently H or CH₃;
R₄, R₅, R₆ and R₇ are each independently H, F, Cl, CH₃ or OCH₃, or each of R₄, R₅, R₆ and R₇ is absent when the respective z to which each is attached is N; and
R₁₁ in each occurrence is independently H, F, Cl, CH₃ or OCH₃.

5. The compound of claim 4 having the structure of Formula IIIb: 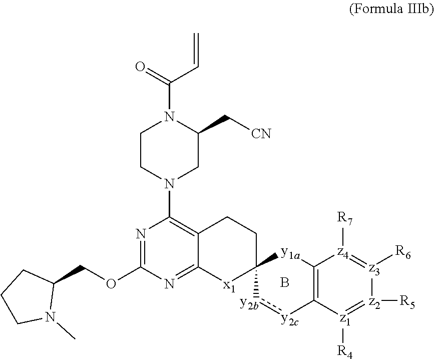
or a pharmaceutically acceptable salt thereof.

6. A compound of claim 4 having the structure of Formula IIIc: 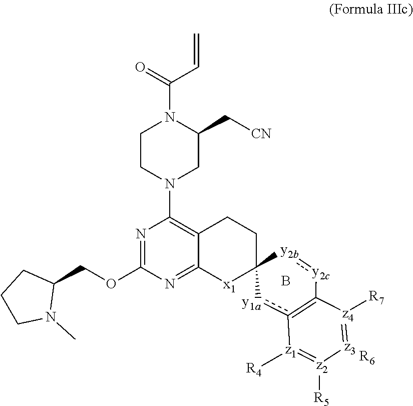
or a pharmaceutically acceptable salt thereof.

7. The compound of claim 1 wherein:
R₈ is C₁-C₃ alkyl substituted with one R₉;
R₉ is cycloalkyl, heterocyclyl, aryl, or heteroaryl, and cycloalkyl, heterocyclyl, aryl, and heteroaryl are optionally substituted with one or more R₁₀; and
R₁₀ in each occurrence is independently halogen, hydroxyl, C₁-C₃ alkyl, alkoxy, haloalkyl, amino, cyano, heteroalkyl or hydroxyalkyl.

8. The compound of claim 7, wherein R₈ is methylene.

9. The compound of claim 7, wherein R₉ is heterocyclyl substituted with one R₁₀, and R₁₀ is methyl.

10. The compound of claim 9, wherein R₉ is pyrrolidine and the N atom of pyrrolidine is methyl substituted.

11. The compound of claim 2 or 3, wherein:
x is C═O or C(R₁)(R₂);
y₁ₐ is CH₂;
y₂ₐ is C(R₁₁)₂, O, N(R₃) or S;
z₁, z₂, z₃ and z₄ are each C;
R₁ and R₂ are H;
R₃ is H or CH₃;
R₄, R₅, R₆ and R₇ are each independently H, F, Cl, CH₃ or OCH₃; and
R₁₁ in each occurrence is independently H, CH₃ or OCH₃.

12. The compound of claim 11, wherein y₂ₐ is C(R₁₁)₂, and R₁₁ is H in one occurrence and is H, CH₃ or OCH₃ in the other.

13. The compound of claim 11, wherein y₂ₐ is O.

14. The compound of claim 11, wherein y₂ₐ is N(R₃) and R₃ is H.

15. The compound of claim 11, wherein y₂ₐ is S.

16. The compound of claim 4, 5 or 6, wherein: [symbol]
is a single bond;
x is C═O or C(R₁)(R₂);
y₁ₐ is C(R₁₁)₂, O, N(R₃) or S;
y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms;
z₁, z₂, z₃ and z₄ are each independently C;
R₁ and R₂ are H;
R₃ in each occurrence is independently H or CH₃;
R₄, R₅, R₆ and R₇ are each independently H, F, Cl, CH₃ or OCH₃; and
R₁₁ in each occurrence is independently H, CH₃ or OCH₃.

17. The compound of claim 16, wherein y₁ₐ is C(R₁₁)₂, and R₁₁ is H in one occurrence and is H, CH₃ or OCH₃ in the other.

18. The compound of claim 16, wherein y₁ₐ is O.

19. The compound of claim 16, wherein y₁ₐ is N(R₃).

20. The compound of claim 16, wherein y₁ₐ is S.

21. The compound of claim 16, wherein y_{2b} is C(R₁₁)₂, and y_{2c} is O, N(R₃) or S.

22. The compound of claim 16, wherein y_{2b} is C(R₁₁)₂, and R₁₁ is H in one occurrence and is H, CH₃ or OCH₃ in the other.

23. The compound of claim 16, wherein y_{2c} is O.

24. The compound of claim 16, wherein y_{2c} is N(R₃).

25. The compound of claim 16, wherein y_{2c} is S.

26. The compound of claim 16, wherein y_{2b} is O, N(R₃) or S, and y_{2c} is C(R₁₁)₂.

27. The compound of claim 16, wherein y_{2c} is C(R₁₁)₂, and R₁ is H in one occurrence and is H, CH₃ or OCH₃ in the other.

28. The compound of claim 16, wherein y_{2b} is O.

29. The compound of claim 16, wherein y_{2b} is N(R₃).

30. The compound of claim 16, wherein y_{2b} is S.

31. The compound of Formula Id, IIa, IIb, IIIa, IIIb or IIIc, wherein:
x is C═O or C(R₁)(R₂);
R₁ and R₂ are H; and
z₁, z₂, z₃ and z₄ are each C.

32. The compound of Formula I having a structure selected from Table 1, or a pharmaceutically acceptable salt thereof.

33. The compound of claim 32, wherein the compound is selected from Compound 1 through Compound 50, or a pharmaceutically acceptable salt thereof.

34. The compound of claim 32, wherein the compound is selected from Compound 1 through Compound 33, or a pharmaceutically acceptable salt thereof.

35. The compound of claim 32, wherein the compound is selected from Compound 7, 9, 11, 13, 14, 17, 21, 22, 25, 26, 27, 29, 30, 31, 33, 35, 36, 42, 44, 46, 47, 50, 51, 55, 58, 63, 70, 71, 73, 77, 87, 88, 91, 93, 95, 96, 98, 99 and 100, or a pharmaceutically acceptable salt thereof.

36. The compound of claim 32, wherein the compound is selected from Compound 7, 9, 11, 13, 17, 21, 22, 25, 26, 30, 31, 33, 35, 36, 42, 44, 46, 47, 50, 51, 55, 58, 63, 70, 71, 73, 77, 87, 88, 91, 93, 95, 96, 98, 99 and 100, or a pharmaceutically acceptable salt thereof.

37. A compound having the structure of Formula I: 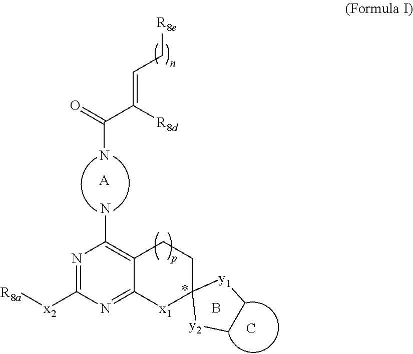
or a pharmaceutically acceptable salt thereof,
wherein:
* is the quaternary carbon atom;
A is a 4-12 membered saturated or partially saturated monocyclic, bridged or spirocyclic ring substituted with one R_{8b} and one R_{8c};
B is a 5-7 membered saturated or partially saturated cycloalkyl or heterocyclyl;
C is an aryl or heteroaryl optionally substituted with one or more R₄;
x₁ is C═O or C(R₁)(R₂);
x₂ is bond, C(R₃)₂, C═O, O, N(R₃), S, S(O), or or S(O)₂;
y₁ is y₁ₐ and y₂ is y₂ₐ; or
y₁ is *-y_{1b}-y_{1c} and y₂ is y₂ₐ; or
y₁ is y₁ₐ and y₂ is *-y_{2b}-y_{2c}; or
y₁ is *-y_{1d}=y₁ₑ and y₂ is y₂ₐ; or
y₁ is y₁ₐ and y₂ is *-y_{2d}=y₂ₑ; or
y₁ is *y₁ₐ-y_{1b}-y_{1c} and y₂ is bond; or
y₁ is bond and y₂ is *y₂ₐ-y_{2b}-y_{2c};
y₁ₐ and y₂ₐ are each independently bond, (C(R₁₁)₂)ₘ, C═CH₂, C═O, O, N(R₃), S, S(O), or S(O)₂;
y_{1b}, y_{1c}, y_{2b} and y_{2c} are each independently bond, (C(R₁₁)₂)ₘ, C═CH₂, C═O, O, N(R₃), S, S(O), or S(O)₂;
y_{1d}, y₁ₑ, y_{2d} and y₂ₑ are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y_{1d} and y₂ₐ cannot be heteroatoms;
with the proviso that both y₁ₐ and y_{2d} cannot be heteroatoms;
with the proviso that both y₁ₐ and y_{1b} cannot be heteroatoms, the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;
R₁ and R₂ are each independently H or F;
R₃ in each occurrence is independently H or C₁-C₄ alkyl;
R₄ in each instance is independently H, OH, F, Cl, Br, N(R₃)₂, CF₃, CH₃, OCFH₂ or OCH₃;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8b} is H, C₁-C₃ alkyl-CN or C₁-C₃ alkyl-OCH₃;
R_{8c} is H or C₁-C₄ alkyl;
R_{8d} is H, cyano, halogen, C₁-C₃ alkyl, haloalkyl, heteroalkyl, hydroxyalkyl or C(O)N(R₃)₂;
R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃;
m in each occurrence is independently 1, 2 or 3;
n is 0, 1, 2 or 3; and
p is 0 or 1.

38. The compound of claim 37, wherein n is 0.

39. The compound of claim 37 or claim 38, wherein p is 1.

40. The compound of any one of claims 37-39, wherein B is a 5-membered saturated or partially saturated cycloalkyl or heterocyclyl.

41. The compound of any one of claims 37-40, wherein the compound of Formula I has the structure of the compound of Formula Ia, 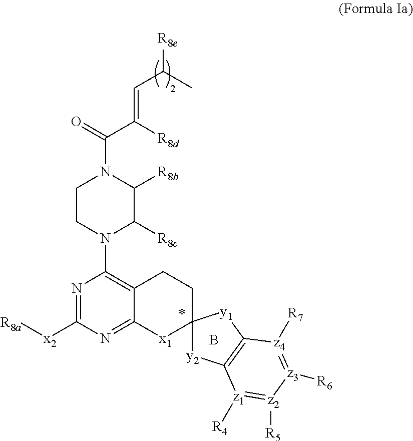
or a pharmaceutically acceptable salt thereof,
wherein:
* is the quaternary carbon atom;
B is a 5-7 membered saturated or partially saturated cycloalkyl or heterocyclyl;
x₁ is C═O or C(R₁)(R₂);
x₂ is bond, C(R₃)₂, C═O, O, N(R₃), S, S(O), or S(O)₂;
y₁ is y₁ₐ and y₂ is y₂ₐ; or
y₁ is *-y_{1b}-y_{1c} and y₂ is y₂ₐ; or
y₁ is y₁ₐ and y₂ is *-y_{2b}-y_{2c}; or
y₁ is *-y_{1d}=y₁ₑ and y₂ is y₂ₐ; or
y₁ is y₁ₐ and y₂ is *-y_{2d}=y₂ₑ; or
y₁ is *y₁ₐ-y_{1b}-y_{1c} and y₂ is bond; or
y₁ is bond and y₂ is *y₂ₐ-y_{2b}-y_{2c};
y₁ₐ and y₂ₐ are each independently bond, (C(R₁₁)₂)ₘ, C═CH₂, C═O, O, N(R₃), S, S(O), or S(O)₂;
y_{1b}, y_{1c}, y_{2b} and y_{2c} are each independently bond, (C(R₁₁)₂)ₘ, C═CH₂, C═O, O, N(R₃), S, S(O), or S(O)₂;
y_{1d}, y₁ₑ, y_{2d} and y₂ₑ are each independently C(R₃) or N;
with the proviso that both y₁ₐ and y₂ₐ cannot be heteroatoms;
with the proviso that both y_{1b} and y₂ₐ cannot be heteroatoms, and the proviso that both y_{1b} and y_{1c} cannot be heteroatoms;
with the proviso that both y₁ₐ and y_{2b} cannot be heteroatoms, and the proviso that both y_{2b} and y_{2c} cannot be heteroatoms;
with the proviso that both y_{1d} and y₂ₐ cannot be heteroatoms;
with the proviso that both y₁ₐ and y_{2d} cannot be heteroatoms;
with the proviso that both y₁ₐ and y_{1b} cannot be heteroatoms, and the proviso that both y_{1b} and y_{1c} cannot be heteroatoms; and
with the proviso that both y₂ₐ and y_{2b} cannot be heteroatoms, the proviso that both y_{2b} and y_{2c} cannot be heteroatoms;
z₁, z₂, z₃ and z₄ are each independently C or N;
R₁ and R₂ are each independently H or F;
R₃ in each occurrence is independently H or C₁-C₄ alkyl;
R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8b} is H, C₁-C₃ alkyl-CN or C₁-C₃ alkyl-OCH₃;
R_{8c} is H or C₁-C₄ alkyl;
R_{8d} is H, cyano, halogen, C₁-C₃ alkyl, haloalkyl, heteroalkyl, hydroxyalkyl or C(O)N(R₃)₂;
R₈ₑ is H, cyano, C₁-C₃ alkyl, hydroxyalkyl, heteroalkyl, C₁-C₃ alkoxy, halogen, haloalkyl, haloalkoxy, (CH₂)ₘN(R₃)₂, N(R₃)₂, C(O)N(R₃)₂, N(H)C(O)C₁-C₃ alkyl, CH₂N(H)C(O)C₁-C₃ alkyl, heteroaryl or heterocyclyl;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃;
m in each occurrence is independently 1, 2 or 3; and
n is 0, 1, 2 or 3.

42. The compound of claim 41, wherein n is 0.

43. The compound of claim 41 or claim 42, wherein B is a 5-membered saturated or partially saturated cycloalkyl or heterocycloalkyl.

44. The compound of any one of claims 41-43, wherein the compound having the structure of Formula Ia has the structure of Formula Ib: 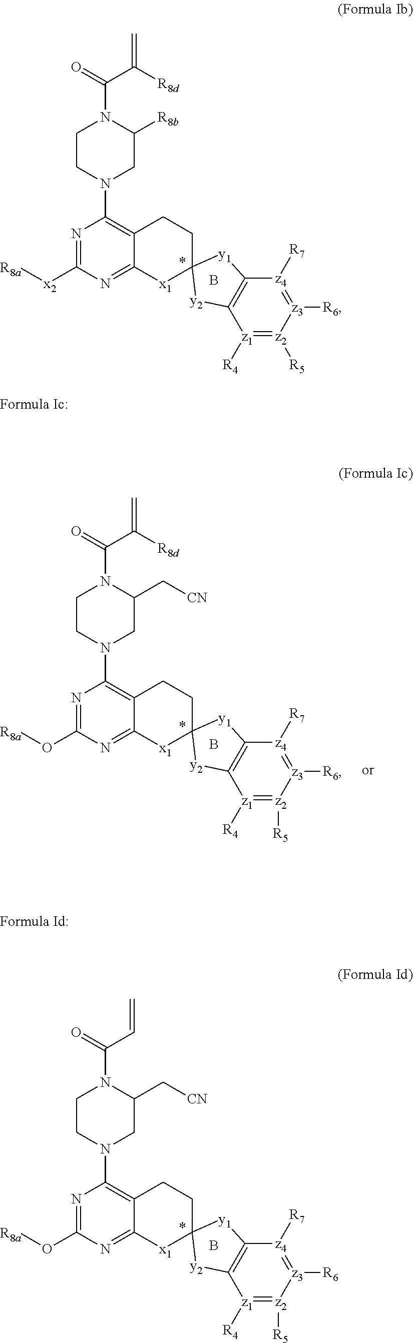
or a pharmaceutically acceptable salt thereof.

45. The compound of any one claims 41-43, wherein:
y₁ is y₁ₐ and y₂ is y₂ₐ, with the proviso that both y₁ₐ and y₂ₐ cannot be heteroatoms, and the further proviso that neither y₁ₐ or y₂ₐ can be a bond when y₁ is y₁ₐ and y₂ is y₂ₐ; or
y₁ is *-y_{1b}-y_{1c} and y₂ is y₂ₐ, with the proviso that both y_{1b} and y₂ₐ cannot be heteroatoms, the proviso that both y_{1b} and y_{1c} cannot be bonds, the proviso that both y_{1b} and y_{1c} cannot be heteroatoms, the proviso that both y_{1b} and y_{1c} cannot be C═O, and the further proviso that both y_{1b} and y_{1c} cannot be C═CH₂; or
y₁ is y₁ₐ and y₂ is *-y_{2b}-y_{2c}, with the proviso that both y₁ₐ and y_{2b} cannot be heteroatoms, the proviso that both y_{2b} and y_{2c} cannot be bonds, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or
y₁ is *y_{1d}=y₁ₑ and y₂ is y₂ₐ, with the proviso that both y_{1d} and y₂ₐ cannot be heteroatoms; or
y₁ is y₁ₐ and y₂ is *-y_{2d}=y₂ₑ, with the proviso that both y₁ₐ and y_{2d} cannot be heteroatoms; or
y₁ is *y₁ₐ-y_{1b}-y_{1c} and y₂ is bond, with the proviso that none of y₁ₐ, y_{1b} and y_{1c} can be a bond, the proviso that both y₁ₐ and y_{1b} cannot be heteroatoms, the proviso that both y_{1b} and y_{1c} cannot be heteroatoms, the proviso that both y₁ₐ and y_{1b} cannot be C═O, the proviso that both y_{1b} and y_{1c} cannot be C═O, the proviso that both y₁ₐ and y_{1b} cannot be C═CH₂, and the further proviso that both y_{1b} and y_{1c} cannot be C═CH₂; or
y₁ is bond and y₂ is *y₂ₐ-y_{2b}-y_{2c}, with the proviso that none of y₂ₐ, y_{2b} and y_{2c} can be a bond, the proviso that both y₂ₐ and y_{2b} cannot be heteroatoms, the proviso that both y_{2b} and y_{2c} cannot be heteroatoms, the proviso that both y₂ₐ and y_{2b} cannot be C═O, the proviso that both y_{2b} and y_{2c} cannot be C═O, the proviso that both y₂ₐ and y_{2b} cannot be C═CH₂, and the further proviso that both y_{2b} and y_{2c} cannot be C═CH₂.

46. The compound of claim 44, wherein the compound of Formula I has the structure of Formula Ia, Ib, Ic, or Id, or a pharmaceutically acceptable salt thereof, wherein:
* is the quaternary carbon atom;
x₁ is C═O or C(R₁)(R₂);
y₁ is y₁ₐ and y₂ is y₂ₐ; or
y₁ is *-y_{1b}-y_{1c} and y₂ is y₂ₐ; or
y₁ is y₁ₐ and y₂ is *y_{2b}-y_{2c}; or
y₁ is *y_{1d}=y₁ₑ and y₂ is y₂ₐ; or
y₁ is y₁ₐ and y₂ is *-y_{2d}=y₂ₑ;
y₁ₐ and y₂ₐ are each independently C(R₁₁)₂, O, N(R₃) or S;
y_{1b}, y_{1c}, y_{2b} and y_{2c} are each independently C(R₁₁)₂, O, N(R₃) or S;
y_{1d}, y₁ₑ, y_{2d} and y₂ₑ are each independently C(R₃) or N;
with the proviso that both y₁ₐ and y₂ₐ cannot be heteroatoms;
with the proviso that both y_{1b} and y₂ₐ cannot be heteroatoms, and the further proviso that both y_{1b} and y_{1c} cannot be heteroatoms;
with the proviso that both y₁ₐ and y_{2b} cannot be heteroatoms, and the further proviso that both y_{2b} and y_{2c} cannot be heteroatoms;
with the proviso that both y_{1d} and y₂ₐ cannot be heteroatoms;
with the proviso that both y₁ₐ and y_{2d} cannot be heteroatoms;
z₁, z₂, z₃ and z₄ are each independently C or N;
R₁ and R₂ are each independently H or F;
R₃ in each occurrence is independently H or CH₃;
R₄, R₅, R₆ and R₇ are each independently H, F, Cl, CH₃ or OCH₃, or each of R₄, R₅, R₆ and R₇ is absent when the respective z to which each is attached is N;
R₈ₐ is H, C₁-C₃ alkyl, cycloalkyl, heterocyclyl, aralkyl, aryl or heteroaryl, wherein each of C₁-C₃ alkyl, cycloalkyl, heterocyclyl, aralkyl, aryl and heteroaryl may be optionally substituted with one or more R₉;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl; and
R₁₁ in each occurrence is independently H, F, Cl, CH₃ or OCH₃.

47. The compound of any one of claims 37-40, wherein:
y₁ is y₁ₐ and y₂ is y₂ₐ, with the proviso that both y₁ₐ and y₂ₐ cannot be heteroatoms, and the further proviso that neither y₁ₐ or y₂ₐ can be a bond when y₁ is y₁ₐ and y₂ is y₂ₐ; or
y₁ is *-y_{1b}-y_{1c} and y₂ is y₂ₐ, with the proviso that both y_{1b} and y₂ₐ cannot be heteroatoms, the proviso that both y_{1b} and y_{1c} cannot be bonds, the proviso that both y_{1b} and y_{1c} cannot be heteroatoms, the proviso that both y_{1b} and y_{1c} cannot be C═O, and the further proviso that both y_{1b} and y_{1c} cannot be C═CH₂; or
y₁ is y₁ₐ and y₂ is *-y_{2b}-y_{2c}, with the proviso that both y₁ₐ and y_{2b} cannot be heteroatoms, the proviso that both y_{2b} and y_{2c} cannot be bonds, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or
y₁ is *y_{1d}=y₁ₑ and y₂ is y₂ₐ, with the proviso that both y_{1d} and y₂ₐ cannot be heteroatoms; or
y₁ is y₁ₐ and y₂ is *-y_{2d}=y₂ₑ, with the proviso that both y₁ₐ and y_{2d} cannot be heteroatoms; or
y₁ is *y₁ₐ-y_{1b}-y_{1c} and y₂ is bond, with the proviso that none of y₁ₐ, y_{1b} and y_{1c} can be a bond, the proviso that both y₁ₐ and y_{1b} cannot be heteroatoms, the proviso that both y_{1b} and y_{1c} cannot be heteroatoms, the proviso that both y₁ₐ and y_{1b} cannot be C═O, the proviso that both y_{1b} and y_{1c} cannot be C═O, the proviso that both y₁ₐ and y_{1b} cannot be C═CH₂, and the further proviso that both y_{1b} and y_{1c} cannot be C═CH₂; or
y₁ is bond and y₂ is *y₂ₐ-y_{2b}-y_{2c}, with the proviso that none of y₂ₐ, y_{2b} and y_{2c} can be a bond, the proviso that both y₂ₐ and y_{2b} cannot be heteroatoms, the proviso that both y_{2b} and y_{2c} cannot be heteroatoms, the proviso that both y₂ₐ and y_{2b} cannot be C═O, the proviso that both y_{2b} and y_{2c} cannot be C═O, the proviso that both y₂ₐ and y_{2b} cannot be C═CH₂, and the further proviso that both y_{2b} and y_{2c} cannot be C═CH₂.

48. A compound having the structure of Formula II: 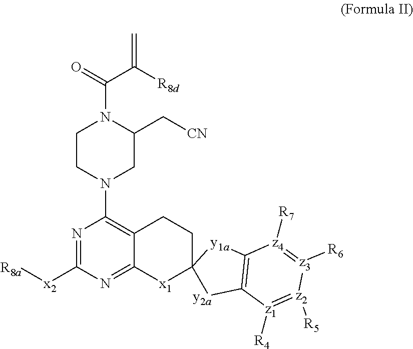
or a pharmaceutically acceptable salt thereof,
wherein:
x₁ is C═O or C(R₁)(R₂);
x₂ is bond, C(R₃)₂, C═O, O, N(R₃), S, S(O), or S(O)₂;
y₁ₐ and y₂ₐ are each independently (C(R₁₁)₂)ₘ, C═CH₂, C═O, O, N(R₃), S, S(O), or S(O)₂, with the proviso that both y₁ₐ and y₂ₐ cannot be heteroatoms;
z₁, z₂, z₃ and z₄ are each independently C or N;
R₁ and R₂ are each independently H or F;
R₃ in each occurrence is independently H or C₁-C₄ alkyl;
R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8d} is H, cyano, halogen, C₁-C₃ alkyl, haloalkyl, heteroalkyl, hydroxyalkyl or C(O)N(R₃)₂;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃; and
m, when present, is 1.

49. The compound of claim 48, wherein R₈ₐ is H or halogen (such as F).

50. The compound of claim 48 or claim 49, wherein the compound of Formula II has the structure of Formula IIa: 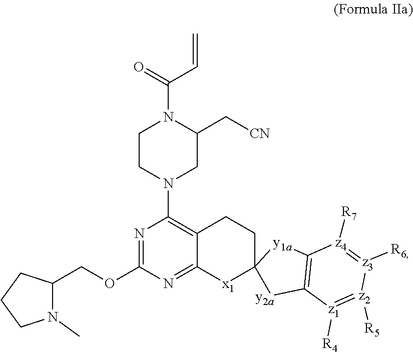
or the structure of Formula IIb: 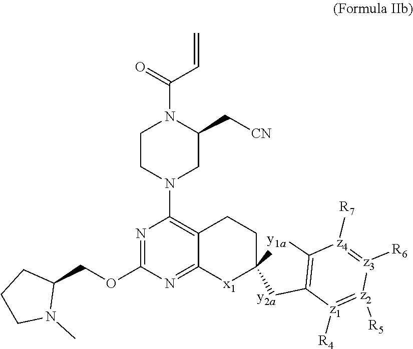
or a pharmaceutically acceptable salt thereof.

51. The compound of any one of claims 48-50, wherein the compound of Formula II has the structure of Formula IIa or IIb, or a pharmaceutically acceptable salt thereof, and further wherein:
x₁ is C═O or C(R₁)(R₂);
y₁ₐ and y₂ₐ are each independently C(R₁₁)₂, O, N(R₃) or S, with the proviso that both y₁ₐ and y₂ₐ cannot be heteroatoms;
z₁, z₂, z₃ and z₄ are each independently C or N;
R₁ and R₂ are each independently H or F;
R₃ is H or CH₃;
R₄, R₅, R₆ and R₇ are each independently H, F, Cl, CH₃ or OCH₃, or each of R₄, R₅, R₆ and R₇ is absent when the respective z to which each is attached is N; and
R₁₁ in each occurrence is independently H, F, Cl, CH₃ or OCH₃.

52. A compound having the structure of Formula III: 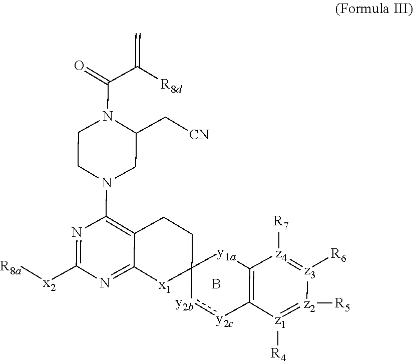
or a pharmaceutically acceptable salt thereof,
wherein:
B is a 5-7 membered saturated or partially saturated cycloalkyl or heterocyclyl;
x₁ is C═O or C(R₁)(R₂);
x₂ is bond, C(R₃)₂, C═O, O, N(R₃), S, S(O), or S(O)₂; [symbol]
is a single or double bond such that all valences are satisfied;
y₁ₐ is bond, (C(R₁₁)₂)ₘ, C═CH₂, C═O, O, N(R₃), S, S(O), or S(O)₂;
when [symbol] is a single bond, y_{2b} and y_{2c} are each independently bond, (C(R₁₁)₂)ₘ, C═CH₂, C═O, O, N(R₃), S, S(O), or S(O)₂, with the proviso that both y₁ₐ and y_{2b} cannot be heteroatoms, and the proviso that both y_{2b} and y_{2c} cannot be heteroatoms; or
when [symbol] is a double bond, y_{2b} and y_{2c} are each independently C(R₃) or N, with the proviso that both y₁ₐ and y_{2b} cannot be heteroatoms;
z₁, z₂, z₃ and z₄ are each independently C or N;
R₁ and R₂ are each independently H or F;
R₃ in each occurrence is independently H or C₁-C₄ alkyl;
R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8d} is H, cyano, halogen, C₁-C₃ alkyl, haloalkyl, heteroalkyl, hydroxyalkyl or C(O)N(R₃)₂;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃; and
m in each occurrence is independently 1, 2 or 3.

53. The compound of claim 52, wherein R_{8d} is H or halogen (such as F).

54. The compound of claim 52 or claim 53, wherein the compound of Formula III has the structure of Formula IIIa: 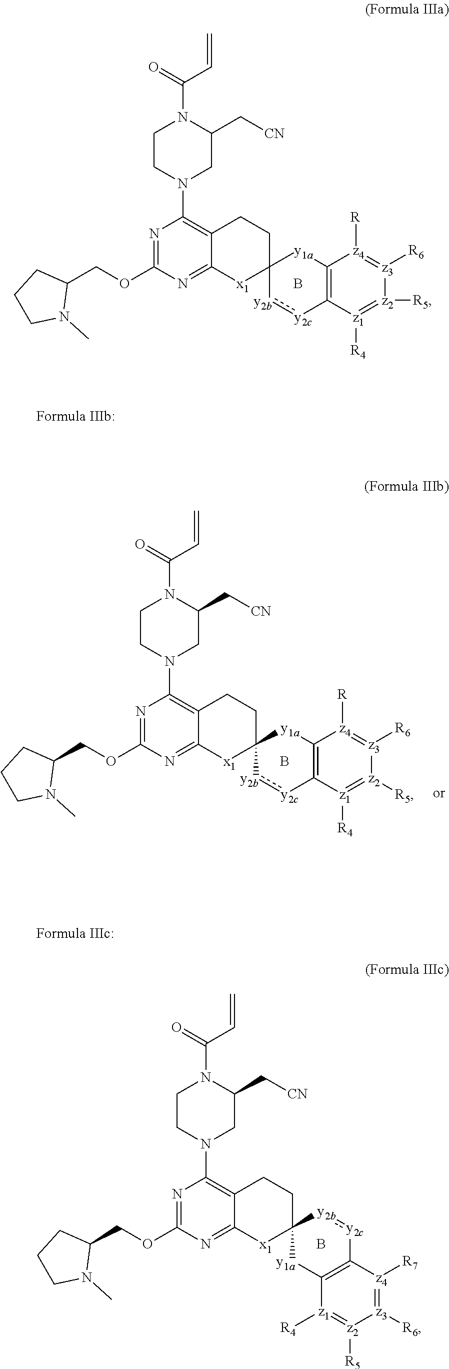
or a pharmaceutically acceptable salt thereof.

55. The compound of any one of claims 52-54, wherein B is a 6-membered saturated or partially saturated cycloalkyl or heterocyclyl.

56. The compound of claim 54, wherein:
x₁ is C═O or C(R₁)(R₂);
y₁ₐ is C(R₁₁)₂, O, N(R₃) or S; [symbol]
is a single or double bond such that all valences are satisfied;
when [symbol] is a single bond, y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms; or
when [symbol] is a double bond, y_{2b} and y_{2c} are each independently C(R₃) or N, with the proviso that both y₁ₐ and y_{2b} cannot be heteroatoms;
z₁, z₂, z₃ and z₄ are each independently C or N;
R₁ and R₂ are each independently H or F;
R₃ in each occurrence is independently H or CH₃;
R₄, R₅, R₆ and R₇ are each independently H, F, Cl, CH₃ or OCH₃, or each of R₄, R₅, R₆ and R₇ is absent when the respective z to which each is attached is N; and
R₁₁ in each occurrence is independently H, F, Cl, CH₃ or OCH₃.

57. The compound of claim 54, wherein the compound of formula III has the structure of Formula IIIa, IIIb, or IIIc, or a pharmaceutically acceptable salt thereof, wherein:
B is a 6 membered saturated cycloalkyl or heterocyclyl;
x₁ C(R₁)(R₂); [symbol]
is a single bond;
y₁ₐ is (C(R₁₁)₂)ₘ;
y_{2b} is (C(R₁₁)₂)ₘ;
y_{2c} is (C(R₁₁)₂)ₘ or N(R₃);
z₁, z₂, z₃ and z₄ are each C;
R₁ and R₂ are each independently H;
R₃ in each occurrence is independently C₁-C₄ alkyl;
R₄, R₅, R₆ and R₇ are each independently H, F or CH₃;
R₁₁ in each occurrence is independently H;
m in each occurrence is independently 1; and
wherein the compound has a KRASG12C k_{obs}/[i] of about 1000 M⁻¹ s⁻¹ or greater.

58. The compound of claim 57, wherein the compound has an average IC₅₀ of greater than 1000 nM for the drug-resistant cell lines of Table 5.

59. The compound of claim 57 or claim 58, wherein the compound has an average IC₅₀ of about 1000 nM or lower for the drug-sensitive cell lines of Table 5.

60. The compound of claim 57, wherein the compound is selected from: 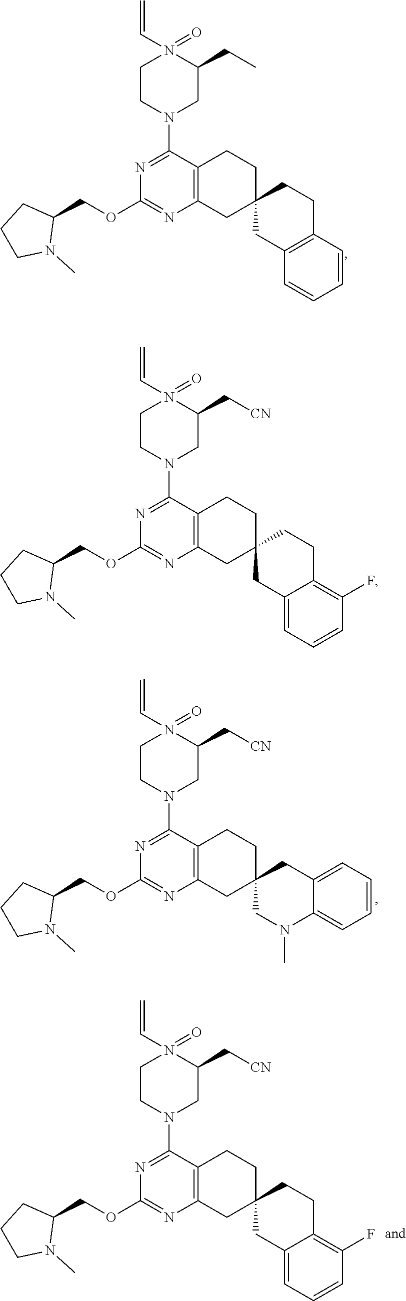 [SMILES: C=C[N+]1([O-])CCN(c2nc(OC[C@@H]3CCCN3C)nc3c2CC[C@@]2(CCc4ccccc4C2)C3)C[C@@H]1CC ; C=C[N+]1([O-])CCN(c2nc(OC[C@@H]3CCCN3C)nc3c2CC[C@]2(CCc4c(F)cccc4C2)C3)C[C@@H]1CC#N ; C=C[N+]1([O-])CCN(c2nc(OC[C@@H]3CCCN3C)nc3c2CC[C@@]2(Cc4ccccc4N(C)C2)C3)C[C@@H]1CC#N ; C=C[N+]1([O-])CCN(c2nc(OC[C@@H]3CCCN3C)nc3c2CC[C@@]2(CCc4c(F)cccc4C2)C3)C[C@@H]1CC#N] 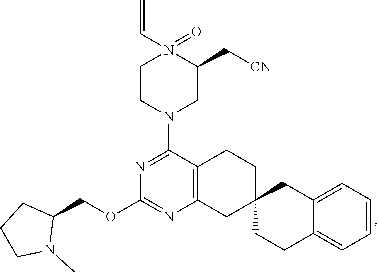 [SMILES: C=C[N+]1([O-])CCN(c2nc(OC[C@@H]3CCCN3C)nc3c2CC[C@]2(CCc4ccccc4C2)C3)C[C@@H]1CC#N]
or a pharmaceutically salt thereof.

61. The compound of any one of claims 52-56, wherein:
when [symbol] is a single bond, y_{2b} and y_{2c} are each independently bond, (C(R₁₁)₂)ₘ, C═CH₂, C═O, O, N(R₃), S, S(O), or S(O)₂, with the proviso that both y₁ₐ and y_{2b} cannot be bonds, the proviso that both y₁ₐ and y_{2b} cannot be heteroatoms, the proviso that both y_{2b} and y_{2c} cannot be heteroatoms, the proviso that both y_{2b} and y_{2c} cannot be C═O, and the further proviso that both y_{2b} and y_{2c} cannot be C═CH₂; or
when [symbol] is a double bond, y_{2b} and y_{2c} are each independently C(R₃) or N, with the proviso that both y₁ₐ and y_{2b} cannot be heteroatoms.

62. A compound having structural formula IV: 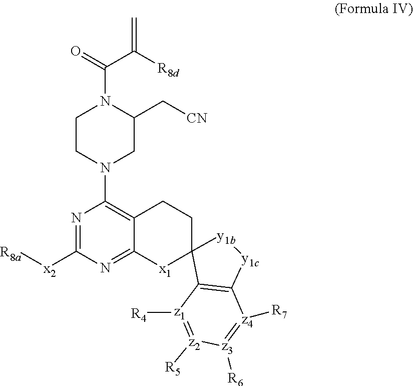
of a pharmaceutically acceptable salt thereof,
wherein:
x₁ is C═O or C(R₁)(R₂);
x₂ is bond, C(R₃)₂, C═O, O, N(R₃), S, S(O), or S(O)₂;
y_{1b} and y_{1c} are each independently (C(R₁₁)₂)ₘ, C═CH₂, C═O, O, N(R₃), S, S(O), or S(O)₂, with the proviso that both y_{1b} and y_{1c} cannot be heteroatoms, the proviso that both y_{1b} and y_{1c} cannot be C═CH₂, and the further proviso that both y_{1b} and y_{1c} cannot be C═O;
z₁, z₂, z₃ and z₄ are each independently C or N;
R₁ and R₂ are each independently H or F;
R₃ in each occurrence is independently H or C₁-C₄ alkyl;
R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8d} is H, cyano, halogen, C₁-C₃ alkyl, haloalkyl, heteroalkyl, hydroxyalkyl or C(O)N(R₃)₂;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃; and
m, when present, is 1.

63. The compound of claim 62, wherein R_{8d} is H or halogen (such as F).

64. The compound of claim 62 or claim 63, wherein the compound of Formula IV has the structure of Formula IVa: 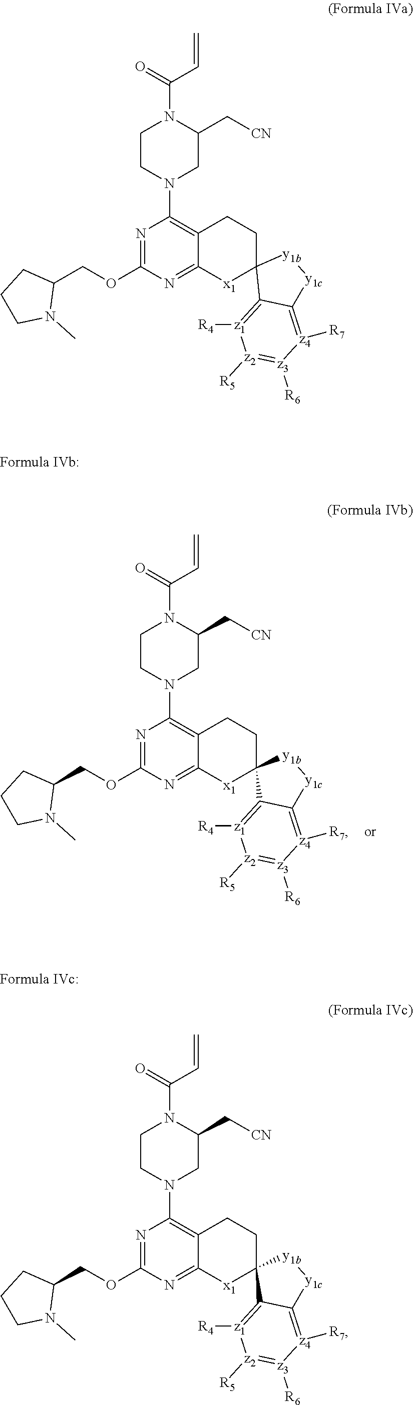
or a pharmaceutically acceptable salt thereof.

65. A compound having the structure of Formula V: 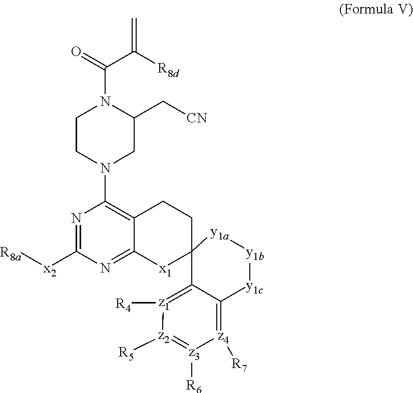
of a pharmaceutically acceptable salt thereof,
wherein:
x₁ is C═O or C(R₁)(R₂);
x₂ is bond, C(R₃)₂, C═O, O, N(R₃), S, S(O), or S(O)₂;
y₁ₐ, y_{1b} and y_{1c} are each independently (C(R₁₁)₂)ₘ, C═CH₂, C═O, O, N(R₃), S, S(O), or S(O)₂, with the proviso that both y₁ₐ and y_{1b} cannot be heteroatoms, the proviso that both y_{1b} and y_{1c} cannot be heteroatoms, the proviso that both y₁ₐ and y_{1b} cannot be C═CH₂, the proviso that both y_{1b} and y_{1c} cannot be C═CH₂, the proviso that both y₁ₐ and y_{1b} cannot be C═O, and the further proviso that both y_{1b} and y_{1c} cannot be C═O;
z₁, z₂, z₃ and z₄ are each independently C or N;
R₁ and R₂ are each independently H or F;
R₃ in each occurrence is independently H or C₁-C₄ alkyl;
R₄, R₅, R₆ and R₇ are each independently H, OH, F, Cl, Br, N(R₃)₂, CF₃, CH₃, OCFH₂ or OCH₃, or each of R₄, R₅, R₆ and R₇ is absent when the respective z to which each is attached is N;
R₈ₐ is H, C₁-C₄ alkyl, cycloalkyl, heterocyclyl, aralkyl, aryl or heteroaryl, wherein each of C₁-C₄ alkyl, cycloalkyl, heterocyclyl, aralkyl, aryl and heteroaryl may be optionally substituted with one or more R₉;
R_{8d} is H, cyano, halogen, C₁-C₃ alkyl, haloalkyl, heteroalkyl, hydroxyalkyl or C(O)N(R₃)₂;
R₉ in each occurrence is independently halogen, hydroxyl, C₁-C₃ alkyl, C₁-C₆ alkyl, cycloalkyl, heterocyclyl, aryl, heteroaryl, alkoxy, haloalkyl, amino, cyano, heteroalkyl or hydroxyalkyl, wherein each of cycloalkyl, heterocyclyl, aryl and heteroaryl may be optionally substituted with one or more R₁₀;
R₁₀ in each occurrence is independently halogen, hydroxyl, C₁-C₃ alkyl, alkoxy, haloalkyl, amino, cyano, heteroalkyl or hydroxyalkyl;
R₁₁ in each occurrence is independently H, F, Cl, C₁-C₃ alkyl or OCH₃; and
m, when present, is 1.

66. The compound of claim 65, wherein R_{8d} is H or halogen (such as F).

67. The compound of claim 65 or claim 66, wherein the compound of Formula V has the structure of Formula Va: 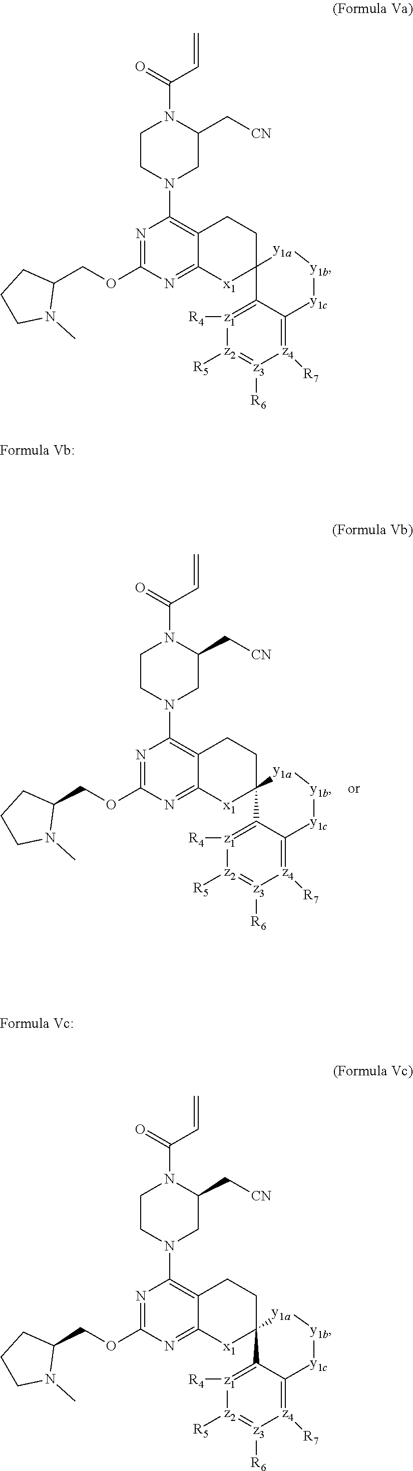
or a pharmaceutically acceptable salt thereof.

68. The compound of any one of claims 37-67, wherein:
R₈ₐ is C₁-C₃ alkyl substituted with one R₉;
R₉ is cycloalkyl, heterocyclyl, aryl, or heteroaryl, and cycloalkyl, heterocyclyl, aryl, and heteroaryl are optionally substituted with one or more R₁₀; and
R₁₀ in each occurrence is independently halogen, hydroxyl, C₁-C₃ alkyl, alkoxy, haloalkyl, amino, cyano, heteroalkyl or hydroxyalkyl.

69. The compound of claim 68, wherein R₈ₐ is C₁-C₃ alkyl, and C₁-C₃ alkyl is methylene.

70. The compound of claim 68 or claim 69, wherein R₉ is heterocyclyl substituted with one R₁₀, and R₁₀ is methyl.

71. The compound of claim 70, wherein heterocyclyl is pyrrolidine wherein the N atom of pyrrolidine is methyl-substituted.

72. The compound of claim 50, wherein the compound of Formula II is a compound of Formula IIa or IIb, and further wherein:
x₁ is C═O or C(R₁)(R₂);
y₁ₐ is CH₂;
y₂ₐ is C(R₁₁)₂, O, N(R₃) or S;
z₁, z₂, z₃ and z₄ are each C;
R₁ and R₂ are H;
R₃ is H or CH₃;
R₄, R₅, R₆ and R₇ are each independently H, F, Cl, CH₃ or OCH₃; and
R₁₁ in each occurrence is independently H, CH₃ or OCH₃.

73. The compound of claim 54, wherein: [symbol]
is a single bond;
x₁ is C═O or C(R₁)(R₂);
y₁ₐ is C(R₁₁)₂, O, N(R₃) or S;
y_{2b} and y_{2c} are each independently C(R₁₁)₂, O, N(R₃) or S, with the proviso that both y₁ₐ and y_{2b} cannot be heteroatoms, and the further proviso that both y_{2b} and y_{2c} cannot be heteroatoms;
z₁, z₂, z₃ and z₄ are each independently C;
R₁ and R₂ are H;
R₃ in each occurrence is independently H or CH₃;
R₄, R₅, R₆ and R₇ are each independently H, F, Cl, CH₃ or OCH₃; and
R₁₁ in each occurrence is independently H, CH₃ or OCH₃.

74. The compound of claim 54, wherein:
B is a 6-membered saturated cycloalkyl or heterocyclyl;
x₁ is C(R₁)(R₂); [symbol]
is a single bond;
y₁ₐ is (C(R₁₁)₂)ₘ;
y_{2b} is (C(R₁₁)₂)ₘ;
y_{2c} is (C(R₁₁)₂)ₘ or N(R₃);
z₁, z₂, z₃ and z₄ are each C;
R₁ and R₂ are each independently H;
R₃ in each occurrence is independently C₁-C₄ alkyl;
R₄, R₅, R₆ and R₇ are each independently H, F or CH₃;
R₁₁ in each occurrence is independently H; and
m in each occurrence is independently 1.

75. The compound of any one of claims 37-67, wherein the compound is a compound of formula I, Ia, Ib, Ic, Id, II, IIa, IIb, III, IIIa, IIIb IIIc, IV, IVa, IVb, IVc, V, Va, Vb, or Vc, or a pharmaceutically acceptable salt thereof, and further wherein:
x₁ is C═O or C(R₁)(R₂);
R₁ is H;
R₂ is H; and
z₁, z₂, z₃, and z₄ are each C.

76. The compound of claim 75, wherein the compound is of formula Id, IIa, IIb, IIIa, IIIb, or IIIc, or a pharmaceutically acceptable salt thereof.

77. The compound of any one of claims 37-67, wherein the compound is of Formula I, Ia, Ib, Ic, III, IIIa, IIIb, or IIIc, or a pharmaceutically acceptable salt thereof, and further wherein B is a 5- or 6-membered cycloalkyl.

78. The compound of any one of claims 37-67, wherein the compound is of Formula I, Ia, Ib, Ic, III, IIIa, IIIb, or IIIc, or a pharmaceutically acceptable salt thereof, and further wherein B is a 5- or 6-membered heterocyclyl.

79. The compound of claim 78, wherein the 5- or 6-membered heterocyclyl is selected from tetrahydrofuranyl, tetrahydrothiophenyl, sulfolanyl, pyrrolidinyl, tetrahydropyranyl, 1,4-dioxanyl, piperidinyl, piperazinyl, thiomorpholinyl, thiomorpholinyl dioxide, morpholinyl, 1,4-dithianyl, thianyl, lactamyl and lactonyl.

80. The compound of any one of claims 37-67, wherein x₂ is O.

81. The compound of any one of claims 37-67, wherein when R₃ is C₁-C₄ alkyl, C₁-C₄ alkyl is methyl or ethyl.

82. The compound of any one of claims 37-67, wherein the compound is of Formula I, Ia, Ib, Ic, II, III, IV, or V, or a pharmaceutically acceptable salt thereof, and further wherein R_{8d} is F.

83. The compound of claim 82, wherein the compound is of Formula I, Ia, or Ib, or a pharmaceutically acceptable salt thereof, wherein R_{8b} is C₁-C₃ alkyl-CN.

84. The compound of claim 82 or claim 83, wherein the compound is of Formula I or Formula Ia, or a pharmaceutically acceptable salt thereof, and further wherein:
R_{8c} is H; and
R₈ₑ is H.

85. The compound of any one of claims 37-67, wherein R₁₁ is C₁-C₃ alkyl.

86. The compound of any one of claims 37-67, wherein the compound is of Formula I, Ia, Ib, Ic, III, IIIa, IIIb, or IIIc, or a pharmaceutically acceptable salt thereof, and further wherein m, in each occurrence, is 1.

87. The compound of any one of claims 37-67, wherein the compound is of formula I or Ia, or a pharmaceutically acceptable salt thereof, and further wherein:
R_{8d} is H, F, methyl, ethyl, OCH₃, CH₂OH or CH₂OCH₃; and
R₈ₑ is H, methyl, ethyl, F, CF₃, CF₂H or CH₂F.

88. The compound of any one of claims 37-67, wherein the compound is of formula Ib, Ic, II, III, IV or V, or a pharmaceutically acceptable salt thereof, and further wherein R_{8d} is H, F, methyl, ethyl, OCH₃, CH₂OH or CH₂OCH₃.

89. The compound of any one of claims 37-67, wherein the compound has a structure selected from: 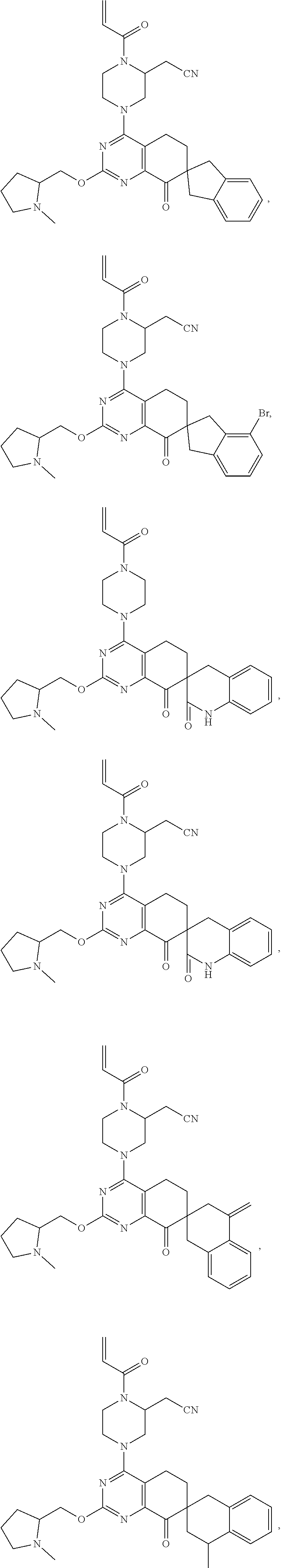 [SMILES: C=CC(=O)N1CCN(c2nc(OCC3CCCN3C)nc3c2CCC2(Cc4ccccc4C2)C3=O)CC1CC#N ; C=CC(=O)N1CCN(c2nc(OCC3CCCN3C)nc3c2CCC2(Cc4cccc(Br)c4C2)C3=O)CC1CC#N ; C=CC(=O)N1CCN(c2nc(OCC3CCCN3C)nc3c2CCC2(Cc4ccccc4NC2=O)C3=O)CC1 ; C=CC(=O)N1CCN(c2nc(OCC3CCCN3C)nc3c2CCC2(Cc4ccccc4NC2=O)C3=O)CC1CC#N ; C=CC(=O)N1CCN(c2nc(OCC3CCCN3C)nc3c2CCC2(CC(=C)c4ccccc4C2)C3=O)CC1CC#N ; C=CC(=O)N1CCN(c2nc(OCC3CCCN3C)nc3c2CCC2(Cc4ccccc4C(C)C2)C3=O)CC1CC#N] 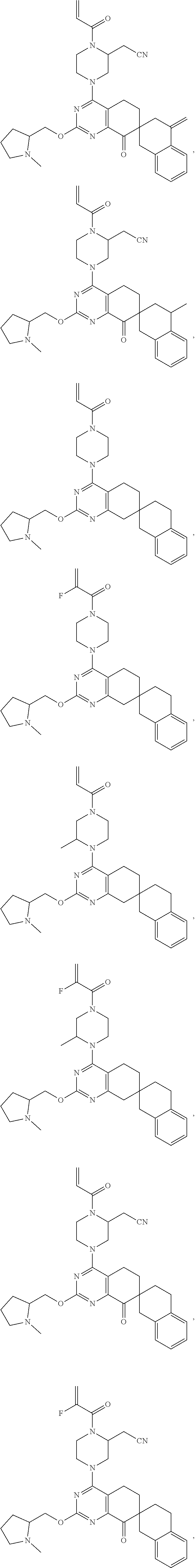 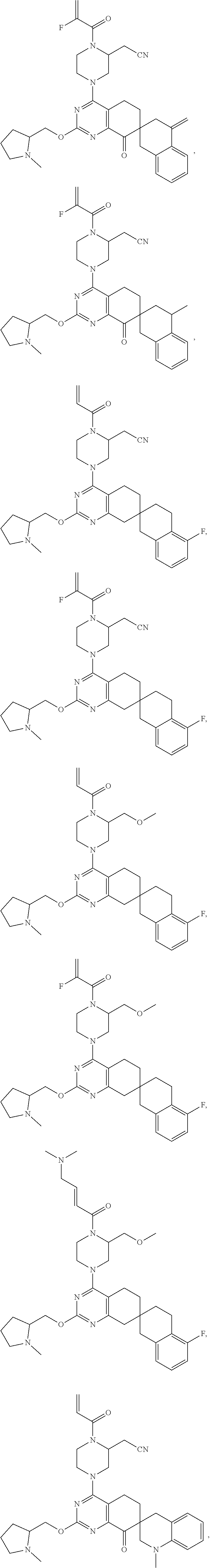 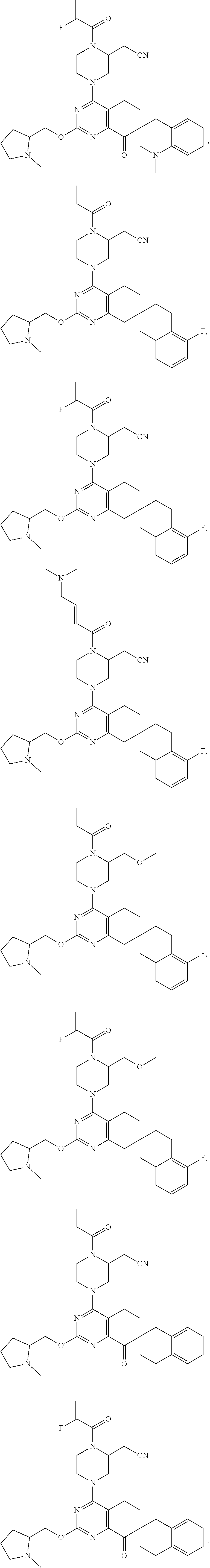 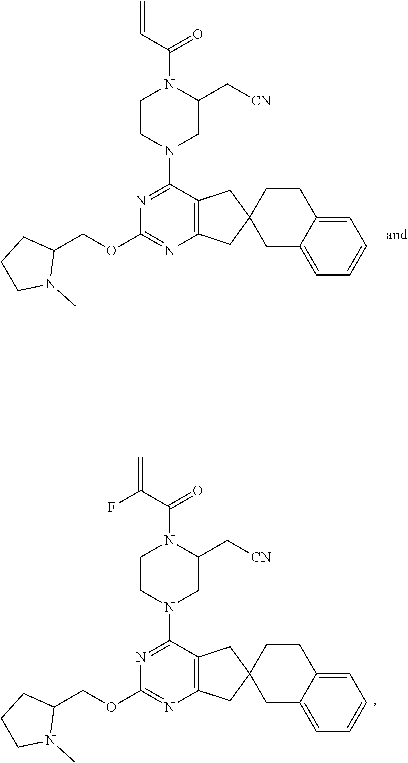 [SMILES: C=CC(=O)N1CCN(c2nc(OCC3CCCN3C)nc3c2CC2(CCc4ccccc4C2)C3)CC1CC#N ; C=C(F)C(=O)N1CCN(c2nc(OCC3CCCN3C)nc3c2CC2(CCc4ccccc4C2)C3)CC1CC#N]
or a pharmaceutically salt thereof.

90. The compound of any one of claims 37-67, wherein the compound has a structure selected from: 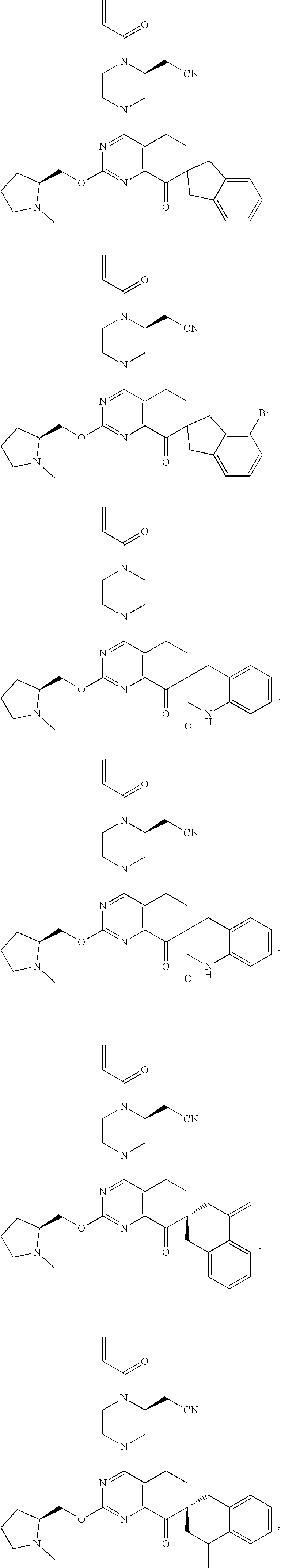 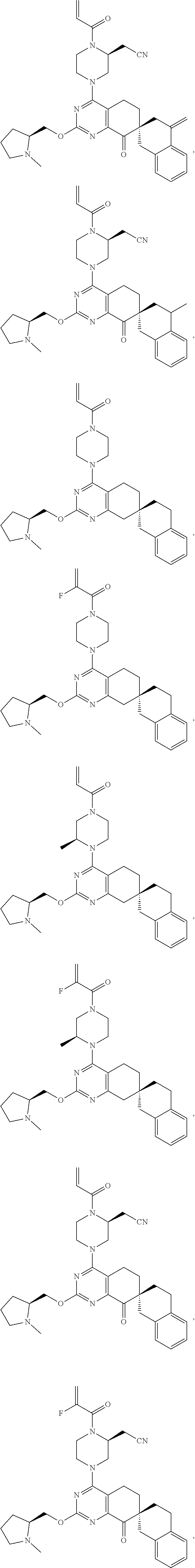 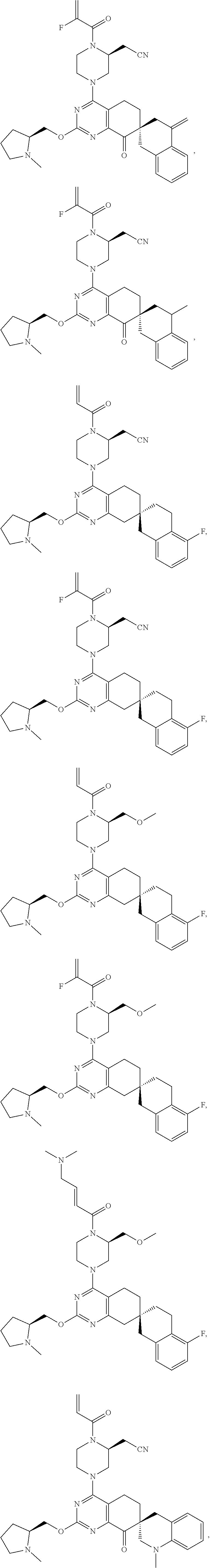 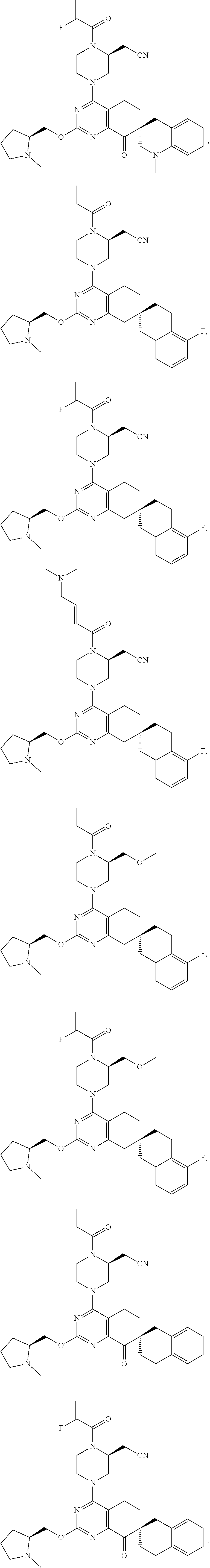 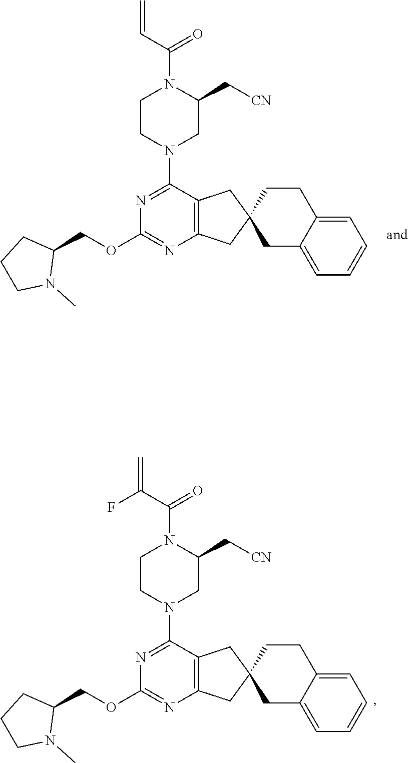 [SMILES: C=CC(=O)N1CCN(c2nc(OC[C@@H]3CCCN3C)nc3c2C[C@]2(CCc4ccccc4C2)C3)C[C@@H]1CC#N ; C=C(F)C(=O)N1CCN(c2nc(OC[C@@H]3CCCN3C)nc3c2C[C@]2(CCc4ccccc4C2)C3)C[C@@H]1CC#N]
or a pharmaceutically salt thereof.

91. The compound of any one of claims 37-67, wherein the compound is selected from: 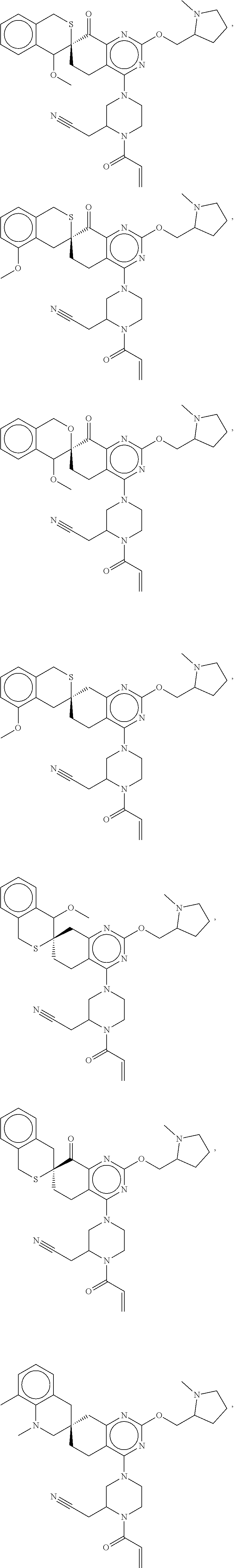 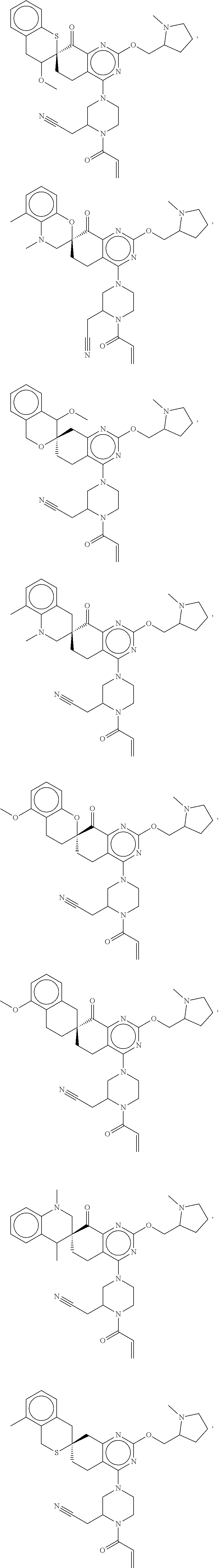 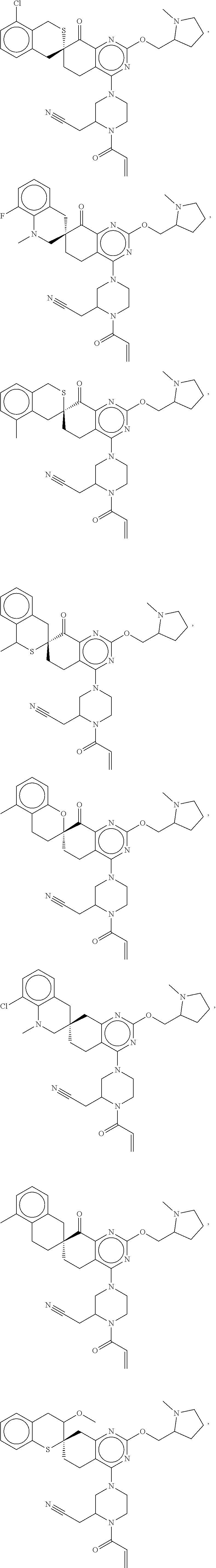 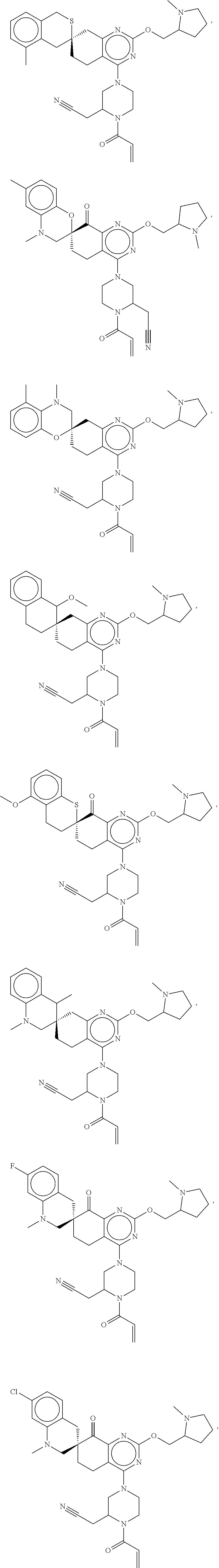 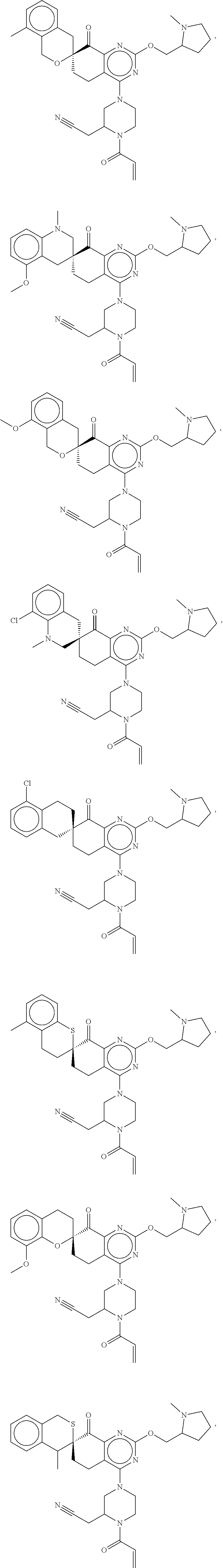 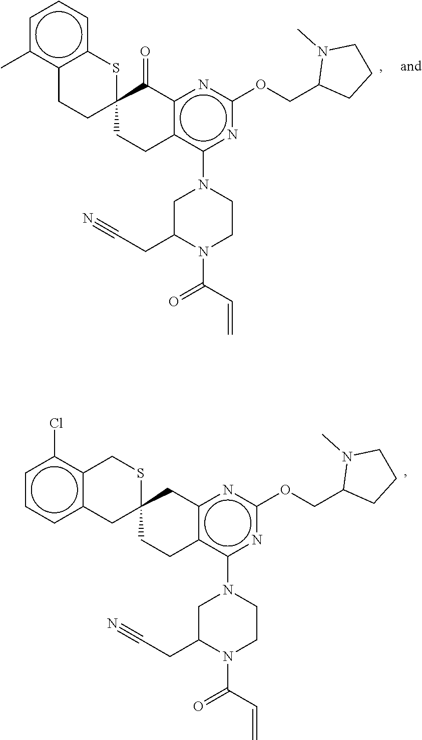
or a pharmaceutically acceptable salt thereof.

92. A pharmaceutical composition comprising a compound from any one of claims 1-91 and a pharmaceutically acceptable diluent or excipient.

93. A method of treating cancer in a subject in need thereof, comprising administering to the subject an effective amount of a compound of any one of claims 1-91.
